SEQUENCE LISTING (59 sequences)

SEQ ID NO: 1 - DNA, 21 bp, Artificial Sequence
  Synthetic construct
  modified_base (1)..(1): n is 2′-fluorouridine
  modified_base (2)..(2): n is 2′-fluorocytidine
  modified_base (3)..(3): n is 2′-methoxyadenosine
  modified_base (4)..(4): n is 2′-fluorouridine
  modified_base (5)..(5): n is 2′-fluorocytidine
  modified_base (6)..(6): n is 2′-methoxyadenosine
  modified_base (7)..(7): n is 2′-fluorocytidine
  modified_base (8)..(8): n is 2′-methoxyadenosine
  modified_base (9)..(9): n is 2′-fluorocytidine
  modified_base (10)..(10): n is 2′-fluorouridine
  modified_base (11)..(11): n is 2′-methoxyguanosine
  modified_base (12)..(13): n is 2′-methoxyadenosine
  modified_base (14)..(14): n is 2′-fluorouridine
  modified_base (15)..(15): n is 2′-methoxyadenosine
  modified_base (16)..(17): n is 2′-fluorocytidine
  modified_base (18)..(19): n is 2′-methoxyadenosine
  modified_base (20)..(20): n is 2′-fluorouridine
  modified_base (21)..(21): n is 2′-methoxythymidine
nnnnnnnnnnnnnnnnnnnnn

SEQ ID NO: 2 - DNA, 21 bp, Artificial Sequence
  Synthetic construct
  modified_base (1)..(2): n is 2′-fluorouridine
  modified_base (3)..(4): n is 2′-methoxyguanosine
  modified_base (5)..(5): n is 2′-fluorouridine
  modified_base (6)..(6): n is 2′-methoxyadenosine
  modified_base (7)..(8): n is 2′-fluorouridine
  modified_base (9)..(9): n is 2′-fluorocytidine
  modified_base (10)..(10): n is 2′-methoxyadenosine
  modified_base (11)..(11): n is 2′-methoxyguanosine
  modified_base (12)..(12): n is 2′-fluorouridine
  modified_base (13)..(13): n is 2′-methoxyguanosine
  modified_base (14)..(14): n is 2′-fluorouridine
  modified_base (15)..(15): n is 2′-methoxyguanosine
  modified_base (16)..(16): n is 2′-methoxyadenosine
  modified_base (17)..(17): n is 2′-fluorouridine
  modified_base (18)..(18): n is 2′-methoxyguanosine
  modified_base (19)..(19): n is 2′-methoxyadenosine
  modified_base (20)..(20): n is 2′-fluorouridine
  modified_base (21)..(21): n is 2′-methoxythymidine
nnnnnnnnnnnnnnnnnnnnn

SEQ ID NO: 3 - RNA, 21 nt, Artificial Sequence
  Synthetic construct
  modified_base (1)..(2): n is 2′-methoxyguanosine
  modified_base (3)..(3): n is 2′-fluorouridine
  modified_base (4)..(4): n is 2′-methoxyuridine
  modified_base (5)..(5): n is 2′-fluoroadenosine
  modified_base (6)..(6): n is 2′-methoxyadenosine
  modified_base (7)..(7): n is 2′-fluorocytidine
  modified_base (8)..(8): n is 2′-methoxyadenosine
  modified_base (9)..(10): n is 2′-fluorocytidine
  modified_base (11)..(11): n is 2′-fluoroadenosine
  modified_base (12)..(12): n is 2′-methoxyuridine
  modified_base (13)..(13): n is 2′-fluorouridine
  modified_base (14)..(14): n is 2′-methoxyuridine
  modified_base (15)..(15): n is 2′-fluoroadenosine
  modified_base (16)..(16): n is 2′-methoxycytidine
  modified_base (17)..(17): n is 2′-fluorouridine
  modified_base (18)..(18): n is 2′-methoxyuridine
  modified_base (19)..(19): n is 2′-fluorocytidine
  modified_base (20)..(21): n is 2′-methoxyadenosine
nnnnnnnnnnnnnnnnnnnnn

SEQ ID NO: 4 - RNA, 23 nt, Artificial Sequence
  Synthetic construct
  modified_base (1)..(2): n is 2′-fluorouridine
  modified_base (3)..(3): n is 2′-methoxyguanosine
  modified_base (4)..(4): n is 2′-fluoroadenosine
  modified_base (5)..(5): n is 2′-methoxyadenosine
  modified_base (6)..(6): n is 2′-fluoroguanosine
  modified_base (7)..(7): n is 2′-methoxyuridine
  modified_base (8)..(8): n is 2′-fluoroadenosine
  modified_base (9)..(9): n is 2′-methoxyadenosine
  modified_base (10)..(10): n is 2′-fluoroadenosine
  modified_base (11)..(11): n is 2′-methoxyuridine
  modified_base (12)..(13): n is 2′-methoxyguanosine
  modified_base (14)..(14): n is 2′-fluorouridine
  modified_base (15)..(15): n is 2′-methoxyguanosine
  modified_base (16)..(16): n is 2′-fluorouridine
  modified_base (17)..(17): n is 2′-methoxyuridine
  modified_base (18)..(18): n is 2′-fluoroadenosine
  modified_base (19)..(19): n is 2′-methoxyadenosine
  modified_base (20)..(20): n is 2′-fluorocytidine
  modified_base (21)..(21): n is 2′-methoxycytidine
  modified_base (22)..(22): n is 2′-methoxyadenosine
  modified_base (23)..(23): n is 2′-methoxyguanosine
nnnnnnnnnnnnnnnnnnnnnnn

SEQ ID NO: 5 - RNA, 21 nt, Artificial Sequence
  Synthetic construct
  modified_base (1)..(1): n is 2′-fluoroguanosine
  modified_base (2)..(2): n is 2′-methoxyguanosine
  modified_base (3)..(3): n is 2′-fluorouridine
  modified_base (4)..(4): n is 2′-methoxyuridine
  modified_base (5)..(5): n is 2′-fluoroadenosine
  modified_base (6)..(6): n is 2′-methoxyadenosine
  modified_base (7)..(7): n is 2′-fluorocytidine
  modified_base (8)..(8): n is 2′-methoxyadenosine
  modified_base (9)..(10): n is 2′-fluorocytidine
  modified_base (11)..(11): n is 2′-fluoroadenosine
  modified_base (12)..(12): n is 2′-methoxyuridine
  modified_base (13)..(13): n is 2′-fluorouridine
  modified_base (14)..(14): n is 2′-methoxyuridine
  modified_base (15)..(15): n is 2′-fluoroadenosine
  modified_base (16)..(16): n is 2′-methoxycytidine
  modified_base (17)..(17): n is 2′-fluorouridine
  modified_base (18)..(18): n is 2′-methoxyuridine
  modified_base (19)..(19): n is 2′-fluorocytidine
  modified_base (20)..(20): n is 2′-methoxyadenosine
  modified_base (21)..(21): n is 2′-fluoroadenosine
nnnnnnnnnnnnnnnnnnnnn

SEQ ID NO: 6 - RNA, 23 nt, Artificial Sequence
  Synthetic construct
  modified_base (1)..(1): n is 2′-methoxyuridine
  modified_base (2)..(2): n is 2′-fluorouridine
  modified_base (3)..(3): n is 2′-methoxyguanosine
  modified_base (4)..(4): n is 2′-fluoroadenosine
  modified_base (5)..(5): n is 2′-methoxyadenosine
  modified_base (6)..(6): n is 2′-fluoroguanosine
  modified_base (7)..(7): n is 2′-methoxyuridine
  modified_base (8)..(8): n is 2′-fluoroadenosine
  modified_base (9)..(9): n is 2′-methoxyadenosine
  modified_base (10)..(10): n is 2′-fluoroadenosine
  modified_base (11)..(11): n is 2′-methoxyuridine
  modified_base (12)..(13): n is 2′-methoxyguanosine
  modified_base (14)..(14): n is 2′-fluorouridine
  modified_base (15)..(15): n is 2′-methoxyguanosine
  modified_base (16)..(16): n is 2′-fluorouridine
  modified_base (17)..(17): n is 2′-methoxyuridine
  modified_base (18)..(18): n is 2′-fluoroadenosine
  modified_base (19)..(19): n is 2′-methoxyadenosine
  modified_base (20)..(20): n is 2′-fluorocytidine
  modified_base (21)..(21): n is 2′-methoxycytidine
  modified_base (22)..(22): n is 2′-methoxyadenosine
  modified_base (23)..(23): n is 2′-methoxyguanosine
nnnnnnnnnnnnnnnnnnnnnnn

SEQ ID NO: 7 - DNA, 21 bp, Artificial Sequence
  Synthetic construct
  modified_base (1)..(1): n is 2′-fluorouridine
  modified_base (2)..(2): n is 2′-fluorocytidine
  modified_base (3)..(3): n is 2′-methoxyadenosine
  modified_base (4)..(4): n is 2′-fluorouridine
  modified_base (5)..(5): n is 2′-fluorocytidine
  modified_base (6)..(6): n is 2′-methoxyadenosine
  modified_base (7)..(7): n is 2′-fluorocytidine
  modified_base (8)..(8): n is 2′-methoxyadenosine
  modified_base (9)..(9): n is 2′-fluorocytidine
  modified_base (10)..(10): n is 2′-fluorouridine
  modified_base (11)..(11): n is 2′-methoxyguanosine
  modified_base (12)..(13): n is 2′-methoxyadenosine
  modified_base (14)..(14): n is 2′-fluorouridine
  modified_base (15)..(15): n is 2′-methoxyadenosine
  modified_base (16)..(17): n is 2′-fluorocytidine
  modified_base (18)..(19): n is 2′-methoxyadenosine
  modified_base (20)..(20): n is 2′-fluorouridine
  modified_base (21)..(21): n is 2′-methoxythymidine
nnnnnnnnnnnnnnnnnnnnn

SEQ ID NO: 8 - DNA, 21 bp, Artificial Sequence
  Synthetic construct
  modified_base (1)..(2): n is 2′-fluorouridine
  modified_base (3)..(3): n is 2′-methoxyguanosine
  modified_base (4)..(4): n is 2′-fluoroguanosine
  modified_base (5)..(5): n is 2′-methoxyuridine
  modified_base (6)..(6): n is 2′-fluoroadenosine
  modified_base (7)..(7): n is 2′-methoxyuridine
  modified_base (8)..(8): n is 2′-fluorouridine
  modified_base (9)..(9): n is 2′-methoxycytidine
  modified_base (10)..(10): n is 2′-fluoroadenosine
  modified_base (11)..(11): n is 2′-methoxyguanosine
  modified_base (12)..(12): n is 2′-fluorouridine
  modified_base (13)..(13): n is 2′-methoxyguanosine
  modified_base (14)..(14): n is 2′-fluorouridine
  modified_base (15)..(15): n is 2′-methoxyguanosine
  modified_base (16)..(16): n is 2′-fluoroadenosine
  modified_base (17)..(17): n is 2′-methoxyuridine
  modified_base (18)..(18): n is 2′-fluoroguanosine
  modified_base (19)..(19): n is 2′-methoxyadenosine
  modified_base (20)..(20): n is 2′-fluorouridine
  modified_base (21)..(21): n is 2′-methoxythymidine
nnnnnnnnnnnnnnnnnnnnn

SEQ ID NO: 9 - DNA, 21 bp, Artificial Sequence
  Synthetic construct
  modified_base (1)..(2): n is 2′-fluorouridine
  modified_base (3)..(4): n is 2′-methoxyguanosine
  modified_base (5)..(5): n is 2′-fluorouridine
  modified_base (6)..(6): n is 2′-methoxyadenosine
  modified_base (7)..(8): n is 2′-fluorouridine
  modified_base (9)..(9): n is 2′-fluorocytidine
  modified_base (10)..(10): n is 2′-methoxyadenosine
  modified_base (11)..(11): n is 2′-methoxyguanosine
  modified_base (12)..(12): n is 2′-fluorouridine
  modified_base (13)..(13): n is 2′-methoxyguanosine
  modified_base (14)..(14): n is 2′-fluorouridine
  modified_base (15)..(15): n is 2′-methoxyguanosine
  modified_base (16)..(16): n is 2′-methoxyadenosine
  modified_base (17)..(17): n is 2′-fluorouridine
  modified_base (18)..(18): n is 2′-methoxyguanosine
  modified_base (19)..(19): n is 2′-methoxyadenosine
  modified_base (20)..(20): n is 2′-methoxyuridine
  modified_base (21)..(21): n is 2′-methoxythymidine
nnnnnnnnnnnnnnnnnnnnn

SEQ ID NO: 10 - RNA, 21 nt, Artificial Sequence
  Synthetic construct
  modified_base (1)..(2): n is 2′-methoxyguanosine
  modified_base (3)..(3): n is 2′-fluorouridine
  modified_base (4)..(4): n is 2′-methoxyuridine
  modified_base (5)..(5): n is 2′-fluoroadenosine
  modified_base (6)..(6): n is 2′-methoxyadenosine
  modified_base (7)..(7): n is 2′-fluorocytidine
  modified_base (8)..(8): n is 2′-methoxyadenosine
  modified_base (9)..(10): n is 2′-fluorocytidine
  modified_base (11)..(11): n is 2′-fluoroadenosine
  modified_base (12)..(12): n is 2′-methoxyuridine
  modified_base (13)..(13): n is 2′-fluorouridine
  modified_base (14)..(14): n is 2′-methoxyuridine
  modified_base (15)..(15): n is 2′-fluoroadenosine
  modified_base (16)..(16): n is 2′-methoxycytidine
  modified_base (17)..(17): n is 2′-fluorouridine
  modified_base (18)..(18): n is 2′-methoxyuridine
  modified_base (19)..(19): n is 2′-fluorocytidine
  modified_base (20)..(21): n is 2′-methoxyadenosine
nnnnnnnnnnnnnnnnnnnnn

SEQ ID NO: 11 - RNA, 23 nt, Artificial Sequence
  Synthetic construct
  modified_base (1)..(2): n is 2′-fluorouridine
  modified_base (3)..(3): n is 2′-methoxyguanosine
  modified_base (4)..(4): n is 2′-fluoroadenosine
  modified_base (5)..(5): n is 2′-methoxyadenosine
  modified_base (6)..(6): n is 2′-fluoroguanosine
  modified_base (7)..(7): n is 2′-methoxyuridine
  modified_base (8)..(8): n is 2′-fluoroadenosine
  modified_base (9)..(9): n is 2′-methoxyadenosine
  modified_base (10)..(10): n is 2′-fluoroadenosine
  modified_base (11)..(11): n is 2′-methoxyuridine
  modified_base (12)..(13): n is 2′-methoxyguanosine
  modified_base (14)..(14): n is 2′-fluorouridine
  modified_base (15)..(15): n is 2′-methoxyguanosine
  modified_base (16)..(16): n is 2′-fluorouridine
  modified_base (17)..(17): n is 2′-methoxyuridine
  modified_base (18)..(18): n is 2′-fluoroadenosine
  modified_base (19)..(19): n is 2′-methoxyadenosine
  modified_base (20)..(20): n is 2′-fluorocytidine
  modified_base (21)..(21): n is 2′-methoxycytidine
  modified_base (22)..(22): n is 2′-methoxyadenosine
  modified_base (23)..(23): n is 2′-methoxyguanosine
nnnnnnnnnnnnnnnnnnnnnnn

SEQ ID NO: 12 - RNA, 21 nt, Artificial Sequence
  Synthetic construct
  modified_base (1)..(1): n is 2′-fluoroguanosine
  modified_base (2)..(2): n is 2′-methoxyguanosine
  modified_base (3)..(3): n is 2′-fluorouridine
  modified_base (4)..(4): n is 2′-methoxyuridine
  modified_base (5)..(5): n is 2′-fluoroadenosine
  modified_base (6)..(6): n is 2′-methoxyadenosine
  modified_base (7)..(7): n is 2′-fluorocytidine
  modified_base (8)..(8): n is 2′-methoxyadenosine
  modified_base (9)..(10): n is 2′-fluorocytidine
  modified_base (11)..(11): n is 2′-fluoroadenosine
  modified_base (12)..(12): n is 2′-methoxyuridine
  modified_base (13)..(13): n is 2′-fluorouridine
  modified_base (14)..(14): n is 2′-methoxyuridine
  modified_base (15)..(15): n is 2′-fluoroadenosine
  modified_base (16)..(16): n is 2′-methoxycytidine
  modified_base (17)..(17): n is 2′-fluorouridine
  modified_base (18)..(18): n is 2′-methoxyuridine
  modified_base (19)..(19): n is 2′-fluorocytidine
  modified_base (20)..(20): n is 2′-methoxyadenosine
  modified_base (21)..(21): n is 2′-fluoroadenosine
nnnnnnnnnnnnnnnnnnnnn

SEQ ID NO: 13 - RNA, 23 nt, Artificial Sequence
  Synthetic construct
  modified_base (1)..(1): n is 2′-methoxyuridine
  modified_base (2)..(2): n is 2′-fluorouridine
  modified_base (3)..(3): n is 2′-methoxyguanosine
  modified_base (4)..(4): n is 2′-fluoroadenosine
  modified_base (5)..(5): n is 2′-methoxyadenosine
  modified_base (6)..(6): n is 2′-fluoroguanosine
  modified_base (7)..(7): n is 2′-methoxyuridine
  modified_base (8)..(8): n is 2′-fluoroadenosine
  modified_base (9)..(9): n is 2′-methoxyadenosine
  modified_base (10)..(10): n is 2′-fluoroadenosine
  modified_base (11)..(11): n is 2′-methoxyuridine
  modified_base (12)..(13): n is 2′-methoxyguanosine
  modified_base (14)..(14): n is 2′-fluorouridine
  modified_base (15)..(15): n is 2′-methoxyguanosine
  modified_base (16)..(16): n is 2′-fluorouridine
  modified_base (17)..(17): n is 2′-methoxyuridine
  modified_base (18)..(18): n is 2′-fluoroadenosine
  modified_base (19)..(19): n is 2′-methoxyadenosine
  modified_base (20)..(20): n is 2′-fluorocytidine
  modified_base (21)..(21): n is 2′-methoxycytidine
  modified_base (22)..(22): n is 2′-methoxyadenosine
  modified_base (23)..(23): n is 2′-methoxyguanosine
nnnnnnnnnnnnnnnnnnnnnnn

SEQ ID NO: 14 - RNA, 23 nt, Artificial Sequence
  Synthetic construct
  modified_base (1)..(2): n is 2′-fluorouridine
  modified_base (3)..(3): n is 2′-methoxyguanosine
  modified_base (4)..(4): n is 2′-fluoroadenosine
  modified_base (5)..(5): n is 2′-methoxyadenosine
  modified_base (6)..(6): n is 2′-fluoroguanosine
  modified_base (7)..(7): n is 2′-methoxyuridine
  modified_base (8)..(8): n is 2′-fluoroadenosine
  modified_base (9)..(9): n is 2′-methoxyadenosine
  modified_base (10)..(10): n is 2′-fluoroadenosine
  modified_base (11)..(11): n is 2′-methoxyuridine
  modified_base (12)..(13): n is 2′-methoxyguanosine
  modified_base (14)..(14): n is 2′-fluorouridine
  modified_base (15)..(15): n is 2′-methoxyguanosine
  modified_base (16)..(16): n is 2′-fluorouridine
  modified_base (17)..(17): n is 2′-methoxyuridine
  modified_base (18)..(18): n is 2′-fluoroadenosine
  modified_base (19)..(19): n is 2′-methoxyadenosine
  modified_base (20)..(20): n is 2′-fluorocytidine
  modified_base (21)..(21): n is 2′-methoxycytidine
  modified_base (22)..(22): n is 2′-methoxyadenosine
  modified_base (23)..(23): n is 2′-methoxyguanosine
nnnnnnnnnnnnnnnnnnnnnnn

SEQ ID NO: 15 - RNA, 21 nt, Artificial Sequence
  Synthetic construct
  modified_base (1)..(1): n is 2′-fluoroguanosine
  modified_base (2)..(2): n is 2′-methoxyguanosine
  modified_base (3)..(3): n is 2′-fluorouridine
  modified_base (4)..(4): n is 2′-methoxyuridine
  modified_base (5)..(5): n is 2′-fluoroadenosine
  modified_base (6)..(6): n is 2′-methoxyadenosine
  modified_base (7)..(7): n is 2′-fluorocytidine
  modified_base (8)..(8): n is 2′-methoxyadenosine
  modified_base (9)..(10): n is 2′-fluorocytidine
  modified_base (11)..(11): n is 2′-fluoroadenosine
  modified_base (12)..(12): n is 2′-methoxyuridine
  modified_base (13)..(13): n is 2′-fluorouridine
  modified_base (14)..(14): n is 2′-methoxyuridine
  modified_base (15)..(15): n is 2′-fluoroadenosine
  modified_base (16)..(16): n is 2′-methoxycytidine
  modified_base (17)..(17): n is 2′-fluorouridine
  modified_base (18)..(18): n is 2′-methoxyuridine
  modified_base (19)..(19): n is 2′-fluorocytidine
  modified_base (20)..(21): n is 2′-methoxyadenosine
nnnnnnnnnnnnnnnnnnnnn

SEQ ID NO: 16 - RNA, 21 nt, Artificial Sequence
  Synthetic construct
  modified_base (1)..(2): n is 2′-methoxyguanosine
  modified_base (3)..(3): n is 2′-fluorouridine
  modified_base (4)..(4): n is 2′-methoxyuridine
  modified_base (5)..(5): n is 2′-fluoroadenosine
  modified_base (6)..(6): n is 2′-methoxyadenosine
  modified_base (7)..(7): n is 2′-fluorocytidine
  modified_base (8)..(8): n is 2′-methoxyadenosine
  modified_base (9)..(10): n is 2′-fluorocytidine
  modified_base (11)..(11): n is 2′-fluoroadenosine
  modified_base (12)..(12): n is 2′-methoxyuridine
  modified_base (13)..(13): n is 2′-fluorouridine
  modified_base (14)..(14): n is 2′-methoxyuridine
  modified_base (15)..(15): n is 2′-methoxyadenosine
  modified_base (16)..(16): n is 2′-fluorocytidine
  modified_base (17)..(17): n is 2′-methoxyuridine
  modified_base (18)..(18): n is 2′-fluorouridine
  modified_base (19)..(19): n is 2′-methoxycytidine
  modified_base (20)..(21): n is 2′-methoxyadenosine
nnnnnnnnnnnnnnnnnnnnn

SEQ ID NO: 17 - RNA, 23 nt, Artificial Sequence
  Synthetic construct
  modified_base (1)..(1): n is 2′-methoxyuridine
  modified_base (2)..(2): n is 2′-fluorouridine
  modified_base (3)..(3): n is 2′-fluoroguanosine
  modified_base (4)..(4): n is 2′-methoxyadenosine
  modified_base (5)..(5): n is 2′-fluoroadenosine
  modified_base (6)..(6): n is 2′-methoxyguanosine
  modified_base (7)..(7): n is 2′-methoxyuridine
  modified_base (8)..(8): n is 2′-fluoroadenosine
  modified_base (9)..(9): n is 2′-methoxyadenosine
  modified_base (10)..(10): n is 2′-fluoroadenosine
  modified_base (11)..(11): n is 2′-methoxyuridine
  modified_base (12)..(13): n is 2′-methoxyguanosine
  modified_base (14)..(14): n is 2′-fluorouridine
  modified_base (15)..(15): n is 2′-methoxyguanosine
  modified_base (16)..(16): n is 2′-fluorouridine
  modified_base (17)..(17): n is 2′-methoxyuridine
  modified_base (18)..(18): n is 2′-fluoroadenosine
  modified_base (19)..(19): n is 2′-methoxyadenosine
  modified_base (20)..(21): n is 2′-methoxycytidine
  modified_base (22)..(22): n is 2′-methoxyadenosine
  modified_base (23)..(23): n is 2′-methoxyguanosine
nnnnnnnnnnnnnnnnnnnnnnn

SEQ ID NO: 18 - DNA, 25 bp, Artificial Sequence
  Synthetic construct
  modified_base (1)..(1): n is 2′-methoxyuridine
  modified_base (2)..(2): n is 2′-fluorouridine
  modified_base (3)..(3): n is 2′-methoxyguanosine
  modified_base (4)..(4): n is 2′-fluoroadenosine
  modified_base (5)..(5): n is 2′-methoxyadenosine
  modified_base (6)..(6): n is 2′-fluoroguanosine
  modified_base (7)..(7): n is 2′-methoxyuridine
  modified_base (8)..(8): n is 2′-fluoroadenosine
  modified_base (9)..(9): n is 2′-methoxyadenosine
  modified_base (10)..(10): n is 2′-fluoroadenosine
  modified_base (11)..(11): n is 2′-methoxyuridine
  modified_base (12)..(13): n is 2′-methoxyguanosine
  modified_base (14)..(14): n is 2′-fluorouridine
  modified_base (15)..(15): n is 2′-methoxyguanosine
  modified_base (16)..(16): n is 2′-fluorouridine
  modified_base (17)..(17): n is 2′-methoxyuridine
  modified_base (18)..(18): n is 2′-fluoroadenosine
  modified_base (19)..(19): n is 2′-methoxyadenosine
  modified_base (20)..(20): n is 2′-fluorocytidine
  modified_base (21)..(21): n is 2′-methoxycytidine
  modified_base (22)..(22): n is 2′-methoxyadenosine
  modified_base (23)..(23): n is 2′-methoxyguanosine
  modified_base (24)..(25): n is 2′-methoxythymidine
nnnnnnnnnnnnnnnnnnnnnnnnn

SEQ ID NO: 19 - DNA, 25 bp, Artificial Sequence
  Synthetic construct
  modified_base (1)..(1): n is 2′-methoxyuridine
  modified_base (2)..(2): n is 2′-fluorouridine
  modified_base (3)..(3): n is 2′-fluoroguanosine
  modified_base (4)..(4): n is 2′-methoxyadenosine
  modified_base (5)..(5): n is 2′-fluoroadenosine
  modified_base (6)..(6): n is 2′-methoxyguanosine
  modified_base (7)..(7): n is 2′-methoxyuridine
  modified_base (8)..(8): n is 2′-fluoroadenosine
  modified_base (9)..(9): n is 2′-methoxyadenosine
  modified_base (10)..(10): n is 2′-fluoroadenosine
  modified_base (11)..(11): n is 2′-methoxyuridine
  modified_base (12)..(13): n is 2′-methoxyguanosine
  modified_base (14)..(14): n is 2′-fluorouridine
  modified_base (15)..(15): n is 2′-methoxyguanosine
  modified_base (16)..(16): n is 2′-fluorouridine
  modified_base (17)..(17): n is 2′-methoxyuridine
  modified_base (18)..(18): n is 2′-fluoroadenosine
  modified_base (19)..(19): n is 2′-methoxyadenosine
  modified_base (20)..(21): n is 2′-methoxycytidine
  modified_base (22)..(22): n is 2′-methoxyadenosine
  modified_base (23)..(23): n is 2′-methoxyguanosine
  modified_base (24)..(25): n is 2′-methoxythymidine
nnnnnnnnnnnnnnnnnnnnnnnnn

SEQ ID NO: 20 - RNA, 21 nt, Artificial Sequence
  Synthetic construct
  modified_base (1)..(1): n is 2′-fluorouridine
  modified_base (2)..(2): n is 2′-methoxyguanosine
  modified_base (3)..(3): n is 2′-fluoroguanosine
  modified_base (4)..(4): n is 2′-methoxyuridine
  modified_base (5)..(5): n is 2′-fluoroadenosine
  modified_base (6)..(6): n is 2′-methoxyuridine
  modified_base (7)..(7): n is 2′-fluorocytidine
  modified_base (8)..(8): n is 2′-fluoroadenosine
  modified_base (9)..(9): n is 2′-fluoroguanosine
  modified_base (10)..(11): n is 2′-fluorocytidine
  modified_base (12)..(12): n is 2′-methoxyadenosine
  modified_base (13)..(13): n is 2′-fluoroadenosine
  modified_base (14)..(14): n is 2′-methoxycytidine
  modified_base (15)..(15): n is 2′-fluorocytidine
  modified_base (16)..(16): n is 2′-methoxyguanosine
  modified_base (17)..(17): n is 2′-fluorocytidine
  modified_base (18)..(18): n is 2′-methoxycytidine
  modified_base (19)..(19): n is 2′-fluorouridine
  modified_base (20)..(20): n is 2′-methoxyuridine
  modified_base (21)..(21): n is 2′-fluorouridine
nnnnnnnnnnnnnnnnnnnnn

SEQ ID NO: 21 - RNA, 23 nt, Artificial Sequence
  Synthetic construct
  modified_base (1)..(1): n is 2′-methoxyadenosine
  modified_base (2)..(2): n is 2′-fluoroadenosine
  modified_base (3)..(3): n is 2′-methoxyadenosine
  modified_base (4)..(4): n is 2′-fluoroguanosine
  modified_base (5)..(5): n is 2′-methoxyguanosine
  modified_base (6)..(6): n is 2′-fluorocytidine
  modified_base (7)..(7): n is 2′-methoxyguanosine
  modified_base (8)..(8): n is 2′-fluoroguanosine
  modified_base (9)..(9): n is 2′-methoxyuridine
  modified_base (10)..(10): n is 2′-fluorouridine
  modified_base (11)..(12): n is 2′-methoxyguanosine
  modified_base (13)..(13): n is 2′-methoxycytidine
  modified_base (14)..(14): n is 2′-fluorouridine
  modified_base (15)..(15): n is 2′-methoxyguanosine
  modified_base (16)..(16): n is 2′-fluoroadenosine
  modified_base (17)..(17): n is 2′-methoxyuridine
  modified_base (18)..(18): n is 2′-fluoroadenosine
  modified_base (19)..(19): n is 2′-methoxycytidine
  modified_base (20)..(20): n is 2′-fluorocytidine
  modified_base (21)..(22): n is 2′-methoxyadenosine
  modified_base (23)..(23): n is 2′-methoxycytidine
nnnnnnnnnnnnnnnnnnnnnnn

SEQ ID NO: 22 - RNA, 21 nt, Artificial Sequence
  Synthetic construct
  modified_base (1)..(1): n is 2′-methoxyadenosine
  modified_base (2)..(2): n is 2′-methoxyuridine
  modified_base (3)..(3): n is 2′-fluorocytidine
  modified_base (4)..(4): n is 2′-methoxyguanosine
  modified_base (5)..(5): n is 2′-fluoroadenosine
  modified_base (6)..(6): n is 2′-methoxyadenosine
  modified_base (7)..(7): n is 2′-fluoroadenosine
  modified_base (8)..(8): n is 2′-methoxyadenosine
  modified_base (9)..(9): n is 2′-fluoroguanosine
  modified_base (10)..(11): n is 2′-fluorocytidine
  modified_base (12)..(12): n is 2′-methoxyadenosine
  modified_base (13)..(13): n is 2′-fluoroadenosine
  modified_base (14)..(14): n is 2′-methoxycytidine
  modified_base (15)..(15): n is 2′-fluoroadenosine
  modified_base (16)..(16): n is 2′-methoxyadenosine
  modified_base (17)..(17): n is 2′-fluoroguanosine
  modified_base (18)..(18): n is 2′-methoxyuridine
  modified_base (19)..(20): n is 2′-fluorocytidine
  modified_base (21)..(21): n is 2′-fluorouridine
nnnnnnnnnnnnnnnnnnnnn

SEQ ID NO: 23 - RNA, 23 nt, Artificial Sequence
  Synthetic construct
  modified_base (1)..(1): n is 2′-methoxyadenosine
  modified_base (2)..(2): n is 2′-fluoroguanosine
  modified_base (3)..(3): n is 2′-methoxyguanosine
  modified_base (4)..(4): n is 2′-fluoroadenosine
  modified_base (5)..(5): n is 2′-methoxycytidine
  modified_base (6)..(6): n is 2′-fluorouridine
  modified_base (7)..(7): n is 2′-methoxyuridine
  modified_base (8)..(8): n is 2′-fluoroguanosine
  modified_base (9)..(9): n is 2′-methoxyuridine
  modified_base (10)..(10): n is 2′-fluorouridine
  modified_base (11)..(12): n is 2′-methoxyguanosine
  modified_base (13)..(13): n is 2′-methoxycytidine
  modified_base (14)..(14): n is 2′-fluorouridine
  modified_base (15)..(15): n is 2′-methoxyuridine
  modified_base (16)..(16): n is 2′-fluorouridine
  modified_base (17)..(17): n is 2′-methoxyuridine
  modified_base (18)..(18): n is 2′-fluorocytidine
  modified_base (19)..(19): n is 2′-methoxyguanosine
  modified_base (20)..(20): n is 2′-methoxyadenosine
  modified_base (21)..(21): n is 2′-methoxyuridine
  modified_base (22)..(22): n is 2′-methoxyadenosine
  modified_base (23)..(23): n is 2′-methoxyguanosine
nnnnnnnnnnnnnnnnnnnnnnn

SEQ ID NO: 24 - RNA, 21 nt, Artificial Sequence
  Synthetic construct
  modified_base (1)..(2): n is 2′-methoxyguanosine
  modified_base (3)..(3): n is 2′-fluorouridine
  modified_base (4)..(4): n is 2′-methoxyuridine
  modified_base (5)..(5): n is 2′-fluoroadenosine
  modified_base (6)..(6): n is 2′-methoxyadenosine
  modified_base (7)..(7): n is 2′-fluorocytidine
  modified_base (8)..(8): n is 2′-methoxyadenosine
  modified_base (9)..(10): n is 2′-fluorocytidine
  modified_base (11)..(11): n is 2′-fluoroadenosine
  modified_base (12)..(12): n is 2′-methoxyuridine
  modified_base (13)..(13): n is 2′-fluorouridine
  modified_base (14)..(14): n is 2′-methoxyuridine
  modified_base (15)..(15): n is 2′-fluoroadenosine
  modified_base (16)..(16): n is 2′-methoxycytidine
  modified_base (17)..(17): n is 2′-fluorouridine
  modified_base (18)..(18): n is 2′-methoxyuridine
  modified_base (19)..(19): n is 2′-fluorocytidine
  modified_base (20)..(21): n is 2′-methoxyadenosine
nnnnnnnnnnnnnnnnnnnnn

SEQ ID NO: 25 - RNA, 21 nt, Artificial Sequence
  Synthetic construct
  modified_base (1)..(2): n is 2′-methoxyguanosine
  modified_base (3)..(3): n is 2′-fluorouridine
  modified_base (4)..(4): n is 2′-methoxyuridine
  modified_base (5)..(5): n is 2′-fluoroadenosine
  modified_base (6)..(6): n is 2′-methoxyadenosine
  modified_base (7)..(7): n is 2′-fluorocytidine
  modified_base (8)..(8): n is 2′-methoxyadenosine
  modified_base (9)..(10): n is 2′-fluorocytidine
  modified_base (11)..(11): n is 2′-fluoroadenosine
  modified_base (12)..(12): n is 2′-methoxyuridine
  modified_base (13)..(13): n is 2′-fluorouridine
  modified_base (14)..(14): n is 2′-methoxyuridine
  modified_base (15)..(15): n is 2′-fluoroadenosine
  modified_base (16)..(16): n is 2′-fluorocytidine
  modified_base (17)..(18): n is 2′-fluorouridine
  modified_base (19)..(19): n is 2′-fluorocytidine
  modified_base (20)..(21): n is 2′-methoxyadenosine
nnnnnnnnnnnnnnnnnnnnn

SEQ ID NO: 26 - RNA, 23 nt, Artificial Sequence
  Synthetic construct
  modified_base (1)..(1): n is 2′-methoxyuridine
  modified_base (2)..(2): n is 2′-fluorouridine
  modified_base (3)..(3): n is 2′-methoxyguanosine
  modified_base (4)..(4): n is 2′-fluoroadenosine
  modified_base (5)..(5): n is 2′-methoxyadenosine
  modified_base (6)..(6): n is 2′-fluoroguanosine
  modified_base (7)..(7): n is 2′-methoxyuridine
  modified_base (8)..(8): n is 2′-fluoroadenosine
  modified_base (9)..(9): n is 2′-methoxyadenosine
  modified_base (10)..(10): n is 2′-fluoroadenosine
  modified_base (11)..(11): n is 2′-methoxyuridine
  modified_base (12)..(13): n is 2′-methoxyguanosine
  modified_base (14)..(14): n is 2′-fluorouridine
  modified_base (15)..(15): n is 2′-methoxyguanosine
  modified_base (16)..(17): n is 2′-fluorouridine
  modified_base (18)..(18): n is 2′-fluoroadenosine
  modified_base (19)..(19): n is 2′-methoxyadenosine
  modified_base (20)..(20): n is 2′-fluorocytidine
  modified_base (21)..(21): n is 2′-methoxycytidine
  modified_base (22)..(22): n is 2′-methoxyadenosine
  modified_base (23)..(23): n is 2′-methoxyguanosine
nnnnnnnnnnnnnnnnnnnnnnn

SEQ ID NO: 27 - RNA, 21 nt, Artificial Sequence
  Synthetic construct
  modified_base (1)..(1): n is 2′-fluoroguanosine
  modified_base (2)..(2): n is 2′-methoxyguanosine
  modified_base (3)..(3): n is 2′-fluorouridine
  modified_base (4)..(4): n is 2′-methoxyuridine
  modified_base (5)..(5): n is 2′-fluoroadenosine
  modified_base (6)..(6): n is 2′-methoxyadenosine
  modified_base (7)..(7): n is 2′-fluorocytidine
  modified_base (8)..(8): n is 2′-methoxyadenosine
  modified_base (9)..(10): n is 2′-fluorocytidine
  modified_base (11)..(11): n is 2′-fluoroadenosine
  modified_base (12)..(12): n is 2′-methoxyuridine
  modified_base (13)..(13): n is 2′-fluorouridine
  modified_base (14)..(14): n is 2′-methoxyuridine
  modified_base (15)..(15): n is 2′-fluoroadenosine
  modified_base (16)..(16): n is 2′-fluorocytidine
  modified_base (17)..(18): n is 2′-fluorouridine
  modified_base (19)..(19): n is 2′-fluorocytidine
  modified_base (20)..(20): n is 2′-methoxyadenosine
  modified_base (21)..(21): n is 2′-fluoroadenosine
nnnnnnnnnnnnnnnnnnnnn

SEQ ID NO: 28 - DNA, 39 bp, Artificial Sequence
  Synthetic construct]
  modified_base (1)..(1): n is 2′-methoxyguanosine
  modified_base (2)..(2): n is LNA
  modified_base (3)..(3): n is 2′-methoxycytidine
  modified_base (4)..(4): n is LNA
  modified_base (5)..(5): n is 2′-methoxyadenosine
  modified_base (6)..(7): n is 2′-methoxythymidine
  modified_base (8)..(9): n is 2′-methoxyguanosine
  modified_base (10)..(10): n is 2′-methoxythymidine
  modified_base (11)..(11): n is 2′-methoxyadenosine
  modified_base (12)..(13): n is 2′-methoxythymidine
  modified_base (14)..(14): n is LNA
  modified_base (15)..(15): n is 2′-methoxycytidine
  modified_base (16)..(16): n is LNA
  modified_base (17)..(17): n is 2′-methoxyadenosine
  modified_base (18)..(18): n is LNA
  modified_base (19)..(19): n is 2′-fluoroguanosine
  modified_base (20)..(20): n is 2′-methoxyguanosine
  modified_base (21)..(21): n is 2′-fluorouridine
  modified_base (22)..(22): n is 2′-methoxyuridine
  modified_base (23)..(23): n is 2′-fluoroadenosine
  modified_base (24)..(24): n is 2′-methoxyadenosine
  modified_base (25)..(25): n is 2′-fluorocytidine
  modified_base (26)..(26): n is 2′-methoxyadenosine
  modified_base (27)..(28): n is 2′-fluorocytidine
  modified_base (29)..(29): n is 2′-fluoroadenosine
  modified_base (30)..(30): n is 2′-methoxyuridine
  modified_base (31)..(31): n is 2′-fluorouridine
  modified_base (32)..(32): n is 2′-methoxyuridine
  modified_base (33)..(33): n is 2′-fluoroadenosine
  modified_base (34)..(34): n is 2′-methoxycytidine
  modified_base (35)..(35): n is 2′-fluorouridine
  modified_base (36)..(36): n is 2′-methoxyuridine
  modified_base (37)..(37): n is 2′-fluorocytidine
  modified_base (38)..(38): n is 2′-methoxyadenosine
  modified_base (39)..(39): n is 2′-fluoroadenosine
nnnnnnnnnnnnnnnnnnnnnnnnnnnnnnnnnnnnnnn

SEQ ID NO: 29 - RNA, 27 nt, Artificial Sequence
  Synthetic construct
  modified_base (1)..(1): n is 2′-methoxyuridine
  modified_base (2)..(2): n is 2′-methoxyadenosine
  modified_base (3)..(3): n is 2′-methoxycytidine
  modified_base (4)..(6): n is 2′-methoxyadenosine
  modified_base (7)..(7): n is 2′-fluoroguanosine
  modified_base (8)..(8): n is 2′-methoxyguanosine
  modified_base (9)..(9): n is 2′-fluorouridine
  modified_base (10)..(10): n is 2′-methoxyuridine
  modified_base (11)..(11): n is 2′-fluoroadenosine
  modified_base (12)..(12): n is 2′-methoxyadenosine
  modified_base (13)..(13): n is 2′-fluorocytidine
  modified_base (14)..(14): n is 2′-methoxyadenosine
  modified_base (15)..(16): n is 2′-fluorocytidine
  modified_base (17)..(17): n is 2′-fluoroadenosine
  modified_base (18)..(18): n is 2′-methoxyuridine
  modified_base (19)..(19): n is 2′-fluorouridine
  modified_base (20)..(20): n is 2′-methoxyuridine
  modified_base (21)..(21): n is 2′-fluoroadenosine
  modified_base (22)..(22): n is 2′-methoxycytidine
  modified_base (23)..(23): n is 2′-fluorouridine
  modified_base (24)..(24): n is 2′-methoxyuridine
  modified_base (25)..(25): n is 2′-fluorocytidine
  modified_base (26)..(26): n is 2′-methoxyadenosine
  modified_base (27)..(27): n is 2′-fluoroadenosine
nnnnnnnnnnnnnnnnnnnnnnnnnnn

SEQ ID NO: 30 - RNA, 23 nt, Artificial Sequence
  Synthetic construct
  modified_base (1)..(1): n is 2′-methoxyuridine
  modified_base (2)..(2): n is 2′-fluorouridine
  modified_base (3)..(3): n is 2′-methoxyguanosine
  modified_base (4)..(5): n is 2′-methoxyadenosine
  modified_base (6)..(6): n is 2′-fluoroguanosine
  modified_base (7)..(7): n is 2′-methoxyuridine
  modified_base (8)..(8): n is 2′-fluoroadenosine
  modified_base (9)..(9): n is 2′-methoxyadenosine
  modified_base (10)..(10): n is 2′-fluoroadenosine
  modified_base (11)..(11): n is 2′-methoxyuridine
  modified_base (12)..(13): n is 2′-methoxyguanosine
  modified_base (14)..(14): n is 2′-fluorouridine
  modified_base (15)..(15): n is 2′-methoxyguanosine
  modified_base (16)..(16): n is 2′-fluorouridine
  modified_base (17)..(17): n is 2′-methoxyuridine
  modified_base (18)..(18): n is 2′-fluoroadenosine
  modified_base (19)..(19): n is 2′-methoxyadenosine
  modified_base (20)..(20): n is 2′-fluorocytidine
  modified_base (21)..(21): n is 2′-methoxycytidine
  modified_base (22)..(22): n is 2′-methoxyadenosine
  modified_base (23)..(23): n is 2′-methoxyguanosine
nnnnnnnnnnnnnnnnnnnnnnn

SEQ ID NO: 31 - RNA, 23 nt, Artificial Sequence
  Synthetic construct
  modified_base (1)..(1): n is 2′-methoxyuridine
  modified_base (2)..(2): n is 2′-fluorouridine
  modified_base (3)..(3): n is 2′-methoxyguanosine
  modified_base (4)..(4): n is 2′-fluoroadenosine
  modified_base (5)..(5): n is 2′-methoxyadenosine
  modified_base (6)..(6): n is 2′-fluoroguanosine
  modified_base (7)..(7): n is 2′-methoxyuridine
  modified_base (8)..(8): n is 2′-fluoroadenosine
  modified_base (9)..(9): n is 2′-methoxyadenosine
  modified_base (10)..(10): n is 2′-fluoroadenosine
  modified_base (11)..(11): n is 2′-methoxyuridine
  modified_base (12)..(13): n is 2′-methoxyguanosine
  modified_base (14)..(14): n is 2′-fluorouridine
  modified_base (15)..(15): n is 2′-methoxyguanosine
  modified_base (16)..(16): n is 2′-fluorouridine
  modified_base (17)..(17): n is 2′-methoxyuridine
  modified_base (18)..(19): n is 2′-methoxyadenosine
  modified_base (20)..(20): n is 2′-fluorocytidine
  modified_base (21)..(21): n is 2′-methoxycytidine
  modified_base (22)..(22): n is 2′-methoxyadenosine
  modified_base (23)..(23): n is 2′-methoxyguanosine
nnnnnnnnnnnnnnnnnnnnnnn

SEQ ID NO: 32 - RNA, 21 nt, Artificial Sequence
  Synthetic construct
  modified_base (1)..(1): n is 2′-fluoroadenosine
  modified_base (2)..(2): n is 2′-methoxyadenosine
  modified_base (3)..(3): n is 2′-fluoroguanosine
  modified_base (4)..(4): n is 2′-methoxycytidine
  modified_base (5)..(5): n is 2′-fluoroadenosine
  modified_base (6)..(6): n is 2′-methoxyadenosine
  modified_base (7)..(7): n is 2′-fluoroguanosine
  modified_base (8)..(8): n is 2′-methoxyadenosine
  modified_base (9)..(9): n is 2′-fluorouridine
  modified_base (10)..(10): n is 2′-fluoroadenosine
  modified_base (11)..(11): n is 2′-fluorouridine
  modified_base (12)..(12): n is 2′-methoxyuridine
  modified_base (13)..(13): n is 2′-fluorouridine
  modified_base (14)..(14): n is 2′-methoxyuridine
  modified_base (15)..(15): n is 2′-fluoroadenosine
  modified_base (16)..(16): n is 2′-methoxyadenosine
  modified_base (17)..(17): n is 2′-fluorouridine
  modified_base (18)..(18): n is 2′-methoxyadenosine
  modified_base (19)..(19): n is 2′-fluoroadenosine
  modified_base (20)..(20): n is 2′-fluorouridine
  modified_base (21)..(21): n is 2′-fluoroadenosine
nnnnnnnnnnnnnnnnnnnnn

SEQ ID NO: 33 - RNA, 23 nt, Artificial Sequence
  Synthetic construct
  modified_base (1)..(1): n is 2′-methoxyuridine
  modified_base (2)..(2): n is 2′-fluoroadenosine
  modified_base (3)..(3): n is 2′-methoxyuridine
  modified_base (4)..(4): n is 2′-fluorouridine
  modified_base (5)..(5): n is 2′-methoxyadenosine
  modified_base (6)..(6): n is 2′-fluorouridine
  modified_base (7)..(7): n is 2′-methoxyadenosine
  modified_base (8)..(8): n is 2′-fluoroadenosine
  modified_base (9)..(9): n is 2′-methoxyadenosine
  modified_base (10)..(10): n is 2′-fluoroadenosine
  modified_base (11)..(11): n is 2′-methoxyadenosine
  modified_base (12)..(12): n is 2′-methoxyuridine
  modified_base (13)..(13): n is 2′-methoxyadenosine
  modified_base (14)..(14): n is 2′-fluorouridine
  modified_base (15)..(15): n is 2′-methoxycytidine
  modified_base (16)..(16): n is 2′-fluorouridine
  modified_base (17)..(17): n is 2′-methoxyuridine
  modified_base (18)..(18): n is 2′-fluoroguanosine
  modified_base (19)..(19): n is 2′-methoxycytidine
  modified_base (20)..(20): n is 2′-fluorouridine
  modified_base (21)..(23): n is 2′-methoxyuridine
nnnnnnnnnnnnnnnnnnnnnnn

SEQ ID NO: 34 - RNA, 21 nt, Artificial Sequence
  Synthetic construct
  modified_base (1)..(2): n is 2′-methoxyadenosine
  modified_base (3)..(3): n is 2′-fluoroguanosine
  modified_base (4)..(4): n is 2′-methoxycytidine
  modified_base (5)..(5): n is 2′-fluoroadenosine
  modified_base (6)..(6): n is 2′-methoxyadenosine
  modified_base (7)..(7): n is 2′-fluoroguanosine
  modified_base (8)..(8): n is 2′-methoxyadenosine
  modified_base (9)..(9): n is 2′-fluorouridine
  modified_base (10)..(10): n is 2′-fluoroadenosine
  modified_base (11)..(11): n is 2′-fluorouridine
  modified_base (12)..(12): n is 2′-methoxyuridine
  modified_base (13)..(13): n is 2′-fluorouridine
  modified_base (14)..(14): n is 2′-methoxyuridine
  modified_base (15)..(15): n is 2′-fluorouridine
  modified_base (16)..(16): n is 2′-methoxyadenosine
  modified_base (17)..(17): n is 2′-fluorouridine
  modified_base (18)..(18): n is 2′-methoxyadenosine
  modified_base (19)..(19): n is 2′-fluoroadenosine
  modified_base (20)..(20): n is 2′-fluorouridine
  modified_base (21)..(21): n is 2′-fluoroadenosine
nnnnnnnnnnnnnnnnnnnnn

SEQ ID NO: 35 - RNA, 21 nt, Artificial Sequence
  Synthetic construct
  modified_base (1)..(2): n is 2′-methoxyadenosine
  modified_base (3)..(3): n is 2′-fluoroguanosine
  modified_base (4)..(4): n is 2′-methoxycytidine
  modified_base (5)..(5): n is 2′-fluoroadenosine
  modified_base (6)..(6): n is 2′-methoxyadenosine
  modified_base (7)..(7): n is 2′-fluoroguanosine
  modified_base (8)..(8): n is 2′-methoxyadenosine
  modified_base (9)..(9): n is 2′-fluorouridine
  modified_base (10)..(10): n is 2′-fluoroadenosine
  modified_base (11)..(11): n is 2′-fluorouridine
  modified_base (12)..(12): n is 2′-methoxyuridine
  modified_base (13)..(13): n is 2′-fluorouridine
  modified_base (14)..(15): n is 2′-methoxyuridine
  modified_base (16)..(16): n is 2′-fluoroadenosine
  modified_base (17)..(17): n is 2′-methoxyuridine
  modified_base (18)..(18): n is 2′-fluoroadenosine
  modified_base (19)..(19): n is 2′-methoxyadenosine
  modified_base (20)..(20): n is 2′-fluorouridine
  modified_base (21)..(21): n is 2′-methoxyadenosine
nnnnnnnnnnnnnnnnnnnnn

SEQ ID NO: 36 - RNA, 23 nt, Artificial Sequence
  Synthetic construct
  modified_base (1)..(1): n is 2′-methoxyuridine
  modified_base (2)..(2): n is 2′-fluoroadenosine
  modified_base (3)..(3): n is 2′-fluorouridine
  modified_base (4)..(4): n is 2′-methoxyuridine
  modified_base (5)..(5): n is 2′-fluoroadenosine
  modified_base (6)..(6): n is 2′-methoxyuridine
  modified_base (7)..(7): n is 2′-methoxyadenosine
  modified_base (8)..(8): n is 2′-fluoroadenosine
  modified_base (9)..(9): n is 2′-methoxyadenosine
  modified_base (10)..(10): n is 2′-fluoroadenosine
  modified_base (11)..(11): n is 2′-methoxyadenosine
  modified_base (12)..(12): n is 2′-methoxyuridine
  modified_base (13)..(13): n is 2′-methoxyadenosine
  modified_base (14)..(14): n is 2′-fluorouridine
  modified_base (15)..(15): n is 2′-methoxycytidine
  modified_base (16)..(16): n is 2′-fluorouridine
  modified_base (17)..(17): n is 2′-methoxyuridine
  modified_base (18)..(18): n is 2′-fluoroguanosine
  modified_base (19)..(19): n is 2′-methoxycytidine
  modified_base (20)..(23): n is 2′-methoxyuridine
nnnnnnnnnnnnnnnnnnnnnnn

SEQ ID NO: 37 - RNA, 21 nt, Artificial Sequence
  Synthetic construct
  modified_base (1)..(2): n is 2′-methoxyadenosine
  modified_base (3)..(3): n is 2′-fluoroguanosine
  modified_base (4)..(4): n is 2′-methoxycytidine
  modified_base (5)..(5): n is 2′-fluoroadenosine
  modified_base (6)..(6): n is 2′-methoxyadenosine
  modified_base (7)..(7): n is 2′-fluoroguanosine
  modified_base (8)..(8): n is 2′-methoxyadenosine
  modified_base (9)..(9): n is 2′-fluorouridine
  modified_base (10)..(10): n is 2′-fluoroadenosine
  modified_base (11)..(11): n is 2′-fluorouridine
  modified_base (12)..(12): n is 2′-methoxyuridine
  modified_base (13)..(13): n is 2′-fluorouridine
  modified_base (14)..(15): n is 2′-methoxyuridine
  modified_base (16)..(16): n is 2′-methoxyadenosine
  modified_base (17)..(17): n is 2′-methoxyuridine
  modified_base (18)..(19): n is 2′-methoxyadenosine
  modified_base (20)..(20): n is 2′-fluorouridine
  modified_base (21)..(21): n is 2′-methoxyadenosine
nnnnnnnnnnnnnnnnnnnnn

SEQ ID NO: 38 - RNA, 23 nt, Artificial Sequence
  Synthetic construct
  modified_base (1)..(1): n is 2′-methoxyuridine
  modified_base (2)..(2): n is 2′-fluorouridine
  modified_base (3)..(3): n is 2′-methoxyguanosine
  modified_base (4)..(4): n is 2′-fluoroadenosine
  modified_base (5)..(5): n is 2′-methoxyadenosine
  modified_base (6)..(6): n is 2′-fluoroguanosine
  modified_base (7)..(7): n is 2′-methoxyuridine
  modified_base (8)..(8): n is 2′-fluoroadenosine
  modified_base (9)..(9): n is 2′-methoxyadenosine
  modified_base (10)..(10): n is 2′-fluoroadenosine
  modified_base (11)..(11): n is 2′-methoxyuridine
  modified_base (12)..(13): n is 2′-methoxyguanosine
  modified_base (14)..(14): n is 2′-fluorouridine
  modified_base (15)..(15): n is 2′-methoxyguanosine
  modified_base (16)..(16): n is 2′-fluorouridine
  modified_base (17)..(17): n is 2′-methoxyuridine
  modified_base (18)..(18): n is 2′-fluoroadenosine
  modified_base (19)..(19): n is 2′-methoxyadenosine
  modified_base (20)..(21): n is 2′-methoxycytidine
  modified_base (22)..(22): n is 2′-methoxyadenosine
  modified_base (23)..(23): n is 2′-methoxyguanosine
nnnnnnnnnnnnnnnnnnnnnnn

SEQ ID NO: 39 - RNA, 23 nt, Artificial Sequence
  Synthetic construct
  modified_base (1)..(1): n is 2′-methoxyuridine
  modified_base (2)..(2): n is 2′-fluorouridine
  modified_base (3)..(3): n is 2′-methoxyguanosine
  modified_base (4)..(4): n is 2′-fluoroadenosine
  modified_base (5)..(5): n is 2′-methoxyadenosine
  modified_base (6)..(6): n is 2′-fluoroguanosine
  modified_base (7)..(7): n is 2′-methoxyuridine
  modified_base (8)..(8): n is 2′-fluoroadenosine
  modified_base (9)..(9): n is 2′-methoxyadenosine
  modified_base (10)..(10): n is 2′-fluoroadenosine
  modified_base (11)..(11): n is 2′-methoxyuridine
  modified_base (12)..(13): n is 2′-methoxyguanosine
  modified_base (14)..(14): n is 2′-fluorouridine
  modified_base (15)..(15): n is 2′-methoxyguanosine
  modified_base (16)..(16): n is 2′-fluorouridine
  modified_base (17)..(17): n is 2′-methoxyuridine
  modified_base (18)..(19): n is 2′-methoxyadenosine
  modified_base (20)..(21): n is 2′-methoxycytidine
  modified_base (22)..(22): n is 2′-methoxyadenosine
  modified_base (23)..(23): n is 2′-methoxyguanosine
nnnnnnnnnnnnnnnnnnnnnnn

SEQ ID NO: 40 - RNA, 21 nt, Artificial Sequence
  Synthetic construct
  modified_base (1)..(1): n is 2′-fluoroadenosine
  modified_base (2)..(2): n is 2′-methoxyadenosine
  modified_base (3)..(3): n is 2′-fluorocytidine
  modified_base (4)..(4): n is 2′-methoxyadenosine
  modified_base (5)..(5): n is 2′-fluoroguanosine
  modified_base (6)..(6): n is 2′-methoxyuridine
  modified_base (7)..(7): n is 2′-fluoroguanosine
  modified_base (8)..(8): n is 2′-methoxyuridine
  modified_base (9)..(9): n is 2′-fluorouridine
  modified_base (10)..(10): n is 2′-fluorocytidine
  modified_base (11)..(11): n is 2′-fluorouridine
  modified_base (12)..(12): n is 2′-methoxyuridine
  modified_base (13)..(13): n is 2′-fluoroguanosine
  modified_base (14)..(14): n is 2′-methoxycytidine
  modified_base (15)..(15): n is 2′-fluorouridine
  modified_base (16)..(16): n is 2′-methoxycytidine
  modified_base (17)..(17): n is 2′-fluorouridine
  modified_base (18)..(18): n is 2′-methoxyadenosine
  modified_base (19)..(19): n is 2′-fluorouridine
  modified_base (20)..(20): n is 2′-methoxyadenosine
  modified_base (21)..(21): n is 2′-fluoroadenosine
nnnnnnnnnnnnnnnnnnnnn

SEQ ID NO: 41 - RNA, 23 nt, Artificial Sequence
  Synthetic construct
  modified_base (1)..(1): n is 2′-methoxyuridine
  modified_base (2)..(2): n is 2′-fluorouridine
  modified_base (3)..(3): n is 2′-methoxyadenosine
  modified_base (4)..(4): n is 2′-fluorouridine
  modified_base (5)..(5): n is 2′-methoxyadenosine
  modified_base (6)..(6): n is 2′-fluoroguanosine
  modified_base (7)..(7): n is 2′-methoxyadenosine
  modified_base (8)..(8): n is 2′-fluoroguanosine
  modified_base (9)..(9): n is 2′-methoxycytidine
  modified_base (10)..(10): n is 2′-fluoroadenosine
  modified_base (11)..(11): n is 2′-methoxyadenosine
  modified_base (12)..(12): n is 2′-methoxyguanosine
  modified_base (13)..(13): n is 2′-methoxyadenosine
  modified_base (14)..(14): n is 2′-fluoroadenosine
  modified_base (15)..(15): n is 2′-methoxycytidine
  modified_base (16)..(16): n is 2′-fluoroadenosine
  modified_base (17)..(17): n is 2′-methoxycytidine
  modified_base (18)..(18): n is 2′-fluorouridine
  modified_base (19)..(19): n is 2′-methoxyguanosine
  modified_base (20)..(20): n is 2′-fluorouridine
  modified_base (21)..(22): n is 2′-methoxyuridine
  modified_base (23)..(23): n is 2′-fluorouridine
nnnnnnnnnnnnnnnnnnnnnnn

SEQ ID NO: 42 - RNA, 21 nt, Artificial Sequence
  Synthetic construct
  modified_base (1)..(1): n is 2′-fluoroadenosine
  modified_base (2)..(2): n is 2′-methoxyadenosine
  modified_base (3)..(3): n is 2′-fluorocytidine
  modified_base (4)..(4): n is 2′-methoxyadenosine
  modified_base (5)..(5): n is 2′-fluoroguanosine
  modified_base (6)..(6): n is 2′-methoxyuridine
  modified_base (7)..(7): n is 2′-fluoroguanosine
  modified_base (8)..(8): n is 2′-methoxyuridine
  modified_base (9)..(9): n is 2′-fluorouridine
  modified_base (10)..(10): n is 2′-fluorocytidine
  modified_base (11)..(11): n is 2′-fluorouridine
  modified_base (12)..(12): n is 2′-methoxyuridine
  modified_base (13)..(13): n is 2′-fluoroguanosine
  modified_base (14)..(14): n is 2′-methoxycytidine
  modified_base (15)..(15): n is 2′-fluorouridine
  modified_base (16)..(16): n is 2′-fluorocytidine
  modified_base (17)..(17): n is 2′-fluorouridine
  modified_base (18)..(18): n is 2′-methoxyadenosine
  modified_base (19)..(19): n is 2′-fluorouridine
  modified_base (20)..(20): n is 2′-methoxyadenosine
  modified_base (21)..(21): n is 2′-fluoroadenosine
nnnnnnnnnnnnnnnnnnnnn

SEQ ID NO: 43 - RNA, 21 nt, Artificial Sequence
  Synthetic construct
  modified_base (1)..(2): n is 2′-methoxyguanosine
  modified_base (3)..(3): n is 2′-fluorouridine
  modified_base (4)..(4): n is 2′-methoxyuridine
  modified_base (5)..(5): n is 2′-fluoroadenosine
  modified_base (6)..(6): n is 2′-methoxyadenosine
  modified_base (7)..(7): n is 2′-fluorocytidine
  modified_base (8)..(8): n is 2′-methoxyadenosine
  modified_base (9)..(10): n is 2′-fluorocytidine
  modified_base (11)..(11): n is 2′-fluoroadenosine
  modified_base (12)..(12): n is 2′-methoxyuridine
  modified_base (13)..(14): n is 2′-fluorouridine
  modified_base (15)..(15): n is 2′-fluoroadenosine
  modified_base (16)..(16): n is 2′-fluorocytidine
  modified_base (17)..(18): n is 2′-fluorouridine
  modified_base (19)..(19): n is 2′-fluorocytidine
  modified_base (20)..(21): n is 2′-methoxyadenosine
nnnnnnnnnnnnnnnnnnnnn

SEQ ID NO: 44 - RNA, 21 nt, Artificial Sequence
  Synthetic construct
  modified_base (1)..(2): n is 2′-methoxyguanosine
  modified_base (3)..(3): n is 2′-fluorouridine
  modified_base (4)..(4): n is 2′-methoxyuridine
  modified_base (5)..(5): n is 2′-fluoroadenosine
  modified_base (6)..(6): n is 2′-methoxyadenosine
  modified_base (7)..(7): n is 2′-fluorocytidine
  modified_base (8)..(8): n is 2′-methoxyadenosine
  modified_base (9)..(10): n is 2′-fluorocytidine
  modified_base (11)..(11): n is 2′-fluoroadenosine
  modified_base (12)..(14): n is 2′-fluorouridine
  modified_base (15)..(15): n is 2′-fluoroadenosine
  modified_base (16)..(16): n is 2′-fluorocytidine
  modified_base (17)..(18): n is 2′-fluorouridine
  modified_base (19)..(19): n is 2′-fluorocytidine
  modified_base (20)..(21): n is 2′-methoxyadenosine
nnnnnnnnnnnnnnnnnnnnn

SEQ ID NO: 45 - RNA, 21 nt, Artificial Sequence
  Synthetic construct
  modified_base (1)..(2): n is 2′-methoxyguanosine
  modified_base (3)..(3): n is 2′-fluorouridine
  modified_base (4)..(4): n is 2′-methoxyuridine
  modified_base (5)..(5): n is 2′-fluoroadenosine
  modified_base (6)..(6): n is 2′-methoxyadenosine
  modified_base (7)..(7): n is 2′-fluorocytidine
  modified_base (8)..(8): n is 2′-methoxyadenosine
  modified_base (9)..(10): n is 2′-fluorocytidine
  modified_base (11)..(11): n is 2′-fluoroadenosine
  modified_base (12)..(12): n is 2′-methoxyuridine
  modified_base (13)..(13): n is 2′-fluorouridine
  modified_base (14)..(14): n is 2′-methoxyuridine
  modified_base (15)..(15): n is 2′-fluoroadenosine
  modified_base (16)..(16): n is 2′-methoxycytidine
  modified_base (17)..(18): n is 2′-fluorouridine
  modified_base (19)..(19): n is 2′-fluorocytidine
  modified_base (20)..(21): n is 2′-methoxyadenosine
nnnnnnnnnnnnnnnnnnnnn

SEQ ID NO: 46 - RNA, 21 nt, Artificial Sequence
  Synthetic construct
  modified_base (1)..(2): n is 2′-methoxyguanosine
  modified_base (3)..(3): n is 2′-fluorouridine
  modified_base (4)..(4): n is 2′-methoxyuridine
  modified_base (5)..(5): n is 2′-fluoroadenosine
  modified_base (6)..(6): n is 2′-methoxyadenosine
  modified_base (7)..(7): n is 2′-fluorocytidine
  modified_base (8)..(8): n is 2′-methoxyadenosine
  modified_base (9)..(10): n is 2′-fluorocytidine
  modified_base (11)..(11): n is 2′-fluoroadenosine
  modified_base (12)..(12): n is 2′-methoxyuridine
  modified_base (13)..(13): n is 2′-fluorouridine
  modified_base (14)..(14): n is 2′-methoxyuridine
  modified_base (15)..(15): n is 2′-fluoroadenosine
  modified_base (16)..(16): n is 2′-fluorocytidine
  modified_base (17)..(18): n is 2′-fluorouridine
  modified_base (19)..(19): n is 2′-fluorocytidine
  modified_base (20)..(21): n is 2′-methoxyadenosine
nnnnnnnnnnnnnnnnnnnnn

SEQ ID NO: 47 - RNA, 21 nt, Artificial Sequence
  Synthetic construct
  modified_base (1)..(1): n is 2′-fluorouridine
  modified_base (2)..(2): n is 2′-methoxyuridine
  modified_base (3)..(3): n is 2′-fluorouridine
  modified_base (4)..(4): n is 2′-methoxyuridine
  modified_base (5)..(5): n is 2′-fluorocytidine
  modified_base (6)..(6): n is 2′-methoxyuridine
  modified_base (7)..(7): n is 2′-fluoroadenosine
  modified_base (8)..(8): n is 2′-methoxyguanosine
  modified_base (9)..(9): n is 2′-fluoroadenosine
  modified_base (10)..(10): n is 2′-methoxycytidine
  modified_base (11)..(11): n is 2′-fluorocytidine
  modified_base (12)..(12): n is 2′-methoxyuridine
  modified_base (13)..(13): n is 2′-fluoroguanosine
  modified_base (14)..(14): n is 2′-methoxyuridine
  modified_base (15)..(17): n is 2′-fluorouridine
  modified_base (18)..(18): n is 2′-methoxyguanosine
  modified_base (19)..(19): n is 2′-fluorocytidine
  modified_base (20)..(21): n is 2′-fluorouridine
nnnnnnnnnnnnnnnnnnnnn

SEQ ID NO: 48 - RNA, 23 nt, Artificial Sequence
  Synthetic construct
  modified_base (1)..(1): n is 2′-methoxyadenosine
  modified_base (2)..(2): n is 2′-fluoroadenosine
  modified_base (3)..(3): n is 2′-methoxyguanosine
  modified_base (4)..(4): n is 2′-fluorocytidine
  modified_base (5)..(5): n is 2′-methoxyadenosine
  modified_base (6)..(6): n is 2′-fluoroadenosine
  modified_base (7)..(7): n is 2′-methoxyadenosine
  modified_base (8)..(8): n is 2′-fluoroadenosine
  modified_base (9)..(9): n is 2′-methoxycytidine
  modified_base (10)..(10): n is 2′-fluoroadenosine
  modified_base (11)..(11): n is 2′-methoxyguanosine
  modified_base (12)..(12): n is 2′-fluoroguanosine
  modified_base (13)..(13): n is 2′-methoxyuridine
  modified_base (14)..(14): n is 2′-fluorocytidine
  modified_base (15)..(15): n is 2′-methoxyuridine
  modified_base (16)..(16): n is 2′-fluoroadenosine
  modified_base (17)..(17): n is 2′-methoxyguanosine
  modified_base (18)..(18): n is 2′-fluoroadenosine
  modified_base (19)..(21): n is 2′-methoxyadenosine
  modified_base (22)..(22): n is 2′-fluoroguanosine
  modified_base (23)..(23): n is 2′-methoxyuridine
nnnnnnnnnnnnnnnnnnnnnnn

SEQ ID NO: 49 - RNA, 21 nt, Artificial Sequence
  Synthetic construct
  modified_base (1)..(1): n is 2′-fluorocytidine
  modified_base (2)..(2): n is 2′-methoxyadenosine
  modified_base (3)..(3): n is 2′-fluoroadenosine
  modified_base (4)..(4): n is 2′-methoxyguanosine
  modified_base (5)..(5): n is 2′-fluorocytidine
  modified_base (6)..(6): n is 2′-methoxyadenosine
  modified_base (7)..(7): n is 2′-fluoroguanosine
  modified_base (8)..(8): n is 2′-methoxyadenosine
  modified_base (9)..(9): n is 2′-fluorocytidine
  modified_base (10)..(10): n is 2′-fluoroadenosine
  modified_base (11)..(11): n is 2′-fluorouridine
  modified_base (12)..(12): n is 2′-methoxyuridine
  modified_base (13)..(13): n is 2′-fluorouridine
  modified_base (14)..(14): n is 2′-methoxyadenosine
  modified_base (15)..(15): n is 2′-fluorouridine
  modified_base (16)..(16): n is 2′-fluorocytidine
  modified_base (17)..(21): n is 2′-fluorouridine
nnnnnnnnnnnnnnnnnnnnn

SEQ ID NO: 50 - RNA, 23 nt, Artificial Sequence
  Synthetic construct
  modified_base (1)..(1): n is 2′-methoxyadenosine
  modified_base (2)..(2): n is 2′-fluoroadenosine
  modified_base (3)..(3): n is 2′-methoxyadenosine
  modified_base (4)..(4): n is 2′-fluoroadenosine
  modified_base (5)..(5): n is 2′-methoxyadenosine
  modified_base (6)..(6): n is 2′-fluoroguanosine
  modified_base (7)..(7): n is 2′-methoxyadenosine
  modified_base (8)..(8): n is 2′-fluorouridine
  modified_base (9)..(9): n is 2′-methoxyadenosine
  modified_base (10)..(10): n is 2′-fluoroadenosine
  modified_base (11)..(11): n is 2′-methoxyadenosine
  modified_base (12)..(12): n is 2′-methoxyuridine
  modified_base (13)..(13): n is 2′-methoxyguanosine
  modified_base (14)..(14): n is 2′-fluorouridine
  modified_base (15)..(15): n is 2′-methoxycytidine
  modified_base (16)..(16): n is 2′-fluorouridine
  modified_base (17)..(17): n is 2′-methoxyguanosine
  modified_base (18)..(18): n is 2′-fluorocytidine
  modified_base (19)..(19): n is 2′-methoxyuridine
  modified_base (20)..(20): n is 2′-fluorouridine
  modified_base (21)..(21): n is 2′-methoxyguanosine
  modified_base (22)..(22): n is 2′-fluorocytidine
  modified_base (23)..(23): n is 2′-methoxyuridine
nnnnnnnnnnnnnnnnnnnnnnn

SEQ ID NO: 51 - RNA, 21 nt, Artificial Sequence
  Synthetic construct
  modified_base (1)..(1): n is 2′-fluorocytidine
  modified_base (2)..(2): n is 2′-methoxyuridine
  modified_base (3)..(3): n is 2′-fluoroadenosine
  modified_base (4)..(4): n is 2′-methoxyguanosine
  modified_base (5)..(5): n is 2′-fluoroadenosine
  modified_base (6)..(6): n is 2′-methoxycytidine
  modified_base (7)..(7): n is 2′-fluorocytidine
  modified_base (8)..(8): n is 2′-methoxyuridine
  modified_base (9)..(9): n is 2′-fluoroguanosine
  modified_base (10)..(11): n is 2′-fluorouridine
  modified_base (12)..(12): n is 2′-methoxyuridine
  modified_base (13)..(13): n is 2′-fluorouridine
  modified_base (14)..(14): n is 2′-methoxyguanosine
  modified_base (15)..(15): n is 2′-fluorocytidine
  modified_base (16)..(19): n is 2′-fluorouridine
  modified_base (20)..(20): n is 2′-methoxyguanosine
  modified_base (21)..(21): n is 2′-fluorouridine
nnnnnnnnnnnnnnnnnnnnn

SEQ ID NO: 52 - RNA, 23 nt, Artificial Sequence
  Synthetic cosntruct
  modified_base (1)..(1): n is 2′-methoxyadenosine
  modified_base (2)..(2): n is 2′-fluorocytidine
  modified_base (3)..(3): n is 2′-methoxyadenosine
  modified_base (4)..(4): n is 2′-fluoroadenosine
  modified_base (5)..(5): n is 2′-methoxyadenosine
  modified_base (6)..(6): n is 2′-fluoroadenosine
  modified_base (7)..(7): n is 2′-methoxyguanosine
  modified_base (8)..(8): n is 2′-fluorocytidine
  modified_base (9)..(9): n is 2′-methoxyadenosine
  modified_base (10)..(10): n is 2′-fluoroadenosine
  modified_base (11)..(12): n is 2′-methoxyadenosine
  modified_base (13)..(13): n is 2′-methoxycytidine
  modified_base (14)..(14): n is 2′-fluoroadenosine
  modified_base (15)..(15): n is 2′-methoxyguanosine
  modified_base (16)..(16): n is 2′-fluoroguanosine
  modified_base (17)..(17): n is 2′-methoxyuridine
  modified_base (18)..(18): n is 2′-fluorocytidine
  modified_base (19)..(19): n is 2′-methoxyuridine
  modified_base (20)..(20): n is 2′-fluoroadenosine
  modified_base (21)..(21): n is 2′-methoxyguanosine
  modified_base (22)..(22): n is 2′-fluoroadenosine
  modified_base (23)..(23): n is 2′-methoxyadenosine
nnnnnnnnnnnnnnnnnnnnnnn

SEQ ID NO: 53 - RNA, 21 nt, Artificial Sequence
  Synthetic construct
  modified_base (1)..(1): n is 2′-fluorocytidine
  modified_base (2)..(2): n is 2′-methoxyuridine
  modified_base (3)..(3): n is 2′-fluoroadenosine
  modified_base (4)..(4): n is 2′-methoxyguanosine
  modified_base (5)..(5): n is 2′-fluoroadenosine
  modified_base (6)..(6): n is 2′-methoxycytidine
  modified_base (7)..(7): n is 2′-fluorocytidine
  modified_base (8)..(8): n is 2′-methoxyuridine
  modified_base (9)..(9): n is 2′-fluoroguanosine
  modified_base (10)..(11): n is 2′-fluorouridine
  modified_base (12)..(12): n is 2′-methoxyuridine
  modified_base (13)..(13): n is 2′-fluorouridine
  modified_base (14)..(14): n is 2′-methoxyguanosine
  modified_base (15)..(15): n is 2′-methoxycytidine
  modified_base (16)..(16): n is 2′-fluorouridine
  modified_base (17)..(17): n is 2′-methoxyuridine
  modified_base (18)..(18): n is 2′-fluorouridine
  modified_base (19)..(19): n is 2′-methoxyuridine
  modified_base (20)..(20): n is 2′-methoxyguanosine
  modified_base (21)..(21): n is 2′-methoxyuridine
nnnnnnnnnnnnnnnnnnnnn

SEQ ID NO: 54 - RNA, 23 nt, Artificial Sequence
  Synthetic construct
  modified_base (1)..(1): n is 2′-methoxyadenosine
  modified_base (2)..(2): n is 2′-fluorocytidine
  modified_base (3)..(3): n is 2′-methoxyadenosine
  modified_base (4)..(4): n is 2′-fluoroadenosine
  modified_base (5)..(6): n is 2′-methoxyadenosine
  modified_base (7)..(7): n is 2′-methoxyguanosine
  modified_base (8)..(8): n is 2′-fluorocytidine
  modified_base (9)..(9): n is 2′-methoxyadenosine
  modified_base (10)..(10): n is 2′-fluoroadenosine
  modified_base (11)..(12): n is 2′-methoxyadenosine
  modified_base (13)..(13): n is 2′-methoxycytidine
  modified_base (14)..(14): n is 2′-fluoroadenosine
  modified_base (15)..(15): n is 2′-methoxyguanosine
  modified_base (16)..(16): n is 2′-fluoroguanosine
  modified_base (17)..(17): n is 2′-methoxyuridine
  modified_base (18)..(18): n is 2′-methoxycytidine
  modified_base (19)..(19): n is 2′-methoxyuridine
  modified_base (20)..(20): n is 2′-fluoroadenosine
  modified_base (21)..(21): n is 2′-methoxyguanosine
  modified_base (22)..(23): n is 2′-methoxyadenosine
nnnnnnnnnnnnnnnnnnnnnnn

SEQ ID NO: 55 - RNA, 23 nt, Artificial Sequence
  Synthetic construct
  modified_base (1)..(1): n is 2′-methoxyadenosine
  modified_base (2)..(2): n is 2′-fluorocytidine
  modified_base (3)..(3): n is 2′-methoxyadenosine
  modified_base (4)..(4): n is 2′-fluoroadenosine
  modified_base (5)..(6): n is 2′-methoxyadenosine
  modified_base (7)..(7): n is 2′-methoxyguanosine
  modified_base (8)..(8): n is 2′-fluorocytidine
  modified_base (9)..(9): n is 2′-methoxyadenosine
  modified_base (10)..(10): n is 2′-fluoroadenosine
  modified_base (11)..(12): n is 2′-methoxyadenosine
  modified_base (13)..(13): n is 2′-methoxycytidine
  modified_base (14)..(14): n is 2′-fluoroadenosine
  modified_base (15)..(15): n is 2′-methoxyguanosine
  modified_base (16)..(16): n is 2′-fluoroguanosine
  modified_base (17)..(17): n is 2′-methoxyuridine
  modified_base (18)..(18): n is 2′-methoxycytidine
  modified_base (19)..(19): n is 2′-methoxyuridine
  modified_base (20)..(20): n is 2′-methoxyadenosine
  modified_base (21)..(21): n is 2′-methoxyguanosine
  modified_base (22)..(23): n is 2′-methoxyadenosine
nnnnnnnnnnnnnnnnnnnnnnn

SEQ ID NO: 56 - DNA, 24 bp, Artificial Sequence
  Synthetic construct
  modified_base (1)..(1): n is 2′-fluoroadenosine
  modified_base (2)..(2): n is 2′-methoxyadenosine
  modified_base (3)..(3): n is 2′-fluorocytidine
  modified_base (4)..(4): n is 2′-methoxyadenosine
  modified_base (5)..(5): n is 2′-fluoroguanosine
  modified_base (6)..(6): n is 2′-methoxyuridine
  modified_base (7)..(7): n is 2′-fluoroguanosine
  modified_base (8)..(8): n is 2′-methoxyuridine
  modified_base (9)..(9): n is 2′-fluorouridine
  modified_base (10)..(10): n is 2′-fluorocytidine
  modified_base (11)..(11): n is 2′-fluorouridine
  modified_base (12)..(12): n is 2′-methoxyuridine
  modified_base (13)..(13): n is 2′-fluoroguanosine
  modified_base (14)..(14): n is 2′-methoxycytidine
  modified_base (15)..(15): n is 2′-fluorouridine
  modified_base (16)..(16): n is 2′-methoxycytidine
  modified_base (17)..(17): n is 2′-fluorouridine
  modified_base (18)..(18): n is 2′-methoxyadenosine
  modified_base (19)..(19): n is 2′-fluorouridine
  modified_base (20)..(20): n is 2′-methoxyadenosine
  modified_base (21)..(21): n is 2′-fluoroadenosine
  modified_base (22)..(24): n is 2′-fluorothymidine
nnnnnnnnnnnnnnnnnnnnnnnn

SEQ ID NO: 57 - DNA, 24 bp, Artificial Sequence
  Synthetic construct
  modified_base (1)..(2): n is 2′-methoxyguanosine
  modified_base (3)..(3): n is 2′-fluorouridine
  modified_base (4)..(4): n is 2′-methoxyuridine
  modified_base (5)..(5): n is 2′-fluoroadenosine
  modified_base (6)..(6): n is 2′-methoxyadenosine
  modified_base (7)..(7): n is 2′-fluorocytidine
  modified_base (8)..(8): n is 2′-methoxyadenosine
  modified_base (9)..(10): n is 2′-fluorocytidine
  modified_base (11)..(11): n is 2′-fluoroadenosine
  modified_base (12)..(12): n is 2′-methoxyuridine
  modified_base (13)..(13): n is 2′-fluorouridine
  modified_base (14)..(14): n is 2′-methoxyuridine
  modified_base (15)..(15): n is 2′-fluoroadenosine
  modified_base (16)..(16): n is 2′-methoxycytidine
  modified_base (17)..(17): n is 2′-fluorouridine
  modified_base (18)..(18): n is 2′-methoxyuridine
  modified_base (19)..(19): n is 2′-fluorocytidine
  modified_base (20)..(21): n is 2′-methoxyadenosine
  modified_base (22)..(24): n is 2′-fluorothymidine
nnnnnnnnnnnnnnnnnnnnnnnn

SEQ ID NO: 58 - RNA, 21 nt, Artificial Sequence
  Synthetic construct
  modified_base (1)..(2): n is 2′-methoxyguanosine
  modified_base (3)..(3): n is 2′-fluorouridine
  modified_base (4)..(4): n is 2′-methoxyuridine
  modified_base (5)..(5): n is 2′-fluoroadenosine
  modified_base (6)..(6): n is 2′-methoxyadenosine
  modified_base (7)..(7): n is 2′-fluorocytidine
  modified_base (8)..(8): n is 2′-methoxyadenosine
  modified_base (9)..(10): n is 2′-fluorocytidine
  modified_base (11)..(11): n is 2′-fluoroadenosine
  modified_base (12)..(12): n is 2′-methoxyuridine
  modified_base (13)..(13): n is 2′-fluorouridine
  modified_base (14)..(14): n is 2′-methoxyuridine
  modified_base (15)..(15): n is 2′-fluoroadenosine
  modified_base (16)..(16): n is 2′-methoxycytidine
  modified_base (17)..(17): n is 2′-fluorouridine
  modified_base (18)..(18): n is 2′-methoxyuridine
  modified_base (19)..(19): n is 2′-fluorocytidine
  modified_base (20)..(20): n is 2′-methoxyethoxyadenosine
  modified_base (21)..(21): n is 2′-methoxyadenosine
nnnnnnnnnnnnnnnnnnnnn

SEQ ID NO: 59 - RNA, 23 nt, Artificial Sequence
  Synthetic construct
  modified_base (1)..(2): n is 2′-fluorouridine
  modified_base (3)..(3): n is 2′-methoxyguanosine
  modified_base (4)..(4): n is 2′-fluoroadenosine
  modified_base (5)..(5): n is 2′-methoxyadenosine
  modified_base (6)..(6): n is 2′-fluoroguanosine
  modified_base (7)..(7): n is 2′-methoxyuridine
  modified_base (8)..(8): n is 2′-fluoroadenosine
  modified_base (9)..(9): n is 2′-methoxyadenosine
  modified_base (10)..(10): n is 2′-fluoroadenosine
  modified_base (11)..(11): n is 2′-methoxyuridine
  modified_base (12)..(13): n is 2′-methoxyguanosine
  modified_base (14)..(14): n is 2′-fluorouridine
  modified_base (15)..(15): n is 2′-methoxyguanosine
  modified_base (16)..(16): n is 2′-fluorouridine
  modified_base (17)..(17): n is 2′-methoxyuridine
  modified_base (18)..(18): n is 2′-fluoroadenosine
  modified_base (19)..(19): n is 2′-methoxyadenosine
  modified_base (20)..(20): n is 2′-fluorocytidine
  modified_base (21)..(21): n is 2′-methoxycytidine
  modified_base (22)..(22): n is 2′-methoxyethoxyadenosine
  modified_base (23)..(23): n is 2′-methoxyguanosine
nnnnnnnnnnnnnnnnnnnnnnn

(19) United States
(12) Patent Application Publication
(10) Pub. No.: US 2019/0194655 A1
(43) Pub. Date: Jun. 27, 2019

(54) POLYNUCLEOTIDE CONSTRUCTS HAVING AN AUXILIARY MOIETY NON-BIOREVERSIBLY LINKED TO AN INTERNUCLEOSIDE PHOSPHATE OR PHOSPHOROTHIOATE

(71) Applicants: Curt W. Bradshaw, San Diego, CA (US); Sukumar Sakamuri, San Diego, CA (US); Bryan R. Meade, San Diego, CA (US); Laxman Eltepu, San Diego, CA (US); Giuseppe Dello Iacono, Oceanside, CA (US); Son Lam, San Diego, CA (US); Joseph Stock, San Diego, CA (US); Dingguo Liu, San Diego, CA (US); Bin Liu, San Diego, CA (US); Solstice Biologics, Ltd., Dublin (IE)

(72) Inventors: Curt W. BRADSHAW, San Diego, CA (US); Sukumar SAKAMURI, San Diego, CA (US); Bryan R. MEADE, San Diego, CA (US); Laxman ELTEPU, San Diego, CA (US); Giuseppe Dello IACONO, Oceanside, CA (US); Son LAM, San Diego, CA (US); Joseph STOCK, San Diego, CA (US); Dingguo LIU, San Diego, CA (US); Bin LIU, San Diego, CA (US)

(21) Appl. No.: 16/060,171

(22) PCT Filed: Dec. 8, 2016
(86) PCT No.: PCT/US16/65639

§ 371 Date: Jun. 7, 2018

Related U.S. Application Data
(60) Provisional application No. 62/264,736, filed on Dec. 8, 2015.

(51) Int. Cl. C12N 15/113

(52) U.S. Cl. CPC C12N 15/113; C12N 2310/351; C12N 2310/312; C12N 2310/311; C12N 2310/315

(57) ABSTRACT

The invention features a hybridized polynucleotide construct including a passenger strand, a guide strand loadable into a RISC complex, and one or more auxiliary moieties. At least one of the auxiliary moieties is non-bioreversibly linked to an internucleoside phosphate or phosphorothioate in the passenger strand. The invention further features methods of delivery a polynucleotide construct to a cell and methods of reducing the expression of a protein in a cell. The methods typically involve contacting the cell with the hybridized polynucleotide construct.

### FIELD OF THE INVENTION

This invention relates to compositions and methods for transfecting cells.

### BACKGROUND

Nucleic acid delivery to cells both in vitro and in vivo has been performed using various recombinant viral vectors, lipid delivery systems and electroporation. Such techniques have sought to treat various diseases and disorders by knocking-out gene expression, providing genetic constructs for gene therapy or to study various biological systems.

Polyanionic polymers such as polynucleotides do not readily diffuse across cell membranes. To overcome this problem for cultured cells, cationic lipids are typically combined with anionic polynucleotides to assist uptake. Unfortunately, this complex is generally toxic to cells, which means that both the exposure time and concentration of cationic lipid must be carefully controlled to insure transfection of viable cells.

The discovery of RNA interference (RNAi) as a cellular mechanism that selectively degrades mRNAs allows for both the targeted manipulation of cellular phenotypes in cell culture and the potential for development of directed therapeutics (Behlke, Mol. Ther. 13, 644-670, 2006; Xie et al., Drug Discov. Today 11, 67-73, 2006). However, because of their size and negative (anionic) charged nature, siRNAs are macromolecules with no ability to enter cells. Indeed, siRNAs are 25× in excess of Lipinski's “Rule of 5s” for cellular delivery of membrane diffusible molecules that generally limits size to less than 500 Da. Consequently, in the absence of a delivery vehicle or transfection agent, naked siRNAs do not enter cells, even at millimolar concentrations (Barquinero et al., Gene Ther. 11 Suppl 1, S3-9, 2004). Significant attention has been focused on the use of cationic lipids that both condense the siRNA and punch holes in the cellular membrane to solve the siRNA delivery problem. Although widely used, transfection reagents fail to achieve efficient delivery into many cell types, especially primary cells and hematopoietic cell lineages (T and B cells, macrophage). Moreover, lipofection reagents often result in varying degrees of cytotoxicity ranging from mild in tumor cells to high in primary cells.

Accordingly, there is a need for polynucleotide constructs with increased ability to transfect cells. Particularly desirable are polynucleotide constructs capable of targeting a predetermined cell population.

### SUMMARY OF THE INVENTION

In general, the invention provides hybridized polynucleotides having an auxiliary moiety linked to a phosphate or a phosphorothioate in one of the strands included in the hybridized polynucleotide.

In a first aspect, the invention provides a hybridized polynucleotide construct containing a passenger strand, a guide strand loadable into a RISC complex, and one or more auxiliary moieties; where at least one of the auxiliary moieties is non-bioreversibly linked to an internucleoside phosphate or phosphorothioate in the passenger strand; where the one or more auxiliary moieties are independently selected from the group consisting of a targeting moiety, a cell penetrating peptide, an endosomal escape moiety, and a neutral organic polymer.

In some embodiments of the first aspect, the hybridized polynucleotide construct contains from 1 to 5 (e.g., from 2 to 5) auxiliary moieties, at least one of the auxiliary moieties being linked non-bioreversibly to an internucleoside phosphate or phosphorothioate in the passenger strand, and the remaining auxiliary moieties being independently linked bioreversibly to a phosphate or phosphorothioate in the guide strand or linked bioreversibly or non-bioreversibly to a phosphate or phosphorothioate in the passenger strand.

In a second aspect, the invention provides a hybridized polynucleotide construct containing a passenger strand, a guide strand loadable into a RISC complex. In some embodiments of the second aspect, the hybridized polynucleotide construct contains at least one of the auxiliary moieties non-bioreversibly linked to a phosphate or a phosphorothioate in the passenger strand and at least one additional auxiliary moiety bioreversibly or non-bioreversibly linked to a phosphate or a phosphorothioate in the passenger strand or the guide strand; where the auxiliary moieties are independently selected from the group consisting of a targeting moiety, a cell penetrating peptide, an endosomal escape moiety, and a neutral organic polymer.

In certain embodiments of the first or second aspect, the auxiliary moieties are the same. In particular embodiments of the first or second aspect, the auxiliary moieties are linked to proximal phosphates or phosphorothioates. In further embodiments of the first or second aspect, the auxiliary moiety (e.g., each of the auxiliary moieties) has a single ligand. In other embodiments of the first or second aspect, the ligand is linked to the internucleoside phosphate or phosphorothioate through a linear oligomeric linker (e.g., a linear oligomeric linker containing poly(ethylene glycol) (e.g., poly(ethylene glycol) having from 2 to 50 repeating units).

In particular embodiments of the first or second aspect, when the auxiliary moiety is linked to a phosphate or a phosphorothioate in the guide strand, the auxiliary moiety is linked bioreversibly.

In some embodiments of the first or second aspect, the guide strand contains at least one internucleoside phosphorothioate linking two of the four 3′-terminal nucleosides in the guide strand. In other embodiments of the first or second aspect, the guide strand contains at least one internucleoside phosphorothioate linking two of the four 5′-terminal nucleosides in the guide strand. In certain other embodiments of the first or second aspect, the guide strand contains at least one internucleoside phosphorothioate linking two of the four 3′-terminal nucleosides in the passenger strand. In yet other embodiments of the first or second aspect, the passenger strand contains at least one internucleoside phosphorothioate linking two of the four 5′-terminal nucleosides in the passenger strand.

In further embodiments of the first or second aspect, the auxiliary moiety is non-bioreversibly linked through a non-bioreversible linker containing a 1,2,3-triazol-diyl or a N-sulfonylamidocarbonyl.

In other embodiments of the first or second aspect, the auxiliary moiety combines with the non-bioreversible linker to form a group that is

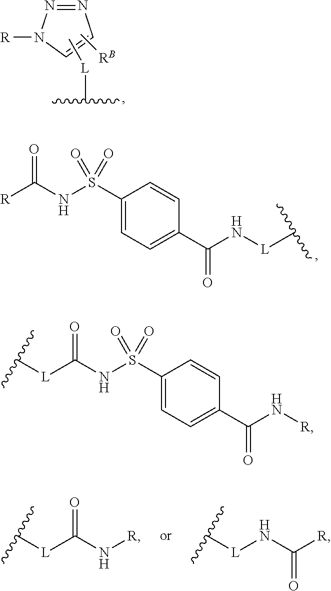

where

R is the auxiliary moiety;

R^{B} is H or C₁₋₆ alkyl; and

L is C₂₋₆ alkylene or —(CH₂CH₂O)ₚ₁(CH₂CH₂)—, where p1 is an integer from 1 to 50.

In certain embodiments of the first or second aspect, at least one of the auxiliary moieties is a targeting moiety (e.g., a targeting moiety containing a ligand that is N-acetyl galactosamine, mannose, folate, prostate specific membrane antigen (PSMA), or an antibody or an antigen-binding fragment thereof). In particular embodiments of the first or second aspect, the targeting moiety contains a ligand that is N-acetyl galactosamine. In other embodiments of the first or second aspect, N-acetyl galactosamine is linked to the phosphate or phosphorothioate through a linker bonded to the anomeric carbon of N-acetyl galactosamine, where the anomeric carbon is part of a hemiaminal group.

In some embodiments of the first or second aspect, at least one of the auxiliary moieties is a cell penetrating peptide.

In further embodiments of the first or second aspect, at least one of the auxiliary moieties is an endosomal escape moiety.

In particular embodiments of the first or second aspect, the guide strand or the passenger strand further contains one or more internucleoside phosphotriesters, internucleoside phosphonates, or internucleoside phosphoramidates. In some embodiments of the first or second aspect, the guide strand or the passenger strand contains one or more of the internucleoside phosphotriesters, where at least one of the internucleoside phosphotriesters is a non-bioreversible phosphotriester.

In certain embodiments of the first or second aspect, at least one (e.g., each) of the non-bioreversible phosphotriesters is a phosphate or a phosphorothioate substituted with a substituent selected independently from the group consisting of optionally substituted C₂₋₁₆ alkyl; optionally substituted C₃₋₁₆ alkenyl; optionally substituted C₃₋₁₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O, and S; optionally substituted C₂₋₉ heterocyclyl having 1 to 4 heteroatoms selected from N, O, and S, where the heterocyclyl does not contain an S—S bond; optionally substituted (C₂₋₉ heterocyclyl)-C₁₋₄-alkyl having 1 to 4 heteroatoms selected from N, O, and S, where the heterocyclyl does not contain an S—S bond; and a group of the following structure:

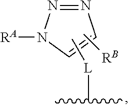

where

L is C₂₋₆ alkylene;

R^{A} is optionally substituted C₂₋₆ alkyl; optionally substituted C₆₋₁₄ aryl; optionally substituted (C₆₋₁₄ aryl)-C₁₋₄-alkyl; optionally substituted C₃₋₈ cycloalkyl; optionally substituted (C₃₋₈ cycloalkyl)-C₁₋₄-alkyl; optionally substituted C₁₋₉ heteroaryl having 1 to 4 heteroatoms selected from the group consisting of N, O, and S; optionally substituted (C₁₋₉ heteroaryl)-C₁₋₄-alkyl having 1 to 4 heteroatoms selected from the group consisting of N, O, and S; optionally substituted C₂₋₉ heterocyclyl having 1 to 4 heteroatoms selected from the group consisting of N, O, and S, where the heterocyclyl does not contain an S—S bond; optionally substituted (C₂₋₉ heterocyclyl)-C₁₋₄-alkyl having 1 to 4 heteroatoms selected from N, O, and S, where the heterocyclyl does not contain an S—S bond; and a poly(ethylene glycol) terminated with —OH, C₁₋₆ alkoxy, or —COOH; and

R^{B} is H or C₁₋₆ alkyl.

In some embodiments of the first or second aspect, at least one (e.g., each) of the non-bioreversible phosphotriesters is a phosphate or a phosphorothioate substituted with a substituent that is

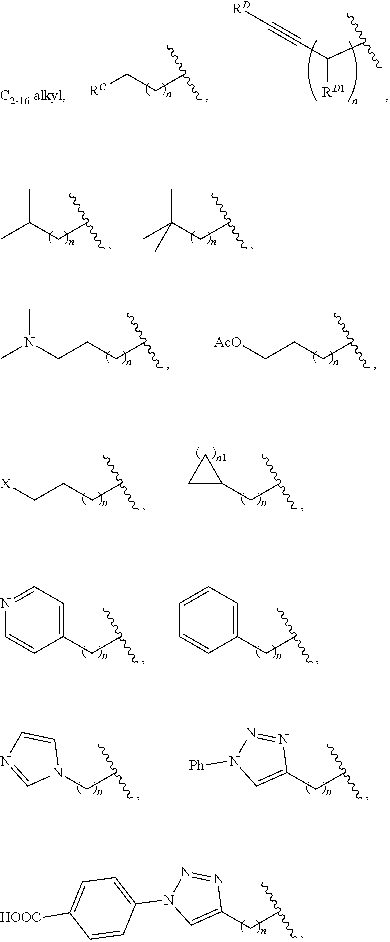

or a group formed by cycloaddition reaction of

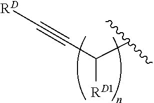

with an azido-containing substrate,

where

n is an integer from 1 to 6;

n1 is an integer from 1 to 6 (e.g., from 1 to 4);

R^{C} is optionally substituted C₆ aryl; optionally substituted C₄₋₅ heteroaryl that is a six member ring containing 1 or 2 nitrogen atoms; or optionally substituted C₄₋₅ heterocyclyl that is a six member ring containing 1 or 2 nitrogen atoms;

R^{D} is H or C₁₋₆ alkyl;

each R^{D1} is independently H or C₁₋₆ alkyl, provided that

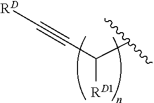

contains 24 carbon atoms or fewer;

X is a halogen, —COOR¹, or —CONR²₂, where each of R¹ and R² is independently H, optionally substituted C₁₋₆ alkyl, optionally substituted C₁₋₁₄ aryl, optionally substituted C₁₋₉ heteroaryl, or optionally substituted C₂₋₉ heterocyclyl; and

the azido-containing substrate is

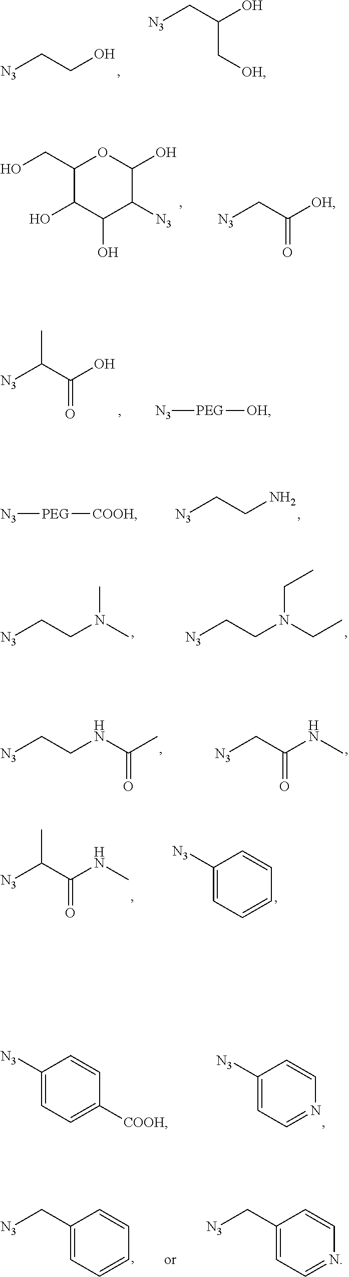

In particular embodiments of the first or second aspect, the guide strand contains from 1 to 5 of the non-bioreversible phosphotriesters.

In some embodiments of the first or second aspect, the non-bioreversible phosphotriesters are disposed outside the seed region. In other embodiments of the first or second aspect, one of the non-bioreversible phosphotriesters connects the second nucleoside and the third nucleoside of the guide strand. In yet other embodiments of the first or second aspect, one of the non-bioreversible phosphotriesters connects the fifth nucleoside and the sixth nucleoside of the guide strand. In still other embodiments of the first or second aspect, one of the non-bioreversible phosphotriesters connects the seventeenth nucleoside and the eighteenth nucleoside of the guide strand. In certain other embodiments of the first or second aspect, one of the non-bioreversible phosphotriesters connects the nineteenth nucleoside and the twentieth nucleoside of the guide strand. In particular embodiments of the first or second aspect, one of the non-bioreversible phosphotriesters connects the twentieth nucleoside and the twenty first nucleoside of the guide strand.

In further embodiments of the first or second aspect, the passenger strand contains from 1 to 5 of the non-bioreversible phosphotriesters.

In certain embodiments of the first or second aspect, the guide strand or the passenger strand contains one or more of the internucleoside phosphotriesters, at least one of the internucleoside phosphotriesters being a bioreversible phosphotriester.

In particular embodiments of the first or second aspect, the bioreversible phosphotriester is a phosphate or a phosphorothioate substituted with -(Link A)-S—S—R^{E}, where

Link A is a divalent or trivalent linker containing an sp³-hybridized carbon atom bonded to the phosphate or phosphorothioate and a carbon atom bonded to —S—S—, where, when Link A is a trivalent linker, the third valency of Link A combines with —S—S— and R^{E} to form optionally substituted C₃₋₉ heterocyclylene, and

R^{E} is optionally substituted C₂₋₈ alkyl; optionally substituted C₃₋₈ alkenyl; optionally substituted C₃₋₈ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O, and S; optionally substituted C₂₋₉ heterocyclyl having 1 to 4 heteroatoms selected from N, O, and S; optionally substituted (C₂₋₉ heterocyclyl)-C₁₋₄-alkyl having 1 to 4 heteroatoms selected from N, O, and S; or, when Link A is a trivalent linker, R^{E} combines with —S—S— and Link A to form optionally substituted C₃₋₉ heterocyclylene.

In some embodiments of the first or second aspect, the bioreversible phosphotriester is a phosphate or a phosphorothioate substituted with a group that is

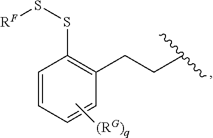

R^{F} is optionally substituted C₁₋₆ alkyl or optionally substituted C₆₋₁₄ aryl (e.g., R^{F} is optionally substituted C₁₋₆ alkyl),

R^{G} is a halogen or optionally substituted C₁₋₆ alkyl, and

q is an integer from 0 to 4 (e.g., q is 0).

In certain embodiments of the first or second aspect, the guide strand or the passenger strand contains one or more phosphonates.

In particular embodiments of the first or second aspect, the guide strand contains 19 or more nucleosides. In other embodiments of the first or second aspect, the guide strand contains fewer than 100 nucleosides (e.g., fewer than 50 nucleosides or fewer than 32 nucleosides). In yet other embodiments of the first or second aspect, the passenger strand contains 19 or more nucleosides. In still other embodiments of the first or second aspect, the passenger strand contains fewer than 100 nucleosides (e.g., fewer than 50 nucleosides or fewer than 32 nucleosides).

In certain embodiments of the first or second aspect, the hybridized polynucleotide construct does not contain a bioreversible group.

In a third aspect, the invention provides a method of delivering a polynucleotide construct to a cell by contacting the cell with the hybridized polynucleotide construct of the first or second aspect, where, after the contacting, the polynucleotide construct resides inside the cell.

In a fourth aspect, the invention provides a method of reducing the expression of a protein in a cell by contacting the cell with the hybridized polynucleotide construct of the first or second aspect, where, after the contacting, expression of the protein in the cell is reduced.

In some embodiments of any aspect, the auxiliary moieties in the hybridized polynucleotide construct are linked to the passenger strand. In particular embodiments, at least some of the auxiliary moieties may be linked to internucleoside phosphates or phosphorothioates in the following pattern: —N-p^{L}-(—N-p-)_{z}-N-p^{L}-(—N-p-)_{z}-N-p^{L}-[(—N-p-)_{z}-N-p^{L}-]_{z1}-, where each N is independently a nucleoside; each p^{L} is a phosphate or phosphorothioate bioreversibly linked to an auxiliary moiety; each p is independently a phosphate, phosphorothioate, phosphoramidate, or phosphonate; each z is independently 0, 1, or 2; and z1 is 0, 1, or 2. In further embodiments, at least some of the auxiliary moieties may be linked to internucleoside phosphates or phosphorothioates in the following pattern: —N-p^{L}-(—N-p-)_{z}-N-p^{L}-(—N-p-)_{z}-N-p^{L}-[(—N-p-)_{z}-N-p^{L}-]_{z1}-, where each N is independently a nucleoside; each p^{L} is a phosphate or phosphorothioate non-bioreversibly linked to an auxiliary moiety; each p is independently a phosphate, phosphorothioate, phosphoramidate, or phosphonate; each z is independently 0, 1, or 2; and z1 is 0, 1, or 2.

### Definitions

The term “about,” as used herein, represents a value that is ±10% of the recited value.

The term “activated carbonyl,” as used herein, represents a functional group having the formula of —C(O)R^{A} where R^{A} is a halogen, optionally substituted C₁₋₆ alkoxy, optionally substituted C₆₋₁₀ aryloxy, optionally substituted C₂₋₉ heteroaryloxy (e.g., —OBt), optionally substituted C₂-C₉ heterocyclyloxy (e.g., —OSu), optionally substituted pyridinium (e.g., 4-dimethylaminopyridinium), or —N(OMe)Me.

The term “activated phosphorus center,” as used herein, represents a trivalent phosphorus (III) or a pentavalent phosphorus (V) center, in which at least one of the substituents is a halogen, optionally substituted C₁₋₆ alkoxy, optionally substituted C₆₋₁₀ aryloxy, phosphate, diphosphate, triphosphate, tetraphosphate, optionally substituted pyridinium (e.g., 4-dimethylaminopyridinium), or optionally substituted ammonium.

The term “activated silicon center,” as used herein, represents a tetrasubstituted silicon center, in which at least one of the substituents is a halogen, optionally substituted C₁₋₆ alkoxy, or amino.

The term “activated sulfur center,” as used herein, represents a tetravalent sulfur where at least one of the substituents is a halogen, optionally substituted C₁₋₆ alkoxy, optionally substituted C₆₋₁₀ aryloxy, phosphate, diphosphate, triphosphate, tetraphosphate, optionally substituted pyridinium (e.g., 4-dimethylaminopyridinium), or optionally substituted ammonium.

The term “alkanoyl,” as used herein, represents a hydrogen or an alkyl group (e.g., a haloalkyl group) that is attached to the parent molecular group through a carbonyl group and is exemplified by formyl (i.e., a carboxaldehyde group), acetyl, propionyl, butyryl, isobutyryl, and the like. Exemplary unsubstituted alkanoyl groups include from 1 to 7 carbons. In some embodiments, the alkyl group is further substituted with 1, 2, 3, or 4 substituents as described herein.

The term “(C_{x1-y1} aryl)-C_{x2-y2}-alkyl,” as used herein, represents an aryl group of x1 to y1 carbon atoms attached to the parent molecular group through an alkylene group of x2 to y2 carbon atoms. Exemplary unsubstituted (C_{x1-y1} aryl)-C_{x2-y2}-alkyl groups are from 7 to 16 carbons. In some embodiments, the alkylene and the aryl each can be further substituted with 1, 2, 3, or 4 substituent groups as defined herein for the respective groups. Other groups followed by “alkyl” are defined in the same manner, where “alkyl” refers to a C₁₋₆ alkylene, unless otherwise noted, and the attached chemical structure is as defined herein.

The term “alkenyl,” as used herein, represents acyclic monovalent straight or branched chain hydrocarbon groups of containing one, two, or three carbon-carbon double bonds. Non-limiting examples of the alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted with 1, 2, 3, or 4 substituent groups selected, independently, from the group consisting of aryl, cycloalkyl, heterocyclyl (e.g., heteroaryl), as defined herein, and the substituent groups described for alkyl. In addition, when an alkenyl group is present in a bioreversible group of the invention it may be substituted with a thioester or disulfide group that is bound to a conjugating moiety, a hydrophilic functional group, or an auxiliary moiety as defined herein.

The term “alkenylene,” as used herein, refers to a straight or branched chain alkenyl group with one hydrogen removed, thereby rendering this group divalent. Non-limiting examples of the alkenylene groups include ethen-1,1-diyl; ethen-1,2-diyl; prop-1-en-1,1-diyl, prop-2-en-1,1-diyl; prop-1-en-1,2-diyl, prop-1-en-1,3-diyl; prop-2-en-1,1-diyl; prop-2-en-1,2-diyl; but-1-en-1,1-diyl; but-1-en-1,2-diyl; but-1-en-1,3-diyl; but-1-en-1,4-diyl; but-2-en-1,1-diyl; but-2-en-1,2-diyl; but-2-en-1,3-diyl; but-2-en-1,4-diyl; but-2-en-2,3-diyl; but-3-en-1,1-diyl; but-3-en-1,2-diyl; but-3-en-1,3-diyl; but-3-en-2,3-diyl; buta-1,2-dien-1,1-diyl; buta-1,2-dien-1,3-diyl; buta-1,2-dien-1,4-diyl; buta-1,3-dien-1,1-diyl; buta-1,3-dien-1,2-diyl; buta-1,3-dien-1,3-diyl; buta-1,3-dien-1,4-diyl; buta-1,3-dien-2,3-diyl; buta-2,3-dien-1,1-diyl; and buta-2,3-dien-1,2-diyl. The alkenylene group may be unsubstituted or substituted (e.g., optionally substituted alkenylene) as described for alkenyl groups.

The term “alkoxy,” as used herein, represents a chemical substituent of formula —OR, where R is a C₁₋₆ alkyl group, unless otherwise specified. In some embodiments, the alkyl group can be further substituted with 1, 2, 3, or 4 substituent groups as defined herein.

The term “alkyl,” as used herein, refers to an acyclic straight or branched chain saturated hydrocarbon group having from 1 to 16 carbons, unless otherwise specified. Alkyl groups are exemplified by methyl; ethyl; n- and iso-propyl; n-, sec-, iso- and tert-butyl; neopentyl, and the like, and may be optionally substituted with one, two, three, or, in the case of alkyl groups of two carbons or more, four substituents independently selected from the group consisting of: (1) alkoxy; (2) alkylsulfinyl; (3) amino; (4) arylalkoxy; (5) (arylalkyl)aza; (6) azido; (7) halo; (8) (heterocyclyl)oxy; (9) (heterocyclyl)aza; (10) hydroxy; (11) nitro; (12) oxo; (13) aryloxy; (14) sulfide; (15) thioalkoxy; (16) thiol; (17) alkanoyl; (18) —CO₂R^{A}, where R^{A} is selected from the group consisting of (a) alkyl, (b) aryl, (c) hydrogen, and (d) arylalkyl; (19) —C(O)NR^{B}R^{C}, where each of R^{B} and R^{C} is, independently, selected from the group consisting of (a) hydrogen, (b) alkyl, (c) aryl, and (d) aryl-alkylene; (20) —SO₂R^{D}, where R^{D} is selected from the group consisting of (a) alkyl, (b) aryl, and (c) aryl-alkylene; (21) —SO₂NR^{E}R^{F}, where each of R^{E} and R^{F} is, independently, selected from the group consisting of (a) hydrogen, (b) alkyl, (c) aryl and (d) arylalkyl; (22) silyl; (23) cyano; and (24) —S(O)R^{H} where R^{H} is selected from the group consisting of (a) hydrogen, (b) alkyl, (c) aryl, and (d) arylalkyl. In some embodiments, each of these groups can be further substituted as described herein. In certain embodiments, the alkyl carbon atom bonding to the parent molecular group is not oxo-substituted.

The term “alkylene,” as used herein, refers to a saturated divalent, trivalent, or tetravalent hydrocarbon group derived from a straight or branched chain saturated hydrocarbon by the removal of at least two hydrogen atoms. Alkylene can be trivalent if bonded to one aza group that is not an optional substituent; alkylene can be trivalent or tetravalent if bonded to two aza groups that are not optional substituents. The valency of alkylene defined herein does not include the optional substituents. Non-limiting examples of the alkylene group include methylene, ethane-1,2-diyl, ethane-1,1-diyl, propane-1,3-diyl, propane-1,2-diyl, propane-1,1-diyl, propane-2,2-diyl, butane-1,4-diyl, butane-1,3-diyl, butane-1,2-diyl, butane-1,1-diyl, and butane-2,2-diyl, butane-2,3-diyl. The term “C_{x-y} alkylene” represents alkylene groups having between x and y carbons. Exemplary values for x are 1, 2, 3, 4, 5, and 6, and exemplary values for y are 2, 3, 4, 5, 6, 7, 8, 9, 10, 11, and 12. In some embodiments, the alkylene can be further substituted with 1, 2, 3, or 4 substituent groups as defined herein for an alkyl group. Similarly, the suffix “ene” designates a divalent radical of the corresponding monovalent radical as defined herein. For example, alkenylene, alkynylene, arylene, aryl alkylene, cycloalkylene, cycloalkyl alkylene, cycloalkenylene, heteroarylene, heteroaryl alkylene, heterocyclylene, and heterocyclyl alkylene are divalent forms of alkenyl, alkynyl, aryl, aryl alkyl, cycloalkyl, cycloalkyl alkyl cycloalkenyl, heteroaryl, heteroaryl alkyl, heterocyclyl, and heterocyclyl alkyl. For aryl alkylene, cycloalkyl alkylene, heteroaryl alkylene, and heterocyclyl alkylene, the two valences in the group may be located in the acyclic portion only or one in the cyclic portion and one in the acyclic portion. In addition, when an alkyl or alkylene, alkenyl or alkenylene, or alkynyl or alkynylene group is present in a bioreversible or a non-bioeversible group, it may be substituted with an ester, thioester, or disulfide group that is bound to a conjugating moiety, a hydrophilic functional group, or an auxiliary moiety as defined herein. For example, the alkylene group of an aryl-C₁-alkylene or a heterocyclyl-C₁-alkylene can be further substituted with an oxo group to afford the respective aryloyl and (heterocyclyl)oyl substituent group.

The term “alkyleneoxy,” as used herein, refers to a divalent group —R—O—, in which R is alkylene.

The term “alkynyl,” as used herein, represents monovalent straight or branched chain hydrocarbon groups of from two to six carbon atoms containing at least one carbon-carbon triple bond and is exemplified by ethynyl, 1-propynyl, and the like. Alkynyl groups may be optionally substituted with 1, 2, 3, or 4 substituent groups that are selected, independently, from aryl, alkenyl, cycloalkyl, heterocyclyl (e.g., heteroaryl), as defined herein, and the substituent groups described for alkyl.

The term “alkynylene,” as used herein, refers to a straight-chain or branched-chain divalent substituent including one or two carbon-carbon triple bonds and containing only C and H when unsubstituted. Non-limiting examples of the alkenylene groups include ethyn-1,2-diyl; prop-1-yn-1,3-diyl; prop-2-yn-1,1-diyl; but-1-yn-1,3-diyl; but-1-yn-1,4-diyl; but-2-yn-1,1-diyl; but-2-yn-1,4-diyl; but-3-yn-1,1-diyl; but-3-yn-1,2-diyl; but-3-yn-2,2-diyl; and buta-1,3-diyn-1,4-diyl. The alkynylene group may be unsubstituted or substituted (e.g., optionally substituted alkynylene) as described for alkynyl groups.

The term “amino,” as used herein, represents —N(R^{N1})₂ or —N(R^{N1})C(NR^{N1})N(R^{N1})₂ where each R^{N1} is, independently, H, OH, NO₂, N(R^{N2})₂, SO₂OR^{N2}, SO₂R^{N2}, SOR^{N2}, an N-protecting group, alkyl, alkenyl, alkynyl, alkoxy, aryl, aryl-alkyl, cycloalkyl, cycloalkylalkyl, heterocyclyl (e.g., heteroaryl), heterocyclylalkyl (e.g., heteroarylalkyl), or two R^{N1} combine to form a heterocyclyl, and where each R^{N2} is, independently, H, alkyl, or aryl. In one embodiment, amino is —NH₂, or —NHR^{N1}, where R^{N1} is, independently, OH, NO₂, NH₂, NR^{N2}₂, SO₂OR^{N2}, SO₂R^{N2}, SOR^{N2}, alkyl, or aryl, and each R^{N2} can be H, alkyl, or aryl. Each R^{N1} group may be independently unsubstituted or substituted as described herein. In addition, when an amino group is present in a bioreversible group of the invention it may be substituted with an ester, thioester, or disulfide group that is bound to a conjugating moiety, a hydrophilic functional group, or an auxiliary moiety as defined herein.

The term “aryl,” as used herein, represents a mono-, bicyclic, or multicyclic carbocyclic ring system having one or two aromatic rings and is exemplified by phenyl, naphthyl, 1,2-dihydronaphthyl, 1,2,3,4-tetrahydronaphthyl, fluorenyl, indanyl, indenyl, and the like, and may be optionally substituted with one, two, three, four, or five substituents independently selected from the group consisting of: (1) alkanoyl (e.g., formyl, acetyl, and the like); (2) alkyl (e.g., alkoxyalkyl, alkylsulfinylalkyl, aminoalkyl, azidoalkyl, acylalkyl, haloalkyl (e.g., perfluoroalkyl), hydroxyalkyl, nitroalkyl, or thioalkoxyalkyl); (3) alkenyl; (4) alkynyl; (5) alkoxy (e.g., perfluoroalkoxy); (6) alkylsulfinyl; (7) aryl; (8) amino; (9) arylalkyl; (10) azido; (11) cycloalkyl; (12) cycloalkylalkyl; (13) cycloalkenyl; (14) cycloalkenylalkyl; (15) halo; (16) heterocyclyl (e.g., heteroaryl); (17) (heterocyclyl)oxy; (18) (heterocyclyl)aza; (19) hydroxy; (20) nitro; (21) thioalkoxy; (22) —(CH₂)_{q}CO₂R^{A}, where q is an integer from zero to four, and R^{A} is selected from the group consisting of (a) alkyl, (b) aryl, (c) hydrogen, and (d) arylalkyl; (23) —(CH₂)_{q}CONR^{B}R^{C}, where q is an integer from zero to four and where R^{B} and R^{C} are independently selected from the group consisting of (a) hydrogen, (b) alkyl, (c) aryl, and (d) arylalkyl; (24) —(CH₂)_{q}SO₂R^{D}, where q is an integer from zero to four and where R^{D} is selected from the group consisting of (a) alkyl, (b) aryl, and (c) arylalkyl; (25) —(CH₂)_{q}SO₂NR^{E}R^{F}, where q is an integer from zero to four and where each of R^{E} and R^{F} is, independently, selected from the group consisting of (a) hydrogen, (b) alkyl, (c) aryl, and (d) arylalkyl; (26) thiol; (27) aryloxy; (28) cycloalkoxy; (29) arylalkoxy; (30) heterocyclylalkyl (e.g., heteroarylalkyl); (31) silyl; (32) cyano; and (33) —S(O)R^{H} where R^{H} is selected from the group consisting of (a) hydrogen, (b) alkyl, (c) aryl, and (d) arylalkyl. In some embodiments, each of these groups can be further substituted as described herein. In addition, when an aryl group is present in a bioreversible group of the invention it may be substituted with an ester, thioester, or disulfide group that is bound to a conjugating moiety, a hydrophilic functional group, or an auxiliary moiety as defined herein.

The term “aryl alkyl,” as used herein, represents an alkyl group substituted with an aryl group. The aryl and alkyl portions may be substituted as the individual groups as described herein.

The term “auxiliary moiety” refers to any moiety, including, but not limited to, a small molecule, a peptide, a carbohydrate, a neutral organic polymer, a positively charged polymer, a therapeutic agent, a targeting moiety, an endosomal escape moiety, and any combination thereof, which can be conjugated to a polynucleotide construct disclosed herein. Generally, but not always the case, an “auxiliary moiety” is linked to a polynucleotide construct disclosed herein by forming one or more covalent bonds to one or more conjugating groups attached to a phosphate or a phosphorothioate in the hybridized polynucleotide construct. However, in alternative embodiments an “auxiliary moiety” may be linked or attached to a polynucleotide construct disclosed herein by forming one or more covalent bonds to any portion of the nucleotide construct in addition to conjugating groups attached to a phosphate or a phosphorothioate in the hybridized polynucleotide construct, such as to the 2′, 3′, or 5′ positions of a nucleotide sugar molecule, or on any portion of a nucleobase. Although the name for a particular auxiliary moiety may imply a free molecule, it will be understood that such a free molecule is attached to a polynucleotide construct. One skilled in the art will readily understand appropriate points of attachment of a particular auxiliary moiety to a nucleotide construct.

The term “aza,” as used herein, represents a divalent —N(R^{N1})— group or a trivalent —N═ group. The aza group may be unsubstituted, where R^{N1} is H or absent, or substituted, where R^{N1} is as defined for “amino.” Aza may also be referred to as “N,” e.g., “optionally substituted N.” Two aza groups may be connected to form “diaza.”

The term “azido,” as used herein, represents an N₃ group.

The term “bioreversible linker,” as used herein, represents a divalent moiety including a functional group that can be actively cleaved intracellularly, e.g., via the action of one or more intracellular enzymes (e.g., an intracellar reductase) or passively cleaved intracellularly, such as by exposing the group to the intracellular environment or a condition present in the cell (e.g., pH, reductive or oxidative environment, or reaction with intracellular species, such as glutathione). Exemplary bioreversible linkers include disulfides. Other exemplary bioreversible groups include thioesters. A first group that is linked bioreversibly to a second group, thus, is linked through a bioreversible linker.

The term “bulky group,” as used herein, represents any substituent or group of substituents as defined herein, in which the radical of the bulky group bears one hydrogen atom or fewer if the radical is sp³-hybridized carbon, bears no hydrogen atoms if the radical is sp²-hybridized carbon. The radical is not sp-hybridized carbon. The bulky group bonds to another group only through a carbon atom. For example, the statements “bulky group bonded to the disulfide linkage,” “bulky group attached to the disulfide linkage,” and “bulky group linked to the disulfide linkage” indicate that the bulky group is bonded to the disulfide linkage through a carbon radical.

The term “carbocyclic,” as used herein, represents an optionally substituted C₃₋₁₂ monocyclic, bicyclic, or tricyclic structure in which the rings, which may be aromatic or non-aromatic, are formed by carbon atoms. Carbocyclic structures include cycloalkyl, cycloalkenyl, and aryl groups.

The term “carbohydrate,” as used herein, represents a compound which comprises one or more monosaccharide units having at least 5 carbon atoms (which may be linear, branched or cyclic) with an oxygen, nitrogen or sulfur atom bonded to each carbon atom. The term “carbohydrate” therefore encompasses monosaccharides, disaccharides, trisaccharides, tetrasaccharides, oligosaccharides, and polysaccharides. Representative carbohydrates include the sugars (mono-, di-, tri- and oligosaccharides containing from about 4-9 monosaccharide units), and polysaccharides such as starches, glycogen, cellulose and polysaccharide gums. Specific monosaccharides include C₅₋₆ sugars; di- and trisaccharides include sugars having two or three monosaccharide units (e.g., C₅₋₆ sugars).

The term “carbonyl,” as used herein, represents a C(O) group. Examples of functional groups which comprise a “carbonyl” include esters, ketones, aldehydes, anhydrides, acyl chlorides, amides, carboxylic acids, and carboxlyates.

The term “complementary” in reference to a polynucleotide, as used herein, means Watson-Crick complementary.

The term “conjugating group,” as used herein, represents a divalent or higher valency group containing one or more conjugating moieties. The conjugating group links one or more auxiliary moieties to a bioreversible group (e.g., a group containing a disulfide moiety).

The term “conjugating moiety,” as used herein, represents a functional group that is capable of forming one or more covalent bonds to another group (e.g., a functional group that is a nucleophile, electrophile, a component in a cycloaddition reaction, or a component in a coupling reaction) under appropriate conditions. The term also refers to the residue of a conjugation reaction, e.g., amide group. Examples of such groups are provided herein.

The term “coupling reaction,” as used herein, represents a reaction of two components in which one component includes a nonpolar a bond such as Si—H or C—H and the second component includes a π bond such as an alkene or an alkyne that results in either the net addition of the a bond across the π bond to form C—H, Si—C, or C—C bonds or the formation of a single covalent bond between the two components. One coupling reaction is the addition of Si—H across an alkene (also known as hydrosilylation). Other coupling reactions include Stille coupling, Suzuki coupling, Sonogashira coupling, Hiyama coupling, and the Heck reaction. Catalysts may be used to promote the coupling reaction. Typical catalysts are those which include Fe(II), Cu(I), Ni(0), Ni(II), Pd(0), Pd(II), Pd(IV), Pt(0), Pt(II), or Pt(IV).

The term “cycloaddition reaction” as used herein, represents reaction of two components in which [4n+2] π electrons are involved in bond formation when there is either no activation, activation by a chemical catalyst, or activation using thermal energy, and n is 1, 2, or 3. A cycloaddition reaction is also a reaction of two components in which [4n] π electrons are involved, there is photochemical activation, and n is 1, 2, or 3. Desirably, [4n+2] π electrons are involved in bond formation, and n=1. Representative cycloaddition reactions include the reaction of an alkene with a 1,3-diene (Diels-Alder reaction), the reaction of an alkene with an α,β-unsaturated carbonyl (hetero Diels-Alder reaction), and the reaction of an alkyne with an azido compound (e.g., Hüisgen cycloaddition).

The term “cycloalkenyl,” as used herein, refers to a non-aromatic carbocyclic group having from three to ten carbons (e.g., a C₃-C₁₀ cycloalkylene), unless otherwise specified. Non-limiting examples of cycloalkenyl include cycloprop-1-enyl, cycloprop-2-enyl, cyclobut-1-enyl, cyclobut-1-enyl, cyclobut-2-enyl, cyclopent-1-enyl, cyclopent-2-enyl, cyclopent-3-enyl, norbornen-1-yl, norbornen-2-yl, norbornen-5-yl, and norbornen-7-yl. The cycloalkenyl group may be unsubstituted or substituted (e.g., optionally substituted cycloalkenyl) as described for cycloalkyl.

The term “cycloalkenylene,” as used herein, refers to a divalent carbocyclic non-aromatic group having from three to ten carbons (e.g., C₃-C₁₀ cycloalkenylene), unless otherwise specified. Non-limiting examples of the cycloalkenylene include cycloprop-1-en-1,2-diyl; cycloprop-2-en-1,1-diyl; cycloprop-2-en-1,2-diyl; cyclobut-1-en-1,2-diyl; cyclobut-1-en-1,3-diyl; cyclobut-1-en-1,4-diyl; cyclobut-2-en-1,1-diyl; cyclobut-2-en-1,4-diyl; cyclopent-1-en-1,2-diyl; cyclopent-1-en-1,3-diyl; cyclopent-1-en-1,4-diyl; cyclopent-1-en-1,5-diyl; cyclopent-2-en-1,1-diyl; cyclopent-2-en-1,4-diyl; cyclopent-2-en-1,5-diyl; cyclopent-3-en-1,1-diyl; cyclopent-1,3-dien-1,2-diyl; cyclopent-1,3-dien-1,3-diyl; cyclopent-1,3-dien-1,4-diyl; cyclopent-1,3-dien-1,5-diyl; cyclopent-1,3-dien-5,5-diyl; norbornadien-1,2-diyl; norbornadien-1,3-diyl; norbornadien-1,4-diyl; norbornadien-1,7-diyl; norbornadien-2,3-diyl; norbornadien-2,5-diyl; norbornadien-2,6-diyl; norbornadien-2,7-diyl; and norbornadien-7,7-diyl. The cycloalkenylene may be unsubstituted or substituted (e.g., optionally substituted cycloalkenylene) as described for cycloalkyl.

The term “cycloalkyl,” as used herein, refers to a cyclic alkyl group having from three to ten carbons (e.g., a C₃-C₁₀ cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be unsubstituted or substituted as defined herein (e.g., optionally substituted cycloalkyl). The cycloalkyl groups of this disclosure can be optionally substituted with: (1) alkanoyl (e.g., formyl, acetyl, and the like); (2) alkyl (e.g., alkoxyalkyl, alkylsulfinylalkyl, aminoalkyl, azidoalkyl, acylalkyl, haloalkyl (e.g., perfluoroalkyl), hydroxyalkyl, nitroalkyl, or thioalkoxyalkyl); (3) alkenyl; (4) alkynyl; (5) alkoxy (e.g., perfluoroalkoxy); (6) alkylsulfinyl; (7) aryl; (8) amino; (9) arylalkyl; (10) azido; (11) cycloalkyl; (12) cycloalkylalkyl; (13) cycloalkenyl; (14) cycloalkenylalkyl; (15) halo; (16) heterocyclyl (e.g., heteroaryl); (17) (heterocyclyl)oxy; (18) (heterocyclyl)aza; (19) hydroxy; (20) nitro; (21) thioalkoxy; (22) —(CH₂)_{q}CO₂R^{A}, where q is an integer from zero to four, and R^{A} is selected from the group consisting of (a) alkyl, (b) aryl, (c) hydrogen, and (d) arylalkyl; (23) —(CH₂)_{q}CONR^{B}R^{C}, where q is an integer from zero to four and where R^{B} and R^{C} are independently selected from the group consisting of (a) hydrogen, (b) alkyl, (c) aryl, and (d) arylalkyl; (24) —(CH₂)_{q}SO₂R^{D}, where q is an integer from zero to four and where R^{D} is selected from the group consisting of (a) alkyl, (b) aryl, and (c) arylalkyl; (25) —(CH₂)_{q}SO₂NR^{E}R^{F}, where q is an integer from zero to four and where each of R^{E} and R^{F} is, independently, selected from the group consisting of (a) hydrogen, (b) alkyl, (c) aryl, and (d) arylalkyl; (26) thiol; (27) aryloxy; (28) cycloalkoxy; (29) arylalkoxy; (30) heterocyclylalkyl (e.g., heteroarylalkyl); (31) silyl; (32) cyano; and (33) —S(O)R^{H} where R^{H} is selected from the group consisting of (a) hydrogen, (b) alkyl, (c) aryl, and (d) arylalkyl. In some embodiments, each of these groups can be further substituted as described herein.

The term “cycloalkyl alkyl,” as used herein, represents an alkyl group substituted with a cycloalkyl group. The cycloalkyl and alkyl portions may be substituted as the individual groups as described herein.

The term “electrophile” or “electrophilic group,” as used herein, represents a functional group that is attracted to electron rich centers and is capable of accepting pairs of electrons from one or more nucleophiles so as to form one or more covalent bonds. Electrophiles include, but are not limited to, cations; polarized neutral molecules; azides; activated silicon centers; activated carbonyls; alkyl halides; alkyl pseudohalides; epoxides; electron-deficient aryls; activated phosphorus centers; and activated sulfur centers. Typically encountered electrophiles include polarized neutral molecules, such as alkyl halides, acyl halides, carbonyl containing compounds, such as aldehydes, and atoms which are connected to good leaving groups, such as mesylates, triflates, and tosylates.

The term “endosomal escape moiety,” as used herein, represents a moiety which enhances the release of endosomal contents or allows for the escape of a molecule from an internal cellular compartment such as an endosome.

The term “halo,” as used herein, represents a halogen selected from bromine, chlorine, iodine, and fluorine.

The term “haloalkyl,” as used herein, represents an alkyl group, as defined herein, substituted by a halogen group (i.e., F, Cl, Br, or I). A haloalkyl may be substituted with one, two, three, or, in the case of alkyl groups of two carbons or more, four halogens, or, when the halogen group is F, haloalkyl group can be perfluoroalkyl. In some embodiments, the haloalkyl group can be further optionally substituted with 1, 2, 3, or 4 substituent groups as described herein for alkyl groups.

The term “heteroaryl,” as used herein, represents that subset of heterocyclyls, as defined herein, which are aromatic: i.e., they contain 4n+2 pi electrons within the mono- or multicyclic ring system. In one embodiment, the heteroaryl is substituted with 1, 2, 3, or 4 substituents groups as defined for a heterocyclyl group.

The term “heteroaryl alkyl,” as used herein, represents an alkyl group substituted with a heteroaryl group. The heteroaryl and alkyl portions may be substituted as the individual groups as described herein.

The term “heterocyclyl,” as used herein, represents a 5-, 6- or 7-membered ring, unless otherwise specified, containing one, two, three, or four heteroatoms independently selected from the group comprising nitrogen, oxygen, and sulfur. The 5-membered ring has zero to two double bonds, and the 6- and 7-membered rings have zero to three double bonds. Certain heterocyclyl groups include from 2 to 9 carbon atoms. Other such groups may include up to 12 carbon atoms. The term “heterocyclyl” also represents a heterocyclic compound having a bridged multicyclic structure in which one or more carbons and/or heteroatoms bridges two non-adjacent members of a monocyclic ring, e.g., a quinuclidinyl group. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring, such as indolyl, quinolyl, isoquinolyl, tetrahydroquinolyl, benzofuryl, benzothienyl and the like. Examples of fused heterocyclyls include tropanes and 1,2,3,5,8,8a-hexahydroindolizine. Heterocyclics include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, thiazolidinyl, isothiazolyl, isoindazoyl, triazolyl, tetrazolyl, oxadiazolyl, purinyl, thiadiazolyl (e.g., 1,3,4-thiadiazole), tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, benzofuranyl, benzothienyl and the like. Still other exemplary heterocyclyls include: 2,3,4,5-tetrahydro-2-oxo-oxazolyl; 2,3-dihydro-2-oxo-1H-imidazolyl; 2,3,4,5-tetrahydro-5-oxo-1H-pyrazolyl (e.g., 2,3,4,5-tetrahydro-2-phenyl-5-oxo-1H-pyrazolyl); 2,3,4,5-tetrahydro-2,4-dioxo-1H-imidazolyl (e.g., 2,3,4,5-tetrahydro-2,4-dioxo-5-methyl-5-phenyl-1H-imidazolyl); 2,3-dihydro-2-thioxo-1,3,4-oxadiazolyl (e.g., 2,3-dihydro-2-thioxo-5-phenyl-1,3,4-oxadiazolyl); 4,5-dihydro-5-oxo-1H-triazolyl (e.g., 4,5-dihydro-3-methyl-4-amino 5-oxo-1H-triazolyl); 1,2,3,4-tetrahydro-2,4-dioxopyridinyl (e.g., 1,2,3,4-tetrahydro-2,4-dioxo-3,3-diethylpyridinyl); 2,6-dioxo-piperidinyl (e.g., 2,6-dioxo-3-ethyl-3-phenylpiperidinyl); 1,6-dihydro-6-oxopyridiminyl; 1,6-dihydro-4-oxopyrimidinyl (e.g., 2-(methylthio)-1,6-dihydro-4-oxo-5-methylpyrimidin-1-yl); 1,2,3,4-tetrahydro-2,4-dioxopyrimidinyl (e.g., 1,2,3,4-tetrahydro-2,4-dioxo-3-ethylpyrimidinyl); 1,6-dihydro-6-oxo-pyridazinyl (e.g., 1,6-dihydro-6-oxo-3-ethylpyridazinyl); 1,6-dihydro-6-oxo-1,2,4-triazinyl (e.g., 1,6-dihydro-5-isopropyl-6-oxo-1,2,4-triazinyl); 2,3-dihydro-2-oxo-1H-indolyl (e.g., 3,3-dimethyl-2,3-dihydro-2-oxo-1H-indolyl and 2,3-dihydro-2-oxo-3,3′-spiropropane-1H-indol-1-yl); 1,3-dihydro-1-oxo-2H-iso-indolyl; 1,3-dihydro-1,3-dioxo-2H-iso-indolyl; 1H-benzopyrazolyl (e.g., 1-(ethoxycarbonyl)-1H-benzopyrazolyl); 2,3-dihydro-2-oxo-1H-benzimidazolyl (e.g., 3-ethyl-2,3-dihydro-2-oxo-1H-benzimidazolyl); 2,3-dihydro-2-oxo-benzoxazolyl (e.g., 5-chloro-2,3-dihydro-2-oxo-benzoxazolyl); 2,3-dihydro-2-oxo-benzoxazolyl; 2-oxo-2H-benzopyranyl; 1,4-benzodioxanyl; 1,3-benzodioxanyl; 2,3-dihydro-3-oxo,4H-1,3-benzothiazinyl; 3,4-dihydro-4-oxo-3H-quinazolinyl (e.g., 2-methyl-3,4-dihydro-4-oxo-3H-quinazolinyl); 1,2,3,4-tetrahydro-2,4-dioxo-3H-quinazolyl (e.g., 1-ethyl-1,2,3,4-tetrahydro-2,4-dioxo-3H-quinazolyl); 1,2,3,6-tetrahydro-2,6-dioxo-7H-purinyl (e.g., 1,2,3,6-tetrahydro-1,3-dimethyl-2,6-dioxo-7H-purinyl); 1,2,3,6-tetrahydro-2,6-dioxo-1H-purinyl (e.g., 1,2,3,6-tetrahydro-3,7-dimethyl-2,6-dioxo-1H-purinyl); 2-oxobenz[c,d]indolyl; 1,1-dioxo-2H-naphth[1,8-c,d]isothiazolyl; and 1,8-naphthylenedicarboxamido. Heterocyclic groups also include groups of the formula

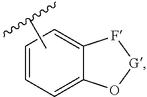

where

F′ is selected from the group consisting of —CH₂—, —CH₂O— and —O—, and G′ is selected from the group consisting of —C(O)— and —(C(R′)(R″))ᵥ—, where each of R′ and R″ is, independently, selected from the group consisting of hydrogen or alkyl of one to four carbon atoms, and v is one to three and includes groups, such as 1,3-benzodioxolyl, 1,4-benzodioxanyl, and the like. Any of the heterocyclyl groups mentioned herein may be optionally substituted with one, two, three, four or five substituents independently selected from the group consisting of: (1) alkanoyl (e.g., formyl, acetyl, and the like); (2) alkyl (e.g., alkoxyalkylene, alkylsulfinylalkylene, aminoalkylene, azidoalkylene, acylalkylene, haloalkylene (e.g., perfluoroalkyl), hydroxyalkylene, nitroalkylene, or thioalkoxyalkylene); (3) alkenyl; (4) alkynyl; (5) alkoxy (e.g., perfluoroalkoxy); (6) alkylsulfinyl; (7) aryl; (8) amino; (9) aryl-alkylene; (10) azido; (11) cycloalkyl; (12) cycloalkyl-alkylene; (13) cycloalkenyl; (14) cycloalkenyl-alkylene; (15) halo; (16) heterocyclyl (e.g., heteroaryl); (17) (heterocyclyl)oxy; (18) (heterocyclyl)aza; (19) hydroxy; (20) oxo; (21) nitro; (22) sulfide; (23) thioalkoxy; (24) —(CH₂)_{q}CO₂R^{A}, where q is an integer from zero to four, and R^{A} is selected from the group consisting of (a) alkyl, (b) aryl, (c) hydrogen, and (d) aryl-alkylene; (25) —(CH₂)_{q}CONR^{B}R^{C}, where q is an integer from zero to four and where R^{B} and R^{C} are independently selected from the group consisting of (a) hydrogen, (b) alkyl, (c) aryl, and (d) aryl-alkylene; (26) —(CH₂)_{q}SO₂R^{D}, where q is an integer from zero to four and where R^{D} is selected from the group consisting of (a) alkyl, (b) aryl, and (c) aryl-alkylene; (27) —(CH₂)_{q}SO₂NR^{E}R^{F}, where q is an integer from zero to four and where each of R^{E} and R^{F} is, independently, selected from the group consisting of (a) hydrogen, (b) alkyl, (c) aryl, and (d) aryl-alkylene; (28) thiol; (29) aryloxy; (30) cycloalkoxy; (31) arylalkoxy; (31) heterocyclyl-alkylene (e.g., heteroaryl-alkylene); (32) silyl; (33) cyano; and (34) —S(O)R^{H} where R^{H} is selected from the group consisting of (a) hydrogen, (b) alkyl, (c) aryl, and (d) aryl-alkylene. In some embodiments, each of these groups can be further substituted as described herein. For example, the alkylene group of an aryl-C₁-alkylene or a heterocyclyl-C₁-alkylene can be further substituted with an oxo group to afford the respective aryloyl and (heterocyclyl)oyl substituent group. In addition, when a heterocyclyl group is present in a bioreversible group of the invention it may be substituted with an ester, thioester, or disulfide group that is bound to a conjugating moiety, a hydrophilic functional group, or an auxiliary moiety as defined herein.

The term “heterocyclyl alkyl,” as used herein, represents an alkyl group substituted with a heterocyclyl group. The heterocyclyl and alkyl portions may be substituted as the individual groups as described herein.

The term “hydrophilic functional group,” as used herein, represents a moiety that confers an affinity to water and increases the solubility of an alkyl moiety in water. Hydrophilic functional groups can be ionic or non-ionic and include moieties that are positively charged, negatively charged, and/or can engage in hydrogen-bonding interactions. Exemplary hydrophilic functional groups include hydroxy, amino, carboxyl, carbonyl, thiol, phosphates (e.g., a mono-, di-, or tri-phosphate), polyalkylene oxides (e.g., polyethylene glycols), and heterocyclyls.

The terms “hydroxyl” and “hydroxy,” as used interchangeably herein, represent an —OH group.

The term “imine,” as used herein, represents a group having a double bond between carbon and nitrogen, which can be represented as “C═N.” In a particular embodiment, where a proton is a to the imine functional group, the imine may also be in the form of the tautomeric enamine. A type of imine bond is the hydrazone bond, where the nitrogen of the imine bond is covalently attached to a trivalent nitrogen (e.g., C═N—N(R)₂). In some embodiments, each R can be, independently, H, OH, optionally substituted C₁₋₆ alkoxy, or optionally substituted C₁₋₆ alkyl.

The term “internucleoside group,” as used herein, represents a group which covalently links two consecutive nucleosides together. The internucleoside group can be a non-bioreversible or a bioreversible group as defined herein. The internucleoside phosphorus (V) group is phosphate or phosphorothioate. One oxygen atom of the internucleoside group is at 3′ position of one nucleoside and another oxygen atom of the internucleoside group is at 5′ position of another adjacent nucleoside.

The term “LNA,” as used herein, refers to a locked nucleic acid, which is known in the art. See, e.g., WO 1999/014226.

The term “loadable into a RISC complex,” as used herein, refers to the capability of a guide strand to be loaded into a RISC complex and the RISC-mediated degradation of a passenger strand hybridized to the guide strand. For example, this polynucleotide includes unsubstituted or bioreversibly substituted phosphate groups between the three contiguous nucleotides including a natural RISC-mediated cleavage site. Certain loadable into a RISC complex guide strands include 5′-terminal nucleoside that is bonded to 5′-terminal or internucleoside phosphates or phosphorothioates that are either unsubstituted or substituted bioreversibly. The preferred natural RISC-mediated cleavage site is located on the passenger strand between two nucleosides that are complementary to the tenth and eleventh nucleotides of the guide strand.

The term “nitro,” as used herein, represents an —NO₂ group.

The term “non-bioreversible linker,” as used herein, refers to a multivalent moiety that is not bioreversible and thus does not include a disulfide or thioester. A first group non-bioreversibly linked to a second group, thus, is linked through the non-bioreversible linker.

A “non-naturally occurring amino acid” is an amino acid not naturally produced or found in a mammal. Non-naturally occurring amino acids are known in the art.

By “nonpolar a bond” is meant a covalent bond between two elements having electronegativity values, as measured according to the Pauling scale, that differ by less than or equal to 1.0 units. Non-limiting examples of nonpolar a bonds include C—C, C—H, Si—H, Si—C, C—Cl, C—Br, C—I, C—B, and C—Sn bonds.

The term “nucleobase,” as used herein, represents a nitrogen-containing heterocyclic ring found at the 1′ position of the sugar moiety of a nucleotide or nucleoside. Nucleobases can be unmodified or modified. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C or m5c), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808; those disclosed in The Concise Encyclopedia Of Polymer Science And Engineering, pages 858-859, Kroschwitz, J. I., ed. John Wiley & Sons, 1990; those disclosed by Englisch et al., Angewandte Chemie, International Edition, 1991, 30, 613; and those disclosed by Sanghvi, Y. S., Chapter 15, Antisense Research and Applications, pages 289 302, (Crooke et al., ed., CRC Press, 1993). Certain nucleobases are particularly useful for increasing the binding affinity of the polymeric compounds of the invention, including 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications 1993, CRC Press, Boca Raton, pages 276-278). These may be combined, in particular embodiments, with 2′-O-methoxyethyl sugar modifications. United States patents that teach the preparation of certain of these modified nucleobases as well as other modified nucleobases include, but are not limited to, the above noted U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; and 5,681,941. For the purposes of this disclosure, “modified nucleobases,” as used herein, further represents nucleobases, natural or nonnatural, which include one or more protecting groups as described herein.

The terms “nucleophile,” as used herein, represent an optionally substituted functional group that engages in the formation of a covalent bond by donating electrons from electron pairs or π bonds. Nucleophiles may be selected from alkenes, alkynes, aryl, heteroaryl, diaza groups, hydroxy groups, alkoxy groups, aryloxy groups, amino groups, alkylamino groups, anilido groups, thio groups, and thiophenoxy groups.

The term “nucleoside,” as used herein, represents a sugar-nucleobase combination. The sugar is a modified sugar containing a nucleobase at the anomeric carbon or a 3,5-dideoxypentafuranose containing a nucleobase at the anomeric carbon and a bond to another group at each position 3 and 5. The pentafuranose may be 3,5-dideoxyribose or 2,3,5-trideoxyribose or a 2 modified version thereof, in which position 2 is substituted with OR, R, halo (e.g., F), SH, SR, NH₂, NHR, NR₂, or CN, where R is an optionally substituted C₁₋₆ alkyl (e.g., (C₁₋₆ alkoxy)-C₁₋₆-alkyl) or optionally substituted (C₆₋₁₄ aryl)-C₁₋₄-alkyl.

The modified sugars are non-ribose sugars, such as mannose, arabinose, glucopyranose, galactopyranose, 4-thioribose, and other sugars, heterocycles, or carbocycles. In some embodiments, the term “nucleoside” refers to a divalent group having the following structure:

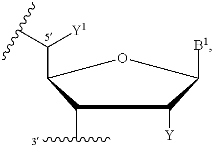

in which B¹ is a nucleobase; Y is H, halogen (e.g., F), hydroxyl, optionally substituted C₁₋₆ alkoxy (e.g., methoxy or methoxyethoxy), or a protected hydroxyl group; Y¹ is H or C₁₋₆ alkyl (e.g., methyl) and each of 3′ and 5′ indicate the position of a bond to another group. Nucleosides also include locked nucleic acids (LNA), glycerol nucleic acids, morpholino nucleic acids, and threose nucleic acids.

The term “nucleotide,” as used herein, refers to a nucleoside that further includes an internucleoside or a terminal phosphorus (V) group covalently linked to the 3′ or 5′ position of the divalent group.

The terms “oxa” and “oxy,” as used interchangeably herein, represents a divalent oxygen atom that is connected to two groups (e.g., the structure of oxy may be shown as —O—).

The term “oxo,” as used herein, represents a divalent oxygen atom that is connected to one group (e.g., the structure of oxo may be shown as ═O).

The term “phosphonate,” as used herein, refers to a monovalent or divalent group having the structure —O—P(═O)(-A)-O—B, where A is alkyl or aryl, and B is a valency, if phosphonate is divalent, or H, if phosphonate is monovalent, or a salt thereof.

The term “phosphoramidate,” as used herein, refers to a monovalent or divalent group having the structure —O—P(═X)(-A)-O—B, where A is amino, X is O or S, and B is a valency, if phosphoramidate is divalent, or H, if phosphoramidate is monovalent, or a salt thereof.

The term “phosphotriester,” as used herein, refers to a phosphate or a phosphorothioate, in which all three valences are substituted.

The term “phosphorus (V) group,” as used herein, refers to a divalent group having the structure —O—P(═Z^{A})(—Z^{B})—O—, in which Z^{A} is O or S, and Z^{B} is OH, SH, amino, alkyl, or aryl, or a salt thereof.

The term “polynucleotide” as used herein, represents a structure containing 11 or more contiguous nucleosides covalently bound together by any combination of internucleotide phosphorus (V), bioreversible, or non-bioreversible groups. Polynucleotides may be linear (i.e., having one 5′-terminus and one 3′-terminus) or circular. Nucleosides within the polynucleotides disclosed herein are numbered starting at 5′-terminus.

The term “peptide,” as used herein, represents two or more amino acid residues linked by peptide bonds. Moreover, for purposes of this disclosure, the term “peptide” and the term “protein” are used interchangeably herein in all contexts. A variety of peptides may be used within the scope of the methods and compositions provided herein. Peptides made synthetically may include substitutions of amino acids known in the art as not naturally encoded by DNA (e.g., a non-naturally occurring amino acid).

The term “Ph,” as used herein, represents phenyl.

The terms “photolytic activation” or “photolysis,” as used herein, represent the promotion or initiation of a chemical reaction by irradiation of the reaction with light. The wavelengths of light suitable for photolytic activation range between 200-500 nm and include wavelengths that range from 200-260 nm and 300-460 nm. Other useful ranges include 200-230 nm, 200-250 nm, 200-275 nm, 200-300 nm, 200-330 nm, 200-350 nm, 200-375 nm, 200-400 nm, 200-430 nm, 200-450 nm, 200-475 nm, 300-330 nm, 300-350 nm, 300-375 nm, 300-400 nm, 300-430 nm, 300-450 nm, 300-475 nm, and 300-500 nm.

The term “protecting group,” as used herein, represents a group intended to protect a functional group (e.g., a hydroxyl, an amino, or a carbonyl) from participating in one or more undesirable reactions during chemical synthesis (e.g., polynucleotide synthesis). The term “O-protecting group,” as used herein, represents a group intended to protect an oxygen containing (e.g., phenol, hydroxyl or carbonyl) group from participating in one or more undesirable reactions during chemical synthesis. The term “N-protecting group,” as used herein, represents a group intended to protect a nitrogen containing (e.g., an amino or hydrazine) group from participating in one or more undesirable reactions during chemical synthesis. Commonly used 0- and N-protecting groups are disclosed in Greene, “Protective Groups in Organic Synthesis,” 3^{rd} Edition (John Wiley & Sons, New York, 1999), which is incorporated herein by reference. Exemplary 0- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl. N-protecting groups useful for protection of amines in nucleobases include phenoxyacetyl and (4-isopropyl)phenoxyacetyl.

The term “proximal,” when used herein in reference to phosphates or phosphorothioates, refers to the phosphate or phosphorothioate being separated from another phosphate or phosphorothioate by one nucleoside or by two nucleosides and an internucleoside moiety.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and aryl-alkylene ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, aryl-alkylene groups such as benzyl, triphenylmethyl, benzyloxymethyl, and the like and silyl groups such as trimethylsilyl, and the like. Useful N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “subject,” as used herein, represents a human or non-human animal (e.g., a mammal).

The term “sulfide” as used herein, represents a divalent —S— or ═S group.

The term “targeting moiety,” as used herein, represents a moiety (e.g., a small molecule, such as a carbohydrate) that specifically binds or reactively associates or complexes with a receptor or other receptive moiety associated with a given target cell population. A targeting moiety contains one or more ligands (e.g., from 1 to 5 ligands, from 1 to 3 ligands, or 1 ligand). The ligand can be an antibody or an antigen-binding fragment or an engineered derivative thereof (e.g., Fcab or a fusion protein (e.g., scFv)).

Alternatively, the ligand is a small molecule (e.g., N-acetylgalactosamine, mannose, or folate).

The term “terminal group,” as used herein, refers to a group located at the first or last nucleoside in a polynucleotide. A 5′-terminal group is a terminal group bonded to 5′-carbon atom of the first nucleoside within a polynucleotide. A 3′-terminal group is a terminal group bonded to 3′-carbon atom of the last nucleoside within a polynucleotide.

The term “terminal nucleoside,” as used herein, refers to a nucleoside that is located within 5 contiguous nucleotides including the nucleoside, in which only one of the 5′ and 3′ positions is attached to a phosphate, phosphorothioate, phosphoramidate, or phosphonate bonded to another nucleotide.

The term “therapeutically effective dose,” as used herein, represents the quantity of an siRNA, or polynucleotide according to the invention necessary to ameliorate, treat, or at least partially arrest the symptoms of a disease or disorder (e.g., to inhibit cellular proliferation). Amounts effective for this use will, of course, depend on the severity of the disease and the weight and general state of the subject.

Typically, dosages used in vitro may provide useful guidance in the amounts useful for in vivo administration of the pharmaceutical composition, and animal models may be used to determine effective dosages for treatment of particular disorders.

The term “thiocarbonyl,” as used herein, represents a C(═S) group. Non-limiting example of functional groups containing a “thiocarbonyl” includes thioesters, thioketones, thioaldehydes, thioanhydrides, thioacyl chlorides, thioamides, thiocarboxylic acids, and thiocarboxylates.

The term “thiol,” as used herein, represents an —SH group.

The term “disorder,” as used herein, is intended to be generally synonymous, and is used interchangeably with, the terms “disease,” “syndrome,” and “condition” (as in a medical condition), in that all reflect an abnormal condition presented by a subject, or one of its parts, that impairs normal functioning, and is typically manifested by distinguishing signs and symptoms.

The term “treating” as used in reference to a disorder in a subject, is intended to refer to reducing at least one symptom of the disorder by administrating a therapeutic (e.g., a nucleotide construct of the invention) to the subject.

As used herein and in the appended claims, the singular forms “a,” “and,” and “the” include plural referents unless the context clearly dictates otherwise. Thus, for example, reference to “a targeting moiety” includes a plurality of such targeting moieties, and reference to “the cell” includes reference to one or more cells known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Similarly, “comprise,” “comprises,” “comprising,” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.” For purposes of this disclosure, any term present in the art which is identical to any term expressly defined in this disclosure, the term's definition presented in this disclosure will control in all respects.

### BRIEF DESCRIPTION OF THE DRAWINGS

FIG. 1 is a chart showing structures of certain phosphotriesters.

FIG. 2 is an image of a gel showing the serum stability of the hybridized polynucleotide constructs including phosphotriesters relative to that of the hybridized polynucleotide constructs lacking phosphotriesters.

FIGS. 3-15 are graphs showing AT3 gene expression levels over time in vivo after 0.5 mg/kg dosing with a hybridized polynucleotide constructs or with saline.

### DETAILED DESCRIPTION

The invention provides a hybridized polynucleotide construct containing a passenger strand, a guide strand loadable into a RISC complex, and one or more auxiliary moieties described herein (e.g., from 1 to 5 or from 1 to 3 auxiliary moieties) linked to a phosphate or a phosphorothioate in the passenger strand or the guide strand. At least one (e.g., all) of the auxiliary moieties may be non-bioreversibly linked to a phosphate or phosphorothioate (e.g., an internucleoside phosphate or phosphorothioate) in the passenger strand. The hybridized polynucleotide construct may include one or more auxiliary moieties (e.g., from 1 to 3 auxiliary moieties) bioreversibly linked to a phosphate or phosphorothioate (e.g., an internucleoside phosphate or phosphorothioate) in the passenger strand. The hybridized polynucleotide construct may include one or more auxiliary moieties (e.g., from 1 to 3 auxiliary moieties) bioreversibly linked to a phosphate or phosphorothioate (e.g., an internucleoside phosphate or phosphorothioate) in the guide strand. In some embodiments, the auxiliary moieties in the hybridized polynucleotide construct are linked to the passenger strand. At least some of the auxiliary moieties may be linked to internucleoside phosphates or phosphorothioates in the following pattern: —N-p^{L}-(—N-p-)_{z}-N-p^{L}-(—N-p-)_{z}-N-p^{L}-[(—N-p-)_{z}-N-p^{L}-]_{z1}-, where each N is independently a nucleoside; each p^{L} is a phosphate or phosphorothioate linked (e.g., non-bioreversibly) to an auxiliary moiety; each p is independently a phosphate, phosphorothioate, phosphoramidate, or phosphonate; each z is independently 0, 1, or 2; and z1 is 0, 1, or 2.

Typically, the hybridized polynucleotide constructs disclosed herein include one or more internucleoside phosphotriesters, internucleoside phosphonates, or internucleoside phosphorothioates connecting two or more of the five contiguous 5′-terminal nucleosides (e.g., in the guide strand or in the passenger strand) and the five contiguous 3′-terminal nucleosides (e.g., in the guide strand or in the passenger strand). Certain hybridized polynucleotide constructs include one or more internucleoside phosphotriesters, internucleoside phosphonates, or internucleoside phosphorothioates connecting two or more of the five contiguous 5′-terminal nucleosides in the passenger strand, the five contiguous 3′-terminal nucleosides in the passenger strand, the five contiguous 5′-terminal nucleosides in the guide strand, and the five contiguous 3′-terminal nucleosides in the guide strand.

The hybridized polynucleotide construct may include at least one (e.g., 1, 2, or 3) internucleoside phosphorothioates, each of the internucleoside phosphorothioates linking two contiguous nucleosides of the four 3′-terminal nucleosides in the guide strand. The hybridized polynucleotide construct may include at least one (e.g., 1, 2, or 3) internucleoside phosphorothioates, each of the internucleoside phosphorothioates linking two contiguous nucleosides of the four 5′-terminal nucleosides in the guide strand. The hybridized polynucleotide construct may include at least one (e.g., 1, 2, or 3) internucleoside phosphorothioates, each of the internucleoside phosphorothioates linking two contiguous nucleosides of the four 3′-terminal nucleosides in the passenger strand. The hybridized polynucleotide construct may include at least one (e.g., 1, 2, or 3) internucleoside phosphorothioates, each of the internucleoside phosphorothioates linking two contiguous nucleosides of the four 5′-terminal nucleosides in the passenger strand.

In some embodiments, each of the passenger strand and the guide strand may independently have the structure of the following formula:

5′-D-(Nuc-E)ₙ-Nuc-F, or a salt thereof,

where

each n is independently an integer from 10 to 150 (e.g., from 14 to 99, from 18 to 49, or from 18 to 31),

each Nuc is independently a nucleoside; and

D of the guide strand is hydroxyl, phosphate, phosphorothioate, or a bioreversible linker bonded to an auxiliary moiety (e.g., a linker containing phosphate or phosphorothioate bonded to Nuc);

D of the passenger strand is H, hydroxyl, optionally substituted C₁₋₆ alkoxy, a protected hydroxyl group, phosphate optionally substituted with C₃₋₈ alkynyl, phosphorothioate optionally substituted with C₃₋₈ alkynyl, diphosphate, triphosphate, tetraphosphate, pentaphosphate, a 5′ cap, an optionally substituted C₁₋₆ alkyl, a biotin containing group, a digoxigenin containing group, a cholesterol containing group, a dye containing group, a quencher containing group, or a non-bioreversible or bioreversible linker bonded to an auxiliary moiety (e.g., a linker containing a phosphate or a phosphorothioate bonded to Nuc);

each E of the passenger strand is independently a phosphate, a phosphorothioate, a phosphoramidate, or a phosphonate, where, optionally, the phosphate or the phosphorothioate is bioreversibly or non-bioreversibly linked to an auxiliary moiety;

each E of the guide strand is independently a phosphate, a phosphorothioate, or a phosphonate, where, optionally, the phosphate or the phosphorothioate is bioreversibly linked to an auxiliary moiety;

each F is independently H, hydroxyl, optionally substituted C₁₋₆ alkoxy, a protected hydroxyl group, phosphate optionally substituted with C₃₋₈ alkynyl, phosphorothioate optionally substituted with C₃₋₈ alkynyl, diphosphate, triphosphate, tetraphosphate, pentaphosphate, an optionally substituted C₁₋₆ alkyl, a biotin containing group, a digoxigenin containing group, a dye containing group, a quencher containing group, or a non-bioreversible or bioreversible linker bonded to an auxiliary moiety (e.g., a linker containing a phosphate or a phosphorothioate bonded to Nuc). At least one E is the phosphate or phosphorothioate non-bioreversibly linked to the auxiliary moiety.

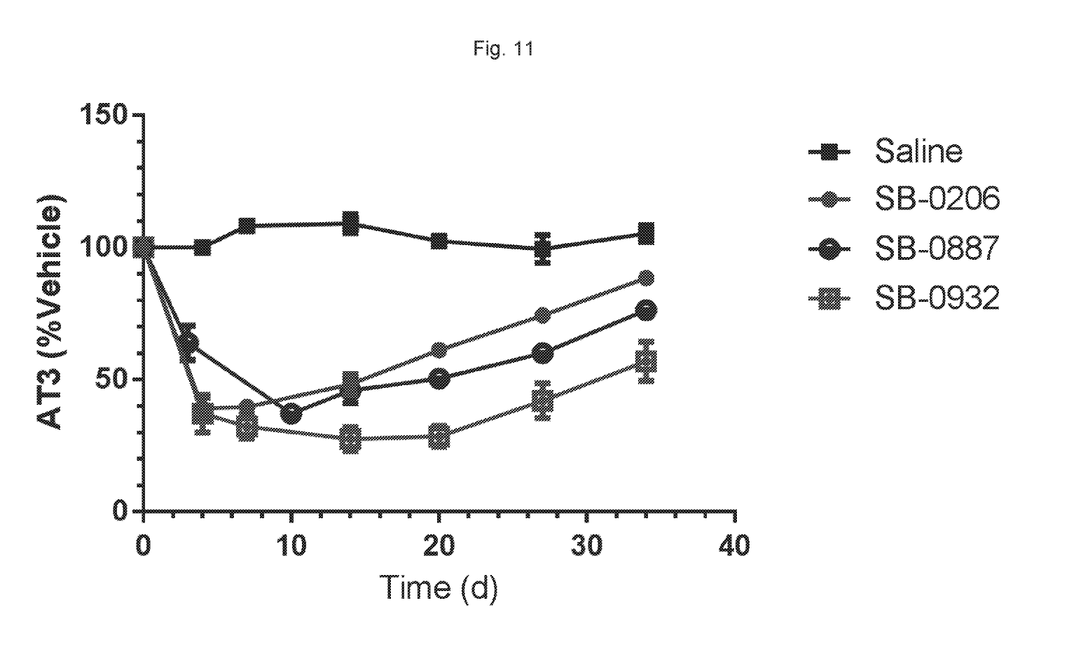

The auxiliary moiety may be a targeting moiety. The targeting moieties having a single ligand are advantageous when multiple (e.g., 3) targeting moieties (e.g., multiple copies of the same targeting moiety) are proximally disposed within the polynucleotide constructs. Such polynucleotide constructs may exhibit prolonged activity relatively to polynucleotide constructs having the same number of ligands disposed within a single targeting moiety. For example, FIG. 11 shows that SB-0932 (SEQ ID NOs.: 25 and 6), which includes three monomeric auxiliary moieties proximally disposed within the passenger strand, exhibits prolonged and potent activity relatively to SB-0206 (SEQ ID NOs.: 3 and 4) and SB-0887 (SEQ ID NOs.: 10 and 6), each of which includes a single trimeric auxiliary moiety (also see Table 4).

The hybridized polynucleotide construct may include a guide strand having 19 or more nucleosides. The guide strand may have fewer than 100 nucleosides (e.g., fewer than 50 nucleosides or fewer than 32 nucleosides). The hybridized polynucleotide construct may include a passenger strand having 19 or more nucleosides. The passenger strand may have fewer than 100 nucleosides (e.g., fewer than 50 nucleosides or fewer than 32 nucleosides). Preferably each of the passenger and guide strand will independently include from 19 to 50 nucleosides (e.g., from 19 to 32 nucleoside). The passenger and guide strands can be complimentary to each other over at least 12 contiguous nucleosides (e.g., over at least 15 contiguous nucleosides).

In addition to the moieties described above, the hybridized polynucleotide construct may contain one or more of non-bioreversible phosphotriesters, bioreversible phosphotriesters, phosphoramidates, and phosphonates.

The 5′- or 3′-terminus or both termini of the passenger strand may include non-bioreversible phosphodiesters, which differ from non-bioreversible phosphotriesters described herein only in that the phosphodiester includes —OH or —O— (e.g., a salt) bonded to the phosphorus atom of the phosphodiester.

The 5′- or 3′-terminus or both termini of the passenger strand may include bioreversible phosphodiesters, which differ from bioreversible phosphotriesters described herein only in that the phosphodiester includes —OH or —O— (e.g., a salt) bonded to the phosphorus atom of the phosphodiester. The 5′- or 3′-terminus or both termini of the guide strand may include non-bioreversible phosphodiesters, which differ from non-bioreversible phosphotriesters described herein only in that the phosphodiester includes —OH or —O— (e.g., a salt) bonded to the phosphorus atom of the phosphodiester. The 5′- or 3′-terminus or both termini of the guide strand may include bioreversible phosphodiesters, which differ from bioreversible phosphotriesters described herein only in that the phosphodiester includes —OH or —O— (e.g., a salt) bonded to the phosphorus atom of the phosphodiester.

The hybridized polynucleotide construct may further include a second passenger strand and optionally a second guide strand. The second passenger strand may be bioreversibly linked to the first passenger strand of the hybridized polynucleotide construct. The second guide strand, when present, may be hybridized to the second passenger strand.

Any nucleic acid, regardless of sequence composition, can be modified. Accordingly, the invention is not limited to any particular sequence (e.g., any particular siRNA). Also, polynucleotide constructs disclosed in WO 2015/069932 and in WO 2015/188197 may be modified to include auxiliary moieties as described herein; the disclosure of polynucleotide constructs disclosed in WO 2015/069932 and in WO 2015/188197 is incorporated herein by reference.

These hybridized polynucleotide constructs may exhibit a superior efficacy in gene silencing relative the hybridized polynucleotide constructs that differ only by the absence of the internucleoside phosphate or phosphorothioate that is non-bioreversibly linked to an auxiliary moiety. Without being bound by theory, the superior efficacy may be due to an improvement in the kinetics of the RISC complex loading or an improvement in the stability of the hybridized polynucleotide construct.

The invention provides compositions and methods to facilitate and improve the cellular uptake of polynucleotides by reducing or neutralizing the charge associated with anionically charged polynucleotides, and adding further functionality to the molecule, e.g., by including one or more auxiliary moieties.

The invention provides compositions and methods for the delivery of sequence specific polynucleotides useful for selectively treating human disorders and for promoting research. The compositions and methods of the invention effectively deliver polynucleotides (e.g., siRNAs) to subjects and to cells. The invention provides compositions and methods which overcome size and charge limitations that make RNAi constructs difficult to deliver into cells or make the constructs undeliverable.

By neutralizing the anionic charge of nucleic acids (e.g., dsRNA), a nucleotide construct comprising a bioreversible group according to the invention can deliver nucleic acids into a cell in vitro and in vivo.

The invention provides compositions and methods for the delivery of nucleotide constructs containing one or more targeting moieties for targeted delivery to specific cells (e.g., cells having asialoglycoprotein receptors on their surface (e.g., hepatocytes), tumor cells (e.g., tumor cells having folate receptors on their surface), cells bearing mannose receptor (e.g., macrophages, dendritic cells, and skin cells (e.g., fibroblasts or keratinocytes))). Non-limiting examples of mannose receptor superfamily include MR, Endo180, PLA2R, MGL, and DEC205. Targeted delivery of the nucleotide constructs of the invention may involve receptor mediated internalization. In some embodiments, targeting moieties may include mannose, N-acetyl galactosamine (GalNAc), or a folate ligand. In other embodiments, a targeting moiety may include one or more (e, g., from 1 to 5 or from 1 to 3) antibodies or antigen-binding fragments thereof. Certain targeting moieties include one antibody or antigen-binding fragment thereof.

The invention provides hybridized polynucleotide constructs having one or more non-bioreversible, and optionally bioreversible, moieties that contribute to chemical and biophysical properties that enhance cellular membrane penetration and resistance to exo- and endonuclease degradation. The invention further provides reagents for the synthesis of the hybridized polynucleotide constructs disclosed herein, e.g., phosphoramidate reagents.

In cells, the bioreversible moieties can be removed by the action of enzymes (e.g., enzymes having thioreductase activity (e.g., protein disulfide isomerase or thioredoxin)) or by exposure to the intracellular conditions (e.g., an oxidizing or reducing environment) or reactants (e.g., glutathione or other free thiol) to yield biologically active polynucleotide compounds that are capable of hybridizing to and/or having an affinity for specific endogenous nucleic acids.

Auxiliary Moieties Non-Bioreversibly Linked to Polynucleotide Constructs

The hybridized polynucleotide constructs of the invention may include an auxiliary moiety (e.g., a targeting moiety) non-bioreversibly linked to a phosphate or a phosphorothioate in the passenger strand of the hybridized polynucleotide construct. The auxiliary moiety can be non-bioreversibly linked to the phosphate or the phosphorothioate by a process described in the sections below. In some instances, the auxiliary moiety may be non-bioreversibly linked to the phosphate or the phosphorothioate through a linker containing 1,2,3-triazole or N-sulfonylamidocarbonyl. For example, the auxiliary moiety may combine with the non-bioreversible linker to form a group that is

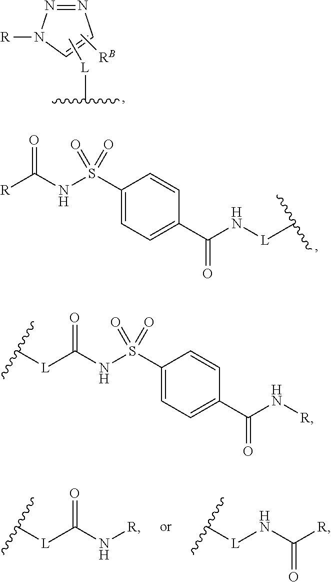

where

R is said auxiliary moiety (e.g., a targeting moiety);

R^{B} is H or C₁₋₆ alkyl; and L is C₂₋₆ alkylene or —(CH₂CH₂O)ₚ₁(CH₂CH₂)—, where p1 is an integer from 1 to 50 (e.g., from 1 to 3, from 1 to 8, from 1 to 10, from 1 to 20, from 1 to 30, or from 1 to 40).

In some embodiments, the auxiliary moieties in the hybridized polynucleotide construct are linked to the passenger strand. At least some of the auxiliary moieties may be linked to internucleoside phosphates or phosphorothioates in the following pattern: —N-p^{L}-(—N-p-)z-N-p^{L}-(—N-p-)z-N-p^{L}-[(—N-p-)z-N-p^{L}]_{z1}-, where each N is independently a nucleoside; each p^{L} is a phosphate or phosphorothioate non-bioreversibly linked to an auxiliary moiety; each p is independently a phosphate, phosphorothioate, phosphoramidate, or phosphonate; each z is independently 0, 1, or 2; and z1 is 0, 1, or 2.

Auxiliary Moieties Bioreversibly Linked to Polynucleotide Constructs

The hybridized polynucleotide constructs of the invention may include an auxiliary moiety bioreversibly linked to the passenger strand or the guide strand (e.g., a targeting moiety bioreversibly linked to a phosphate or phosphorothioate). The bioreversible linker connecting the auxiliary moiety to the passenger strand or the guide strand may include —S—S—. For example, the bioreversible linker may combine with the auxiliary moiety to form R-(Link C)—S—S-(Link A)-, where R is the auxiliary moiety; Link A is a divalent or a trivalent linker containing an sp³-hybridized carbon atom bonded to the phosphate or phosphorothioate and a carbon atom bonded to —S—S—, where the shortest chain of atoms between —S—S— and the phosphate or the phosphorothioate is at least 3 atoms long (e.g., the shortest chain of atoms between —S—S— and the phosphate or the phosphorothioate is from 3 to 6 (e.g., 4) atoms long); and Link C is a bond or a divalent or a trivalent linker having a molecular weight of from 13 Da to 1 kDa;
where, when Link A is a trivalent linker, Link C is a trivalent linker and the third valency of Link A combines with —S—S— and Link C to form optionally substituted C₃₋₉ heterocyclylene or optionally substituted (C₃₋₉ heterocyclyl)-C₁₋₄-alkylene.

In some embodiments, the auxiliary moieties in the hybridized polynucleotide construct are linked to the passenger strand. At least some of the auxiliary moieties may be linked to internucleoside phosphates or phosphorothioates in the following pattern: —N-p^{L}-(—N-p-)_{z}-N-p^{L}-(—N-p-)_{z}-N-p^{L}-[(—N-p-)_{z}-N-p^{L}]_{z1}-, where each N is independently a nucleoside; each p^{L} is a phosphate or phosphorothioate bioreversibly linked to an auxiliary moiety; each p is independently a phosphate, phosphorothioate, phosphoramidate, or phosphonate; each z is independently 0, 1, or 2; and z1 is 0, 1, or 2.

Link A can be optionally substituted C₃₋₆ alkylene, optionally substituted (C₆₋₁₄ aryl)-C₁₋₄ alkylene, optionally substituted (C₁₋₉ heteroaryl)-C₁₋₄ alkylene, or optionally substituted (C₂₋₉ heterocyclyl)-C₁₋₄ alkylene. For example, Link A can be

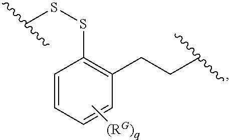

where

R^{G} is a halogen or optionally substituted C₁₋₆ alkyl, and

q is an integer from 0 to 4 (e.g., q is 0).

Link C can include 1,2,3-triazole bonding to R. For example, Link C can combine with R, —S—S—, and Link A to form:

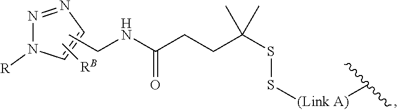

where R^{B} is H or C₁₋₆ alkyl.

Including sterically-hindered disulfides in the bioreversible linkers is particularly advantageous. Disulfides bonded to at least one bulky group exhibit greater stability during the polynucleotide construct synthesis compared to disulfides that are not bonded to at least one bulky group, as the latter may react with a phosphorus (III) atom of the nucleotide construct to cleave the disulfide bond.

Non-Bioreversible Phosphotriesters

The hybridized polynucleotide constructs of the invention may also include a non-bioreversible phosphotriester (e.g., a phosphate or a phosphorothioate that is substituted with a group that does not include a disulfide or a thioester). The non-bioreversible phosphotriester can be an internucleoside non-bioreversible phosphotriester (e.g., a non-bioreversible phosphotriester disposed outside the seed region of the hybridized polynucleotide construct). Preferred positions for internucleoside non-bioreversible phosphotriesters in the guide strand are those between the second and third nucleosides, the fifth and the sixth nucleosides, the seventeenth and the eighteenth nucleosides, the nineteeneth and the twentieth nucleosides, or the twentieth and the twenty first nucleosides (the count starts at 5′-terminus of the guide strand). Preferred positions for the non-bioreversible phosphotriesters in the passenger strand are those that do not connect two contiguous nucleosides at the natural RISC-mediated cleavage site.

The non-bioreversible phosphotriester may be a phosphate or a phosphorothioate substituted with a substituent selected independently from the group consisting of optionally substituted C₂₋₁₆ alkyl; optionally substituted C₃₋₁₆ alkenyl; optionally substituted C₃₋₁₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O, and S; optionally substituted C₂₋₉ heterocyclyl having 1 to 4 heteroatoms selected from N, O, and S, where the heterocyclyl does not contain an S—S bond; optionally substituted (C₂₋₉ heterocyclyl)-C₁₋₄-alkyl having 1 to 4 heteroatoms selected from N, O, and S, where the heterocyclyl does not contain an S—S bond; and a group of the following structure:

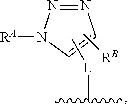

where
L is optionally substituted C₂₋₁₆ alkylene;
R^{A} is optionally substituted C₂₋₆ alkyl; optionally substituted C₆₋₁₄ aryl; optionally substituted (C₆₋₁₄ aryl)-C₁₋₄-alkyl; optionally substituted C₃₋₈ cycloalkyl; optionally substituted (C₃₋₈ cycloalkyl)-C₁₋₄-alkyl; optionally substituted C₁₋₉ heteroaryl having 1 to 4 heteroatoms selected from the group consisting of N, O, and S; optionally substituted (C₁₋₉ heteroaryl)-C₁₋₄-alkyl having 1 to 4 heteroatoms selected from the group consisting of N, O, and S; optionally substituted C₂₋₉ heterocyclyl having 1 to 4 heteroatoms selected from the group consisting of N, O, and S, wherein said heterocyclyl does not comprise an S—S bond; optionally substituted (C₂₋₉ heterocyclyl)-C₁₋₄-alkyl having 1 to 4 heteroatoms selected from N, O, and S, wherein said heterocyclyl does not comprise an S—S bond; and a poly(ethylene glycol) terminated with —OH, C₁₋₆ alkoxy, or —COOH; and
R^{B} is H or optionally substituted C₁₋₆ alkyl.

The non-bioreversible phosphotriester may be a phosphate or a phosphorothioate substituted with a substituent that is

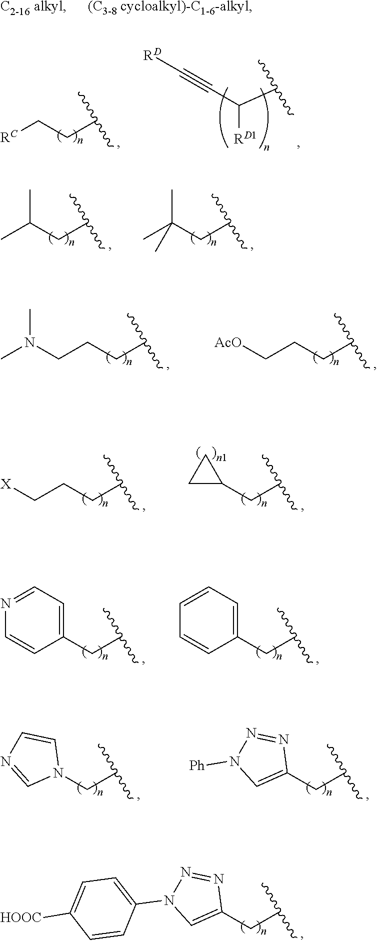

or a group formed by cycloaddition reaction of

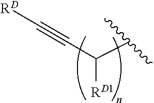

with an azido-containing substrate,

where

n is an integer from 1 to 6;

n1 is an integer from 1 to 6 (e.g., from 1 to 4);

R^{C} is optionally substituted C₆ aryl; optionally substituted C₄₋₅ heteroaryl that is a six member ring comprising 1 or 2 nitrogen atoms; or optionally substituted C₄₋₅ heterocyclyl that is a six member ring comprising 1 or 2 nitrogen atoms;

R^{D} is H or C₁₋₆ alkyl;

each R^{D1} is independently H or C₁₋₆ alkyl, provided that

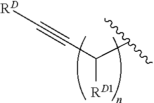

n contains 24 carbon atoms or fewer;

X is a halogen, COOR¹, or —CONR²₂, where each of R¹ and R² is independently H, optionally substituted C₁₋₆ alkyl, optionally substituted C₆₋₁₄ aryl, optionally substituted C₁₋₉ heteroaryl, or optionally substituted C₂₋₉ heterocyclyl; and

the azido-containing substrate is

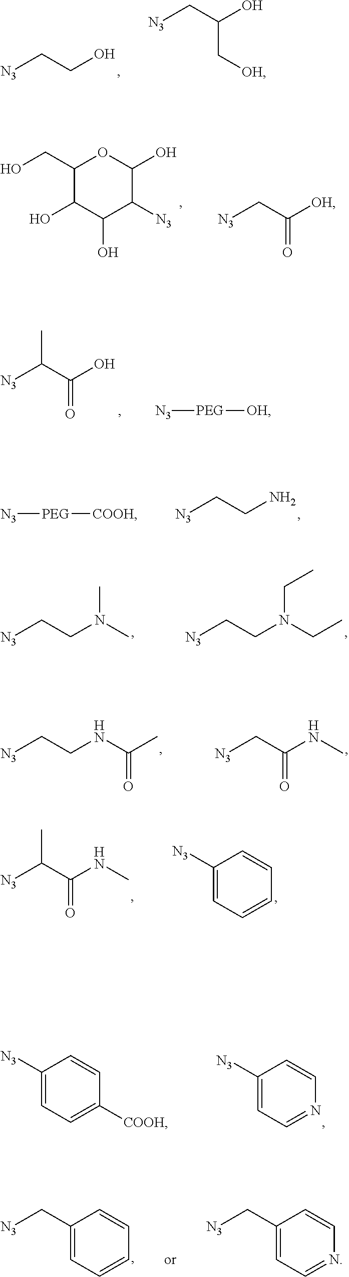

Bioreversible Phosphotriesters

The hybridized polynucleotide constructs of the invention may also include a bioreversible phosphotriester (e.g., a phosphate or a phosphorothioate that is substituted with a group that includes a disulfide linked to the phosphate or the phosphorothioate through a linker includes sp³-carbon bonded to the phosphate and that includes the shortest chain of atoms of 3 to 6 atoms between disulfide and the phosphate or the phosphorothioate).

The bioreversible phosphotriester may be a phosphate or a phosphorothioate substituted with -(Link A)-S—S—R^{E}, in which

Link A is a divalent or a trivalent linker containing an sp³-hybridized carbon atom bonded to the phosphate or phosphorothioate and a carbon atom bonded to —S—S—, where, when Link A is a trivalent linker, the third valency of Link A combines with —S—S— and R^{E} to form optionally substituted 03-9 heterocyclylene, and

R^{E} is optionally substituted C₂₋₈ alkyl; optionally substituted C₃₋₈ alkenyl; optionally substituted C₃₋₈ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O, and S; optionally substituted C₂₋₉ heterocyclyl having 1 to 4 heteroatoms selected from N, O, and S; optionally substituted (C₂₋₉ heterocyclyl)-C₁₋₄-alkyl having 1 to 4 heteroatoms selected from N, O, and S; or, when Link A is a trivalent linker, R^{E} combines with —S—S— and Link A to form optionally substituted C₃₋₉ heterocyclyl.

The bioreversible phosphotriester may be a phosphate or a phosphorothioate substituted with a group that is

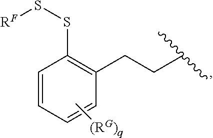

where

R^{F} is optionally substituted C₁₋₆ alkyl or optionally substituted C₆₋₁₄ aryl (e.g., R^{F} is optionally substituted C₁₋₆ alkyl),

R^{G} is a halogen or optionally substituted C₁₋₆ alkyl, and

q is an integer from 0 to 4 (e.g., q is 0).

In any of the bioreversible linkers described herein, —S—S— may be replaced with —C(O)—S—.

Including sterically-hindered disulfides in the bioreversible phosphotriesters is particularly advantageous. Disulfides bonded to at least one bulky group exhibit greater stability during the nucleotide construct synthesis compared to disulfides that are not bonded to at least one bulky group, as the latter may react with a phosphorus (III) atom of the nucleotide construct to cleave the disulfide bond.

Auxiliary Moieties

Various auxiliary moieties can be conjugated to the polynucleotide constructs of the invention, and the auxiliary moieties can provide desirable biological or chemical effects. Biological effects include, but are not limited to, inducing intracellularization, binding to a cell surface, targeting a specific cells type, allowing endosomal escape, altering the half-life of the polynucleotide in vivo, and providing a therapeutic effect. Chemical effects include, but are not limited to, changing the solubility, charge, size, and reactivity.

Targeting Moieties

The hybridized polynucleotide constructs disclosed herein may include one or more targeting moieties as auxiliary moieties. A targeting moiety is selected based on its ability to target constructs of the invention to a desired or selected cell population that expresses the corresponding binding partner (e.g., either the corresponding receptor or ligand) for the selected targeting moiety. For example, a construct of the invention could be targeted to hepatocytes expressing asialoglycoprotein (ASGP-R) by selecting a targeting moiety containing N-acetyl galactosamine (GalNAc) as the ligand. A targeting moiety (i.e., an intracellular targeting moiety) that targets a desired site within the cell (e.g., endoplasmic reticulum, Golgi apparatus, nucleus, or mitochondria) may be included in the hybridized polynucleotide constructs disclosed herein. Non-limiting examples of the intracellular targeting moieties are provided in WO 2015/069932 and in WO 2015/188197; the disclosure of the intracellular targeting moieties in WO 2015/069932 and in WO 2015/188197 is incorporated herein by reference.

A polynucleotide construct of the invention, thus, may include one or more targeting moieties selected from the group consisting of intracellular targeting moieties, extracellular targeting moieties, and combinations thereof. Thus, the inclusion of one or more targeting moieties (e.g., extracellular targeting moieties including ligands independently selected from the group consisting of folate, mannose, N-acetyl galactosamine, or prostate specific membrane antigen) and one or more intracellular targeting moiety (e.g., a moiety targeting endoplasmic reticulum, Golgi apparatus, nucleus, or mitochondria) in the polynucleotide construct of the invention can facilitate the delivery of the polynucleotides to a specific site within the specific cell population. In some embodiments, the targeting moiety contains one or more mannose carbohydrates. Mannose targets the mannose receptor, which is a 175 KDa membrane-associated receptor that is expressed on sinusoidal liver cells and antigen presenting cells (e.g., macrophages and dendritic cells). It is a highly effective endocytotic/recycling receptor that binds and internalizes mannosylated pathogens and proteins (Lennartz et. al. J. Biol. Chem. 262:9942-9944, 1987; Taylor et. al. J. Biol. Chem. 265:12156-62, 1990).

Some of the targeting moieties of the invention are described herein. In some embodiments, the targeting moiety contains or specifically binds to a protein selected from the group including insulin, insulin-like growth factor receptor 1 (IGF1R), IGF2R, insulin-like growth factor (IGF; e.g., IGF 1 or 2), mesenchymal epithelial transition factor receptor (c-met; also known as hepatocyte growth factor receptor (HGFR)), hepatocyte growth factor (HGF), epidermal growth factor receptor (EGFR), epidermal growth factor (EGF), heregulin, fibroblast growth factor receptor (FGFR), platelet-derived growth factor receptor (PDGFR), platelet-derived growth factor (PDGF), vascular endothelial growth factor receptor (VEGFR), vascular endothelial growth factor (VEGF), tumor necrosis factor receptor (TNFR), tumor necrosis factor alpha (TNF-α), TNF-β, folate receptor (FOLR), folate, transferrin, transferrin receptor (TfR), mesothelin, Fc receptor, c-kit receptor, c-kit, an integrin (e.g., an α4 integrin or a β-1 integrin), P-selectin, sphingosine-1-phosphate receptor-1 (S1PR), hyaluronate receptor, leukocyte function antigen-1 (LFA-1), CD4, CD11, CD18, CD20, CD25, CD27, CD52, CD70, CD80, CD85, CD95 (Fas receptor), CD106 (vascular cell adhesion molecule 1 (VCAM1), CD166 (activated leukocyte cell adhesion molecule (ALCAM)), CD178 (Fas ligand), CD253 (TNF-related apoptosis-inducing ligand (TRAIL)), ICOS ligand, CCR2, CXCR3, CCR5, CXCL12 (stromal cell-derived factor 1 (SDF-1)), interleukin 1 (IL-1), IL-1ra, IL-2, IL-3, IL-4, IL-6, IL-7, IL-8, CTLA-4, MART-1, gp100, MAGE-1, ephrin (Eph) receptor, mucosal addressin cell adhesion molecule 1 (MAdCAM-1), carcinoembryonic antigen (CEA), Lewis^{Y}, MUC-1, epithelial cell adhesion molecule (EpCAM), cancer antigen 125 (CA125), prostate specific membrane antigen (PSMA), TAG-72 antigen, and fragments thereof. In further embodiments, the targeting moiety contains erythroblastic leukemia viral oncogene homolog (ErbB) receptor (e.g., ErbB1 receptor; ErbB2 receptor; ErbB3 receptor; and ErbB4 receptor). In some embodiments, the targeting moiety contains one or more (e.g., from 1 to 6) N-acetyl galactosamines (GalNAc). In certain embodiments, the targeting moiety contains one or more (e.g., from 1 to 6) mannoses. In other embodiments, the targeting moiety contains a folate ligand. The folate ligand has the structure:

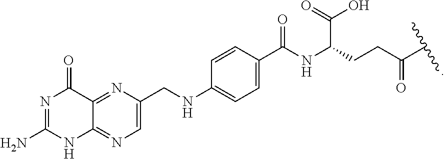 [SMILES: *C(=O)CC[C@H](NC(=O)c1ccc(NCc2cnc3[nH]c(N)nc(=O)c3n2)cc1)C(=O)O]

Certain targeting moieties may include bombesin, gastrin, gastrin-releasing peptide, tumor growth factors (TGF) (e.g., TGF-α or TGF-β), or vaccinia virus growth factor (VVGF). Non-peptidyl ligands can also be used in the targeting moieties and may include, for example, steroids, carbohydrates, vitamins, and lectins. Some targeting moieties may include a polypeptide, such as somatostatin or somatostatin analog (e.g., octreotide or lanreotide), bombesin, or an antibody or antigen-binding fragment thereof. Antibodies may be of any recognized class or subclass, e.g., IgG, IgA, IgM, IgD, or IgE. Typical are those antibodies which fall within the IgG class. The antibodies can be derived from any species according techniques known in the art. Typically, however, the antibody is of human, murine, or rabbit origin. In addition, the antibody may be polyclonal or monoclonal, but is typically monoclonal. Human or chimeric (e.g., humanized) antibodies may be used in targeting moieties. Targeting moieties may include an antigen-binding fragment of an antibody. Such antibody fragments may include, for example, the Fab′, F(ab′)2, Fv, or Fab fragments, singledomain antibody, ScFv, or other antigen-binding fragments. Fc fragments may also be employed in targeting moieties. Such antibody fragments can be prepared, for example, by proteolytic enzyme digestion, for example, by pepsin or papain digestion, reductive alkylation, or recombinant techniques. The materials and methods for preparing antibody fragments are well-known to those skilled in the art. See, e.g., Parham, J. Immunology, 131:2895, 1983; Lamoyi et al., J. Immunological Methods, 56:235, 1983.

Other peptides for use as a targeting auxiliary moiety in nucleotide constructs of the invention can be selected from KiSS peptides and analogs, urotensin II peptides and analogs, GnRH I and II peptides and analogs, depreotide, vapreotide, vasoactive intestinal peptide (VIP), cholecystokinin (CCK), RGD-containing peptides, melanocyte-stimulating hormone (MSH) peptide, neurotensin, calcitonin, glutathione, YIGSR (leukocyte-avid peptides, e.g., P483H, which contains the heparin-binding region of platelet factor-4 (PF-4) and a lysine-rich sequence), atrial natriuretic peptide (ANP), β-amyloid peptides, delta-opioid antagonists (such as ITIPP(psi)), annexin-V, endothelin, leukotriene B4 (LTB4), chemotactic peptides (e.g., N-formyl-methionyl-leucyl-phenylalanine-lysine (fMLFK), GP Ilb/Illa receptor antagonists (e.g., DMP444), human neutrophil elastase inhibitor (EPI-HNE-2 and EPI-HNE-4), plasmin inhibitor, antimicrobial peptides, apticide (P280 and P274), thrombospondin receptor (including analogs such as TP-1300), bitistatin, pituitary adenylyl cyclase type I receptor (PAC1), fibrin α-chain, peptides derived from phage display libraries, and conservative substitutions thereof.

The targeting moiety can include a non-bioreversible linker linking ligand(s) in the targeting moiety to the conjugating moiety or to the reaction product thereof (e.g., 1,2,3-triazole). The non-bioreversible linker can include one or more monomers, where each monomer is independently optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; imino; optionally substituted N; O; or S(O)ₘ, wherein m is 0, 1, or 2. In some embodiments, each monomer is independently optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; imino; optionally substituted N; O; or S(O)ₘ, where m is 0, 1, or 2 (e.g., m is 2). In certain embodiments, each monomer is independently optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; O; or S(O)ₘ, where m is 0, 1, or 2 (e.g., m is 2). The non-bioreversible linker connecting the ligand to the conjugating moiety or to the reaction product thereof can include from 2 to 500 (e.g., from 2 to 300 or from 2 to 200) of such monomers. The non-bioreversible linker may include a poly(alkylene oxide) (e.g., polyethylene oxide, polypropylene oxide, poly(trimethylene oxide), polybutylene oxide, poly(tetramethylene oxide), and diblock or triblock copolymers thereof). In some embodiments, the non-bioreversible linker includes polyethylene oxide (e.g., poly(ethylene oxide) having a molecular weight of less than 1 kDa).

In some embodiments, the targeting moiety includes one or more (e.g., from 1 to 6 or from 1 to 3) GalNAc ligands. GalNAc ligand may be attached to a linker (e.g., as a ketal or a hemiaminal) which is further attached to a conjugating moiety or a reaction product thereof (e.g., 1,2,3-triazole). The linker can be as described herein. GalNAc ligands attached to a linker through a hemiaminal may produce hybridized polynucleotide constructs having superior efficacy in gene silencing as compared to hybridized polynucleotide constructs having GalNAc ligand(s) attached to a linker through a ketal.

Endosomal Escape Moieties

The invention provides for one or more endosomal escape moieties which can be attached to a nucleotide construct disclosed herein as an auxiliary moiety, for example, as an endosomal escape auxiliary moiety. Exemplary endosomal escape moieties include chemotherapeutics (e.g., quinolones such as chloroquine); fusogenic lipids (e.g., dioleoylphosphatidyl-ethanolamine (DOPE)); and polymers such as polyethylenimine (PEI); poly(beta-amino ester)s; peptides or polypeptides such as polyarginines (e.g., octaarginine) and polylysines (e.g., octalysine); proton sponges, viral capsids, and peptide transduction domains as described herein. For example, fusogenic peptides can be derived from the M2 protein of influenza A viruses; peptide analogs of the influenza virus hemagglutinin; the HEF protein of the influenza C virus; the transmembrane glycoprotein of filoviruses; the transmembrane glycoprotein of the rabies virus; the transmembrane glycoprotein (G) of the vesicular stomatitis virus; the fusion protein of the Sendai virus; the transmembrane glycoprotein of the Semliki forest virus; the fusion protein of the human respiratory syncytial virus (RSV); the fusion protein of the measles virus; the fusion protein of the Newcastle disease virus; the fusion protein of the visna virus; the fusion protein of murine leukemia virus; the fusion protein of the HTL virus; and the fusion protein of the simian immunodeficiency virus (SIV). Other moieties that can be employed to facilitate endosomal escape are described in Dominska et al., Journal of Cell Science, 123(8):1183-1189, 2010. Specific examples of endosomal escape moieties including moieties suitable for conjugation to the hybridized polynucleotide constructs disclosed herein are provided, e.g., in PCT/US2015/034749; the disclosure of these endosomal escape moieties is incorporated by reference herein.

An endosomal escape moiety can include a non-bioreversible linker attaching the endosomal escape moiety to the conjugating moiety or a reaction product thereof (e.g., 1,2,3-triazole). The linker can be as described above for targeting moieties.

Cell Penetrating Peptides

The hybridized polynucleotide constructs disclosed herein may include a cell penetrating peptide (CPP) bioreversibly or non-bioreversibly linked to the hybridized polynucleotide construct. The CPP can be linked to the hybridized polynucleotide bioreversibly through a disulfide linkage, as disclosed herein. Thus, upon delivery to a cell, the CPP can be cleaved intracellularly, e.g., by an intracellular enzyme (e.g., protein disulfide isomerase, thioredoxin, or a thioesterase) and thereby release the polynucleotide.

CPPs are known in the art (e.g., TAT or Arg8) (Snyder and Dowdy, 2005, Expert Opin. Drug Deliv. 2, 43-51). Specific examples of CPPs including moieties suitable for conjugation to the hybridized polynucleotide constructs disclosed herein are provided, e.g., in PCT/US2015/034749; the disclosure of these CPPs is incorporated by reference herein.

CPPs are positively charged peptides that are capable of facilitating the delivery of biological cargo to a cell. It is believed that the cationic charge of the CPPs is essential for their function. Moreover, the transduction of these proteins does not appear to be affected by cell type, and these proteins can efficiently transduce nearly all cells in culture with no apparent toxicity (Nagahara et al., Nat. Med. 4:1449-52, 1998). In addition to full-length proteins, CPPs have also been used successfully to induce the intracellular uptake of DNA (Abu-Amer, supra), antisense polynucleotides (Astriab-Fisher et al., Pharm. Res, 19:744-54, 2002), small molecules (Polyakov et al., Bioconjug. Chem. 11:762-71, 2000) and even inorganic 40 nm iron particles (Dodd et al., J. Immunol. Methods 256:89-105, 2001; Wunderbaldinger et al., Bioconjug. Chem. 13:264-8, 2002; Lewin et al., Nat. Biotechnol. 18:410-4, 2000; Josephson et al., Bioconjug. Chem. 10:186-91, 1999) suggesting that there is considerable flexibility in particle size in this process.

In one embodiment, a CPP useful in the methods and compositions of the invention includes a peptide featuring substantial alpha-helicity. It has been discovered that transfection is optimized when the CPP exhibits significant alpha-helicity. In another embodiment, the CPP includes a sequence containing basic amino acid residues that are substantially aligned along at least one face of the peptide. A CPP useful in the invention may be a naturally occurring peptide or a synthetic peptide.

CPPs can be linked through a non-bioreversible linker to the conjugating moiety or a reaction product thereof (e.g., 1,2,3-triazole).

Polymers

The nucleotide constructs described herein can also include covalently attached neutral polymer-based auxiliary moieties. Neutral polymers include poly(C₁₋₆ alkylene oxide), e.g., poly(ethylene glycol) and poly(propylene glycol) and copolymers thereof, e.g., di- and triblock copolymers. Other examples of polymers include esterified poly(acrylic acid), esterified poly(glutamic acid), esterified poly(aspartic acid), poly(vinyl alcohol), poly(ethylene-co-vinyl alcohol), poly(N-vinyl pyrrolidone), poly(ethyloxazoline), poly(alkylacrylates), poly(acrylamide), poly(N-alkylacrylamides), poly(N-acryloylmorpholine), poly(lactic acid), poly(glycolic acid), poly(dioxanone), poly(caprolactone), styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyurethane, N-isopropylacrylamide polymers, and poly(N,N-dialkylacrylamides). Exemplary polymer auxiliary moieties may have molecular weights of less than 100, 300, 500, 1000, or 5000 Da (e.g., greater than 100 Da). Other polymers are known in the art.

The polymers can be attached to a conjugating moiety or a reaction product thereof (e.g., 1,2,3-triazole).

Preparation of the Polynucleotide Constructs

The invention further provides methods for manufacturing the polynucleotide constructs of the invention. Methods for the preparation of nucleotides and polynucleotides are known in the art. For example, the practice of phosphoramidite chemistry to prepare polynucleotides is known from the published work of Caruthers and Beaucage and others. See, e.g., U.S. Pat. Nos. 4,458,066; 4,500,707; 5,132,418; 4,415,732; 4,668,777; 4,973,679; 5,278,302, 5,153,319; 5,218,103; 5,268,464; 5,000,307; 5,319,079; 4,659,774; 4,672,110; 4,517,338; 4,725,677; and RE34,069, each of which is herein incorporated by reference, describe methods of polynucleotide synthesis. Additionally, the practice of phosphoramidite chemistry has been systematically reviewed by Beaucage et al., Tetrahedron, 48: 2223-2311, 1992; and Beaucage et al., Tetrahedron, 49:6123-6194, 1993, as well as references referred to therein, all of which are herein incorporated by reference. Synthesis principles useful in the synthesis of the polynucleotide constructs of the invention are disclosed in PCT/US2014/064401 and in PCT/US2015/034749; the disclosure of syntheses of polynucleotide constructs in PCT/US2014/064401 and in PCT/US2015/034749 is incorporated herein by reference.

Nucleic acid synthesizers are commercially available, and their use is generally understood by persons of ordinary skill in the art as being effective in generating nearly any polynucleotide of reasonable length which may be desired.

In practicing phosphoramidite chemistry, useful 5′OH sugar blocking groups are trityl, monomethoxytrityl, dimethoxytrityl and trimethoxytrityl, especially dimethoxytrityl (DMTr). In practicing phosphoramidite chemistry, useful phosphite activating groups are dialkyl substituted nitrogen groups and nitrogen heterocycles. One approach includes the use of the di-isopropylamino activating group.

Polynucleotides can be synthesized by a Mermade-6 solid phase automated polynucleotide synthesizer or any commonly available automated polynucleotide synthesizer. Triester, phosphoramidite, or hydrogen phosphonate coupling chemistries (described in, for example, M. Caruthers, Oligonucleotides: Antisense Inhibitors of Gene Expression, pp. 7-24, J. S. Cohen, ed. (CRC Press, Inc. Boca Raton, Fla., 1989); Oligonucleotide synthesis, a practical approach, Ed. M. J. Gait, IRL Press, 1984; and Oligonucleotides and Analogues, A Practical Approach, Ed. F. Eckstein, IRL Press, 1991) are employed by these synthesizers to provide the desired polynucleotides. The Beaucage reagent, as described in, for example, Journal of American Chemical Society, 112:1253-1255, 1990, or elemental sulfur, as described in Beaucage et al., Tetrahedron Letters 22:1859-1862, 1981, is used with phosphoramidite or hydrogen phosphonate chemistries to provide substituted phosphorothioate polynucleotides.

For example, the reagents containing the protecting groups recited herein can be used in numerous applications where protection is desired. Such applications include, but are not limited to, both solid phase and solution phase, polynucleotide synthesis and the like.

For instance, structural groups are optionally added to the ribose or base of a nucleoside for incorporation into a polynucleotide, such as a methyl, propyl or allyl group at the 2′-0 position on the ribose, or a fluoro group which substitutes for the 2′-O group, or a bromo group on the ribonucleoside base. For use with phosphoramidite chemistry, various phosphoramidite reagents are commercially available, including 2′-deoxy phosphoramidites, 2′-O-methyl phosphoramidites and 2′-O-hydroxyl phosphoramidites. Any other means for such synthesis may also be employed. The actual synthesis of the polynucleotides is well within the talents of those skilled in the art. It is also well known to use similar techniques to prepare other polynucleotides such as the phosphorothioates, methyl phosphonates and alkylated derivatives. It is also well known to use similar techniques and commercially available modified phosphoramidites and controlled-pore glass (CPG) products such as biotin, Cy3, fluorescein, acridine or psoralen-modified phosphoramidites and/or CPG (available from Glen Research, Sterling Va.) to synthesize fluorescently labeled, biotinylated or other conjugated polynucleotides.

The phosphoramidite reagents useful for the preparation of the polynucleotide constructs of the invention can be of the following structure:

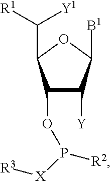

or a salt thereof,

B¹ is a nucleobase;

X is O, S, or optionally substituted N;

Y is a H, hydroxyl, halogen, optionally substituted C₁₋₆ alkoxy, or a protected hydroxyl group;

Y¹ is independently H or optionally substituted C₁₋₆ alkyl (e.g., methyl);

R¹ is protected hydroxyl (e.g., hydroxyl protected with 4,4′-dimethoxytrityl group (DMT));

R² is —N(R⁴)R⁶ or —N(C₁₋₆ alkyl)₂ (e.g., —N(iPr)₂); and

R³ is optionally substituted C₂₋₁₆ alkyl; optionally substituted C₃₋₁₆ alkenyl; optionally substituted C₃₋₁₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O, and S; optionally substituted C₂₋₉ heterocyclyl having 1 to 4 heteroatoms selected from N, O, and S, where the heterocyclyl does not comprise an S—S bond; optionally substituted (C₂₋₉ heterocyclyl)-C₁₋₄-alkyl having 1 to 4 heteroatoms selected from N, O, and S, where the heterocyclyl does not comprise an S—S bond, or a group that is

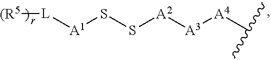

where A¹ is a bond or a linker containing or consisting of one or more of optionally substituted N, O, S, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from nitrogen, oxygen, and sulfur; optionally substituted (C₁₋₉ heteroaryl)-C₁₋₄-alkylene having 1 to 4 heteroatoms selected from nitrogen, oxygen; optionally substituted C₂₋₉ heterocyclylene having 1 to 4 heteroatoms selected from nitrogen, oxygen, and sulfur; and optionally substituted (C₂₋₉ heterocyclyl)-C₁₋₄-alkylene having 1 to 4 heteroatoms selected from nitrogen, oxygen, and sulfur, provided that when A¹ comprises one or more of amino, O, and S, none of the amino, O, and S is directly bonded to the disulfide; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from nitrogen, oxygen, and sulfur; and optionally substituted C₂₋₉ heterocyclylene having 1 to 4 heteroatoms selected from nitrogen, oxygen, and sulfur; or A¹ and A², together with —S—S—, join to form an optionally substituted 5 to 16 membered ring;

A³ is selected from the group consisting of a bond, optionally substituted C₁₋₆ alkylene; optionally substituted C₃₋₈ cycloalkylene; optionally substituted C₃₋₈ cycloalkenylene; optionally substituted C₆₋₁₄ arylene, optionally substituted C₁₋₉ heteroarylene having 1 to 4 heteroatoms selected from nitrogen, oxygen, and sulfur; optionally substituted C₂₋₉ heterocyclylene having 1 to 4 heteroatoms selected from nitrogen, oxygen, and sulfur; 0; optionally substituted N; and S;

A⁴ is selected from the group consisting of optionally substituted C₁₋₆ alkylene; optionally substituted C₃₋₈ cycloalkylene; and optionally substituted C₂₋₉ heterocyclylene having 1 to 4 heteroatoms selected from nitrogen, oxygen, and sulfur;

L is a bond or a conjugating group including or consisting of one or more conjugating moieties;

R⁵ is hydrogen, optionally substituted C₁₋₆ alkyl, a hydrophilic functional group, or a group comprising an auxiliary moiety selected from the group consisting of a small molecule, a peptide, a carbohydrate, a neutral organic polymer, a positively charged polymer, a therapeutic agent, a targeting moiety, an endosomal escape moiety, and combination thereof;

r is an integer from 1 to 10;

where A², A³, and A⁴ combine to form a group having at least three atoms (e.g., from three to six (e.g., four)) in the shortest chain connecting —S—S— and X; and

each R⁴ and R⁶ is independently selected from the group consisting of hydrogen; optionally substituted C₁₋₆ alkyl; optionally substituted C₂₋₇ alkanoyl; hydroxyl; optionally substituted C₁₋₆ alkoxy; optionally substituted C₃₋₈ cycloalkyl; optionally substituted C₃₋₈ cycloalkenyl; optionally substituted C₆₋₁₄ aryl; optionally substituted C₆₋₁₅ aryloyl; optionally substituted C₂₋₉ heterocyclyl having 1 to 4 heteroatoms selected from nitrogen, oxygen, and sulfur; and optionally substituted C₃-1₀ (heterocycle)oyl having 1 to 4 heteroatoms selected from nitrogen, oxygen, and sulfur.

The invention further provides methods to process a polynucleotide construct synthesized by using a method of manufacture disclosed herein. For example, post synthesis of the polynucleotide construct, if a nucleobase contains one or more protecting groups, the protecting groups may be removed; and/or for any -L-A¹-S—S-A²-A³-A⁴-containing a hydrophilic functional group or conjugating moiety that is protected by a protecting group, then the protecting group may be removed.

Additionally, post synthesis of the polynucleotide construct, a group containing one or more auxiliary moieties can be linked to one or more conjugating moieties of one or more bioreversible groups.

Conjugation

Preparation of polynucleotide constructs of the invention may involve conjugating an auxiliary moiety to a non-bioreversible or bioreversible linker attached to a phosphate or a phosphorothioate in the polynucleotide construct. The auxiliary moiety and the linker include complementary conjugating moieties. The location of attachment in a polynucleotide construct is determined by the positioning of the phosphates or phosphorothioates bearing the linker. Thus, a polynucleotide construct containing one more conjugating moieties will react, under appropriate conditions, with one or more complementary conjugating moieties on auxiliary moieties. The auxiliary moiety may intrinsically possess the conjugating moiety, e.g., terminal or lysine amine groups and thiol groups in peptides, or it may be modified to include a small linking group to introduce the conjugating moiety. Introduction of such linking groups is well known in the art. It will be understood that an auxiliary moiety attached to a nucleotide construct of the invention includes any necessary linking group.

Diverse bond-forming methods can be used to conjugate the auxiliary moiety to the nucleotide constructs described herein. Exemplary reactions include: cycloaddition between an azide and an alkyne to form a triazole; the Diels-Alder reaction between a dienophile and a diene/hetero-diene; bond formation via other pericyclic reactions such as the ene reaction; amide or thioamide bond formation; sulfonamide bond formation; alcohol or phenol alkylation (e.g., with diazo compounds), condensation reactions to form oxime, hydrazone, or semicarbazide group, conjugate addition reactions by nucleophiles (e.g., amines and thiols), disulfide bond formation, and nucleophilic substitution at a carboxylic functionality (e.g., by an amine, thiol, or hydroxyl nucleophile). Other exemplary methods of bond formation are described herein and known in the art.

Nucleophile/Electrophile Reactions

Nucleophiles and electrophiles can engage in bond forming reactions selected from, without limitation, insertion by an electrophile into a C—H bond, insertion by an electrophile into an O—H bond, insertion by an electrophile into an N—H bond, addition of the electrophile across an alkene, addition of the electrophile across an alkyne, addition to electrophilic carbonyl centers, substitution at electrophilic carbonyl centers, addition to ketenes, nucleophilic addition to isocyanates, nucleophilic addition to isothiocyanates, nucleophilic substitution at activated silicon centers, nucleophilic displacement of an alkyl halide, nucleophilic displacement at an alkyl pseudohalide, nucleophilic addition/elimination at an activated carbonyl, 1,4-conjugate addition of a nucleophile to an α,β-unsaturated carbonyl, nucleophilic ring opening of an epoxide, nucleophilic aromatic substitution of an electron deficient aromatic compound, a nucleophilic addition to activated phosphorus centers, nucleophilic substitution at activated phosphorous centers, nucleophilic addition to activated sulfur centers, and nucleophilic substitution at activated sulfur centers.

A nucleophilic conjugating moiety may be selected from optionally substituted alkenes, optionally substituted alkynes, optionally substituted aryl, optionally substituted heterocyclyl, hydroxyl groups, amino groups, alkylamino groups, anilido groups, and thio groups.

An electrophilic conjugating moiety may be selected from azides, activated silicon centers, activated carbonyls, anhydrides, isocyanates, thioisocyanates, succinimidyl esters, sulfosuccinimidyl esters, maleimides, alkyl halides, alkyl pseudohalides, epoxides, episulfides, aziridines, electron-deficient aryls, activated phosphorus centers, and activated sulfur centers.

For example, conjugation can occur via a condensation reaction to form a linkage that is a hydrazone bond.

Conjugation via the formation of an amide bond can be mediated by activation of a carboxyl-based conjugating moiety and subsequent reaction with a primary amine-based conjugating moiety. Activating agents can be various carbodiimides like: EDC (1-Ethyl-3-(3-dimethylaminopropyl)carbodiimide hydrochloride), EDAC (1-ethyl-3(3-dimethylaminopropyl)carbodiimide hydrochloride), DCC (dicyclohexyl carbodiimide), CMC (1-Cyclohexyl-3-(2-morpholinoethyl) carbodiimide), DIC (diisopropyl carbodiimide) or Woodward's reagent K (N-ethyl-3-phenylisoxazolium-3′-sulfonate). Reaction of an activated NHS-Ester-based conjugating moiety with a primary amine-based conjugating moiety also results in formation of an amide bond.

Ether formation can also be used to conjugate auxiliary moieties to the nucleotide constructs of the invention. Conjugation via ether linkages can be mediated by reaction of an epoxide-based conjugating moiety with a hydroxy-based conjugating moiety.

Thiols can also be used as conjugating moieties. For example, conjugation via the formation of disulfide bonds can be accomplished by pyridyldisulfide mediated thiol-disulfide exchange. Introduction of sulfhydryl-based conjugating moieties is mediated for instance by Traut's Reagent (2-iminothiolane) SATA (N-succinimidyl S-acetylthioacetate, SATP (succinimidyl acetylthiopropionate), SPDP (N-succinimidyl 3-(2-pyridyldithio)propionate, SMPT (succinimidyloxycarbonyl-α-methyl-α-(2-pyridyldithio)toluene), N-acetylhomocysteinethiolactone, SAMSA (S-acetylmercaptosuccinic anhydride), AMBH (2-Acedamido-4-mercaptobuturic acid hydrazide), and cystamine (2,2′-dithiobis(ethylamine).

Conjugation via the formation of thioether linkages can be performed by reacting a sulfhydryl based conjugating moieties with maleimide- or iodoacetyl-based conjugating moieties or by reacting with epoxide-based conjugating moieties. Maleimide-based conjugating moieties can be introduced by SMCC (succinimidyl-4-(N-maleimidomethyl)cyclohexane-1-carboxylate), sulfo-SMCC (sulfosuccinimidyl 4-(N-maleidomethyl)-cyclohexane-1-carboxylate), MBS (m-Maleimidobenzoyl-N-hydroxysuccinimide ester), sulfo-MBS (m-Maleimidobenzoyl-N-sulfohydroxy succinimide ester), SMPB (Succinimidyl-4-(p-maleidophenyl)butyrate), sulfo-SMPB (sulfosuccinimidyl 4-(p-maleimidophenyl)butyrate), GMBS (N-α-maleimidobuturyl-oxysuccinimide ester), sulfo GMBS (N-α-maleimidobuturyl-oxysulfosuccinimide ester).

Conjugation via the formation of a carbamate linkage can be performed by reaction of a hydroxy-based conjugating moiety with CDI (N,N′-carbonyldiimidazole) or DSC (N,N′-disuccinimidyl carbonate) or N-hydroxysuccinimidylchloroformate and subsequent reaction with an amine-based conjugating moiety.

Photolytic and Thermolytic Conjugation

Alternatively, the conjugating moiety can employ photolytic or thermolytic activation in order to form the desired covalent bond. Conjugating moieties that include azido functionality are one example. Thus, conjugation can also be achieved by the introduction of a photoreactive conjugating moiety. Photoreactive conjugating moieties are aryl azides, halogenated aryl azides, benzophenones certain diazo compounds and diazirine derivatives. They react with amino-based conjugating moieties or with conjugating moieties that have activated hydrogen bonds.

The azido-based conjugating moieties are UV labile and, upon photolysis, can lead to the formation of nitrene electrophiles that can react with nucleophilic conjugating moieties such as aryl-based conjugating moieties or alkenyl-based conjugating moieties. Alternatively, the heating of these azido compounds can also result in nitrene formation.

Cycloaddition Reactions

Cycloaddition reactions can be used to form the desired covalent bond. Representative cycloaddition reactions include, but are not limited to, the reaction of an alkene-based conjugating moiety with a 1,3-diene-based conjugating moiety (Diels-Alder reaction), the reaction of an alkene-based conjugating moiety with an α,β-unsaturated carbonyl-based conjugating moiety (hetero Diels-Alder reaction), and the reaction of an alkyne-based conjugating moiety with an azido-based conjugating moiety (Hüisgen cycloaddition). Selected, non-limiting examples of conjugating moieties that include reactants for cycloaddition reactions are: alkenes, alkynes, 1,3-dienes, α,β-unsaturated carbonyls, and azides. For example, the Hüisgen cycloaddition (click reaction) between azides and alkynes has been used for the functionalization of diverse biological entities.

### Pharmaceutical Compositions

Delivery of a nucleotide construct of the invention can be achieved by contacting a cell with the construct using a variety of methods known to those of skill in the art. In particular embodiments, a nucleotide construct of the invention is formulated with various carriers, dispersion agents and the like, as are described more fully elsewhere herein.

A pharmaceutical composition according to the invention can be prepared to include a nucleotide construct disclosed herein, into a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers.

Preservatives include antimicrobial, anti-oxidants, chelating agents, and inert gases. Other pharmaceutically acceptable carriers include aqueous solutions, non-toxic excipients, including salts, preservatives, buffers and the like, as described, for instance, in Remington: The Science and Practice of Pharmacy, 21^{st} Ed., Gennaro, Ed., Lippencott Williams & Wilkins (2005), and The United States Pharmacopeia: The National Formulary (USP 36 NF31), published in 2013. The pH and exact concentration of the various components of the pharmaceutical composition are adjusted according to routine skills in the art. See Goodman and Gilman's, The Pharmacological Basis for Therapeutics.

The pharmaceutical compositions according to the invention may be administered locally or systemically. The therapeutically effective amounts will vary according to factors, such as the degree of infection in a subject, the age, sex, and weight of the individual. Dosage regimes can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g., subcutaneous, intravenous, intraorbital, and the like), oral administration, ophthalmic application, inhalation, transdermal application, topical application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically the composition will be stable under the conditions of manufacture and storage and preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the subject's diet. For oral therapeutic administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit. The tablets, troches, pills, capsules, and the like can also contain the following: a binder, such as gum tragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic in the amounts employed. In addition, the pharmaceutical composition can be incorporated into sustained-release preparations and formulations.

Thus, a pharmaceutically acceptable carrier is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

It is especially advantageous to formulate parenteral compositions in dosage unit form for ease of administration and uniformity of dosage. Dosage unit form as used herein, refers to physically discrete units suited as unitary dosages for the subject to be treated; each unit containing a predetermined quantity of pharmaceutical composition is calculated to produce the desired therapeutic effect in association with the required pharmaceutical carrier. The specification for the dosage unit forms of the invention are related to the characteristics of the pharmaceutical composition and the particular therapeutic effect to be achieve. The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the ingredients.

For topical formulations, the base composition can be prepared with any solvent system, such as those Generally Regarded as Safe (GRAS) by the U.S. Food & Drug Administration (FDA). GRAS solvent systems include many short chain hydrocarbons, such as butane, propane, n-butane, or a mixture thereof, as the delivery vehicle, which are approved by the FDA for topical use. The topical compositions can be formulated using any dermatologically acceptable carrier. Exemplary carriers include a solid carrier, such as alumina, clay, microcrystalline cellulose, silica, or talc; and/or a liquid carrier, such as an alcohol, a glycol, or a water-alcohol/glycol blend. The compounds may also be administered in liposomal formulations that allow compounds to enter the skin. Such liposomal formulations are described in U.S. Pat. Nos. 5,169,637; 5,000,958; 5,049,388; 4,975,282; 5,194,266; 5,023,087; 5,688,525; 5,874,104; 5,409,704; 5,552,155; 5,356,633; 5,032,582; 4,994,213; and PCT Publication No. WO 96/40061. Examples of other appropriate vehicles are described in U.S. Pat. Nos. 4,877,805, 4,980,378, 5,082,866, 6,118,020 and EP Publication No. 0586106A1. Suitable vehicles of the invention may also include mineral oil, petrolatum, polydecene, stearic acid, isopropyl myristate, polyoxyl 40 stearate, stearyl alcohol, or vegetable oil.

Topical compositions can be provided in any useful form. For example, the compositions of the invention may be formulated as solutions, emulsions (including microemulsions), suspensions, creams, foams, lotions, gels, powders, balm, or other typical solid, semi-solid, or liquid compositions used for application to the skin or other tissues where the compositions may be used. Such compositions may contain other ingredients typically used in such products, such as colorants, fragrances, thickeners, antimicrobials, solvents, surfactants, detergents, gelling agents, antioxidants, fillers, dyestuffs, viscosity-controlling agents, preservatives, humectants, emollients (e.g., natural or synthetic oils, hydrocarbon oils, waxes, or silicones), hydration agents, chelating agents, demulcents, solubilizing excipients, adjuvants, dispersants, skin penetration enhancers, plasticizing agents, preservatives, stabilizers, demulsifiers, wetting agents, sunscreens, emulsifiers, moisturizers, astringents, deodorants, and optionally including anesthetics, anti-itch actives, botanical extracts, conditioning agents, darkening or lightening agents, glitter, humectants, mica, minerals, polyphenols, silicones or derivatives thereof, sunblocks, vitamins, and phytomedicinals.

In some formulations, the composition is formulated for ocular application. For example, a pharmaceutical formulation for ocular application can include a polynucleotide construct as described herein in an amount that is, e.g., up to 99% by weight mixed with a physiologically acceptable ophthalmic carrier medium such as water, buffer, saline, glycine, hyaluronic acid, mannitol, and the like. For ophthalmic delivery, a polynucleotide construct as described herein may be combined with ophthalmologically acceptable preservatives, co-solvents, surfactants, viscosity enhancers, penetration enhancers, buffers, sodium chloride, or water to form an aqueous, sterile ophthalmic suspension or solution. Ophthalmic solution formulations may be prepared by dissolving the polynucleotide construct in a physiologically acceptable isotonic aqueous buffer. Further, the ophthalmic solution may include an ophthalmologically acceptable surfactant to assist in dissolving the inhibitor. Viscosity building agents, such as hydroxymethyl cellulose, hydroxyethyl cellulose, methylcellulose, polyvinylpyrrolidone, or the like may be added to the compositions of the invention to improve the retention of the compound.

Topical compositions can be delivered to the surface of the eye, e.g., one to four times per day, or on an extended delivery schedule such as daily, weekly, bi-weekly, monthly, or longer, according to the routine discretion of a skilled clinician. The pH of the formulation can range from about pH 4-9, or about pH 4.5 to pH 7.4.

For nucleotide constructs of the invention, suitable pharmaceutically acceptable salts include (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, nitric acid and the like; (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (iv) salts formed from elemental anions such as chlorine, bromine, and iodine.

While the nucleotide constructs described herein may not require the use of a carrier for delivery to the target cell, the use of carriers may be advantageous in some embodiments. Thus, for delivery to the target cell, the nucleotide construct of the invention can non-covalently bind a carrier to form a complex. The carrier can be used to alter biodistribution after delivery, to enhance uptake, to increase half-life or stability of the polynucleotide (e.g., improve nuclease resistance), and/or to increase targeting to a particular cell or tissue type.

Exemplary carriers include a condensing agent (e.g., an agent capable of attracting or binding a nucleic acid through ionic or electrostatic interactions); a fusogenic agent (e.g., an agent capable of fusing and/or being transported through a cell membrane); a protein to target a particular cell or tissue type (e.g., thyrotropin, melanotropin, lectin, glycoprotein, surfactant protein A, or any other protein); a lipid; a lipopolysaccharide; a lipid micelle or a liposome (e.g., formed from phospholipids, such as phosphotidylcholine, fatty acids, glycolipids, ceramides, glycerides, cholesterols, or any combination thereof); a nanoparticle (e.g., silica, lipid, carbohydrate, or other pharmaceutically-acceptable polymer nanoparticle); a polyplex formed from cationic polymers and an anionic agent (e.g., a CRO), where exemplary cationic polymers include polyamines (e.g., polylysine, polyarginine, polyamidoamine, and polyethylene imine); cholesterol; a dendrimer (e.g., a polyamidoamine (PAMAM) dendrimer); a serum protein (e.g., human serum albumin (HSA) or low-density lipoprotein (LDL)); a carbohydrate (e.g., dextran, pullulan, chitin, chitosan, inulin, cyclodextrin, or hyaluronic acid); a lipid; a synthetic polymer, (e.g., polylysine (PLL), polyethylenimine, poly-L-aspartic acid, poly-L-glutamic acid, styrene-maleic acid anhydride copolymer, poly(L-lactide-co-glycolic) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacrylic acid), N-isopropylacrylamide polymer, pseudopeptide-polyamine, peptidomimetic polyamine, or polyamine); a cationic moiety (e.g., cationic lipid, cationic porphyrin, quaternary salt of a polyamine, or alpha helical peptide); a multivalent sugar (e.g., multivalent lactose, multivalent galactose, N-acetyl-galactosamine, N-acetyl-glucosamine, multivalent mannose, or multivalent fucose); a vitamin (e.g., vitamin A, vitamin E, vitamin K, vitamin B, folic acid, vitamin B12, riboflavin, biotin, or pyridoxal); a cofactor; or a drug to disrupt cellular cytoskeleton to increase uptake (e.g., taxol, vincristine, vinblastine, cytochalasin, nocodazole, japlakinolide, latrunculin A, phalloidin, swinholide A, indanocine, or myoservin).

Other therapeutic agents as described herein may be included in a pharmaceutical composition of the invention in combination with a nucleotide construct of the invention.

### Intracellular Activity of the Hybridized Polynucleotide Constructs

The invention provides compositions and methods for delivering hybridized polynucleotide constructs disclosed herein. The invention therefore provides methods and compositions useful for delivery of non-coding nucleotide constructs that exert a regulating effect on gene or protein expression through RNA interference (RNAi). RNA interference (RNAi) is the process whereby messenger RNA (mRNA) is degraded by small interfering RNA (siRNA) derived from double-stranded RNA (dsRNA) containing an identical or very similar nucleotide sequence to that of a target gene to be silenced. This process prevents the production of a protein encoded by the targeted gene through post-transcriptional, pre-translational manipulation. Accordingly, silencing of dominant disease genes or other target genes can be accomplished.

In vivo RNAi proceeds by a process in which the dsRNA is cleaved into short interfering RNAs (siRNAs) by an enzyme called Dicer, a dsRNA endoribonuclease, (Bernstein et al., 2001; Hamilton & Baulcombe, 1999, Science 286: 950; Meister and Tuschl, 2004, Nature 431, 343-9), thus producing multiple molecules from the original single dsRNA. siRNAs are loaded into the multimeric RNAi Silencing Complex (RISC) resulting in both catalytic activation and mRNA target specificity (Hannon and Rossi, Nature 431, 371-378, 2004; Novina and Sharp, Nature 430, 161-164, 2004). During siRNA loading into RISC, the antisense or guide strand is separated from the siRNA and remains docked in Argonaute-2 (Ago2), the RISC catalytic subunit (Leuschner et al., EMBO Rep. 7, 314-320, 2006). Certain cellular compartments, such as endoplasmic reticulum (ER), Golgi apparatus, ER-Golgi intermediate compartment (ERGIC), P-bodies, and early endosomes are enriched in Ago2. mRNAs exported from the nucleus into the cytoplasm are thought to pass through activated RISCs prior to ribosomal arrival, thereby allowing for directed, post-transcriptional, pre-translational regulation of gene expression. In theory, each and every cellular mRNA can be regulated by induction of a selective RNAi response.

The ability of siRNAs to efficiently induce an RNAi response in mammalian cells in vitro is known (Sontheimer, Nat. Rev. Mol. Cell. Biol. 6, 127-138, 2005). Typically, the IC₅₀ for siRNAs is in the 10-100 pM range, significantly below the best drugs with IC₅₀ values in the 1-10 nM range. Consequently, due to its exquisite selectivity, RNAi has become a corner-stone for directed manipulation of cellular phenotypes, mapping genetic pathways, discovering and validating therapeutic targets, and has significant therapeutic potential.

Aspects of RNAi include (1) dsRNA is the interfering agent; (2) the process can be sequence-specific and is remarkably potent (only a few dsRNA molecules per cell are required for effective interference); (3) the interfering activity (and presumably the dsRNA) can cause interference in cells and tissues far removed from the site of introduction. However, effective delivery of dsRNA is difficult. For example, a 21 bp dsRNA with a molecular weight of 13,860 Daltons cannot traverse the cell membrane to enter the cytoplasm, due to (1) the size and (2) the accumulation of negative charges on the RNA molecule at physiologically relevant pH levels. The methods and compositions of the invention provide the delivery of nucleotide constructs, such as dsRNA, into a cell through charge neutralization and improved uptake.

dsRNA including siRNA sequences that are complementary to a nucleotide sequence of the target gene can be prepared in any number of methods, e.g., those described herein. Methods and techniques for identifying siRNA sequences are known in the art. The siRNA nucleotide sequence can be obtained from the siRNA Selection Program, Whitehead Institute for Biomedical Research, Massachusetts Institute of Technology, Cambridge, Mass. (currently available at http:[//]jura.wi.mit.edu/bioc/siRNAext/; note that brackets have been added to remove hyperlinks) after supplying the Accession Number or GI number from the National Center for Biotechnology Information website (available on the World Wide Web at ncbi.nlm.nih.gov). Alternatively, dsRNA containing appropriate siRNA sequences can be ascertained using the strategy of Miyagishi and Taira (2003). Commercially available RNAi designer algorithms also exist (http:[//]rnaidesigner.invitrogen.com/rnaiexpress/). Preparation of RNA to order is commercially available.

Polynucleotide constructs of the invention may also act as miRNA to induce cleavage of mRNA. Alternatively, nucleotide constructs of the invention may act as antisense agents to bind to mRNA, either to induce cleavage by RNase or to sterically block translation.

Exemplary methods by which the nucleotide constructs of the invention can be transported into a cell are described herein.

### Therapeutic Methods

Proprotein Convertase Subtilisin/Kexin Type 9 (PCSK9)

PCSK9 is an enzyme encoded by PCSK9 gene in humans. This enzyme binds to the receptor for low-density lipoprotein particles (LDLR). LDLR binds and mediates cellular ingestion of LDL particles, thus reducing extracellular LDL particle concentration. After ingestion, LDLR is recycled back to the cell surface, where it can bind and mediate the ingestion of more LDL particles. When PCSK9 is bound to LDLR, upon ingestion of LDLR and LDL, LDLR is degraded and is not recycled back to the cell surface and is degraded instead. Reduction in the PCSK9 levels can thus lead to an increase in the LDLR recycling, thereby reducing the extracellular LDL particles concentration. Accordingly, PCSK9 has been targeted for the development of therapeutics for hypercholesterolemia.

The polynucleotide constructs disclosed herein having a sequence complementary to a portion of a PCSK9 transcript (e.g., a portion that is at least 12 (e.g., 13, 14, 15, 16, 17, 18, 19, 20, 21, or 22) nucleotides long) may be used in a method of reducing low density lipoprotein levels in a subject in need thereof by administering an effective amount of the polynucleotide construct disclosed herein (e.g., a hybridized polynucleotide construct including a passenger strand and a guide strand containing a sequence complementary to a portion of a PCSK9 transcript) to the subject. PCSK9 gene and its transcripts are known in the art. The polynucleotide constructs disclosed herein having a sequence complementary to a portion of a PCSK9 transcript (e.g., to a portion that is at least 12 (e.g., 13, 14, 15, 16, 17, 18, 19, 20, 21, or 22) nucleotides long) may also be used in a method of treating hypercholesterolemia by administering an effective amount of the polynucleotide construct disclosed herein (e.g., a hybridized polynucleotide construct including a passenger strand and a guide strand containing a sequence complementary to a portion of a PCSK9 transcript) to the subject.

Transthyretin (TTR)

Transthyretin (TTR) is a transport protein in the serum and cerebrospinal fluid that carries thyroxine and retinol-binding protein bound to retinol. TTR misfolding and aggregation is often associated with amyloid diseases (e.g., familial amyloid polyneuropathy and senile systemic amyloidosis). Accordingly, TTR has been targeted for the development of therapeutics for TTR-mediated amyloid diseases.

The polynucleotide constructs disclosed herein having a sequence complementary to a portion of a TTR transcript (e.g., to a portion that is at least 12 (e.g., 13, 14, 15, 16, 17, 18, 19, 20, 21, or 22) nucleotides long) may be used in a method of reducing transthyretin levels in a subject in need thereof by administering an effective amount of the polynucleotide construct disclosed herein (e.g., a hybridized polynucleotide construct including a passenger strand and a guide strand containing a sequence complementary to a portion of a TTR transcript) to the subject. TTR gene and its transcripts are known in the art. The polynucleotide constructs disclosed herein having a sequence complementary to a portion of a TTR transcript (e.g., a portion that is at least 12 (e.g., 13, 14, 15, 16, 17, 18, 19, 20, 21, or 22) nucleotides long) may also be used in a method of treating hypercholesterolemia by administering an effective amount of the polynucleotide construct disclosed herein (e.g., a hybridized polynucleotide construct including a passenger strand and a guide strand containing a sequence complementary to a portion of a TTR transcript) to the subject.

The following examples are meant to illustrate the invention. They are not meant to limit the invention in any way.

### EXAMPLES

### Example 1. Synthesis and Purification of the Nucleotides and Polynucleotides of the Invention

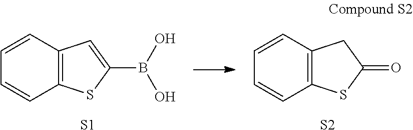

To a solution of compound S1 (30.0 g, 168.5 mmol) in EtOH (120 mL) was added 30% hydrogen peroxide (50 mL) dropwise over 45 min (caution: exothermic). Reaction mixture became turbid with white precipitate. TLC showed completion of reaction at 3 hr, and the reaction mixture was diluted with water (300 mL) and carefully extracted with dichloromethane (200 mL×3). The combined organic layer was dried over anhydrous sodium sulfate and concentrated in vacuo to afford crude product. This was purified by flash silica gel column (220 g) chromatography using ISCO companion (ethyl acetate/hexane, 0-20% over 15 column volumes) to give 23.5 g (92%) of compound S2 as a light yellow oil which became solid on standing at room temperature. ¹H NMR (500 MHz, CDCl₃): δ7.34 (1H, dd, J 8.0 Hz), 7.31-7.28 (2H, m), 7.22 (1H, td, J 8.0, 1.0 Hz), 3.98 (2H, s).

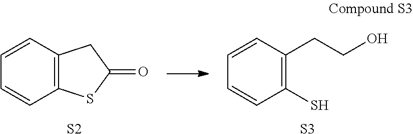

To an ice cold solution of LiAlH₄ (7.4 g, 200 mmol) in diethyl ether (200 mL) was added dropwise a solution of compound S2 (15.0 g, 100 mmol) in diethyl ether over 1 h (caution: gas evolution and exothermic). The reaction mixture was allowed to reach room temperature, and stirring continued overnight. Reaction was carefully quenched with aq. Sodium sulfate until gas evolution stopped to give a white precipitate. To this 100 mL of 10% H₂SO₄ was carefully added and the organic layer separated. Aqueous layer extracted with 3×75 mL ether and the combined organic layers washed with water, brine, dried over sodium sulfate and evaporated to give compound S3 (14.6 g, 95%) as colorless oil which was used in the next reaction without further purification. ¹H NMR (500 MHz, CDCl₃): δ7.31 (1H, dd, J 7.5, 1.5 Hz), 7.20 (1H, dd, J 7.5, 1.5 Hz), 7.16-7.08 (2H, m), 3.91 (2H, t, J 6.5 Hz), 3.41 (1H, s), 2.98 (1H, J 6.5 Hz).

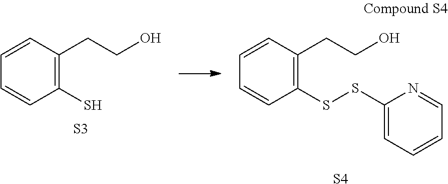

To a solution of dithiodipyridine (52.0 g, 236.3 mmol) and acetic acid (3.0 mL) in methanol (200 mL) at room temperature was added a solution of compound S3 (14.6 g, 94.5 mmol) in methanol (50 mL) and stirred overnight. Volatiles were removed to produce a residue, to which 100 mL of diethyl ether were added, and the separated solids were filtered and washed with diethyl ether (3×50 mL). The combined ether washes were evaporated to give crude product, which, on flash silica gel column purification using ISCO companion (ethyl acetate/hexane, 0-50%), gave 14.1 g (57%) of compound S4. ¹H NMR (500 MHz, CDCl₃): δ8.48 (1H, d, J 5.0 Hz), 7.65-7.60 (3H, m), 7.25-7.18 (3H, m), 7.13-7.10 (1H, m), 3.96 (2H, t, J 6.5 Hz), 3.17 (1H, t, J 6.5 Hz).

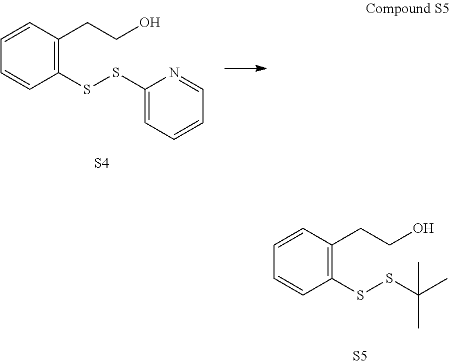 [SMILES: OCCc1ccccc1SSc1ccccn1 ; "S4" ; CC(C)(C)SSc1ccccc1CCO ; "S5"]

To a solution of compound S4 (4.5 g, 17.0 mmol) in 30.0 mL of dichloromethane at room temperature, MeOTf was added dropwise and stirred for 10 minutes followed by tert-butyl mercaptan (1.9 mL, 17.0 mmol) and N,N-diisopropylethylamine (6.0 mL, 34.0 mmol) addition. The reaction mixture was stirred for another 30 min at room temperature before being condensed in vacuo. The crude mixture was purified by silica gel column chromatography using ethyl acetate/hexane (0-30% gradient on Combi Flash Rf Instrument) to give product S5 as colorless oil (2.5 g, 61%). ¹H NMR (500 MHz): δ7.84 (d, J 5.0 Hz, 1H), 7.25-7.13 (m, 3H), 3.92 (t, J 7.0 Hz, 2H), 3.12 (t, J 7.0 Hz, 2H), 1.30 (s, 9H).

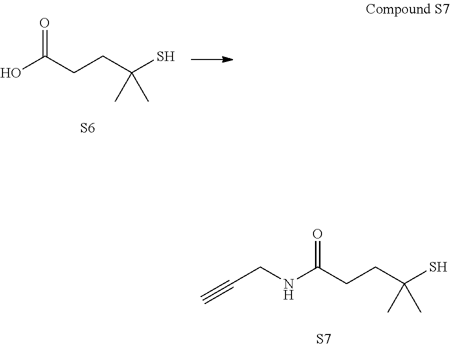

To a mixture of 2-methyl-2-mercaptopentanoic acid (S6, 0.74 g, 5.0 mmol) and acetic anhydride (0.52 mL, 5.5 mmol) in acetonitrile (10.0 mL) were added triethylamine (1.39 mL, 10.0 mmol) and DMAP (5 mg). The mixture was stirred for 1 hour, and propargylamine (0.69 g, 12.5 mmol) was added to the mixture, and stirring continued overnight. The volatiles were removed under vacuum to give a residue, which was subjected to flash silica gel column purification on an ISCO companion (ethyl acetate/hexane, 5-55%) to give 0.72 g (59%) of the title compound S7 as a white solid. ¹H NMR (500 MHz, CDCl₃): δ5.66 (1H, s), 4.06 (2H, dd, J 5.0, 2.5 Hz), 2.41-2.37 (2H, m), 2.23 (1H, t, J 2.5 Hz), 1.95-1.91 (2H, m), 1.39 (6H, s).

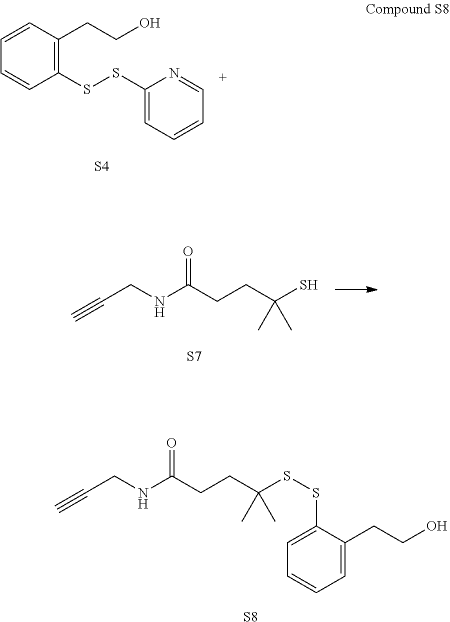

To a solution of compound S4 (1.0 g, 3.8 mmol) in dichloromethane (12 mL) was added MeOTf (0.6 g, 3.8 mmol). The mixture was stirred for 15 min, at which time, S7 (0.57 g, 3.04 mmol) and N,N-diisopropylethylamine (1.0 mL) were added, and the resulting mixture was stirred for additional 30 min. Evaporation of the reaction mixture afforded a residue which was subjected to flash silica gel column chromatography on an ISCO companion (ethyl acetate/hexane 5-60% to give compound S8 (0.99 g, 78%) as a colorless oil. ¹H NMR (500 MHz, CDCl₃): 57.83 (1H, d, J 8.0 Hz), 7.30-7.16 (3H, m), 5.05 (1H, s), 3.95 (2H, t, J 6.5 Hz), 3.88 (2H, dd, J 5.5, 2.5 Hz), 3.15 (2H, t, J 6.5 Hz), 2.23 (1H, t, J 2.5 Hz), 2.10-2.04 (2H, m), 1.83-1.79 (2H, m), 1.28 (6H, s).

### Phosphoramidite Synthesis:

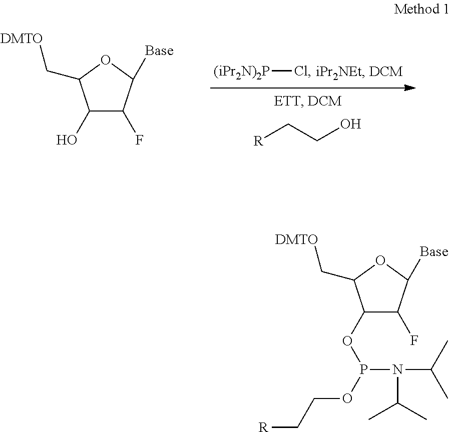

To a −78° C. solution of appropriately protected nucleoside (20.7 mmol) and N,N-diisopropylethylamine (22.7 mmol) in 100 mL of dry dichloromethane under argon, a solution of bis-(N,N-diisopropylamino)-chlorophosphine (22.7 mmol) in 20 mL of dichloromethane was slowly added. The reaction mixture was allowed to warm to room temperature over 1 hour while stirring was maintained. To this, a solution of appropriate alcohol (20.7 mmol) in 15 mL of dry dichloromethane was added, the resulting mixture was stirred for 10 minutes, at which time, 0.25M ETT in acetonitrile (12.42 mmol) was added dropwise. The reaction mixture was stirred for additional 16 hours at room temperature. The crude mixture was diluted with 200 mL of dichloromethane, washed sequentially with saturated NaHCO₃ solution (50 mL) and brine (50 mL), and then dried over anhydrous Na₂SO₄. The volatiles were evaporated in vacuo, and the resulting mixture was purified by silica gel column chromatography using ethyl acetate/hexane (0-30% gradient on Combi Flash Rf Instrument) to give a diastereomeric mixture of phosphoramidite as a white powder.

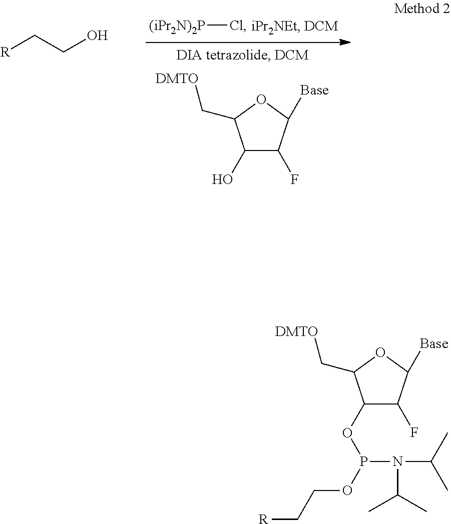

To a −78° C. solution of appropriate alcohol (7.46 mmol) and N,N-diisopropylethylamine (7.78 mmol) in dry dichloromethane (15 mL) under argon, a solution of bis-(N,N-diisopropylamino)-chlorophosphine (7.78 mmol) in dichloromethane (5.0 mL) was added. The reaction mixture was allowed to warm to room temperature over 1 hour, and the resulting solution was added dropwise to a dichloromethane (15 mL) suspension of appropriately protected nucleoside (3.73 mmol) and diisopropylammonium tetrazolide (7.46 mmol). The reaction continued for additional 16 hours at room temperature. The reaction mixture was diluted with 15 mL of dichloromethane, washed sequentially with saturated NaHCO₃ solution (10 mL) and brine (10 mL), and then dried over anhydrous Na₂SO₄. The volatiles were evaporated in vacuo, and the mixture was purified by silica gel column chromatography using ethyl acetate/hexane (0-60% gradient on Combi Flash Rf Instrument) to give a diastereomeric mixture of phosphoramidite as a white powder.

The phosphoramidite monomers shown in Table 1 were synthesized using the standard synthetic procedures described herein.

TABLE 1

| Compound # | Structure | ³¹P NMR (δ, ppm) | Synthesis | Yield (%) |
|---|---|---|---|---|
| U1 | 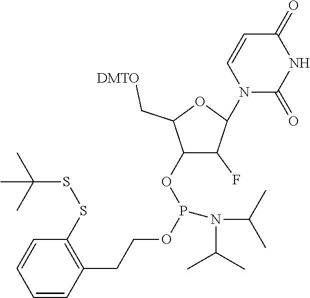 | 154.73 (d, J 8.90 Hz) 154.52 (d, J 7.70 Hz) | Method 1 | 45 |
| U2 | 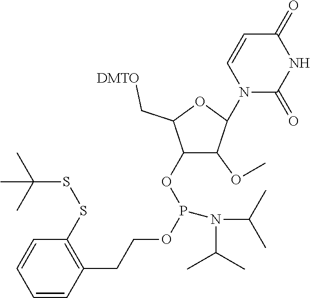 | 149.30 (s) 149.05 (s) | Method 1 | 41 |
| U3 | 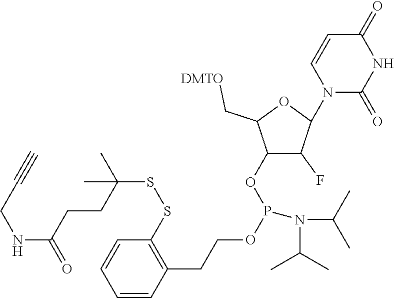 | 154.75 (d, J 5.60 Hz) 154.36 (d, J 10.3 Hz) | Method 1 | 21 |
| U4 | 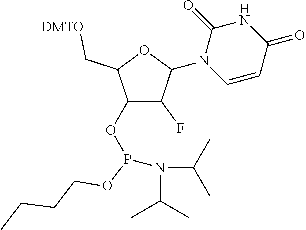 | 154.98 (d, J 8.08 Hz) 154.74 (d, J 8.08 Hz) | Method 1 | 54 |
| U5 | 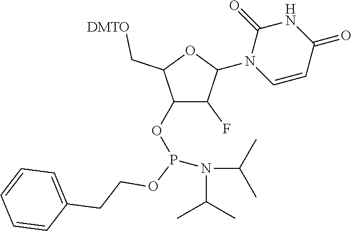 | 154.84 (d, J 12.12 Hz) 154.50 (d, J 8.08 Hz) | Method 1 | 53 |
| U6 | 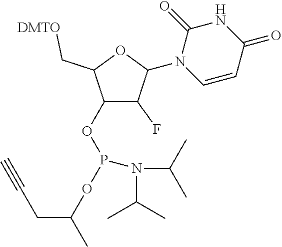 | 154.6 (d, J 6.6 Hz) 154.5 (d, J 8.5 Hz) 154.2 (d, J 9.3 Hz) 152.8 (d, J 10.1 Hz) | Method 1 | 60 |
| U7 | 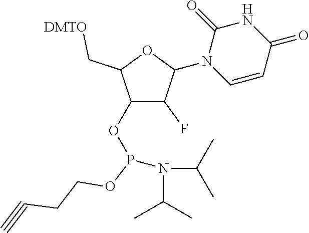 | 155.27 (d, J 6.06 Hz) 155.05 (d, J 8.08 Hz) | Method 1 | 50 |
| U8 | 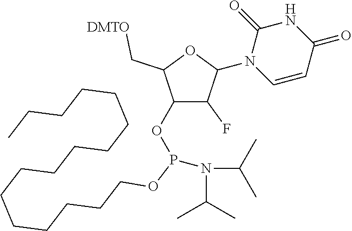 | 155.17 (d, J 8.08 Hz) 154.67 (d, J 10.1 Hz) | Method 1 | 48 |
| U9 | 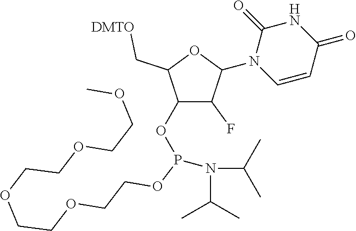 | 155.83 (d, J 6.06 Hz) 155.34 (d, J 10.1 Hz) | Method 1 | 55 |
| U10 | 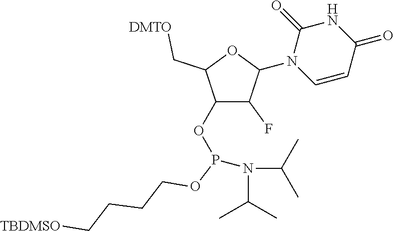 | 155.4 (d, J 7.9 Hz) 154.7 (d, J 9.7 Hz) | Method 1 | 54 |
| C1 | 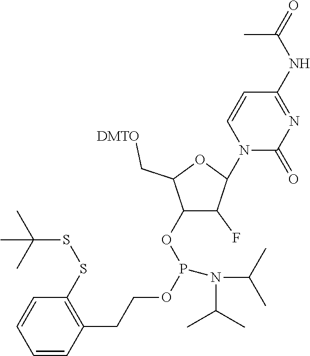 | | Method 1 | |
| C2 | 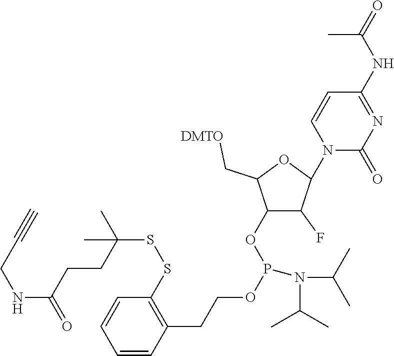 | | Method 1 | |
| C3 | 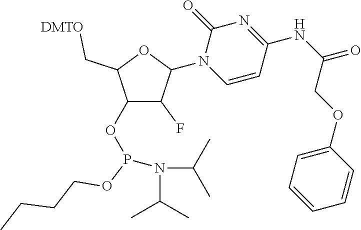 | 154.80 (d, J 8.08 Hz) 154.71 (d, J 6.06 Hz) | Method 1 | 51 |
| C4 | 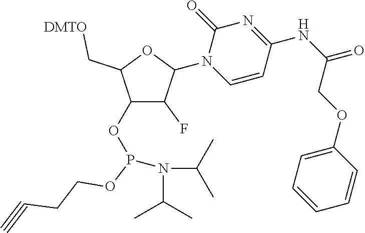 | 155.18 (d, J 6.06 Hz) 154.79 (d, J 8.08 Hz) | Method 1 | 55 |
| C5 | 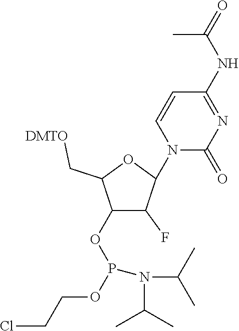 | 151.9 (d, J 6.2 Hz) 151.2 (d, J 8.3 Hz) | Method 1 | 28 |
| C6 | 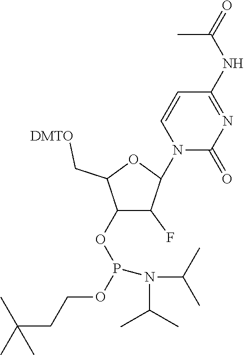 | 149.3 (d, J 6.0 Hz) 149.1 (d, J 8.3 Hz) | Method 1 | 35 |
| C7 | 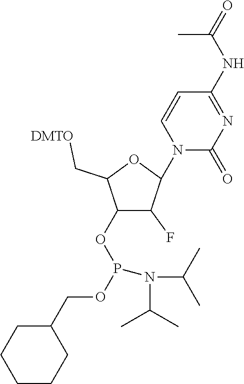 | 149.4 (s) 149.1 (d, J 8.3 Hz) | Method 1 | 21 |
| C8 | 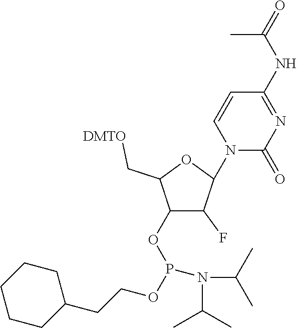 | 149.7 (d, J 8.3 Hz) | Method 1 | 36 |
| C9 | 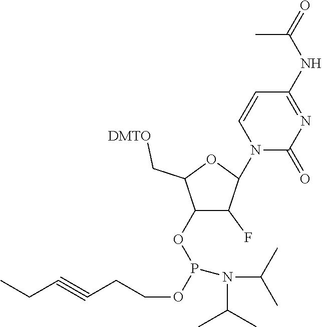 | 150.2 (s) 150.0 (s) | Method 1 | 27 |
| C10 | 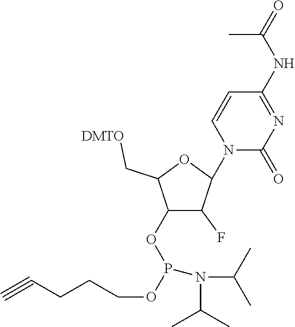 | 150.6 (d, J 8.3 Hz) 150.1 (s) | Method 1 | 37 |
| C11 | 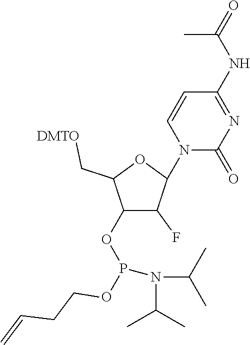 | 149.7 (d, J 8.3 Hz) | Method 1 | 41 |
| C12 | 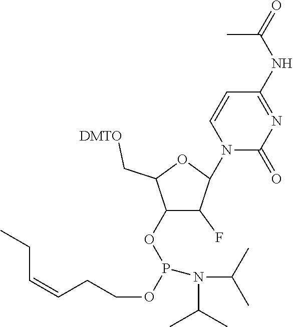 | 149.9 (d, J 8.3 Hz) 149.6 (s) | Method 1 | 48 |
| C13 | 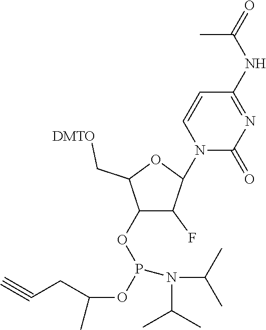 | 149.6 (s) 149.5 (d, J 8.3 Hz) 149.1 (s) 147.8 (s) | Method 1 | 37 |
| C14 | 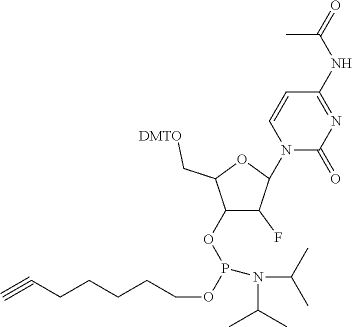 | 150.2 (d, J 8.3 Hz) 149.8 (s) | Method 1 | 14 |
| C15 | 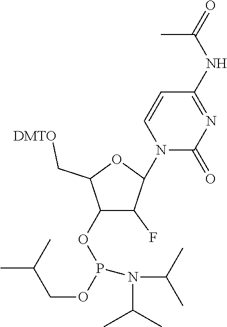 | 150.2 (d, J 8.3 Hz) 150.1 (d, J 6.2 Hz) | Method 1 | 48 |
| C16 | 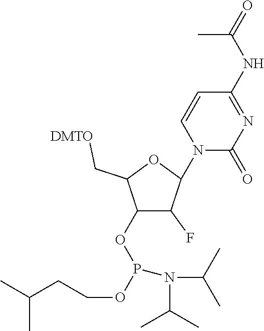 | 150.2 (d, J 10.3 Hz) 149.8 (s) | Method 1 | 47 |
| C17 | 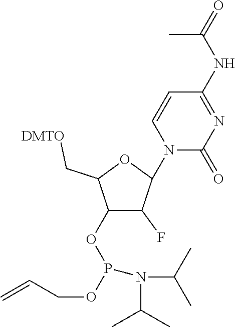 | 150.9 (d, J 8.3 Hz) 150.6 (s) | Method 1 | 52 |
| C18 | 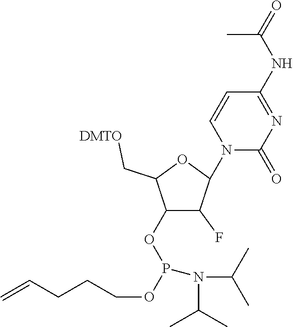 | 150.2 (d, J 6.2 Hz) 149.9 (d, J 4.2 Hz) | Method 1 | 41 |
| C19 | 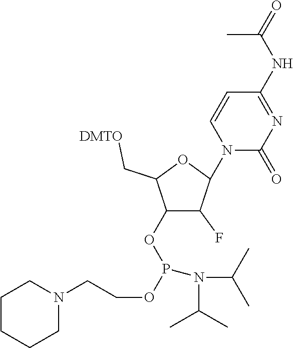 | 150.0 (s) 149.5 (s) | Method 1 | 10 |
| C20 | 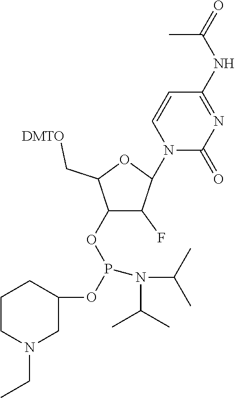 | 149.3 (s) 148.8 (s) 147.6 (s) 146.7 (s) | Method 1 | 4 |
| C21 | 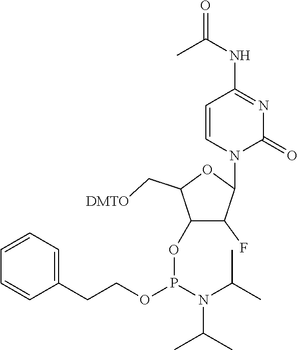 | 149.80 (d, J 8.1 Hz) 149.55 (s) | Method 1 | 47 |
| C22 | 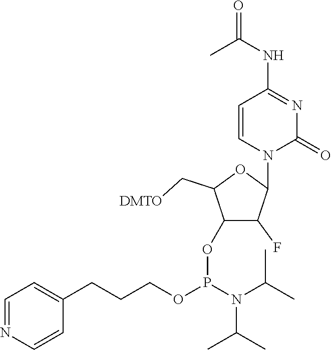 | 150.46 (d, J 8.1 Hz) | Method 1 | 22 |
| A1 | 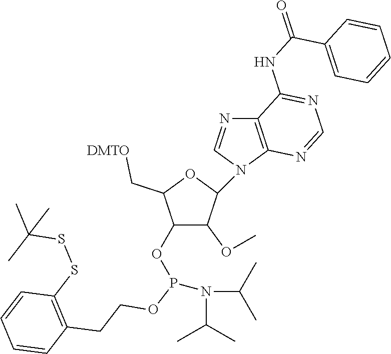 | 154.80 (s) 154.0 (s) | Method 1 | 40 |
| A2 | 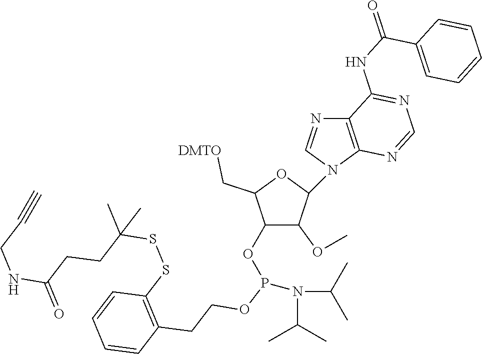 | | Method 1 | |
| A3 | 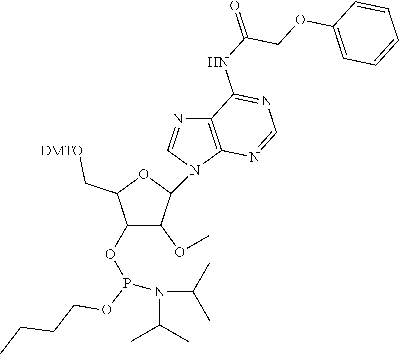 | 154.79 (s) 154.01 (s) | Method 1 | 34 |
| A4 | 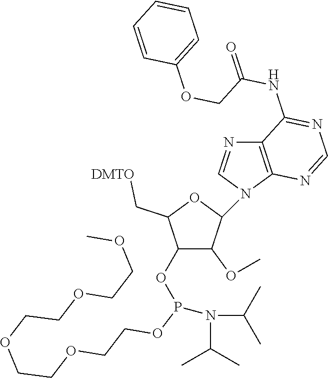 | 155.85 (s) 155.09 (s) | Method 1 | 25 |
| A5 | 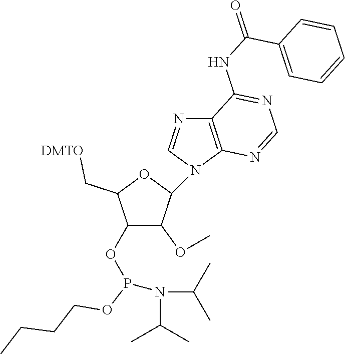 | 154.81 (s) 153.99 (s) | Method 1 | 55 |
| A6 | 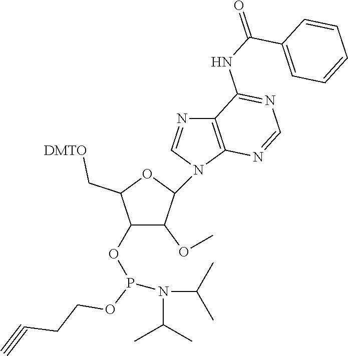 | 150.24 (s) 149.63 (s) | Method 1 | 39 |
| A7 | 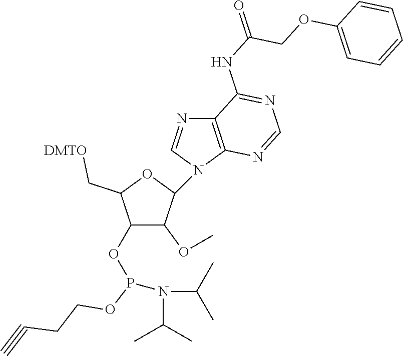 | 155.20 (s) 154.60 (s) | Method 1 | 56 |
| A8 | 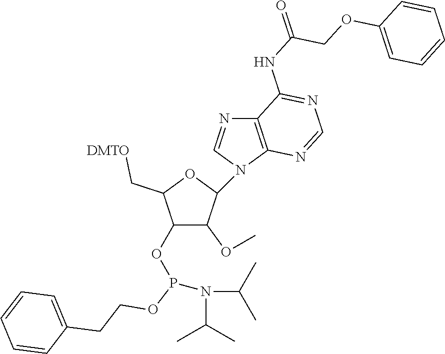 | 155.4 (s) 154.0 (s) | Method 1 | 80 |
| G1 | 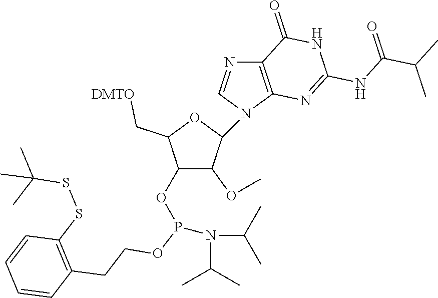 | 150.63 (s) 149.20 (s) | Method 1 | 36 |
| G2 | 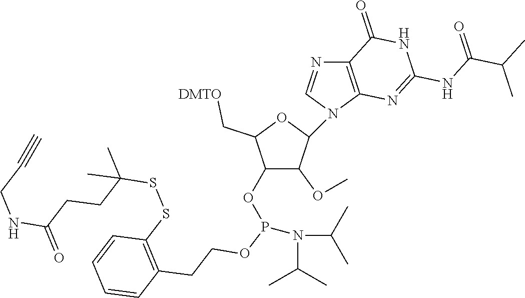 | | Method 2 | |
| G3 | 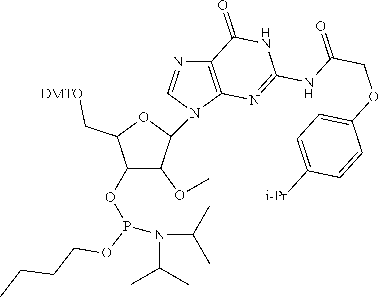 | 154.91 (s) 154.36 (s) | Method 2 | 55 |
| G4 | 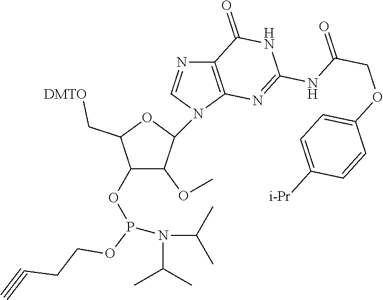 | 155.29 (s) 154.85 (s) | Method 2 | 29 |
| G5 | 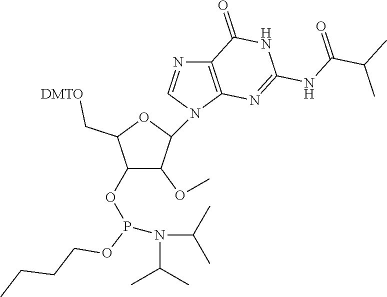 | 154.94 (s) 154.05 (s) | Method 2 | 18 |

### Synthesis of Targeting Moieties

### GalNAc (NAG) Targeting Moiety Synthesis:

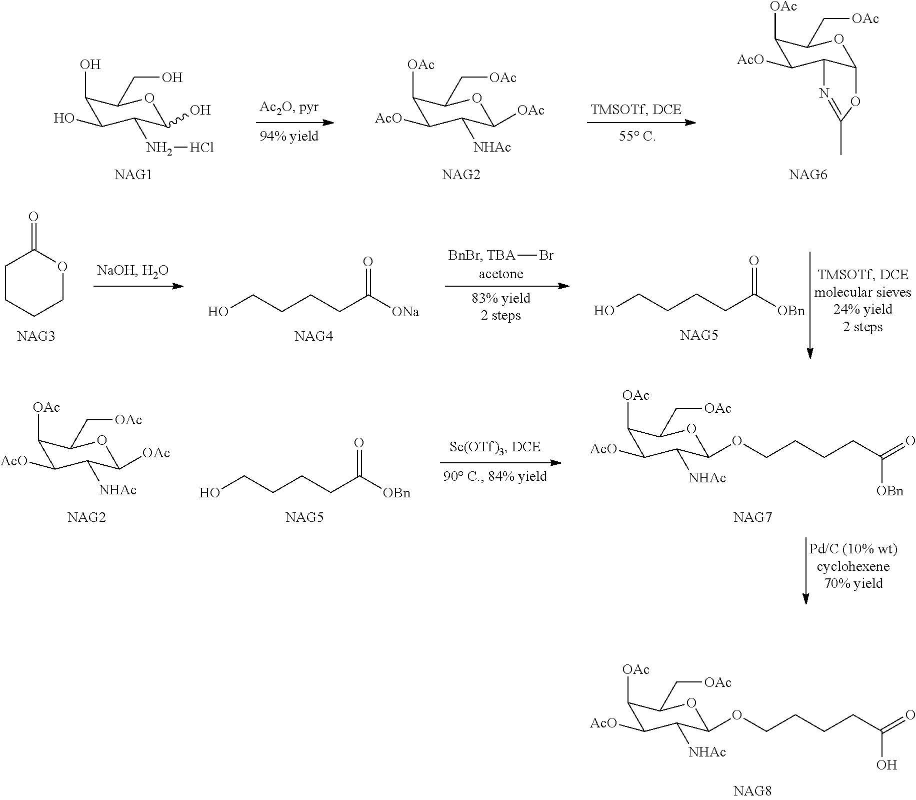

Preparation of D-galactosamine pentaacetate (NAG2).

D-Galactosamine (25.0 g, 116 mmol) was suspended in anhydrous pyridine (250 mL) and cooled to 0° C. under an inert atmosphere. Acetic anhydride (120 mL, 1160 mmol) was added over the course of 2 h. After stirring overnight, the reaction mixture was concentrated in vacuo. Upon addition of methanol, a white solid precipitated and was collected via filtration to provide the desired product (42.1 g, 93% yield). ¹H NMR (CDCl₃, 500 MHz): δ 5.69 (d, 1H, J 9.0 Hz), 5.40 (m, 1H), 5.37 (d, 1H, J 3.0 Hz), 5.08 (dd, 1H, J 3.0 Hz, 11 Hz), 4.44 (dt, 1H, J 9.5 Hz, 11 Hz), 4.17 (dd, 1H, J 7.0 Hz, 11.5 Hz), 4.11 (dd, 1H, J 7.0 Hz, 11.5 Hz), 4.01 (t, 1H, J 7.0 Hz), 2.17 (s, 3H), 2.13 (s, 3H), 2.05 (s, 3H), 2.02 (s, 3H), 1.94 (s, 3H), 1.57 (s, 3H).

Preparation of benzyl 5-hydroxy pentanoate (NAG5).

A solution of delta-valerolactone (10.0 g, 100 mmol) and NaOH (4.00 g, 100 mmol) in water (100 mL) was stirred overnight at 70° C. The reaction mixture was cooled to rt and concentrated in vacuo to give white solid NAG4. This solid was suspended in acetone (100 mL) and refluxed overnight with benzyl bromide (20.5 g, 120 mmol) and tetrabutylammonium bromide (1.61 g, 0.50 mmol). Acetone was removed in vacuo to afford an oily residue, which was dissolved in EtOAc and washed with sat NaHCO₃(aq.) and brine. The organic layer was dried over Na₂SO₄ and concentrated in vacuo give the oily product NAG5 (17.1 g, 82% yield). ¹H NMR (CDCl₃, 500 MHz): δ 7.35 (m, 5H), 3.64 (q, 2H, J 6 Hz, 11.5 Hz), 2.41 (t, 2H, J 7.5 Hz), 1.75 (m, 2H), 1.60 (m, 2H), 1.44 (t, 1H, J 6 Hz).

Preparation of benzyloxycarbonylbutyl 2-deoxy 2-N-acetyl-3,4,6-tri-O-acetyl-β-D-galactopyranoside (NAG7)—Method A.

Under an inert atmosphere, TMSOTf (8.56 g, 38.4 mmol) was added to a solution of NAG2 (10.0 g, 25.6 mmol) in DCE (100 mL) at ambient temperature. The mixture was stirred at 55° C. for 2 h, removed from heat, and stirred overnight. The reaction mixture was poured onto ice cold sat. NaHCO₃(aq.) and extracted with CH₂Cl₂. The separated organic layer was dried over Na₂SO₄ and concentrated in vacuo to give syrup NAG6. A solution NAG6 in DCE (60 mL) was charged with alcohol NAG5 (8.00 g, 38.4 mmol) and molecular sieves. The mixture was placed under an inert atmosphere, treated with TMSOTf (2.85 g, 12.8 mmol), and stirred overnight at room temperature. The mixture was poured over ice cold sat. NaHCO₃ (aq.) and extracted with CH₂Cl₂. The organic layer was dried over Na₂SO₄ and concentrated in vacuo to give a crude material as syrup. This crude material was purified by SiO₂ gel chromatography to afford glycoside NAG7 (3.3 g, 24% yield). ¹H NMR (CDCl₃, 500 MHz): δ 7.35 (m, 5H), 5.98 (d, 1H, J 7.0 Hz), 5.57 (m, 1H), 5.34 (d, 1H, J 3.0 Hz), 5.25 (dd, 1H, J 3.0 Hz, 11 Hz), 5.10 (s, 2H), 4.63 (d, 1H, J 8.5 Hz), 4.11 (m, 2H), 3.95 (m, 1H), 3.88 (m, 2H), 3.49 (m, 1H), 2.37 (m, 2H), 2.13 (s, 3H), 2.03 (s, 3H), 1.99 (s, 3H), 1.90 (s, 3H), 1.70 (m, 2H), 1.61 (m, 2H).

Preparation of benzyloxycarbonylbutyl 2-deoxy 2-N-acetyl-3,4,6-tri-O-acetyl-3-D-galactopyranoside (NAG7)—Method B.

To a solution of NAG2 (5.00 g, 12.8 mmol) and alcohol NAG5 (5.33 g, 25.6 mmol) in DCE (50 mL) was added Sc(OTf)₃ (0.44 g, 0.90 mmol) in one portion. The mixture was placed under an inert atmosphere and refluxed for 3 h. Upon cooling, the mixture was diluted with CH₂Cl₂, washed with sat NaHCO₃(aq.), dried over MgSO₄, and concentrated in vacuo. Purification by SiO₂ gel chromatography afforded glycoside NAG7 (5.53 g, 80% yield).

Preparation of carboxybutyl 2-deoxy 2-N-acetyl-3,4,6-tri-O-acetyl-f-D-galactopyranoside (NAG8).

A solution of glycoside NAG7 (1.50 g, 2.41 mmol) in EtOH (25 mL) was degassed under vacuum and purged with Argon. The Palladium catalyst (10% wt. on activated carbon, 0.50 g) was added in one portion and the mixture was degassed under vacuum purged with argon. The heterogeneous mixture was charged with cyclohexene (25 mL) and refluxed for 6 h. Upon cooling, the catalyst was removed by filtration and the mother liquor concentrated in vacuo. The crude was purified by SiO₂ gel chromatography to afford a white foam NAG8 (0.76 g, 70% yield). 1H NMR (CDCl₃, 500 MHz): δ 5.72 (d, 1H, J 8.5 Hz), 5.35 (d, 1H, J 3.5 Hz), 5.26 (dd, 1H, J 3.5 Hz, 11.5 Hz), 4.67 (d, 1H, J 8.5 Hz), 4.17 (dd, 1H, J 6.5 Hz, 11.5 Hz), 4.12 (dd, 1H, 6.5 Hz, 11.5 Hz), 4.00 (dt, 1H, J 8.5 Hz, 11.5 Hz), 3.92 (m, 2H), 3.53 (m, 1H), 2.39 (m, 2H), 2.15 (s, 3H), 2.05 (s, 3H), 2.01 (s, 3H), 1.97 (s, 3H), 1.71 (m, 2H), 1.65 (m, 2H).

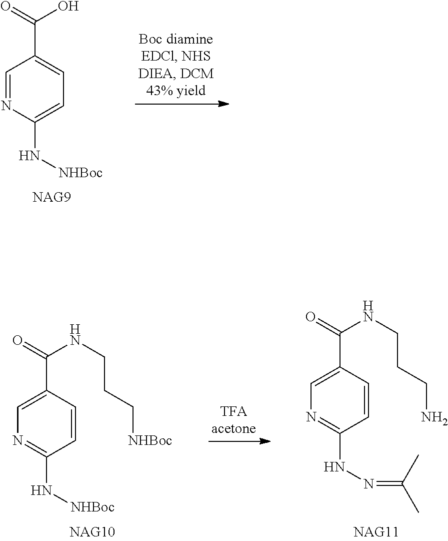 [SMILES: CC(C)(C)OC(=O)NNc1ccc(C(=O)O)cn1 ; CC(C)(C)OC(=O)NCCCNC(=O)c1ccc(NNC(=O)OC(C)(C)C)nc1 ; CC(C)=NNc1ccc(C(=O)NCCCN)cn1]

Preparation of aminopropyl 6-hydrazinonicotamide acetone hydrazone (NAG11).

Boc 6-hydrazinonicotinic acid (520 mg, 2.1 mmol) in DCM (20 mL) was treated to EDCI (440 mg, 2.3 mmol), N-hydroxysuccinimide (NHS; 260 mg, 2.3 mmol), Boc-diamine (650 mg, 2.6 mmol), and DIEA (1.1 mL, 6.2 mmol) for 3 h. The reaction was concentrated in vacuo and purified by silica gel chromatography to afford NAG10 (364 mg, 43% yield). ¹H NMR (CDCl₃, 500 MHz): δ 8.55 (br, 1H), 7.93 (d, 2H, J 7.5 Hz), 7.45 (br, 1H), 7.12 (br, 1H), 6.62 (d, 1H, J 8.5 Hz), 5.17 (br, 1H), 3.42 (m, 2H), 3.13 (m, 2H), 1.65 (m, 2H), 1.41 (s, 18H). The HyNic acetone hydrazone was formed through treatment of NAG10 (160 mg, 0.4 mmol) with TFA (9 mL) and acetone (1 mL) for 1 h. The reaction mixture was concentrated in vacuo and placed on the high vacuum to afford NAG11.

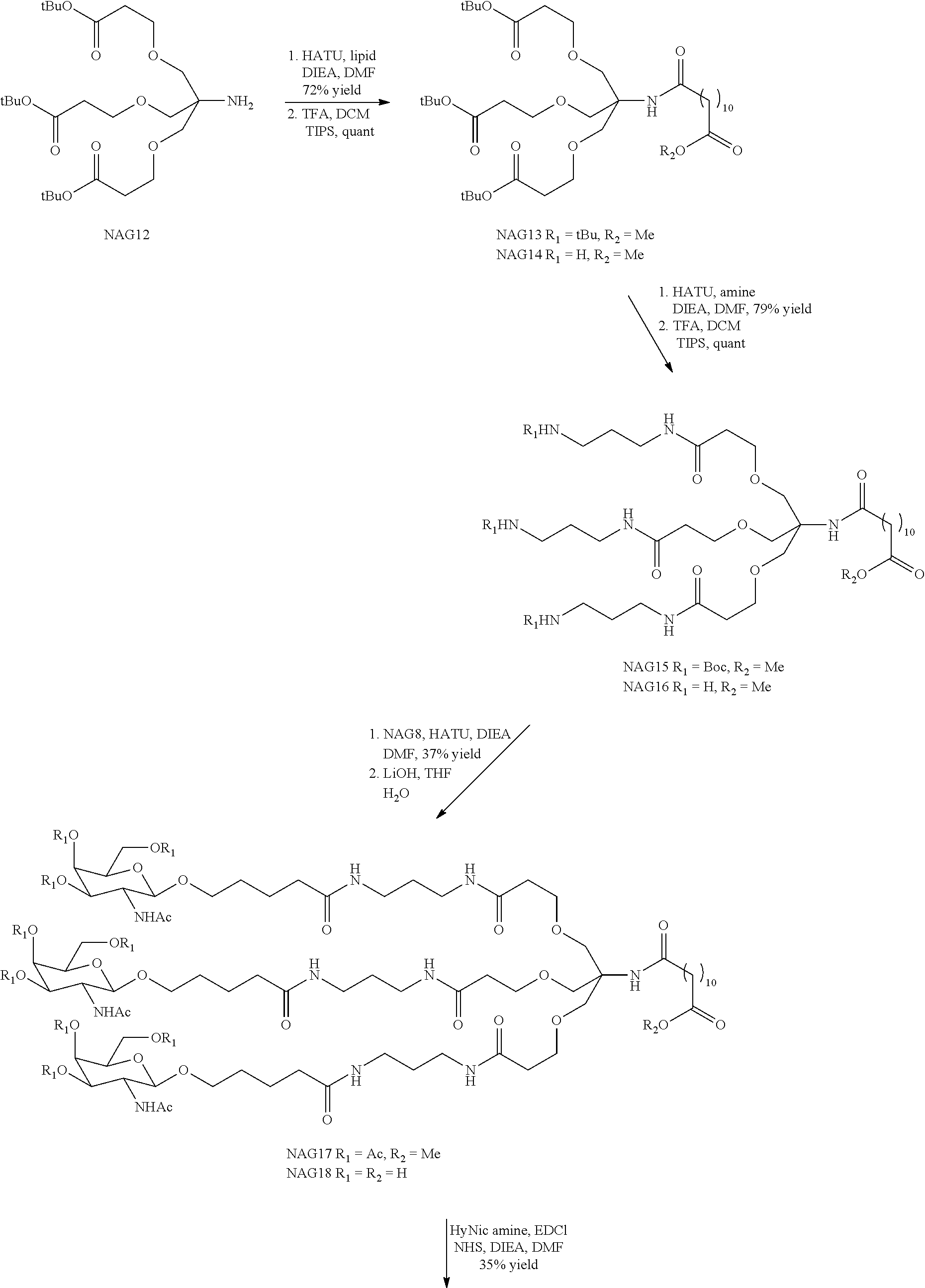

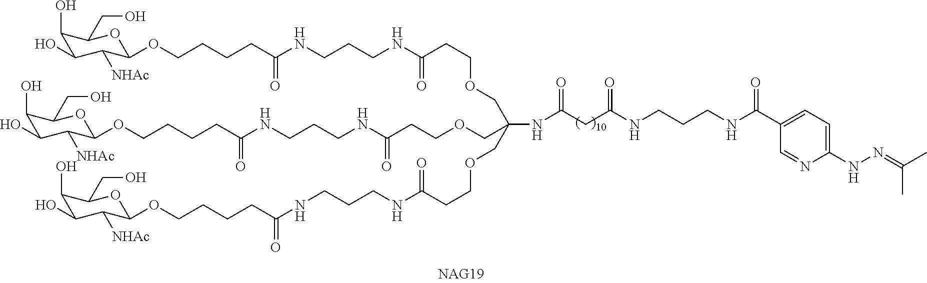

Preparation of tris-(carboxyethoxymethyl)-methylamido-dodecanedioate methyl ester (NAG14).

To a solution of dodecanedioic acid methyl ester (211 mg, 0.42 mmol) activated with HATU (122 mg, 0.50 mmol) and DIEA (218 μL, 1.25 mmol) in DMF (2 mL) was added tris linker NAG12. After 1 h, the reaction mixture was concentrated in vacuo and purified by SiO₂ gel chromatography to afford NAG13 (214 mg, 70% yield). MALDI-TOF mass calcd C₃₈H₆₉NO₁₂: 731.48, Found: 755.10 [M+Na]. Tris t-butyl ester NAG13 was hydrolyzed with a TFA:TIPS:DCM (9:0.25:1) cocktail (10.25 mL) for 4 h and concentrated in vacuo to give tris acid NAG14. MALDI-TOF mass calcd C₂₆H₄₅NO₁₂: 563.29, Found: 565.33 [M+H].

Preparation of tris-(aminopropamido-ethoxymethyl)-methylamido-dodecanedioate methyl ester (NAG16).

To a solution of tris acid NAG14 (230 mg, 0.41 mmol) activated with HATU (557 mg, 1.35 mmol) and DIEA (470 μL, 2.70 mmol) in DMF (4 mL) was added monoBoc 1,3-diaminopropane (250 mg, 1.44 mmol). After 1 h, the reaction was concentrated in vacuo and purified by SiO₂ gel chromatography to afford NAG15 (335 mg, 79% yield). MALDI-TOF mass calcd C₅₀H₉₃N₇O₁₅: 1031.67, Found: 1056.40 [M+Na]. Tris Boc linker NAG15 was treated with a TFA:TIPS:DCM (9:0.25:1) cocktail (10.25 mL) for 1 h and concentrated in vacuo to give tris amine NAG16. MALDI-TOF mass calcd C₃₅H₆₉N₇O₉: 731.51, Found: 733.18 [M+H].

Preparation of tris-Ga/NAc (NAG18):

Monosaccharide NAG8 (192 mg, 0.43 mmol) was treated with HATU (163 mg, 0.43 mmol) and DIEA (150 μL, 0.86 mmol) in DMF (2 mL). After 30 min, a solution of NAG16 (80 mg, 0.11 mmol) in DMF (1 mL) was added and the mixture stirred for 1 h. The crude mixture was purified by SiO₂ gel chromatography to afford NAG17 (82 mg, 37% yield). Mass calcd C₉₂H₁₅₀N₁₀O₃₉: 2019.00, Found: 2041.85 [M+Na]. The peracetylated trimer GalNAc (82 mg, 0.04 mmol) was hydrolyzed upon treatment with LiOH.H₂O (34 mg, 0.81 mmol) in a THF:H₂O (3:1) solution (8 mL) to afford NAG18. MALDI-TOF mass calcd C₇₃H₁₃₀N₁₀O₃₀: 1626.89, Found: 1634.52 [M+Li].

Preparation of HyNic trimer Ga/NAc (NAG19).

A solution GalNAc trimer NAG18 (32 mg, 0.02 mmol) and HyNic amine NAG11 (20.0 mg, 0.08 mmol) in DMF (1 mL) was treated with EDCI (16.2 mg, 0.08 mmol), NHS (2.5 mg, 0.02 mmol), and DIEA (28 μL, 0.16 mmol) and stirred for 4 h. Upon concentration in vacuo, the crude was dissolved in DMSO and purified by RP-HPLC to afford NAG19 (12.6 mg, 35% yield). MALDI-TOF mass calcd C₈₅H₁₄₇N₁₅O₃₀: 1858.04, Found: 1859.83 [M+H].

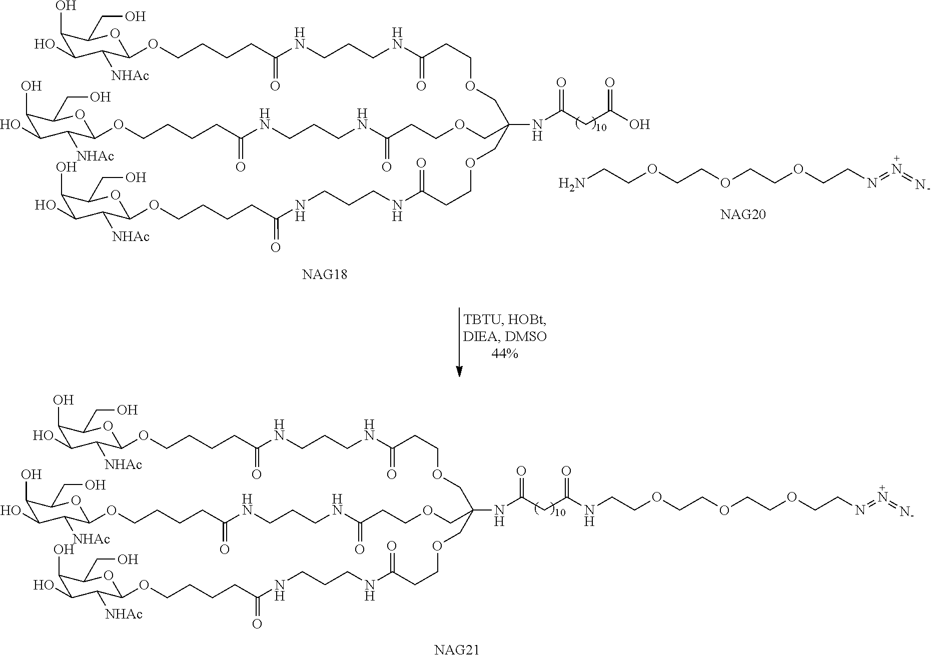

Preparation of azido-PEG3-trimer Ga/NAc (NAG21).

GalNAc trimer carboxylic acid NAG18 (60 mg, 0.03 mmol), azido-PEG3-amine NAG20 (45.6 mg, 0.21 mmol), TBTU (23.8 mg, 0.07 mmol), HOBt (11.5 mg, 0.03 mmol), and DIEA (34 μL) were dissolved in DMSO (0.5 mL) and stirred 2 h. The base was removed in vacuo and the crude purified by RP-HPLC to afford NAG21 (24 mg, 44%). AP-ESI+ Mass calcd C₈₁H₁₄₆N₁₄O₃₂: 1827.02, Found: 914.8 [M+2H]²⁺.

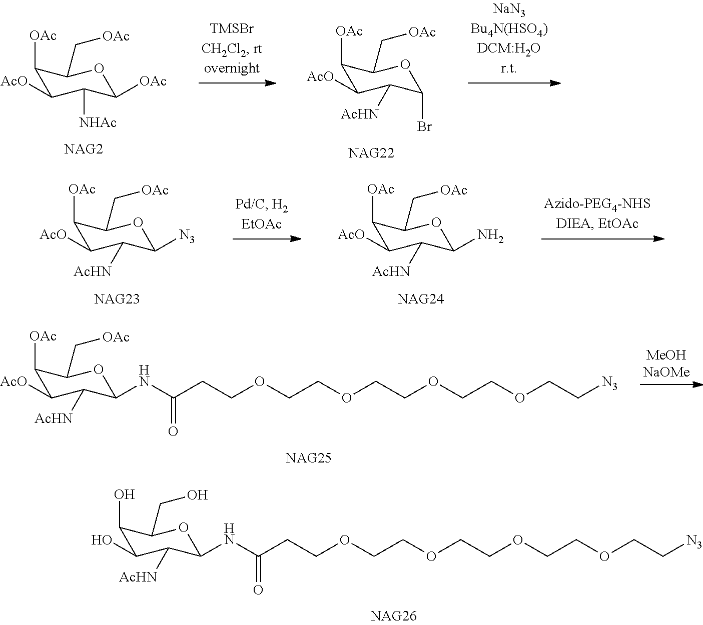 [SMILES: CC(=O)NC1C(OC(C)=O)OC(COC(C)=O)C(OC(C)=O)C1OC(C)=O ; CC(=O)NC1C(Br)OC(COC(C)=O)C(OC(C)=O)C1OC(C)=O ; CC(=O)NC1C(N=[N+]=[N-])OC(COC(C)=O)C(OC(C)=O)C1OC(C)=O ; CC(=O)NC1C(N)OC(COC(C)=O)C(OC(C)=O)C1OC(C)=O ; CC(=O)NC1C(NC(=O)CCOCCOCCOCCOCCN=[N+]=[N-])OC(COC(C)=O)C(OC(C)=O)C1OC(C)=O ; CC(=O)NC1C(NC(=O)CCOCCOCCOCCOCCN=[N+]=[N-])OC(CO)C(O)C1O]

Preparation of 1-bromo 2-deoxy-2-acetamido 3,4,6-tri-O-acetyl-Pi-D-galactopyranoside (NAG22).

To a D-galactosamine pentaacetate (NAG2, 10.0 g, 1 eq, 25.8 mmol) suspension in DCM (90 mL) at 0° C. in an ice bath under an argon balloon was added bromotrimethylsilane (4.1 mL, 1.2 eq, 31 mmol) drop wise with stirring. Ice bath was removed after 10 minutes and the reaction was allowed to stir at room temperature overnight. Reaction was checked by TLC (Hanessians Stain) in 75% hexanes:ethyl acetate. Reaction was concentrated in vacuo, azeotroped with cyclohexane (3×50 mL). Dried on high vacuum overnight and used as is.

Preparation of 1-azido 2-deoxy-2-acetamido 3,4,6-tri-O-acetyl-β-D-galactopyranoside (NAG23).

NAG22 (10.6 g, 1.0 eq, 25.8 mmol) was dissolved in DCM (100 mL). To this solution was added sodium azide (4.86 g, 2.9 eq, 74.8 mmol) in water (100 mL) and tetrabutylammonium bisulfate (8.32 g, 0.95 eq, 24.5 mmol). The reaction mixture was stirred vigorously for 1 hour. Reaction was checked by TLC (Hanessians Stain) in 75% Hexanes:Ethyl Acetate. Reaction was extracted with DCM (2×50 mL). The organic layer was dried over anhydrous MgSO₄ and concentrated in vacuo. The material was then purified by silica gel flash chromatography (3:1 Hexanes:Ethyl acetate). Proton NMR of the material collected was consistent with the published structure. M+H=373.0

Preparation of 1-amino 2-acetamido 1,2-dideoxy 3,4,6-tri-O-acetyl-β-D-galactopyranose (NAG24).

To NAG23 (0.26 g, 1 eq, 0.7 mmol) dissolved in ethyl acetate (25 mL) was added palladium on carbon (˜26 mg). Next a hydrogen balloon and vacuum line were inserted. The reaction was evacuated 3× and purged with hydrogen after each evacuation. Reaction was stirred at room temperature for 1 hour. LC/MS after 1 hour confirmed the formation of the product. Reaction was filtered over a bed of celite. Wash the celite 3×10 mL of EtOAc. Reaction was concentrated in vacuo and used in the next step as is. M+H=346.6

Preparation of 1-amino (15′-azido-tetraethyleneglycol propanoyl) 2-acetamido 1,2-dideoxy-β-D-galactopyranoside (NAG26).

To NAG24 (0.24 g, 1 eq, 0.7 mmol) dissolved in ethyl acetate (45 mL) and DIEA (0.24 mL, 2 eq, 1.4 mmol) was added azido-PEG4-NHS (0.41 g, 1.5 eq, 1.05 mmol) in ethyl acetate (5 mL) drop wise with stirring under an argon balloon. The reaction was allowed to stir at room temperature overnight. Completion of the reaction was verified by LC/MS. M+H=619.5. Ethyl acetate was removed in vacuo and use as is in the next step. To NAG 25 (0.43 g, 1 eq, 0.7 mmol) dissolved in MeOH (10 mL) was added 100 μL of a 25% sodium methoxide solution in methanol. Reaction stirred at room temperature for 1 hour under argon balloon. LC/MS after 1 hour showed only starting material. Added 500 μL of a 25% sodium methoxide solution in methanol. LC/MS after 1 hour showed formation of product and disappearance of starting material. Dowex resin was added until pH of solution reached 7. The resin was removed Filter off resin, remove solvent in vacuo and purify by reverse phase HPLC. M+H=493.7.

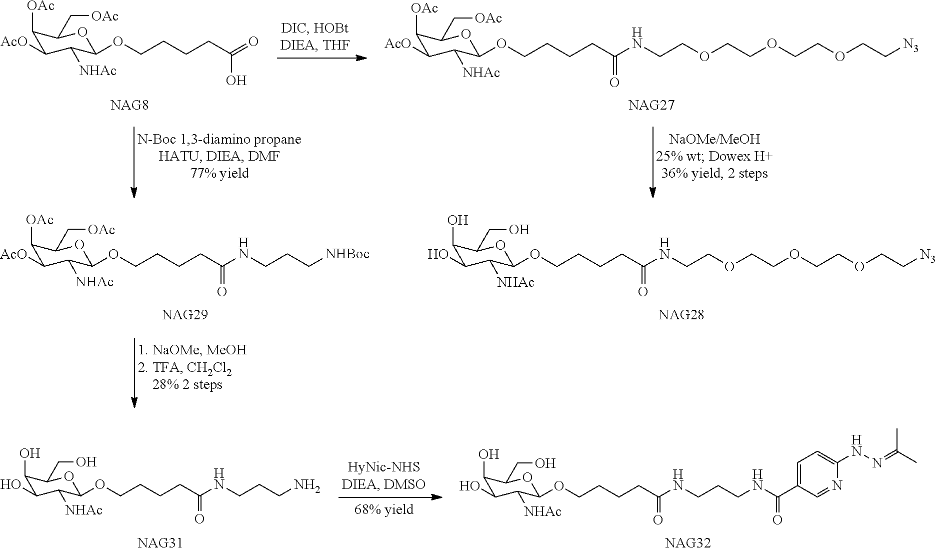 [SMILES: CC(=O)NC1C(OCCCCC(=O)O)OC(COC(C)=O)C(OC(C)=O)C1OC(C)=O ; CC(=O)NC1C(OCCCCC(=O)NCCOCCOCCOCCN=[N+]=[N-])OC(COC(C)=O)C(OC(C)=O)C1OC(C)=O ; CC(=O)NC1C(OCCCCC(=O)NCCCNC(=O)OC(C)(C)C)OC(COC(C)=O)C(OC(C)=O)C1OC(C)=O ; CC(=O)NC1C(OCCCCC(=O)NCCOCCOCCOCCN=[N+]=[N-])OC(CO)C(O)C1O ; CC(=O)NC1C(OCCCCC(=O)NCCCN)OC(CO)C(O)C1O ; CC(=O)NC1C(OCCCCC(=O)NCCCNC(=O)c2ccc(NN=C(C)C)nc2)OC(CO)C(O)C1O]

Preparation of [5-(2-{2-[2-(2-azidoethoxy)ethoxy]ethoxy}ethylamino)-5-oxopentanoyl] 2-deoxy 2-N-acetyl-3,4,6-tri-O-acetyl-β-D-galactopyranoside (NAG27).

To a solution of NAG8 (1.00 g, 2.24 mmol) in THF (8 mL) was added DIC (0.56 g, 4.48 mmol) and HOBt (0.25 g, 2.17 mmol). After 1 h, white precipitate had formed and the reaction was cooled to 0° C. A solution azido-PEG3-amine (0.63 g, 2.91 mmol) in THF (2 mL) was added and the reaction was stirred for an additional 1 h. RP-HPLCMS showed formation of the desired NAG27. ESI MS+ mass calculated C₂₇H₄₅N₅O₁₃: 647.7, Found: 647.8 [M+H]. The precipitate was removed by filtration and the reaction concentrated in vacuo to give thick syrup.

Preparation of [5-(2-{2-[2-(2-azidoethoxy)ethoxy]ethoxy}ethylamino)-5-oxopentanoyl] 2-deoxy 2-N-acetyl -β-D-galactopyranoside (NAG28).

Crude NAG27 was dissolved in anhydrous methanol (10 mL) and treated with NaOMe in MeOH (25 wt %, 250 μL). The reaction was stirred overnight at room temperature. After which, RP-HPLCMS showed consumption of NAG27 and formation of the NAG28. ESI MS+ mass calculated C₂₁H₃₁N₅O₁₀: 521.6, Found: 522.3 [M+H]. Dowex H+ resin was added to neutralize the base, the resin was removed by filtration, and the liquor was concentrated in vacuo. Crude NAG28 was purified by RP-HPLC to afford 0.42 g, 36% yield over two steps.

Preparation of ([3-(tert-butoxycarbonylamino)propylamino])-5-oxopentanoyl] 2-deoxy 2-N-acetyl-3,4,6-tri-O-acetyl-β-D-galactopyranoside (NAG29).

To a solution of NAG8 (0.29 g, 0.65 mmol) in DMF (3 mL) was activated with HATU (0.25 g, 0.65 mmol) and DIEA (0.34 mL, 1.95 mmol). After 10 min, NH-Boc 1,3-diaminopropane (0.13 g, 0.72 mmol) was added and stirred for 2 h. The mixture was concentrated in vacuo and purified by SiO₂ chromatography to provide NAG29 (0.30 g, 77% yield). ESI MS+ mass calculated C₂₇H₄₅N₃O₁₂: 603.7, Found: 626.8 [M+Na].

Preparation of ([3-(amino)propylamino])-5-oxopentanoyl] 2-deoxy 2-N-acetyl-β-D-galactopyranoside (NAG31).

A solution of NAG29 (0.30 g, 0.50 mmol) in anhydrous methanol was treated with NaOMe in MeOH (25 wt %, 50 μL). After 20 min, TLC showed complete consumption of NAG29. Dowex strong H+ resin was added to acidify the reaction and stirred for 30 min. The resin was filtered off and washed with 1% TEA in MeOH and 1M NaOH (aq). The filtrate was neutralized with 1M HCl (aq) and concentrated in vacuo to give NAG31 (0.052 g, 28% yield). ESI MS+ mass calculated C₁₆H₃₁N₃O₇: 377.4, Found: 377.6 [M+H].

Preparation of ({3-[6-(isopropylidenehydrazino)-nicotinoylamino]propylamino}-5-oxopentanoyl) 2-deoxy 2-N-acetyl-β-D-galactopyranoside (NAG32).

A solution NAG31 (0.009 g, 22 umol) in DMSO (1 mL) was treated with HyNic-sulfo-NHS (0.007 g, 18 umol) and DIEA (9.4 μL, 54 umol) for 1 h and purified by RP-HPLC to afford NAG32 TFA salt (0.010 g, 68% yield). ESI MS+ mass calculated C₂₅H₄₀N₆O₈: 552.6, Found: 554.0 [M+H].

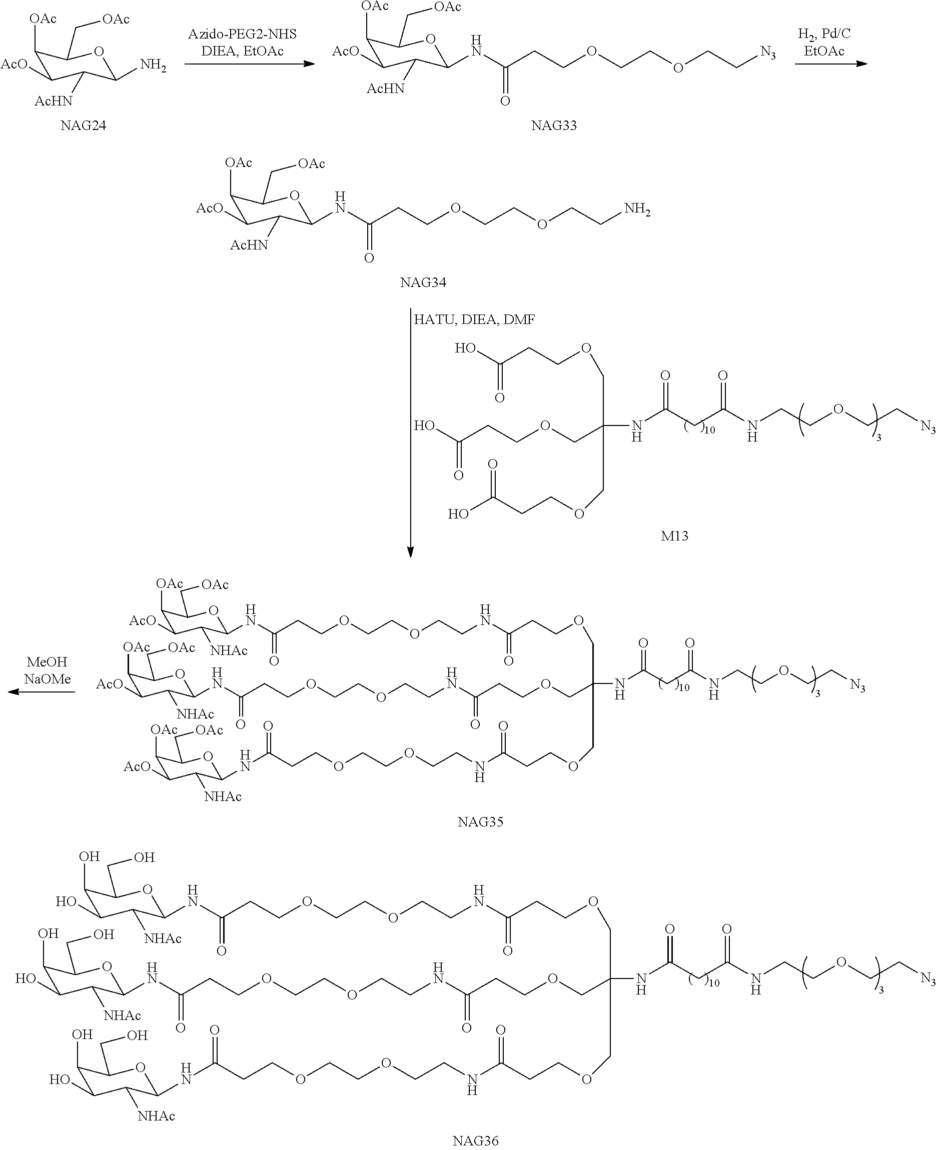

Preparation of 1-amino-(15′-azido-diethyleneglycol propranoyl) 2-acetamido 1,2-dideoxy 3,4,6-tetra-O-acetyl-β-D-galactopyranoside (NAG33).

To NAG24 (2.3 g, 6.7 mmol) dissolved in ethyl acetate (90 mL) and DIEA (1.7 mL, 9.9 mmol) was added azido-PEG2-NHS (1.0 g, 3.3 mol) in ethyl acetate (10 mL) drop wise with stirring under an argon balloon. The reaction was allowed to stir at room temperature overnight. Completion of the reaction was verified by LC/MS. ESI MS+. The crude product was purified by RP-HPLC to afford NAG33 (1.2 g). Mass calcd C₂₁H₃₃N₅O₁₁: 531.22, Found: 532.3 [M+H]+.

Preparation of 1-amino-[2-(2-aminoethoxy)ethoxy]propionyl] 2-acetamido 1,2-dideoxy 3,4,6-tetra-O-acetyl f-D-galactopyranose (NAG34).

To NAG33 (1.2 g, 2.3 mmol) dissolved in ethyl acetate (25 mL) was added palladium (10% Pd on activated carbon, 122 mg). Next a hydrogen balloon and vacuum line were inserted. The reaction was evacuated and purged with hydrogen gas (3 cycles). After stirring at room temperature for 1 hour, RP-HPLC/MS confirmed the formation of the product. Reaction was filtered over a bed of celite and washed with EtOAc (3×10 mL). Concentration in vacuo afforded in quantitative yield. ESI MS+ Mass calcd C₂₁H₃₅N₃O₁₁: 505.23, Found: 506.3 [M+H]⁺.

Preparation of peracetylated azido PEG2 N-GalNAc trimer (NAG36).

To tri-acid linker M13 (52.5 mg, 0.07 mmol), DIEA (73.2 μL, 0.42 mmol) in DMF (2 mL) was added HATU (119.8 mg, 0.32 mmol) in DMF (2 mL). The reaction was allowed to stir for 10 minutes at room temperature. Next NAG34 (159 mg, 0.32 mmol) in DMF (1 mL) was added, and the mixture was stirred overnight at room temperature. LC/MS after 18 hour confirmed the formation of the product. Water (10 mL) was added, and the resulting mixture was washed with DCM (3×5 mL). The separated organic layers were dried over Na₂SO₄, filtered, and concentrated in vacuo to afford NAG35 (155 mg), ESI MS+ mass calcd C₉₆H₁₅₈N₁₄O₄₄: 2210.5, Found: 1106.8 [M+2H]²⁺. To a solution of NAG35 (155 mg, 70 μmol) in MeOH (5 mL) was added sodium methoxide (25% wt MeOH, 100 μL). The reaction mixture was stirred at room temperature for 1 hour under argon balloon. LC/MS after 1 hour showed product formation and disappearance of starting material. Dowex H+ strongly acidic resin was added to neutralize the reaction. The resulting mixture was filtered to remove resin, and the filtrate was concentrated in vacuo to give a crude product, which, upon purification by reverse phase HPLC, afforded NAG36 (10 mg). ESI MS+ Mass calcd C₇₈H₁₄₀N₁₄O₃₅: 1832.96, Found: 917.7 [M+2H]²⁺.

Compounds NAG37, NAG38, and NAG39 were prepared in a manner similar to the synthesis of NAG26.

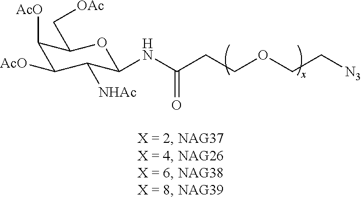

TABLE 2

| Compound # | Peg-X | Molecular formula | MS calc | MS found (ESI+) |
|---|---|---|---|---|
| NAG37 | 2 | C₁₅H₂₇N₅O₈ | 405.4 | 406.2 |
| NAG26 | 4 | C₁₉H₃₅N₅O₁₀ | 493.5 | 493.7 |
| NAG38 | 6 | C₂₃H₄₃N₅O₁₂ | 581.6 | 581.8 |
| NAG39 | 8 | C₂₇H₅₁N₅O₁₄ | 669.7 | 670 |

### Phosphotriester Oligonucleotide Synthesis:

### General Scheme:

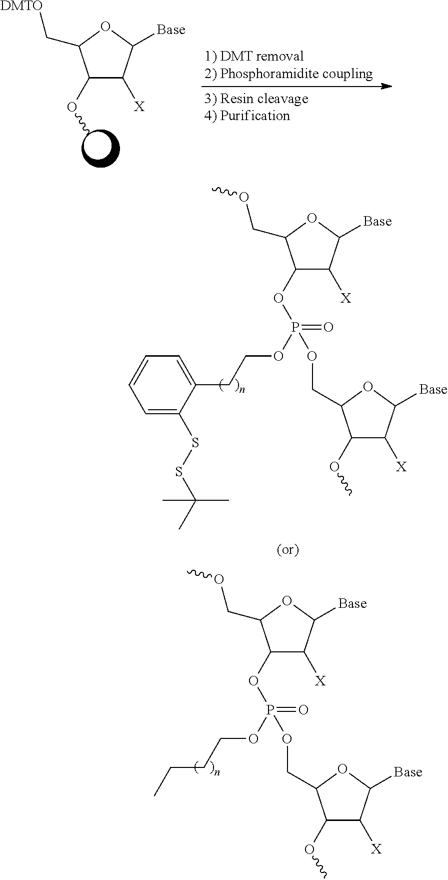

### Experimental Details:

All the oligonucleotide sequences synthesized were modified at 2′-ribose sugar position with 2′-F and 2′-OMe modifications to improve serum stability and to minimize off-target effects. Automated oligonucleotide synthesis (1 μmol scale) was carried out with the following reagents/solvents:
Oxidizer—0.02 M I₂ in THF/pyridine/H₂O (60 s oxidation per cycle)
Deblocking agent—3% trichloroacetic acid (2×40 s deblocks per cycle)
Cap Mix A—THF/pyridine/Pac₂O (60 s capping per cycle)
Cap Mix B—16% methyl imidazole in THF (60 s capping per cycle)
Sulfurization—0.05 M sulfurizing Reagent 3-((N,N-dimethylaminomethylidene)amino)-3H-1,2,4-dithiazole-5-thione, DDTT in 60% pyridine/40% acetonitrile (360 s sulfurization per cycle)
   Exceptions to standard oligonucleotide synthesis conditions were as follows:
Controlled Pore Glass (CPG) supports with Q-linkers (hydroquinone-O,O′-diacetic acid linker arm) for milder deprotection were used
All disulfide phosphoramidites were resuspended to 100 mM in 100% anhydrous acetonitrile prior to synthesis
Phosphoramidite activation was performed with 2.5-fold molar excess of 5-benzylthio-1-H-tetrazole (BTT). Activated phosphoramidites were coupled for 2×3 minute coupling steps per insertion.

### Oligonucleotide Deprotection & Purification Protocol:

Following automated oligonucleotide synthesis, phosphotriester oligonucleotides were cleaved and deprotected in 1 mL of 10% diisopropylamine in methanol (10% DIA/MeOH) for 4 h at room temperature. Following the 4 h deprotection, oligo samples were dried by centrifugal evaporation.

Oligo synthesis using phosphoramidite monomers having standard protecting groups (such as A-Bz, C—Ac and G-iBu etc.), phosphotriester oligonucleotides were cleaved and deprotected in 1.0 mL of AMA (1:1 ratio of 36% aq. ammonia and 40% methylamine in methanol) for 2 h at room temperature followed by centrifugal evaporation.

Crude oligo pellets were resuspended in 100 μL of 50% acetonitrile briefly heated to 65° C., and vortexed thoroughly. Total 100 μL crude oligo samples were injected onto RP-HPLC with the following buffers/gradient:
Buffer A=50 mM TEAA in Water
Buffer B=90% Acetonitrile
Flow Rate=1 mL/min
Gradient:
   0-2 min (100% Buffer A/0% Buffer B)
   2-42 min (0% to 60% Buffer B)
   42-55 min (60% to 100% Buffer B)
Across the dominant RP-HPLC peaks, 0.5 mL fractions were collected and analyzed by MALDI-TOF mass spectrometry to confirm presence of desired mass. Purified fractions containing correct mass were frozen and lyophilized. Once dry, fractions were resuspended, combined with corresponding fractions, frozen and lyophilized for final product.

Triester insertions requiring additional deprotection were initially isolated as described above followed by the necessary secondary deprotection steps (see below):

### Secondary Deprotection of Phosphotriester Oligonucleotide Having Acetal Group:

RP-HPLC purified oligo products were resuspended in 100 μL of 80% formic acid. Reaction was allowed to proceed at room temperature for ˜1 h per aldehyde modification. Reaction was monitored by MALDI-TOF mass spectrometry to confirm complete deprotection. Once deprotection was complete, samples were frozen and lyophilized until dry. Lyophilized samples were then resuspended in 1 mL of 20% acetonitrile and gel-filtered for isolation of final oligo product.

### Secondary Deprotection of Phosphotriester Oligonucleotide Having TBDMS Protection:

RP-HPLC purified oligo products were resuspended in 219 μL of anhydrous DMSO, heated briefly to 65° C., and vortexed thoroughly. To the DMSO solution, 31 μL of 6.1 M triethylamine trihydrofluoride (TEA.3HF) was added to give a final concentration of 0.75 M. Reaction was allowed to proceed at room temperature for ˜1 h per TBDMS-protected hydroxyl modification. Reaction was monitored by MALDI-TOF mass spectrometry to confirm complete deprotection. Once deprotection was complete, 35 μL of 3 M sodium acetate followed by 1 mL of butanol were added. Samples were vortexed thoroughly and placed at −80° C. for 2 h. After 2 h, samples were centrifuged to pellet oligonucleotides. Butanol layer was removed, and the oligo pellet was resuspended in 1 mL of 20% acetonitrile. Samples were gel-filtered for isolation of final oligo product.

### Conjugation Methods

### Click Reaction:

### Copper—THPTA Complex Preparation:

A 5 mM aqueous solution of copper sulfate pentahydrate (CuSO₄.5H₂O) and a 10 mM aqueous solution of tris(3-hydroxypropyltriazolylmethyl)amine (THPTA) were mixed 1:1 (v/v) (1:2 molar ratio) and allowed to stand at room temperature for 1 hour. This complex can be used to catalyze Huisgen cycloaddition for example See General Conjugation Schemes 1-6.

### General Procedure (100 nM Scale):

To a solution of 710 μL of water and 100 μL tert-butanol (10% of final volume) in a 1.7 mL Eppendorf tube was added 60 μL of the copper-THPTA complex followed by 50 μL of a 2 mM solution of the oligo, 60 μL of a 20 mM aqueous sodium ascorbate solution and 20 μL of a 10 mM solution of GalNAc-azide. After thorough mixing the solution was allowed to stand at room temperature for 1 hour. Completion of the reaction was confirmed by gel analysis.

The reaction mixture is added to a screw cap vial containing 5-10 fold molar excess of SiliaMetS® TAAcONa (resin bound EDTA sodium salt). The mixture is stirred for 1 hour. This mixture is then eluted through an Illustra™Nap™-10 column Sephadex™. The solution is then frozen and lyophilized overnight.

### General Conjugation Scheme 1:

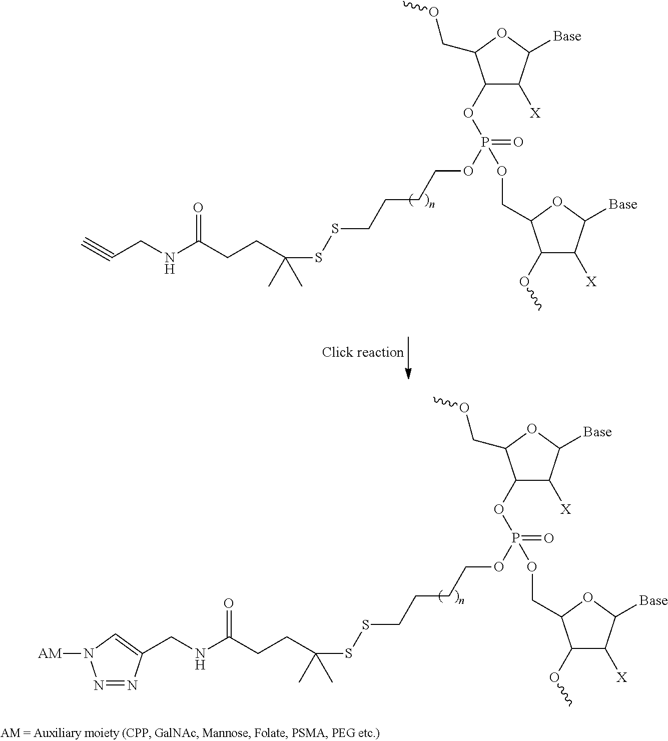

### General Conjugation Scheme 2:

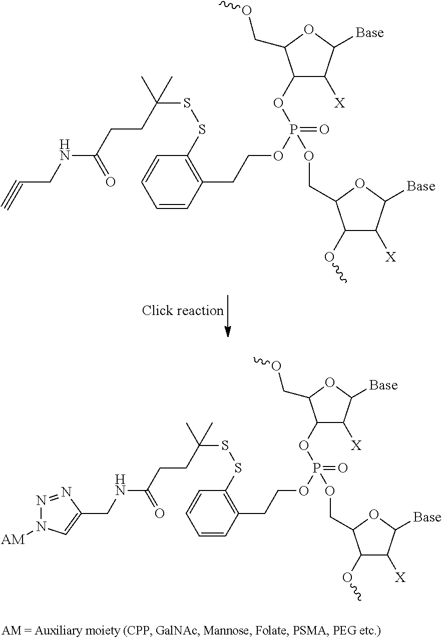

### General Conjugation Scheme 3:

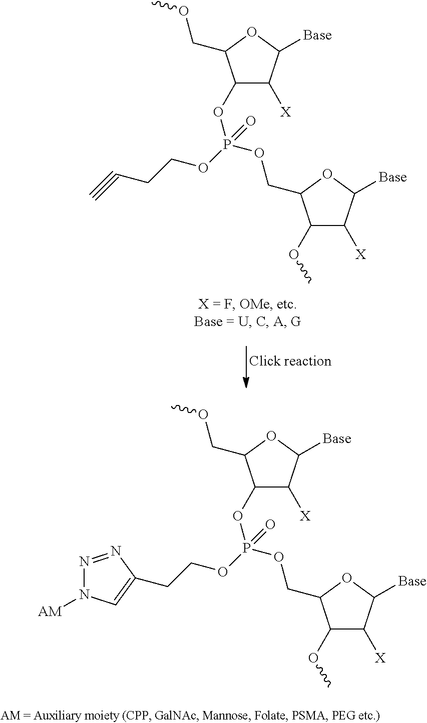

### General Conjugation Scheme 4:

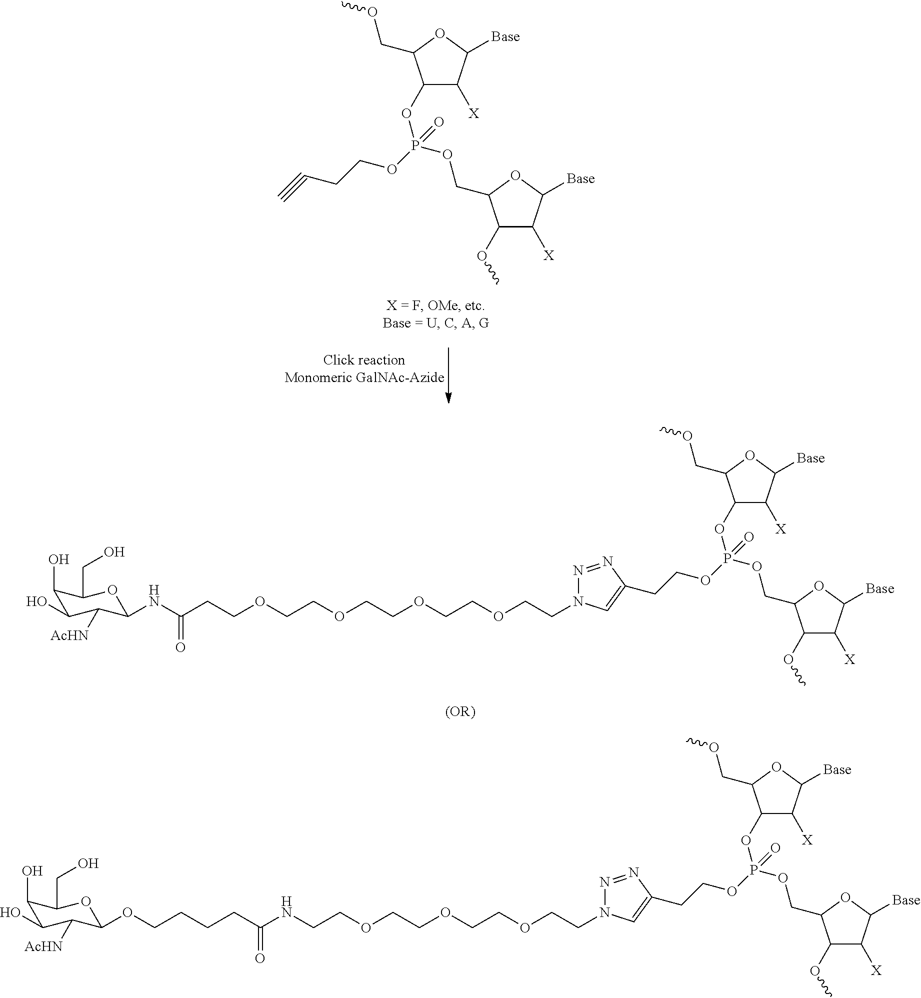

### General Conjugation Scheme 5:

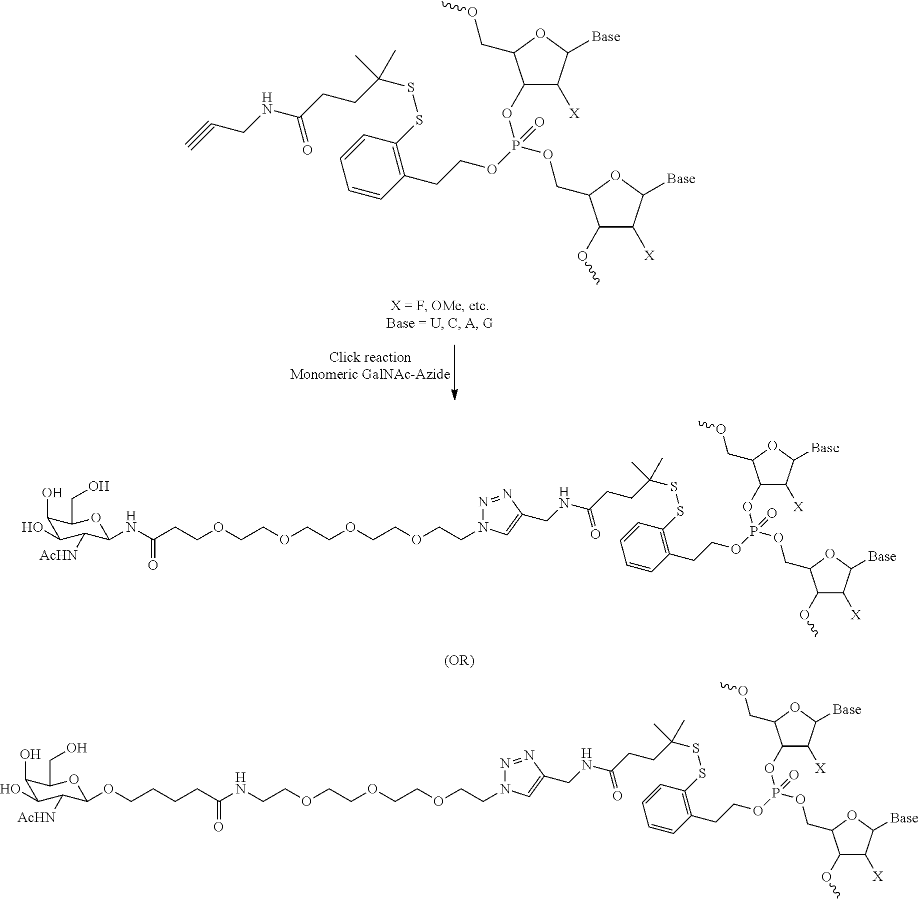

### General Conjugation Scheme 6:

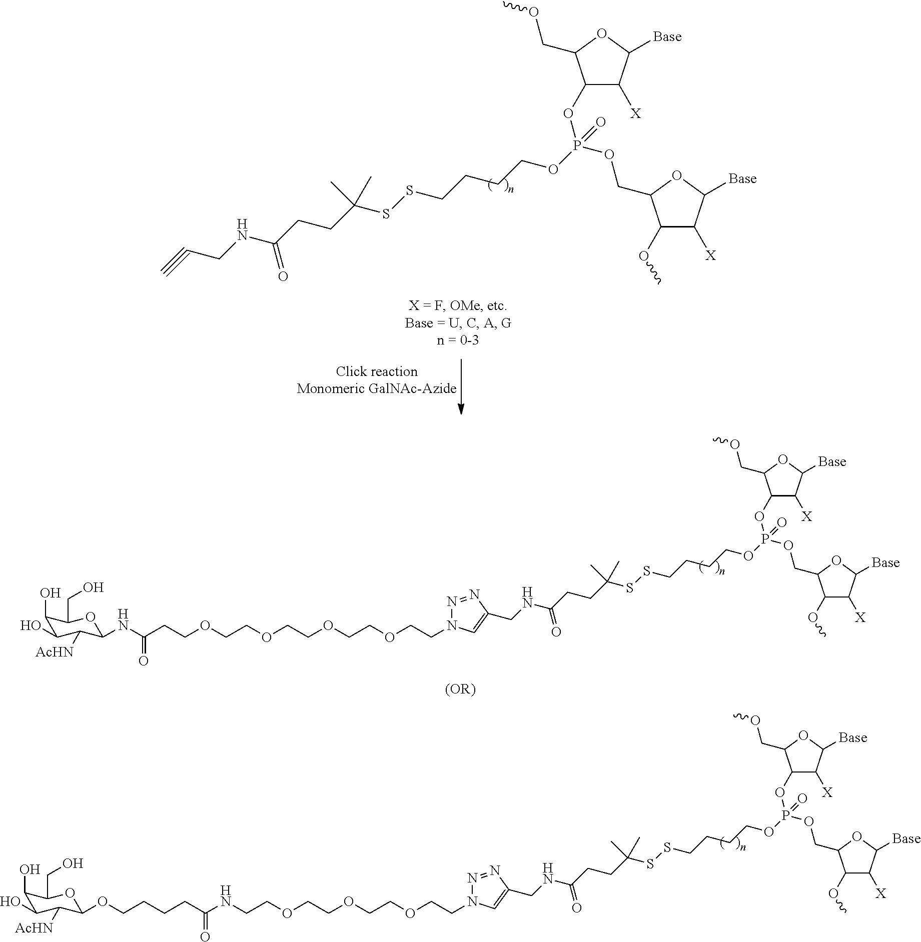

### General Conjugation Scheme 7:

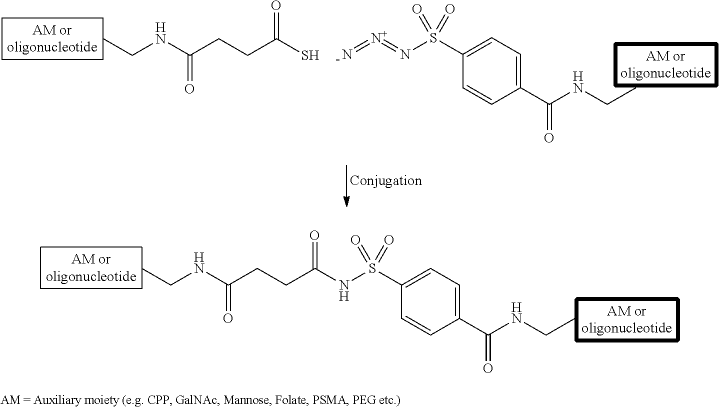

In general conjugation scheme 7, the conjugation product contains one and only one AM and one and only one oligonucleotide.

The conjugation schemes described herein are also applicable to non-bioreversible groups and differ from those showing bioreversible groups in that the non-bioreversible groups do not include the disulfide.

TABLE 3

| Compound | | | SEQ ID | | | Conjugation |
|---|---|---|---|---|---|---|
| # | Ligand | Target | NO: | Strand | Sequences (5′-3′) | Linker |
| SB-0097 | NAG21 | ApoB | 1 | P | UCaUCaCaCUgaaUaCCaaUt | Alkyne-Disulfide (ortho) |
| | | | 2 | G | UUggUaUUCagUgUgaUgaUt | |
| SB-0206 | NAG21 | AT3 | 3 | P | gpgUuAaCaCCAuUuAcUuCaa | Homopropargyl |
| | | | 4 | G | UUgAaGuAaAuggUgUuAaCcag | |
| SB-0209 | NAG21 | AT3 | 5 | P | GsgsUuAaCaCCAuUuAcUuCaA-Alk | 3′ Alkyne |
| | | | 6 | G | usUsgAaGuAaAuggUgUuAaCcsasg | |
| SB-0823 | NAG36 | AT3 | 3 | P | gpgUuAaCaCCAuLhaAcUuCama | Homopropargyl |
| | | | 4 | G | UUgAaGuAaAuggUgUuAaCcamg | |
| SB-0824 | NAG36 | AT3 | 3 | P | gpgUuAaCaCCAuLhaAcUuCaBa | Homopropargyl |
| | | | 4 | G | UUgAaGuAaAuggUgUuAaCcaBg | |
| SB-0825 | NAG36 | AT3 | 58 | P | gpgUuAaCaCCAuLhaAcUuCaMa | Homopropargyl |
| | | | 59 | G | UUgAaGuAaAuggUgUuAaCcaMg | |
| SB-0826 | NAG36 | AT3 | 3 | P | gpgUuAaCaCCAuLhaAcUuCaa | Homopropargyl |
| | | | 4 | G | UUgAaGuAaAuggUgUuAaCcag | |
| SB-0827 | NAG21 | ApoB | 7 | P | UpCaUCaCaCUgaaUaCCaaUt | Homopropargyl |
| | | | 8 | G | UUgGuAuUcAgUgUgAuGaUt | |
| SB-0828 | NAG36 | ApoB | 7 | P | UpCaUCaCaCUgaaUaCCaaUt | Homopropargyl |
| | | | 8 | G | UUgGuAuUcAgUgUgAuGaUt | |
| SB-0829 | NAG36 | ApoB | 7 | P | UpCaUCaCaCUgaaUaCCaaumt | Homopropargyl |
| | | | 9 | G | UUggUaUUCagUgUgaUgaumt | |
| SB-0830 | NAG21 | ApoB | 1 | P | UCaUCaCaCUgaaUaCCaaUt | Alkyne-Disulfide (ortho) |
| | | | 8 | G | UUgGuAuUcAgUgUgAuGaUt | |
| SB-0875 | NAG21 | AT3 | 10 | P | gsgsUuAaCaCCAuUuAcUuCsasa | |
| | | | 11 | G | UUgAaGuAaAuggUgUuAaCcsasg | Alkyne-Disulfide (ortho) |
| SB-0876 | NAG21 | AT3 | 10 | P | gsgUuAaCaCCAuUuAcUuCasa | |
| | | | 11 | G | UUgAaGuAaAuggUgUuAaCcasg | Alkyne-Disulfide (ortho) |
| SB-0877 | NAG21 | AT3 | 12 | P | GsgsUuAaCaCCAuUuAcUuCsasA | |
| | | | 11 | G | UUgAaGuAaAuggUgUuAaCcsasg | Alkyne-Disulfide (ortho) |
| SB-0884 | NAG21 | AT3 | 3 | P | gpgUuAaCaCCAuUuAcUuCaa | Homopropargyl |
| | | | 13 | G | uUgAaGuAaAuggUgUuAaCcag | |
| SB-0886 | NAG21 | AT3 | 3 | P | gpgUuAaCaCCAuUuAcUuCaa | Homopropargyl |
| | | | 13 | G | uUgAaGuAaAuggUgUuAaCcag | |
| SB-0887 | NAG21 | AT3 | 10 | P | gsgsUuAaCaCCAuUuAcUuCapa | Homopropargyl |
| | | | 6 | G | usUsgAaGuAaAuggUgUuAaCcsasg | |
| SB-0888 | NAG21 | AT3 | 12 | P | GsgsUuAaCaCCAuUuAcUuCapA | Homopropargyl |
| | | | 6 | G | usUsgAaGuAaAuggUgUuAaCcsasg | |
| SB-0889 | NAG21 | AT3 | 10 | P | gsgsUuAaCaCCAuUuAcUuCsasa | |
| | | | 14 | G | UUgAaGuAaAuggugUuAaCcsasg | Alkyne-Disulfide (ortho) |
| SB-0894 | NAG21 | AT3 | 10 | P | gsgsUuAaCaCCAuUuAcUuCaa | |
| | | | 14 | G | UUgAaGuAaAuggUgUuAaCcsasg | Alkyne-Disulfide (ortho) |
| SB-0895 | NAG21 | AT3 | 15 | P | GsgsUuAaCaCCAuUuAcUuCaa | |
| | | | 14 | G | UUgAaGuAaAuggUgUuAaCcsasg | Alkyne-Disulfide (ortho) |
| SB-0896 | NAG21 | AT3 | 16 | P | gsgsUuAaCaCCAuUuaCuUcapa | Homopropargyl |
| | | | 17 | G | usUsGaAguAaAuggUgUuAaccsasg | |
| SB-0897 | NAG21 | AT3 | 16 | P | gpgUuAaCaCCAuUuaCuUcaa | Homopropargyl |
| | | | 17 | G | uUGaAguAaAuggUgUuAaccag | |
| SB-0898 | NAG21 | AT3 | 10 | P | gsgsUuAaCaCCAuUuAcUuCapa | Homopropargyl |
| | | | 18 | G | uUgAaGuAaAuggUgUuAaCcsasgtt | |
| SB-0899 | NAG21 | AT3 | 10 | P | gsgsUuAaCaCCAuUuAcUuCapa | Homopropargyl |
| | | | 18 | G | uUgAaGuAaAuggUgUuAaCcsasgtst | |
| SB-0900 | NAG21 | AT3 | 16 | P | gsgsUuAaCaCCAuUuaCuUcapa | Homopropargyl |
| | | | 19 | G | uUGaAguAaAuggUgUuAaccsasgtt | |
| SB-0901 | NAG21 | AT3 | 16 | P | gsgsUuAaCaCCAuUuaCuUcapa | Homopropargyl |
| | | | 19 | G | uUGaAguAaAuggUgUuAaccsasgtst | |
| SB-0902 | NAG21 | AT3 | 10 | P | gsgsUuAaCaCCAuUuAcUuCapa | Homopropargyl |
| | | | 18 | G | uUgAaGuAaAuggUgUuAaCcsasgstst | |
| SB-0922 | NAG21 | AT3 | 20 | P | UpgGuAuCaGCCaAcCgCcUuU | Homopropargyl |
| | | | 21 | G | aAaGgCgGuUggcUgAuAcCaac | |
| SB-0923 | NAG21 | AT3 | 22 | P | apuCgAaAaGCCaAcAaGuCCU | Homopropargyl |
| | | | 23 | G | aGgAcUuGuUggcUuUuCgAuag | |
| SB-0924 | NAG21 | AT3 | 24 | P | gpgUuAaCaCCAuUuAcUuCaa-C3 | Homopropargyl |
| | | | 13 | G | uUgAaGuAaAuggUgUuAaCcag | |
| SB-0925 | NAG21 | AT3 | 3 | P | gpgUuAaCaCCAuUuAcUuCaa | Homopropargyl |
| | | | 13 | G | uUgAaGuAaAuggUgUuAaCcag-C3 | |
| SB-0926 | NAG21 | AT3 | 24 | P | gpgUuAaCaCCAuUuAcUuCaa-C3 | Homopropargyl |
| | | | 13 | G | uUgAaGuAaAuggUgUuAaCcag-C3 | |
| SB-0927 | NAG21 | AT3 | 24 | P | gsgsUuAaCaCCAuUuAcUuCsasa-C3 | |
| | | | 14 | G | uUgAaGuAaAuggUgUuAaCcsasg | Alkyne-Disulfide (ortho) |
| SB-0928 | NAG21 | AT3 | 10 | P | gsgsUuAaCaCCAuUuAcUuCsasa | |
| | | | 13 | G | uUgAaGuAaAuggUgUuAaCcsasg-C3 | Alkyne-Disulfide (ortho) |
| SB-0929 | NAG21 | AT3 | 24 | P | gsgsUuAaCaCCAuUuAcUuCsasa-C3 | |
| | | | 13 | G | uUgAaGuAaAuggUgUuAaCcsasg-C3 | Alkyne-Disulfide (ortho) |
| SB-0930 | NAG26 | AT3 | 10 | P | qpgpUpuAaCaCCAuUuAcUuCaa | Homopropargyl |
| | | | 6 | G | usUsgAaGuAaAuggUgUuAaCcsasg | |
| SB-0931 | NAG26 | AT3 | 25 | P | gsgsUuAaCaCCAuUuACpUUpCapa | Homopropargyl |
| | | | 13 | G | uUgAaGuAaAuggUgUuAaCcag | |
| SB-0932 | NAG26 | AT3 | 25 | P | gsgsUuAaCaCCAuUuACpUUpCapa | Homopropargyl |
| | | | 6 | G | usUsgAaGuAaAuggUgUuAaCcsasg | |
| SB-0933 | NAG26 | AT3 | 25 | P | gsgsUuAaCaCCAuUuACpUUpCapa-C3 | Homopropargyl |
| | | | 6 | G | usUsgAaGuAaAuggUgUuAaCcsasg | |
| SB-0934 | NAG26 | AT3 | 25 | P | gsgsUuAaCaCCAuUuACpUUpCapa | Homopropargyl |
| | | | 14 | G | usUsgAaGuAaAuggUgUuAaCcsasg-C3 | |
| SB-0935 | NAG26 | AT3 | 25 | P | gsgsUuAaCaCCAuUuACpUUpCapa-C3 | Homopropargyl |
| | | | 14 | G | usUsgAaGuAaAuggUgUuAaCcsasg-C3 | |
| SB-0936 | NAG26 | AT3 | 25 | P | gsgsUuAaCaCCAuUuACpUUpCapa | Homopropargyl |
| | | | 14 | G | usUsgAaGuAaAuggUgUuAaCcsasgs-C3 | |
| SB-0937 | NAG26 | AT3 | 25 | P | gsgsUuAaCaCCAuUuACpUUpCapa | Homopropargyl |
| | | | 14 | G | C3-usUsgAaGuAaAuggUgUuAaCcsasg | |
| SB-0938 | NAG26 | AT3 | 24 | P | gsgsUuAaCaCCAuUuAcUuCpapa | Homopropargyl |
| | | | 14 | G | uUgAaGuAaAuggUgUuAaCcsasg | Alkyne-Disulfide (ortho) |
| SB-0939 | NAG26 | AT3 | 24 | P | gsgsUuAaCaCCAuUuAcUuCpapa-C3 | Homopropargyl |
| | | | 14 | G | uUgAaGuAaAuggUgUuAaCcsasg | Alkyne-Disulfide (ortho) |
| SB-0940 | NAG26 | AT3 | 24 | P | gsgsUuAaCaCCAuUuAcUuCpapa | Homopropargyl |
| | | | 14 | G | uUgAaGuAaAuggUgUuAaCcsasg-C3 | Alkyne-Disulfide (ortho) |
| SB-0941 | NAG26 | AT3 | 24 | P | gsgsUuAaCaCCAuUuAcUuCpapa-C3 | Homopropargyl |
| | | | 14 | G | uUgAaGuAaAuggUgUuAaCcsasg-C3 | Alkyne-Disulfide (ortho) |
| SB-0952 | NAG26 | AT3 | 25 | P | gsgsUuAaCaCCAuUuACpUUpCapa | Homopropargyl |
| | | | 14 | G | usUbsgAaGuAaAuggUgUuAaCcsasg | |
| SB-0953 | NAG26 | AT3 | 25 | P | gsgsUuAaCaCCAuUuACpUUpCapa | Homopropargyl |
| | | | 14 | G | usUsgAabGuAaAuggUgUuAaCcsasg | |
| SB-0954 | NAG26 | AT3 | 25 | P | gsgsUuAaCaCCAuUuACpUUpCapa | Homopropargyl |
| | | | 26 | G | usUsgAaGuAaAuggUgUUbAaCcsasg | |
| SB-0962 | NAG26 | AT3 | 27 | P | GsgsUuAaCaCCAuUuACpUUpCapa | Homopropargyl |
| | | | 6 | G | usUsgAaGuAaAuggUgUuAaCcsasg | |
| SB-0963 | NAG26 | AT3 | 25 | P | gsgsUuAaCaCCAuUuACpUUpCapa | Alkyne-Disulfide (ortho); |
| | | | | | | Homopropargyl |
| | | | 6 | G | usUsgAaGuAaAuggUgUuAaCcsasg | |
| SB-0964 | NAG26 | AT3 | 25 | P | gsgsUuAaCaCCAuUuACUUCapa | Alkyne-Disulfide (ortho); |
| | | | | | | Homopropargyl |
| | | | 6 | G | usUsgAaGuAaAuggUgUuAaCcsasg | |
| SB-0965 | NAG26 | AT3 | 25 | P | gsgsUuAaCaCCAuUuACUUCaa | Alkyne-Disulfide (ortho) |
| | | | 6 | G | usUsgAaGuAaAuggUgUuAaCcsasg | |
| SB-0966 | NAG26 | AT3 | 25 | P | gsgsUbuAaCaCCAuUuACUUCaa | Alkyne-Disulfide (ortho) |
| | | | 6 | G | usUsgAaGuAaAuggUgUuAaCcsasg | |
| SB-0967 | NAG26 | AT3 | 24 | P | gpgpUpuAaCaCCAuUuAcUuCsasa | Homopropargyl |
| | | | 6 | G | usUsgAaGuAaAuggUgUuAaCcsasg | |
| SB-0968 | NAG21 | AT3 | 28 | P | gLscLsaststsgsgstsaststLscLsaLs- | Homopropargyl |
| | | | | | S-S-GsgsUuAaCaCCAuUuAcUuCapA | |
| | | | 6 | G | usUsgAaGuAaAuggUgUuAaCcsasg | |
| SB-0969 | NAG21 | AT3 | 29 | P | usascsasasas-S-S- | Homopropargyl |
| | | | | | GsgsUuAaCaCCAuUuAcUuCapA | |
| | | | 6 | G | usUsgAaGuAaAuggUgUuAaCcsasg | |
| SB-0970 | NAG26 | AT3 | 25 | P | gsgsUuAaCaCCAuUuACpUUpCapa | Homopropargyl |
| | | | 14 | G | usUpsgAaGuAaAuggUgUuAaCcsasg | |
| SB-0971 | NAG26 | AT3 | 25 | P | gsgsUuAaCaCCAuUuACpUUpCapa | Homopropargyl |
| | | | 14 | G | usUsgpAaGuAaAuggUgUuAaCcsasg | |
| SB-0972 | NAG26 | AT3 | 25 | P | gsgsUuAaCaCCAuUuACpUUpCapa | Homopropargyl |
| | | | 30 | G | usUsgapaGuAaAuggUgUuAaCcsasg | |
| SB-0973 | NAG26 | AT3 | 25 | P | gsgsUuAaCaCCAuUuACpUUpCapa | Homopropargyl |
| | | | 14 | G | usUsgAapGuAaAuggUgUuAaCcsasg | |
| SB-0974 | NAG26 | AT3 | 25 | P | gsgsUuAaCaCCAuUuACpUUpCapa | Homopropargyl |
| | | | 26 | G | usUsgAaGuAaAuggUgUUpAaCcsasg | |
| SB-0975 | NAG26 | AT3 | 25 | P | gsgsUuAaCaCCAuUuACpUUpCapa | Homopropargyl |
| | | | 31 | G | usUsgAaGuAaAuggUgUuapaCcsasg | |
| SB-0976 | NAG26 | AT3 | 25 | P | gsgsUuAaCaCCAuUuACpUUpCapa | Homopropargyl |
| | | | 14 | G | usUsgAaGuAaAuggUgUuAapCcsasg | |
| SB-0977 | NAG26 | AT3 | 25 | P | gsgsUuAaCaCCAuUuACpUUpCapa | Homopropargyl |
| | | | 14 | G | usUsgAaGuAaAuggUgUuAaCpcsasg | |
| SB-0978 | NAG26 | AT3 | 25 | P | gsgsUuAaCaCCAuUuACpUUpCapa | Homopropargyl |
| | | | 14 | G | usUsgAaGuAaAuggUgUuAaCcspasg | |
| SB-0979 | NAG26 | AT3 | 25 | P | gsgsUuAaCaCCAuUuACpUUpCapa | Homopropargyl |
| | | | 14 | G | usUsgAaGuAaAuggUgUuAaCcsaspg | |
| SB-0991 | NAG21 | AT3 | 10 | P | gsgsUuAaCaCCAuUuAcUuCapa | Homopropargyl |
| | | | 26 | G | usUsgAaGuAaAuggUgUUpAaCcsasg | |
| SB-0992 | NAG21 | AT3 | 10 | P | gsgsUuAaCaCCAuUuAcUuCapa | Homopropargyl |
| | | | 31 | G | usUsgAaGuAaAuggUgUuapaCcsasg | |
| SB-0993 | NAG21 | AT3 | 10 | P | gsgsUuAaCaCCAuUuAcUuCapa | Homopropargyl |
| | | | 14 | G | usUsgAaGuAaAuggUgUuAapCcsasg | |
| SB-0994 | NAG21 | AT3 | 10 | P | gsgsUuAaCaCCAuUuAcUuCapa | Homopropargyl |
| | | | 14 | G | usUsgAaGuAaAuggUgUuAaCpcsasg | |
| SB-0995 | NAG21 | AT3 | 10 | P | gsgsUuAaCaCCAuUuAcUuCapa | Homopropargyl |
| | | | 14 | G | usUsgAaGuAaAuggUgUuAaCcspasg | |
| SB-0996 | NAG21 | AT3 | 10 | P | gsgsUuAaCaCCAuUuAcUuCapa | Homopropargyl |
| | | | 14 | G | usUsgAaGuAaAuggUgUuAaCcsaspg | |
| SB-1010 | NAG21 | C5 | 32 | P | AsasGcAaGaUAUuUuUaUaAUpA | Homopropargyl |
| | | | 33 | G | usAsuUaUaAaAauaUcUuGcUususu | |
| SB-1011 | NAG21 | C5 | 34 | P | apaGcAaGaUAUuUuUaUaAUA | Homopropargyl |
| | | | 33 | G | uAuUaUaAaAauaUcUuGcUuuu | |
| SB-1012 | NAG21 | C5 | 35 | P | asasGcAaGaUAUuUuuAuAaUpa | Homopropargyl |
| | | | 36 | G | usAsUuAuaAaAauaUcUuGcuususu | |
| SB-1013 | NAG21 | C5 | 35 | P | apaGcAaGaUAUuUuuAuAaUa | Homopropargyl |
| | | | 36 | G | uAUuAuaAaAauaUcUuGcuuuu | |
| SB-1031 | NAG21 | AT3 | 10 | P | gsgsUuAaCaCCAuUuAcUuCapa | Homopropargyl |
| | | | 14 | G | usUsgAaGuAaAuggUgUuAaCpcsasg | |
| SB-1057 | NAG21 | C5 | 35 | P | asasGcAaGaUAUuUuuAuAaUpa | Homopropargyl |
| | | | 36 | G | usAsUuAuaAaAauaUcUuGcupususu | |
| SB-1058 | NAG36 | C5 | 35 | P | asasGcAaGaUAUuUuuAuAaUpa | Homopropargyl |
| | | | 36 | G | usAsUuAuaAaAauaUcUuGcupususu | |
| SB-1059 | NAG26 | C5 | 37 | P | asasGcAaGaUAUuUuuapuapaUpa | Homopropargyl |
| | | | 36 | G | usAsUuAuaAaAauaUcUuGcuususu | |
| SB-1060 | NAG26 | C5 | 37 | P | asasGcAaGaUAUuUuuapuapaUpa | Homopropargyl |
| | | | 36 | G | usAsUuAuaAaAauaUcUuGcupususu | |
| SB-1091 | NAG26 | AT3 | 25 | P | gsgsUuAaCaCCAuUuACpUUpCapa | Homopropargyl |
| | | | 14 | G | usUsgAaGuAaAuggUgUuAaCbcsasg | |
| SB-1092 | NAG26 | AT3 | 25 | P | gsgsUuAaCaCCAuUuACpUUpCapa | Homopropargyl |
| | | | 14 | G | usUsgAaGuAaAuggUgUuAaCcsasg | |
| SB-1093 | NAG26 | AT3 | 25 | P | gsgsUuAaCaCCAuUuACpUUpCapa | Homopropargyl |
| | | | 14 | G | usUsgAaGuAaAuggUgUuAaCcsasg | |
| SB-1094 | NAG26 | AT3 | 25 | P | gsgsUuAaCaCCAuUuACpUUpCapa | Homopropargyl |
| | | | 14 | G | usUsgAaGuAaAuggUgUuAaChcsasg | |
| SB-1095 | NAG26 | AT3 | 25 | P | gsgsUuAaCaCCAuUuACpUUpCapa | Homopropargyl |
| | | | 14 | G | usUsgAaGuAaAuggUgUuAaCdcsasg | |
| SB-1096 | NAG26 | AT3 | 25 | P | gsgsUuAaCaCCAuUuACpUUpCapa | Homopropargyl |
| | | | 14 | G | usUsgAaGuAaAuggUgUuAaC(Hex)csasg | |
| SB-1097 | NAG26 | AT3 | 25 | P | gsgsUuAaCaCCAuUuACpUUpCapa | Homopropargyl |
| | | | 14 | G | usUsgAaGuAaAuggUgUuAaC(Hexe)csasg | |
| SB-1098 | NAG26 | AT3 | 25 | P | gsgsUuAaCaCCAuUuACpUUpCapa | Homopropargyl |
| | | | 14 | G | usUsgAaGuAaAuggUgUuAaCncsasg | |
| SB-1099 | NAG26 | AT3 | 25 | P | gsgsUuAaCaCCAuUuACpUUpCapa | Homopropargyl |
| | | | 14 | G | usUsgAaGuAaAuggUgUuAaCPcsasg | |
| SB-1214 | NAG36 | AT3 | 5 | P | GsgsUuAaCaCCAuUuAcUuCaA-Alk | 3′ Alkyne |
| | | | 6 | G | usUsgAaGuAaAuggUgUuAaCcsasg | |
| SB-1215 | NAG36 | AT3 | 10 | P | gsgsUuAaCaCCAuUuAcUuCapa | Homopropargyl |
| | | | 6 | G | usUsgAaGuAaAuggUgUuAaCcsasg | |
| SB-1216 | NAG28 | AT3 | 25 | P | gsgsUuAaCaCCAuUuACpUUpCapa | Homopropargyl |
| | | | 6 | G | usUsgAaGuAaAuggUgUuAaCcsasg | |
| SB-1232 | NAG21 | AT3 | 16 | P | gsgsUuAaCaCCAuUuaCsuUscapa | Homopropargyl |
| | | | 25 | G | usUsGaAguAaAuggUgUuAaccsasg | |
| SB-1250 | NAG21 | AT3 | 10 | P | gsgsUuAaCaCCAuUuAcUuCapa | Homopropargyl |
| | | | 38 | G | usUsgAaGuAaAuggUgUuAaccsasg | |
| SB-1251 | NAG21 | AT3 | 10 | P | gsgsUuAaCaCCAuUuAcUuCapa | Homopropargyl |
| | | | 31 | G | usUsgAaGuAaAuggUgUuaaCcsasg | |
| SB-1252 | NAG21 | AT3 | 10 | P | gsgsUuAaCaCCAuUuAcUuCapa | Homopropargyl |
| | | | 39 | G | usUsgAaGuAaAuggUgUuaaccsasg | |
| SB-1253 | NAG21 | AT3 | 10 | P | gsgsUuAaCaCCAuUuAcUuCapa | Homopropargyl |
| | | | 14 | G | usUsgAaGuAaAuggUgUuAaCscsasg | |
| SB-1254 | NAG21 | AT3 | 10 | P | gsgsUuAaCaCCAuUuAcUuCapa | Homopropargyl |
| | | | 14 | G | usUsgAaGuAaAuggUgUuAsaCcsasg | |
| SB-1255 | NAG21 | AT3 | 10 | P | gsgsUuAaCaCCAuUuAcUuCapa | Homopropargyl |
| | | | 14 | G | usUsgAaGuAaAuggUgUuAsaCscsasg | |
| SB-1256 | NAG21 | AT3 | 10 | P | gsgsUuAaCaCCAuUuAcUuCapa | Homopropargyl |
| | | | 14 | G | usUsgAaGuAaAuggUgUuApaCpcsasg | |
| SB-1276 | NAG21 | TTR | 40 | P | AsasCaGuGuUCUuGcUcUaUapA | Homopropargyl |
| | | | 41 | G | usUsaUaGaGcAagaAcAcUgUususU | |
| SB-1277 | NAG21 | TTR | 40 | P | AsasCaGuGuUCUuGcUcUaUapA | Homopropargyl |
| | | | 41 | G | usUsaUaGaGcAagaAcAcUgUpususU | |
| SB-1278 | NAG26 | TTR | 42 | P | AsasCaGuGuUCUuGcUCpUapUapA | Homopropargyl |
| | | | 41 | G | usUsaUaGaGcAagaAcAcUgUususU | |
| SB-1279 | NAG26 | TTR | 42 | P | AsasCaGuGuUCUuGcUCpUapUapA | Homopropargyl |
| | | | 41 | G | usUsaUaGaGcAagaAcAcUgUpususU | |
| SB-1284 | NAG26 | AT3 | 43 | P | gsgsUuAaCaCCAuUUpACpUUpCapa | Homopropargyl |
| | | | 6 | G | usUsgAaGuAaAuggUgUuAaCcsasg | |
| SB-1285 | NAG26 | AT3 | 44 | P | gsgsUuAaCaCCAUpUUpACpUUpCapa | Homopropargyl |
| | | | 6 | G | usUsgAaGuAaAuggUgUuAaCcsasg | |
| SB-1286 | NAG26 | AT3 | 44 | P | gsgsUuAaCaCCAUUUACpUUpCapa | Alkyne-Disulfide (ortho); |
| | | | | | | Homopropargyl |
| | | | 6 | G | usUsgAaGuAaAuggUgUuAaCcsasg | |
| SB-1289 | NAG26 | AT3 | 25 | P | gsgsUuAaCaCCAuUuACpUUpCapa | Homopropargyl |
| | | | 14 | G | usUsgAaGuAaAuggUgUuAasCcsasg | |
| SB-1290 | NAG26 | AT3 | 25 | P | gsgsUuAaCaCCAuUuACpUUpCapa | Homopropargyl |
| | | | 14 | G | usUsgAaGuAaAuggUgUuAaCscsasg | |
| SB-1291 | NAG26 | AT3 | 25 | P | gsgsUuAaCaCCAuUuACpUUpCapa | Homopropargyl |
| | | | 14 | G | usUsgAaGuAaAuggUgUuAsaCscsasg | |
| SB-1292 | NAG26 | AT3 | 25 | P | gsgsUuAaCaCCAuUuACpUUpCapa | Homopropargyl |
| | | | 14 | G | usUsgAaGuAaAuggUgUuApaCpcsasg | |
| SB-1293 | NAG26 | AT3 | 25 | P | gsgsUuAaCaCCAuUuACpUUpCapa | Homopropargyl |
| | | | 31 | G | usUsgAaGuAaAuggUgUuaaCcsasg | |
| SB-1294 | NAG21 | AT3 | 45 | P | gsgsUuAaCaCCAuUuAcUUbCapa | Homopropargyl |
| | | | 6 | G | usUsgAaGuAaAuggUgUuAaCcsasg | |
| SB-1295 | NAG21 | AT3 | 46 | P | gsgsUuAaCaCCAuUuACbUUbCapa | Homopropargyl |
| | | | 6 | G | usUsgAaGuAaAuggUgUuAaCcsasg | |
| SB-1296 | NAG26 | AT3 | 25 | P | gsgsUuAaCaCCAuUuACpUUpCapa | Homopropargyl |
| | | | 14 | G | usUsgAaGuAaAuggUgUuAaCXcsasg | X = 4-Pentyn-2-yl |
| | | | | | | Phosphotriester |
| SB-1297 | NAG26 | AT3 | 25 | P | gsgsUuAaCaCCAuUuACpUUpCapa | Homopropargyl |
| | | | 14 | G | usUsgAaGuAaAuggUgUuAaCXcsasg | X = 2-Methyl-Propyl |
| | | | | | | Phosphotriester |
| SB-1298 | NAG26 | AT3 | 25 | P | gsgsUuAaCaCCAuUuACpUUpCapa | Homopropargyl |
| | | | 14 | G | usUsgAaGuAaAuggUgUuAaCXcsasg | X = Phenethyl |
| | | | | | | Phosphotriester |
| SB-1299 | NAG26 | AT3 | 25 | P | gsgsUuAaCaCCAuUuACpUUpCapa | Homopropargyl |
| | | | 14 | G | usUsgAaGuAaAuggUgUuAaCXcsasg | X = Pyrid-4-ylethyl |
| | | | | | | Phosphotriester |
| SB-1300 | NAG26 | AT3 | 25 | P | gsgsUuAaCaCCAuUuACpUUpCapa | Homopropargyl |
| | | | 14 | G | usUsgAaGuAaAuggUgUuAaCXcsasg | X = Pyrid-4-ylmethyl |
| | | | | | | Phosphotriester |
| SB-1301 | NAG26 | AT3 | 25 | P | gsgsUuAaCaCCAuUuACpUUpCapa | Homopropargyl |
| | | | 14 | G | usUsgAaGuAaAuggUgUuAaCXcsasg | X = Imidazolylethyl |
| | | | | | | Phosphotriester |
| SB-1302 | NAG26 | AT3 | 25 | P | gsgsUuAaCaCCAuUuACpUUpCapa | Homopropargyl |
| | | | 14 | G | usUsgAaGuAaAuggUgUuAaCXcsasg | X = Cyclopropylethyl |
| | | | | | | Phosphotriester |
| SB-1303 | NAG26 | AT3 | 25 | P | gsgsUuAaCaCCAuUuACpUUpCapa | Homopropargyl |
| | | | 14 | G | usUsgAaGuAaAuggUgUuAaCXcsasg | X = Cyclopropylmethyl |
| | | | | | | Phosphotriester |
| SB-1304 | NAG26 | AT3 | 25 | P | gsgsUuAaCaCCAuUuACpUUpCapa | Homopropargyl |
| | | | 14 | G | usUsgAaGuAaAuggUgUuAaCXcsasg | X = hydroxycarbonyl- |
| | | | | | | phenyltriazolylethyl |
| | | | | | | Phosphotriester |
| SB-1305 | NAG26 | AT3 | 25 | P | gsgsUuAaCaCCAuUuACpUUpCapa | Homopropargyl |
| | | | 14 | G | usUsgAaGuAaAuggUgUuAaCXcsasg | X = phenyltriazolylethyl |
| | | | | | | Phosphotriester |
| SB-1309 | NAG26 | AT3 | 46 | P | gsgsUuAaCaCCAuUuACUUCapa | Homopropargyl |
| | | | 6 | G | usUsgAaGuAaAuggUgUuAaCcsasg | |
| SB-1310 | NAG26 | AT3 | 45 | P | gsgsUuAaCaCCAuUuAcUUpCpapa | Homopropargyl |
| | | | 6 | G | usUsgAaGuAaAuggUgUuAaCcsasg | |
| SB-1311 | NAG26 | AT3 | 45 | P | gsgsUuAaCaCCAuUuAcUpUpCpapa | Homopropargyl |
| | | | 6 | G | usUsgAaGuAaAuggUgUuAaCcsasg | |
| SB-1312 | NAG26 | AT3 | 46 | P | gsgsUuAaCaCCAuUuACpUpUpCpapa | Homopropargyl |
| | | | 6 | G | usUsgAaGuAaAuggUgUuAaCcsasg | |
| SB-1313 | NAG26 | AT3 | 43 | P | gsgsUuAaCaCCAuUUpACpUUpCapa | Homopropargyl |
| | | | 6 | G | usUsgAaGuAaAuggUgUuAaCcsasg | |
| SB-1314 | NAG26 | AT3 | 44 | P | gsgsUuAaCaCCAUpUUpACpUUpCapa | Homopropargyl |
| | | | 6 | G | usUsgAaGuAaAuggUgUuAaCcsasg | |
| SB-1315 | NAG26 | AT3 | 44 | P | gsgsUuAaCaCCAUUUACpUUpCapa | Homopropargyl |
| | | | 6 | G | usUsgAaGuAaAuggUgUuAaCcsasg | |
| SB-1316 | NAG37 | AT3 | 25 | P | gsgsUuAaCaCCAuUuACpUUpCapa | Homopropargyl |
| | | | 6 | G | usUsgAaGuAaAuggUgUuAaCcsasg | |
| SB-1317 | NAG37 | AT3 | 46 | P | gsgsUuAaCaCCAuUuACUUCapa | Homopropargyl |
| | | | 6 | G | usUsgAaGuAaAuggUgUuAaCcsasg | |
| SB-1318 | NAG37 | AT3 | 45 | P | gsgsUuAaCaCCAuUuAcUUpCpapa | Homopropargyl |
| | | | 6 | G | usUsgAaGuAaAuggUgUuAaCcsasg | |
| SB-1376 | NAG26 | PCSK9 | 47 | P | UsusUuCuAgAcCuGuUUpUgpCUpU | Homopropargyl |
| | | | 48 | G | asAsgCaAaAcAgGuCuAgAaAasGsu | |
| SB-1377 | NAG26 | PCSK9 | 47 | P | UsusUuCuAgAcCuGuUUpUgpCUpU | Homopropargyl |
| | | | 48 | G | asAsgCaAaAcAgGuCuAgAaapasGsu | |
| SB-1378 | NAG26 | PCSK9 | 49 | P | CsasAgCaGaCAUuUaUCpUUpUUpU | Homopropargyl |
| | | | 50 | G | asAsaAaGaUaAaugUcUgCuUgsCsu | |
| SB-1379 | NAG26 | PCSK9 | 49 | P | CsasAgCaGaCAUuUaUCpUUpUUpU | Homopropargyl |
| | | | 50 | G | asAsaAaGaUaAaugUcUgCuUpgsCsu | |
| SB-1380 | NAG26 | PCSK9 | 51 | P | CsusAgAcCuGUUuUgCUpUUpUgpU | Homopropargyl |
| | | | 52 | G | asCsaAaAgCaAaacAgGuCuAgsAsa | |
| SB-1381 | NAG26 | PCSK9 | 51 | P | CsusAgAcCuGUUuUgCUpUUpUgpU | Homopropargyl |
| | | | 52 | G | asCsaAaAgCaAaacAgGuCuApgsAsa | |
| SB-1382 | NAG26 | PCSK9 | 53 | P | CsusAgAcCuGUUuUgCUpUUpUgpU | Homopropargyl |
| | | | 54 | G | asCsaAaagCaAaacAgGucuAgsasa | |
| SB-1383 | NAG26 | PCSK9 | 53 | P | CsusAgAcCuGUUuUgCUpUUpUgpU | Homopropargyl |
| | | | 55 | G | asCsaAaagCaAaacAgGucuapgsasa | |
| SB-1409 | NAG26 | TTR | 56 | P | AsasCaGuGuUCUuGcUcUaUaATpTpTp | Homopropargyl |
| | | | 41 | G | usUsaUaGaGcAagaAcAcUgUpususU | |
| SB-1445 | NAG26 | AT3 | 57 | P | gsgsUuAaCaCCAuUuAcUuCaaTpTpTp | Homopropargyl |
| | | | 14 | G | usUsgAaGuAaAuggUgUuAaCpcsasg | |

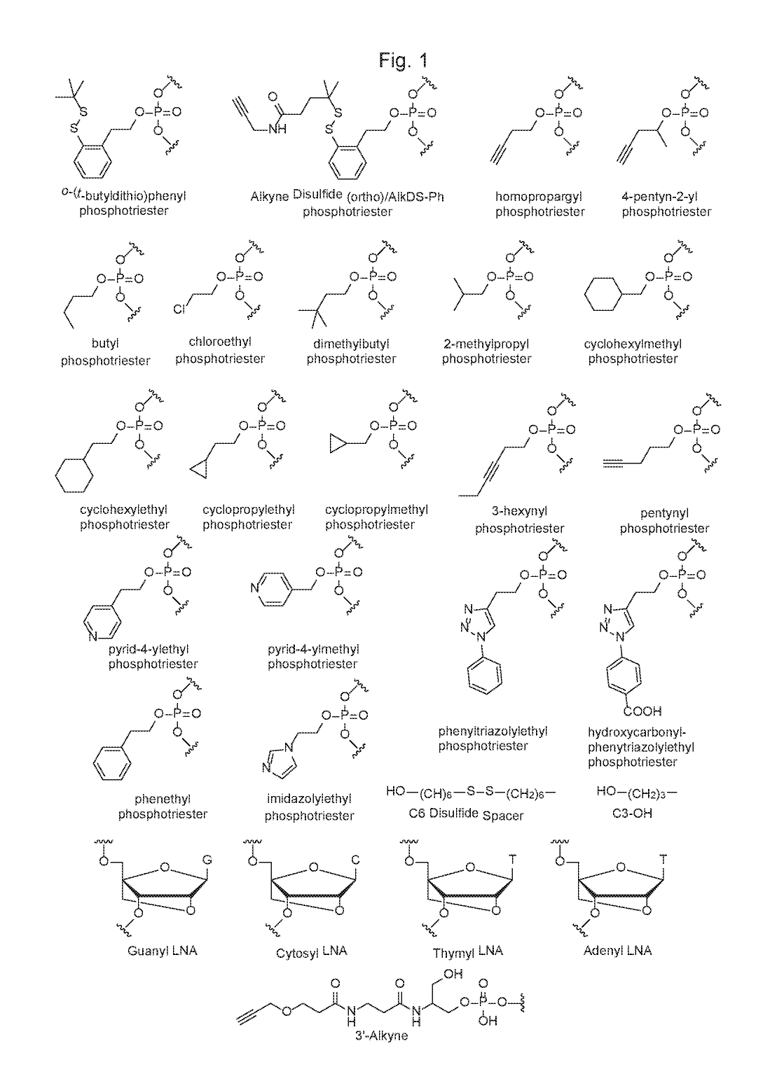

In Table 3,
UNDERLINE = conjugation location;
UPPER CASE = a 2′-fluoro;
lower case = 2′-methoxy;
aM = 2′-methoxyethoxyadenosine;
italics = o-(t-butyldithio)phenyl;
s = phosphorothioate;
Alk = 3′alkyne;
underlined italics = alkyne disulfide (ortho)/AlkDS-Ph;
S-S = C₆ Disulfide Spacer;
gL = guanyl LNA;
gC = cytosyl LNA;
tL = thymyl LNA;
aL = adenyl LNA;
C3 = 3′ or 5′ C3-OH;
p = homopropargyl phosphotriester;
b = n-butylphosphotriester;
h = chloroethyl phosphotriester;
d = dimethylbutyl phosphotriester;
Hex = cyclohexylmethyl phosphotriester;
Hexe = cyclohexylethyl phosphotriester;
n = 3-hexynyl phosphotriester;
P = pentynyl phosphotriester;
m = methylphosphonate;
and
B = butylphosphoramidate.
The structures of these groups and of the phosphotriesters listed in Table 3 are provided in FIG. 1. A nucleoside identified by a bold, underlined letter followed by p indicates that the phosphate bonded to the 3′-position of the nucleoside is the conjugation location.

### Example 3. Stability in Serum

Triester containing oligonucleotide (single and double-strand) serum stability was carried out as described below.

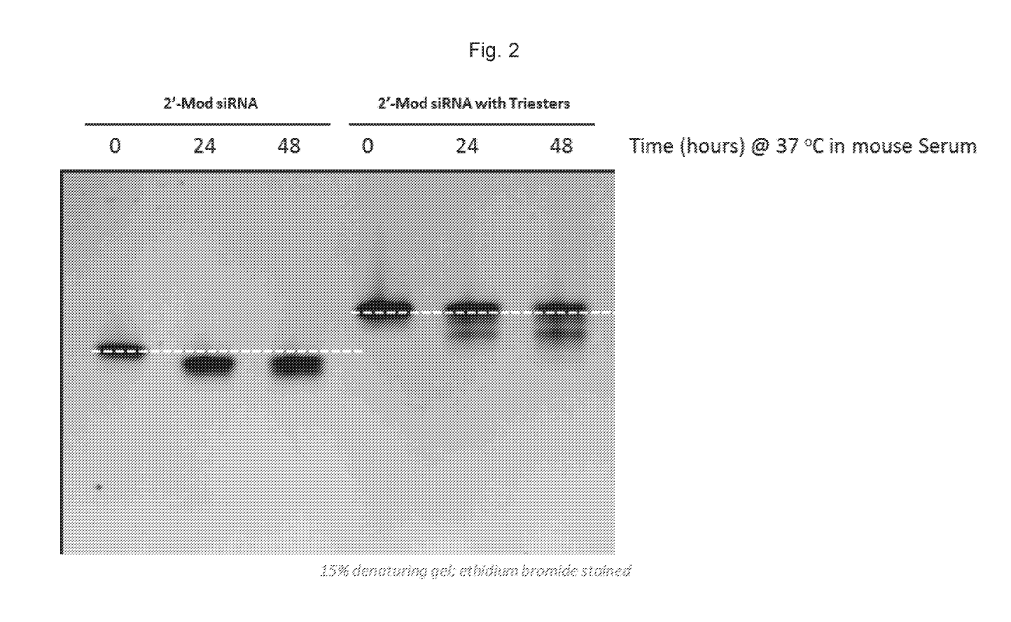

20 μL of 250 μM dsRNA stocks were made up; 4 μL from each were removed and placed in 16 μL of fresh mouse serum; 20 μL samples were placed in PCR plates and heated on thermocycler at 37° C.; 2 μL were removed at indicated time points, added to 18 μL of formamide loading buffer and frozen prior to gel analysis; 2 μL were loaded per well for gel analysis. Representative gel demonstrating the improved serum stability of triester containing oligonucleotides is provided in FIG. 2.

### Example 4. Mouse Primary Hepatocyte Isolation and In Vitro Experiments

Primary mouse hepatocytes were isolated using the standard two-step collagenase perfusion technique (Li et. al. Methods Mol. Biol., 633:185-196; 2010). Briefly, a 6-10 week old female C57/Bl6 mouse was anesthetized by intraperitoneal injection of a mixture of ketamine (80-100 mg/kg)/xylazine (5-10 mg/kg). The abdominal cavity was then exposed and the visceral vena cava was cannulated using a 22G needle. The hepatic vein was severed and the liver was immediately perfused for 5-10 min using a solution of phosphate-buffered saline (PBS) containing 0.5 mM ETDA. This solution was immediately switched to a solution of collagenase (100 IU/mL) in Dulbecco's Modified Eagle's Medium (DMEM, Gibco) for another 5-10 min. At the end of perfusion, the liver was removed and the hepatocytes were collected in DMEM containing 10% fetal bovine serum at 4° C. The cells were then filtered through a 70 μm sterile filter, washed three times in the same solution, and cell viability was assessed using Trypan Blue staining. Cells were then seeded in 96-well plates coated with 0.1% rat tail collagen or 2% matrigel and incubated for 3-4 hours at 37 C° in a 5% CO₂ incubator. Test compounds were then added to cells and incubated at 37° C. in a 5% CO₂ incubator. At the end of the incubation period, the cells were lysed, the mRNA was isolated and the expression of the target gene was measured by qPCR and normalized to a house-keeping gene using standard protocols.

### Example 5. In Vivo Experiments

Test compounds were administered to female C57Bl6 mice via either subcutaneous or intravenous (lateral tail vein) injection (200 μL; 3 mice/treatment). At the appropriate time point post injection, mice were sacrificed and blood samples were collected by cardiac puncture. An approximately 50-100 mg piece of liver sample was collected and immediately frozen in liquid nitrogen. Total mRNA was isolated from liver homogenates using standard protocols, and the expression of target gene was quantified by qPCR and normalized to a house-keeping gene using standard protocols.

In another format, blood was collected from mice at different time points post dosing using sodium citrate as an anticoagulant. Plasma AT3 protein was measured using a commercially available chromogenic assay that assesses the heparin cofactor activity of AT3 using an anti-factor Xa method.

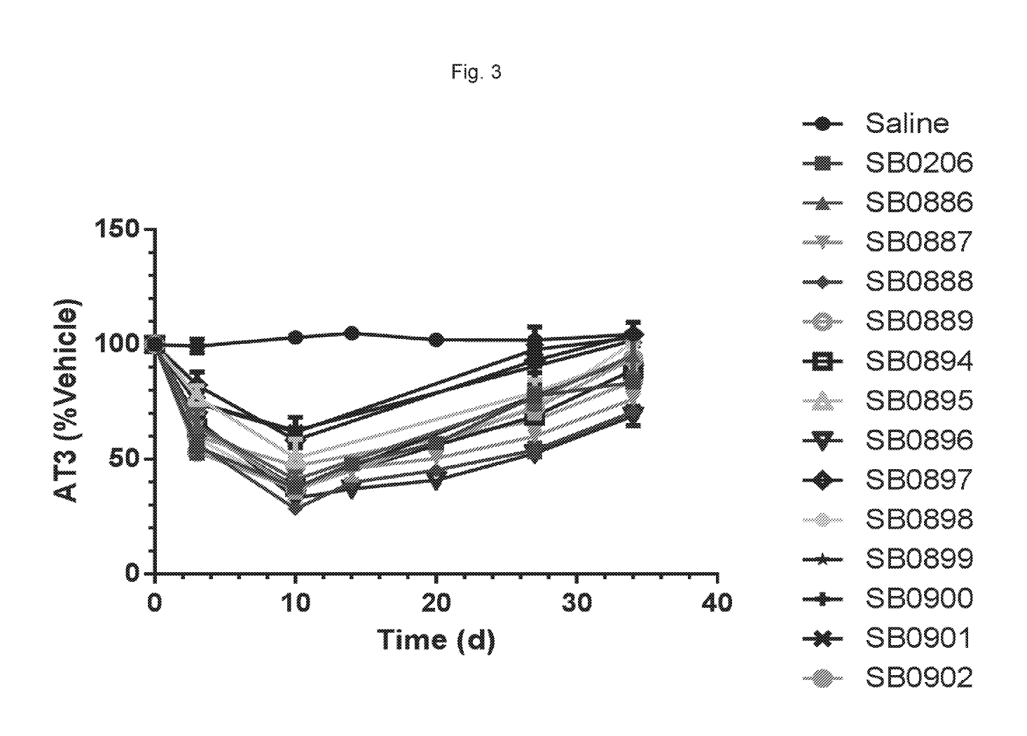

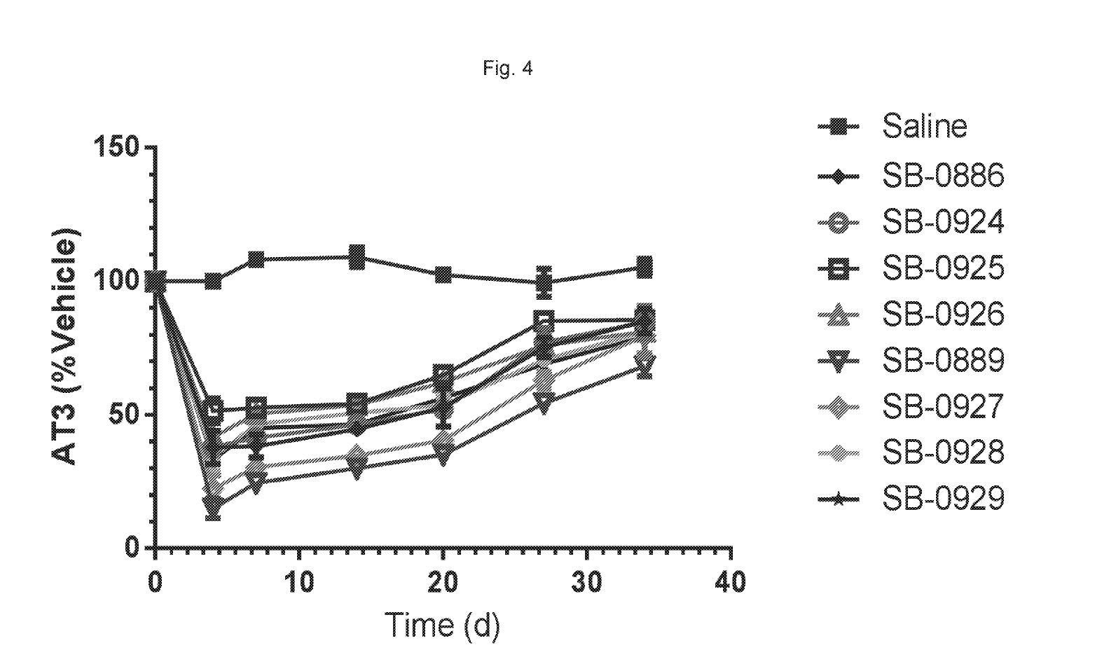

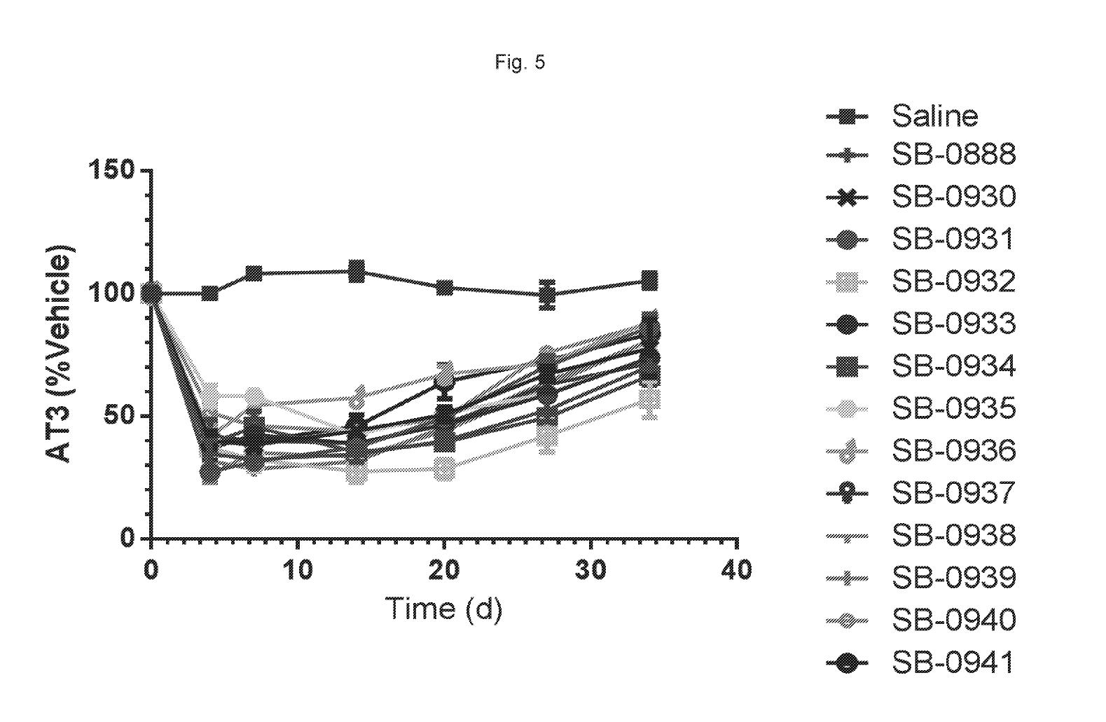

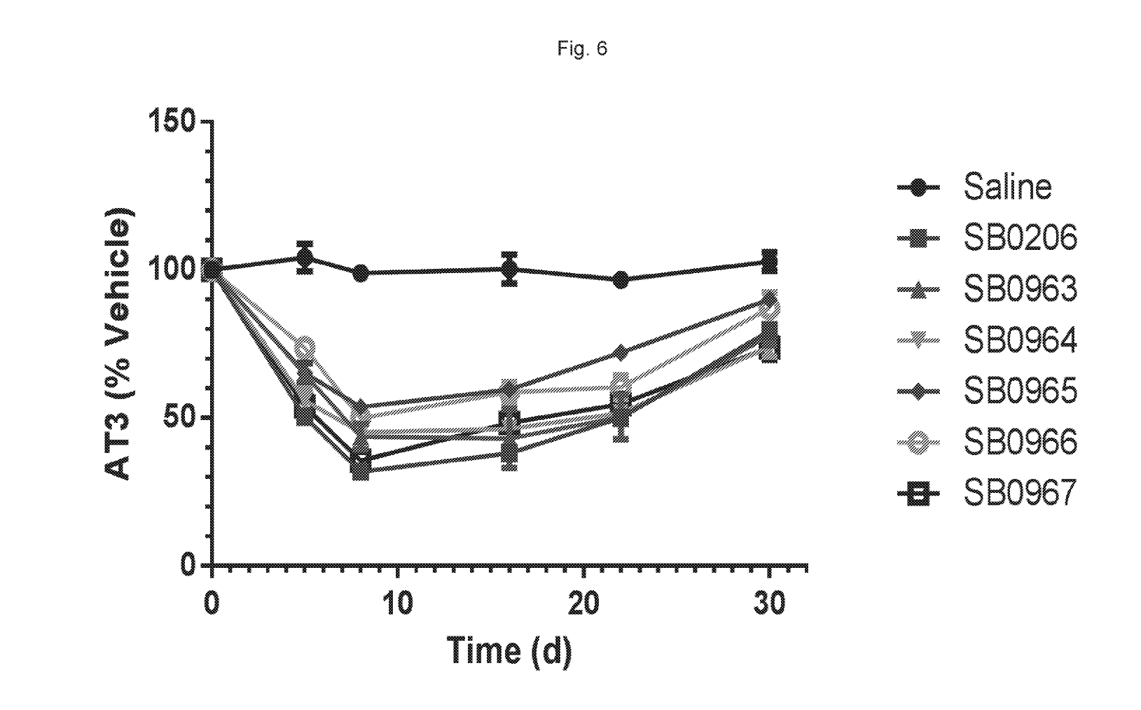

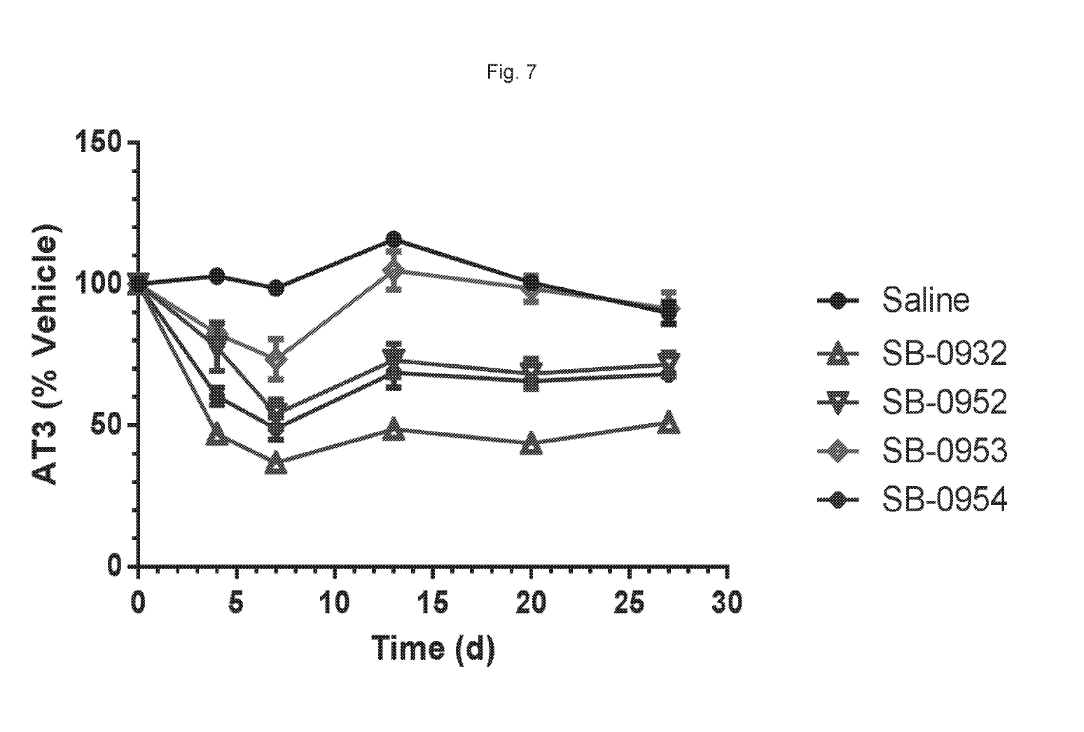

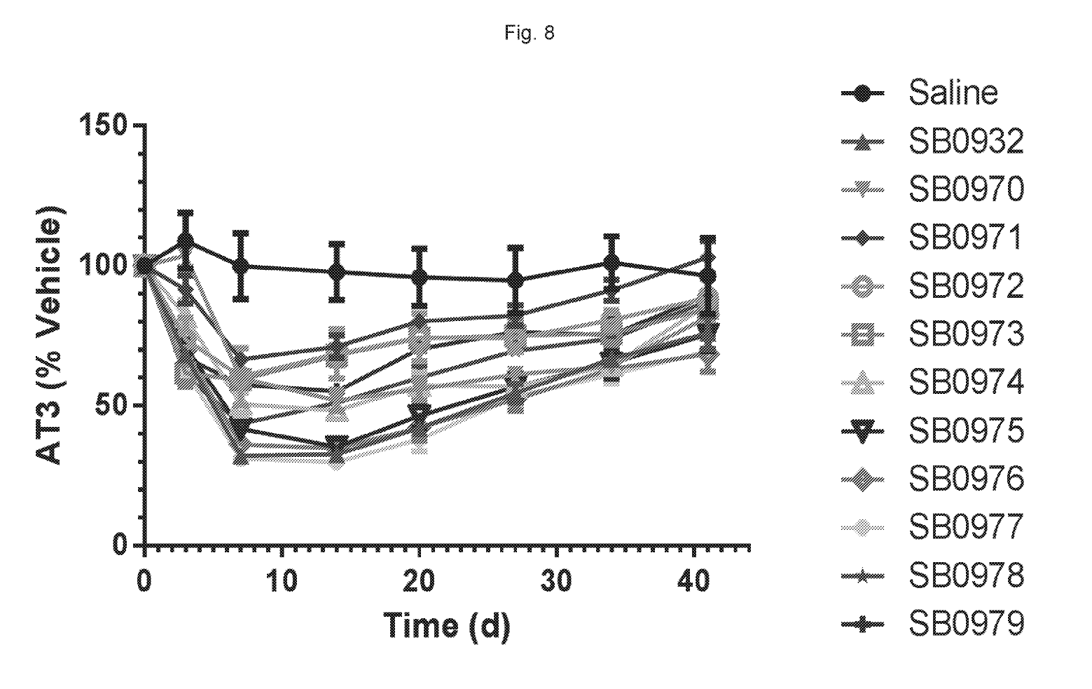

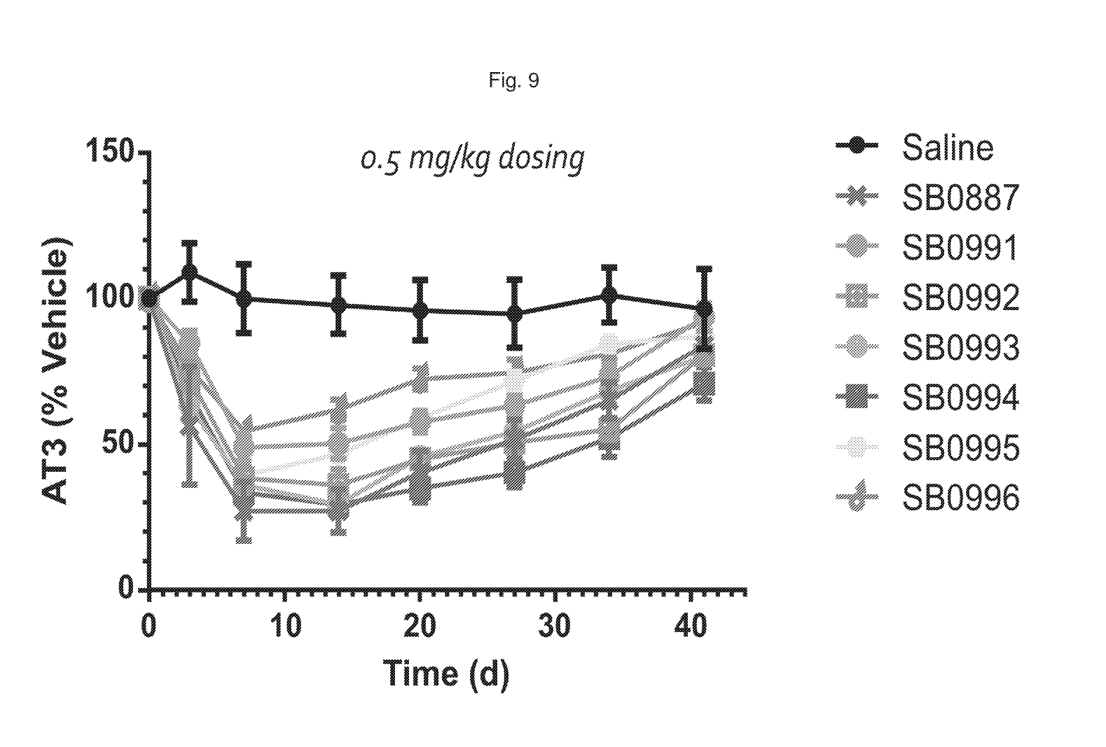

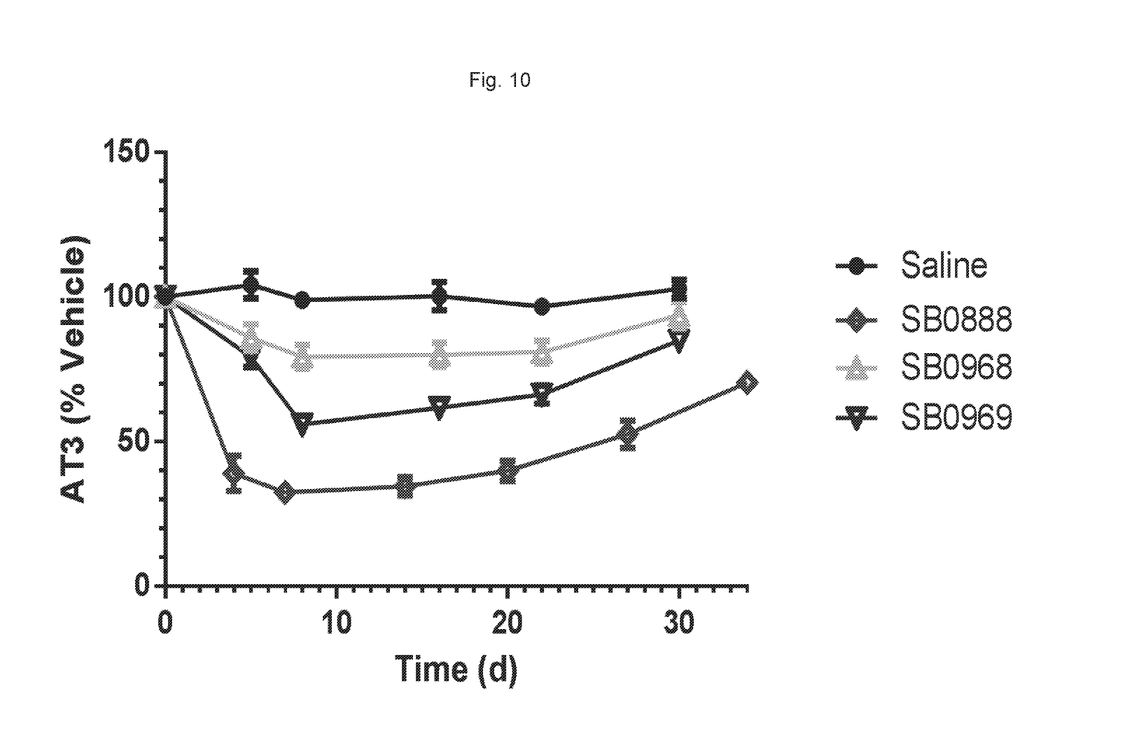

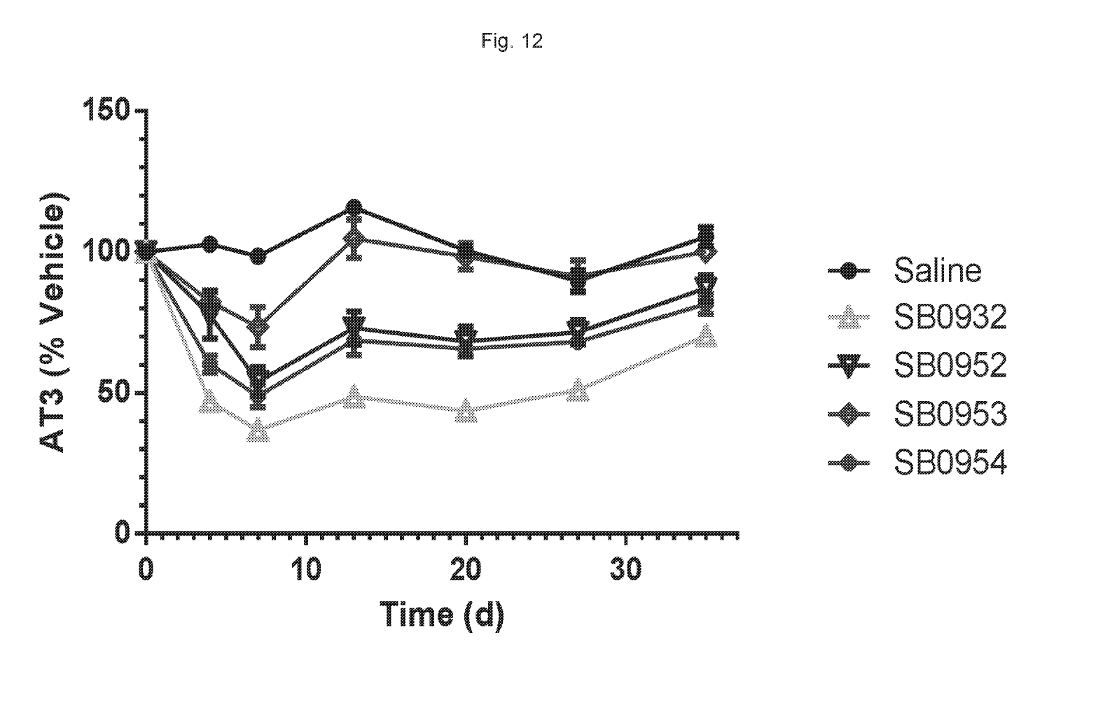

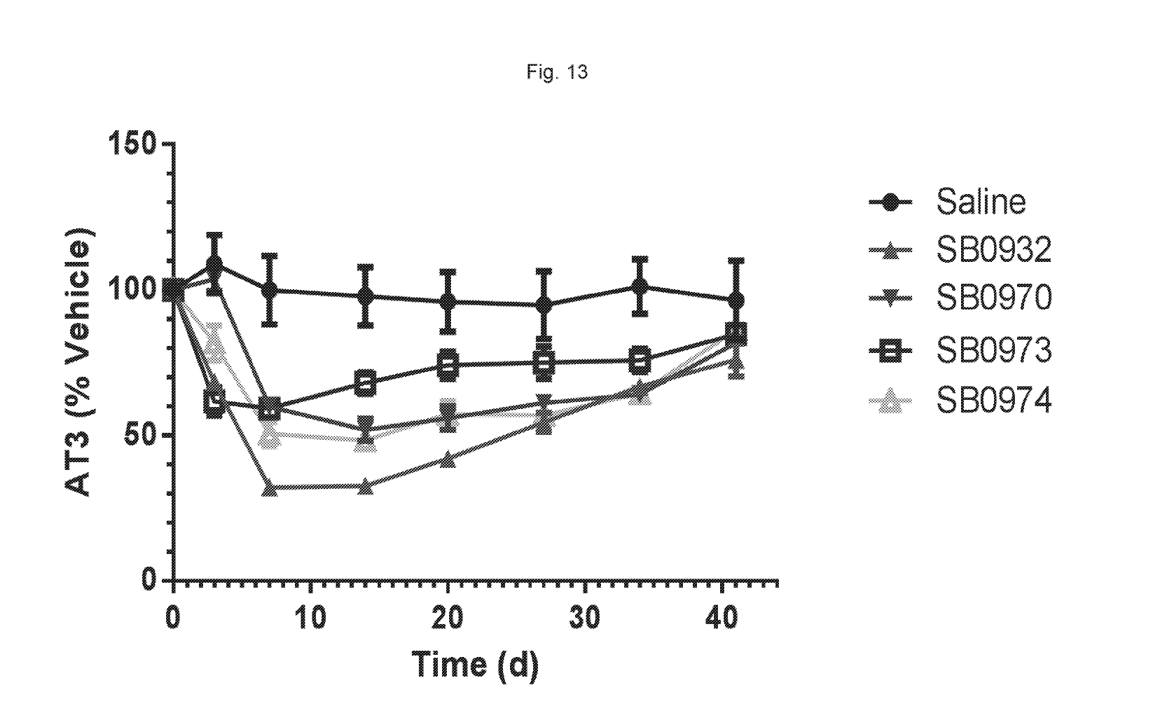

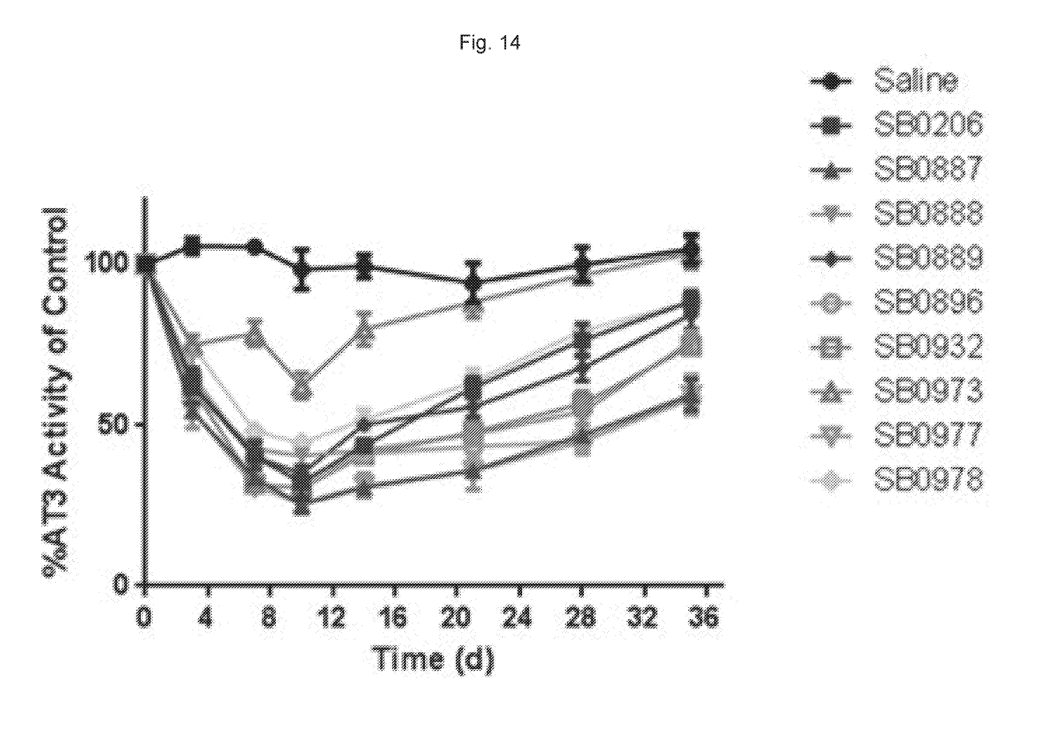

The results of these experiments are provided in FIGS. 3-15 and in Table 4.

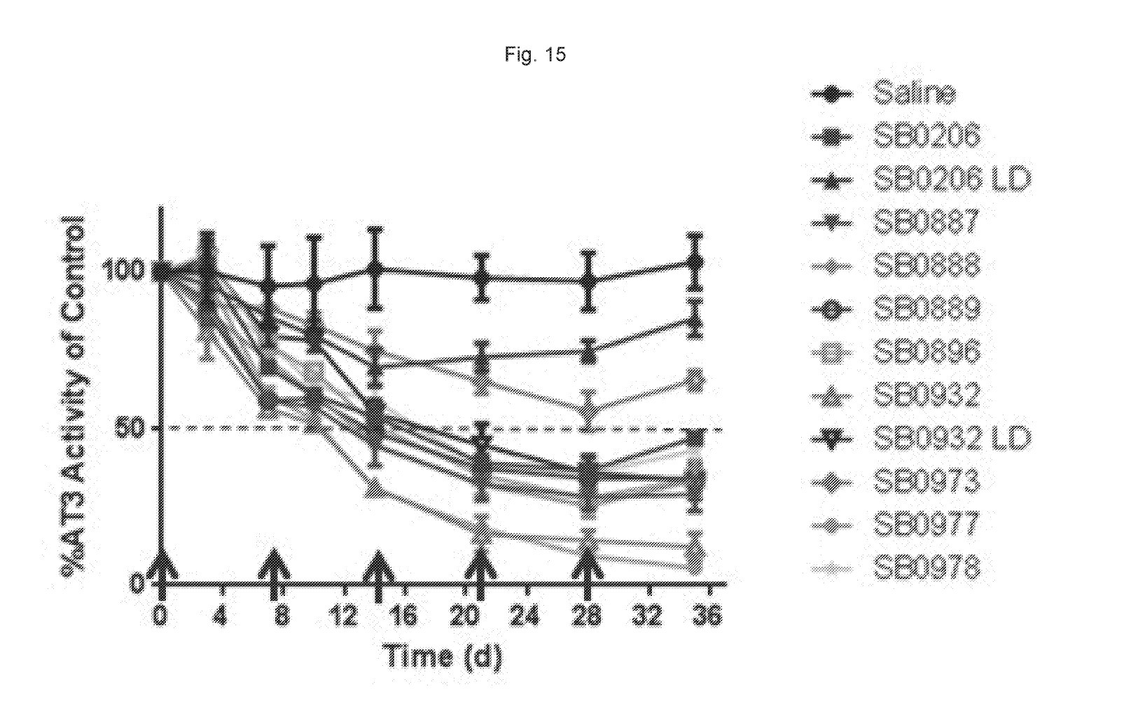

The data in FIGS. 3-14 were obtained following administration of a single dose of the indicated hybridized polynucleotide construct at 0.5 mg/kg. The data in FIG. 15 were obtained following administration of multiple doses, designated in FIG. 15 as arrows, of the indicated hybridized polynucleotide construct at 0.25 mg/kg or at 0.125 mg/kg (designated LD in FIG. 15).

TABLE 4

Remaining plasma AT3 protein

| Compound # | Day 7 (mean/SEM) | Day 28 (mean/SEM) |
|---|---|---|
| SB-0206 | 36.7/2.0 | 74.3/1.6 |
| SB-0823 | 60.6/3.2 | |
| SB-0824 | 63.0/2.9 | |
| SB-0825 | 24.6/0.6 | |
| SB-0826 | 26.7/3.8 | |
| SB-0875 | 40.0/5.6 | |
| SB-0876 | 55.6/0.7 | |
| SB-0877 | 56.1/3.4 | |
| SB-0884 | 44.4/7.9 | |
| SB-0886 | 41.2/2.8 | 76.5/4.5 |
| SB-0887 | 37.2/2.3 | 60.1/2.2 |
| SB-0888 | 28.4/2.1 | 54.0/2.9 |
| SB-0889 | 37.0/2.8 | 72.3/4.3 |
| SB-0894 | 38.0/1.9 | 68.8/1.5 |
| SB-0895 | 50.6/6.8 | 79.2/5.4 |
| SB-0896 | 33.0/0.9 | 52.5/1.3 |
| SB-0897 | 58.5/2.7 | 93.1/5.3 |
| SB-0898 | 36.5/4.2 | 73.0/6.3 |
| SB-0899 | 37.3/3.5 | 77.6/4.5 |
| SB-0900 | 62.6/4.7 | 90.5/7.4 |
| SB-0901 | 61.7/1.0 | 97.7/3.3 |
| SB-0902 | 47.1/3.7 | 66.8/0.4 |
| SB-0922 | 95/1.5 | 96.4/1.0 |
| SB-0923 | 97.8/1.0 | 103.5/2.3 |
| SB-0924 | 38.2/2.7 | 77.1/5.3 |
| SB-0925 | 48.6/1.0 | 85.2/0.8 |
| SB-0926 | 46.6/2.0 | 76.1/4.6 |
| SB-0927 | 28.0/1.1 | 62.9/3.2 |
| SB-0928 | 43.0/1.4 | 70.6/2.3 |
| SB-0929 | 41.6/4.0 | 69.0/4.0 |
| SB-0930 | 35.3/2.8 | 67.2/3.0 |
| SB-0931 | 29.1/1.0 | 70.6/4.1 |
| SB-0932 | 29.8/4.3 | 42.0/6.6 |
| SB-0933 | 39.1/1.8 | 58.8/0.9 |
| SB-0934 | 42.2/5.9 | 49.5/1.7 |
| SB-0935 | 53.8/1.4 | 59.4/1.8 |
| SB-0936 | 50.3/2.5 | 71.5/4.3 |
| SB-0937 | 37.0/3.3 | 62.3/2.5 |
| SB-0938 | 32.5/2.9 | 62.3/2.5 |
| SB-0939 | 26.3/1.0 | 64.2/1.8 |
| SB-0940 | 42.5/1.7 | 75.9/0.5 |
| SB-0941 | 36.6/4.9 | 72.8/2.9 |
| SB-0952 | 54.0/5.1 | 71.5/4.2 |
| SB-0953 | 73.4/7.1 | 91.4/5.6 |
| SB-0954 | 48.9/4.0 | 68.1/1.6 |
| SB-0963 | 43.6/4.3 | 77.8/3.2 |
| SB-0964 | 45.1/1.5 | 74.0/3.3 |
| SB-0965 | 53.7/2.4 | 90.1/2.4 |
| SB-0966 | 50.0/2.7 | 87.0/5.4 |
| SB-0967 | 35.5/0.3 | 73.7/4.1 |
| SB-0968 | 79.2/4.1 | 93.7/4.6 |
| SB-0969 | 55.9/1.9 | 84.8/2.3 |
| SB-0970 | 59.9/2.0 | 61.1/2.6 |
| SB-0971 | 66.6/2.8 | 82.3/3.6 |
| SB-0972 | 60.4/10.2 | 74.1/4.9 |
| SB-0973 | 59.3/2.4 | 75.1/5.5 |
| SB-0974 | 50.5/4.0 | 56.9/0.8 |
| SB-0975 | 41.8/1.0 | 56.8/4.6 |
| SB-0976 | 36.1/2.9 | 52.2/4.0 |
| SB-0977 | 31.1/1.0 | 53.3/3.3 |
| SB-0978 | 43.5/1.6 | 69.7/0.3 |
| SB-0979 | 57.5/2.0 | 76.4/6.5 |
| SB-0991 | 48.9/4.7 | 63.3/3.1 |
| SB-0992 | 38.2/4.7 | 50.7/4.1 |
| SB-0993 | 35.9/1.1 | 54.4/3.6 |
| SB-0994 | 33.3/3.7 | 40.1/5.0 |
| SB-0995 | 39.4/4.5 | 71.6/3.1 |
| SB-0996 | 54.6/2.4 | 74.4/4.5 |
| SB-1013 | 28.8/1.5 | 40.7/5.5 |
| SB-1214 | | |
| SB-1215 | 43.9/8 | 71.6/5 |
| SB-1216 | 39.9/3 | 66.2/2 |
| SB-1232 | | |
| SB-1250 | 88.8/2 | 58.3/4 |
| SB-1251 | 85.5/5 | 60.8/5 |
| SB-1252 | 83.7/5 | 62.9/6 |
| SB-1253 | 79.8/1 | 52.6/6 |
| SB-1254 | 78.8/4 | 63.6/7 |
| SB-1255 | 90.1/4 | 43.2/2 |
| SB-1256 | 84.8/6 | 56.4/3 |
| SB-1276 | | |
| SB-1277 | | |
| SB-1278 | | |
| SB-1279 | | |
| SB-1284 | | |
| SB-1285 | | |
| SB-1286 | | |
| SB-1289 | 44.0/8 | 67.1/10 |
| SB-1290 | 44.0/5 | 56.5/5 |
| SB-1291 | 57.3/5 | 62.5/10 |
| SB-1292 | 60.5/5 | 61.3/6 |
| SB-1293 | 44.8/3 | 50.6/4 |
| SB-1294 | 81.1/5 | 52.2/4 |
| SB-1295 | 82.5/4 | 64.5/2 |
| SB-1296 | 46.2/3 | 49.8/3 |
| SB-1297 | 39.6/4 | 55.8/4 |
| SB-1298 | 36.2/3 | 56.5/5 |
| SB-1299 | 38.4/3 | 59.3/5 |
| SB-1300 | 44.2/5 | 63.3/5 |
| SB-1301 | 51.6/6 | 71.5/3 |
| SB-1302 | 52.5/6 | 67.1/2 |
| SB-1303 | 57.5/5 | 84.0/4 |
| SB-1304 | 57.5/3 | 79.5/4 |
| SB-1305 | 67.5/3 | 88.6/4 |
| SB-1309 | 78.2/6 | 87.5/5 |
| SB-1310 | 30.9/4 | 47.0/6 |
| SB-1311 | 37.2/10 | 58.8/12 |
| SB-1312 | 66.6/8 | 89.2/4 |
| SB-1313 | 36.9/3 | 47.0/3 |
| SB-1314 | 82.3/4 | 99.9/6 |
| SB-1315 | 82.4/2 | 90.7/10 |
| SB-1316 | 43.4/6 | 63.4/4 |
| SB-1317 | | |
| SB-1318 | | |
| SB-1376 | | |
| SB-1377 | | |
| SB-1378 | | |
| SB-1379 | | |
| SB-1380 | | |
| SB-1381 | | |
| SB-1382 | | |
| SB-1383 | | |
| SB-1409 | | |
| SB-1445 | 57.0/3 | 78.7/8 |

### Other Embodiments

Various modifications and variations of the described invention and methods of use of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A hybridized polynucleotide construct comprising a passenger strand, a guide strand loadable into a RISC complex, and one or more auxiliary moieties;
wherein at least one of said auxiliary moieties is non-bioreversibly linked to an internucleoside phosphate or phosphorothioate in said passenger strand; wherein said auxiliary moieties are independently selected from the group consisting of a targeting moiety, a cell penetrating peptide, an endosomal escape moiety, and a neutral organic polymer.

2. The hybridized polynucleotide construct of claim 1, comprising 5 or fewer auxiliary moieties, at least one of said auxiliary moieties being linked non-bioreversibly to an internucleoside phosphate or phosphorothioate in said passenger strand, and the remaining of said auxiliary moieties being independently linked bioreversibly to a phosphate or phosphorothioate in said guide strand or linked bioreversibly or non-bioreversibly to a phosphate or phosphorothioate in said passenger strand.

3. The hybridized polynucleotide construct of claim 1, wherein said passenger strand comprises a pattern —N-p^{L}-(—N-p-)_{z}-N-p^{L}-(—N-p-)_{z}-N-p^{L}-[(—N-p-)_{z}-N-p^{L}-]_{z1}-,
wherein
each N is independently a nucleoside;
each p^{L} is a phosphate or phosphorothioate linked non-bioreversibly to an auxiliary moiety;
each p is independently a phosphate, phosphorothioate, phosphoramidate, or phosphonate;
each z is independently 0, 1, or 2; and
z1 is 0, 1, or 2.

4. The hybridized polynucleotide construct of claim 1, wherein said auxiliary moieties are the same.

5. The hybridized polynucleotide construct of claim 1, wherein said auxiliary moieties are linked to proximal phosphates or phosphorothioates.

6. The hybridized polynucleotide construct of claim 1, wherein each of said auxiliary moieties is a targeting moiety comprising a single ligand.

7. The hybridized polynucleotide construct of claim 6, wherein said ligand is linked to said internucleoside phosphate or phosphorothioate through a linear oligomeric linker.

8. The hybridized polynucleotide construct of claim 7, wherein said linear oligomeric linker comprises poly(ethylene glycol).

9. The hybridized polynucleotide of claim 1, wherein said hybridized polynucleotide comprises at least two auxiliary moieties linked to phosphates or phosphorothioates in said guide strand or said passenger strand.

10. (canceled)

11. (canceled)

12. (canceled)

13. (canceled)

14. (canceled)

15. (canceled)

16. (canceled)

17. The hybridized polynucleotide construct of claim 1, wherein
said guide strand comprises at least one internucleoside phosphorothioate linking two of the four 3′-terminal nucleosides in said guide strand;
said guide strand comprises at least one internucleoside phosphorothioate linking two of the four 5′-terminal nucleosides in said guide strand; and/or
said passenger strand comprises at least one internucleoside phosphorothioate linking two of the four 5′-terminal nucleosides in said passenger strand.

18. (canceled)

19. (canceled)

20. The hybridized polynucleotide construct of claim 1, wherein said auxiliary moiety is non-bioreversibly linked through a non-bioreversible linker comprising a 1,2,3-triazole, a N-sulfonylamidocarbonyl, or an amide.

21. The hybridized polynucleotide construct of claim 20, wherein said auxiliary moiety combines with said non-bioreversible linker to form a group that is 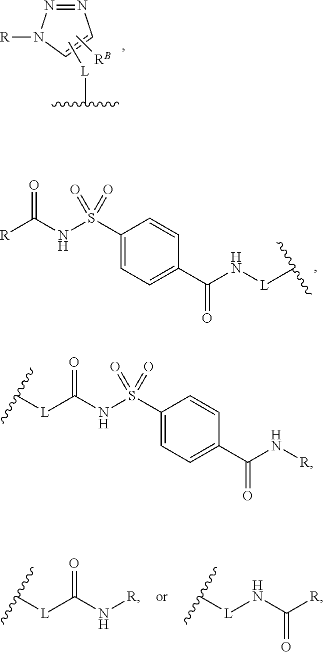
wherein
R is said auxiliary moiety;
R^{B} is H or C₁₋₆ alkyl; and
L is C₂₋₆ alkylene or —(CH₂CH₂O)ₚ₁(CH₂CH₂)—, wherein p1 is an integer from 1 to 50.

22. The hybridized polynucleotide construct of claim 1, wherein at least one of said auxiliary moieties is a targeting moiety.

23. The hybridized polynucleotide construct of claim 22, wherein said targeting moiety comprises a ligand that is N-acetyl galactosamine, mannose, folate, prostate specific membrane antigen (PSMA), or an antibody or an antigen-binding fragment thereof.

24. The hybridized polynucleotide construct of claim 23, wherein said targeting moiety comprises a ligand that is N-acetyl galactosamine linked to said phosphate or phosphorothioate through a linker bonded to the anomeric carbon of N-acetyl galactosamine, wherein said anomeric carbon is part of a hemiaminal group.

25. (canceled)

26. (canceled)

27. (canceled)

28. The hybridized polynucleotide construct of claim 1, wherein said guide strand or said passenger strand further comprises one or more internucleoside phosphotriesters, internucleoside phosphonates, or internucleoside phosphoramidates.

29. The hybridized polynucleotide construct of claim 28, wherein said guide strand or said passenger strand comprises one or more of said internucleoside phosphotriesters, wherein at least one of said internucleoside phosphotriesters is a non-bioreversible phosphotriester.

30. The hybridized polynucleotide construct of claim 29, wherein each of said non-bioreversible phosphotriesters is a phosphate or a phosphorothioate substituted with a substituent selected independently from the group consisting of optionally substituted C₂₋₁₆ alkyl; optionally substituted C₃₋₁₆ alkenyl; optionally substituted C₃₋₁₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O, and S; optionally substituted C₂₋₉ heterocyclyl having 1 to 4 heteroatoms selected from N, O, and S, wherein said heterocyclyl does not comprise an S—S bond; optionally substituted (C₂₋₉ heterocyclyl)-C₁₋₄-alkyl having 1 to 4 heteroatoms selected from N, O, and S, wherein said heterocyclyl does not comprise an S—S bond; and a group of the following structure: 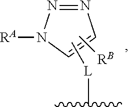
wherein
L is optionally substituted C₂₋₁₆ alkylene;
R^{A} is optionally substituted C₂₋₆ alkyl; optionally substituted C₆₋₁₄ aryl; optionally substituted (C₆₋₁₄ aryl)-C₁₋₄-alkyl; optionally substituted C₃₋₈ cycloalkyl; optionally substituted (C₃₋₈ cycloalkyl)-C₁₋₄-alkyl; optionally substituted C₁₋₉ heteroaryl having 1 to 4 heteroatoms selected from the group consisting of N, O, and S; optionally substituted (C₁₋₉ heteroaryl)-C₁₋₄-alkyl having 1 to 4 heteroatoms selected from the group consisting of N, O, and S; optionally substituted C₂₋₉ heterocyclyl having 1 to 4 heteroatoms selected from the group consisting of N, O, and S, wherein said heterocyclyl does not comprise an S—S bond; optionally substituted (C₂₋₉ heterocyclyl)-C₁₋₄-alkyl having 1 to 4 heteroatoms selected from N, O, and S, wherein said heterocyclyl does not comprise an S—S bond; and a poly(ethylene glycol) terminated with —OH, C₁₋₆ alkoxy, or —COOH; and
R^{B} is H or optionally substituted C₁₋₆ alkyl.

31. The hybridized polynucleotide construct of claim 29, wherein each of said non-bioreversible phosphotriesters is a phosphate or a phosphorothioate substituted with a substituent that is 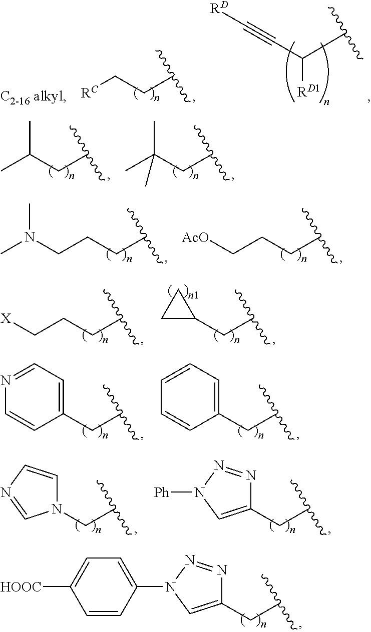
or a group formed by cycloaddition reaction of 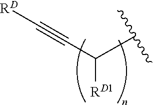
with an azido-containing substrate,
wherein
n is an integer from 1 to 6;
n1 is an integer from 1 to 6;
R^{C} is optionally substituted C₆ aryl; optionally substituted C₄₋₅ heteroaryl that is a six member ring comprising 1 or 2 nitrogen atoms; or optionally substituted C₄₋₅ heterocyclyl that is a six member ring comprising 1 or 2 nitrogen atoms;
R^{D} is H or 01-6 alkyl;
each R^{D1} is independently H or C₁₋₆ alkyl, provided that 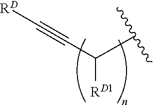
contains 24 carbon atoms or fewer;
X is a halogen, —COOR¹, or —CONR²₂, wherein each of R¹ and R² is independently H, optionally substituted C₁₋₆ alkyl, optionally substituted C₆₋₁₄ aryl, optionally substituted C₁₋₉ heteroaryl, or optionally substituted C₂₋₉ heterocyclyl; and
said azido-containing substrate is

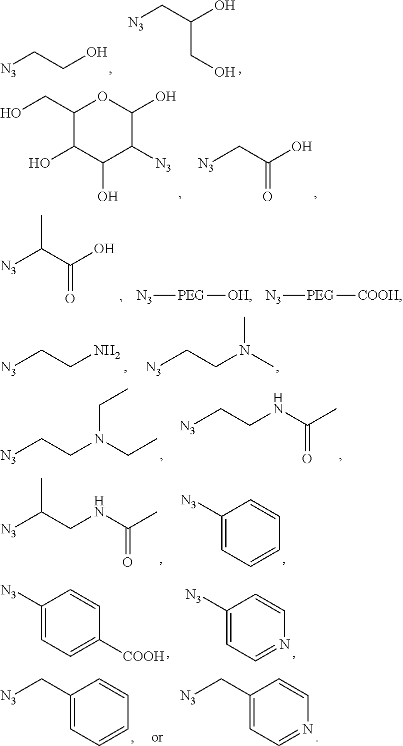

32. The hybridized polynucleotide construct of claim 28, wherein said guide strand comprises from 1 to 5 of said non-bioreversible phosphotriesters.

33. The hybridized polynucleotide construct of claim 28, wherein
one of said non-bioreversible phosphotriesters connects the second nucleoside and the third nucleoside of said guide strand;
one of said non-bioreversible phosphotriesters connects the fifth nucleoside and the sixth nucleoside of said guide strand;
one of said non-bioreversible phosphotriesters connects the seventeenth nucleoside and the eighteenth nucleoside of said guide strand;
one of said non-bioreversible phosphotriesters connects the nineteenth nucleoside and the twentieth nucleoside of said guide strand; and/or
one of said non-bioreversible phosphotriesters connects the twentieth nucleoside and the twenty first nucleoside of said guide strand.

34. (canceled)

35. (canceled)

36. (canceled)

37. (canceled)

38. The hybridized polynucleotide construct of claim 28, wherein said passenger strand comprises from 1 to 5 of said non-bioreversible phosphotriesters.

39. The hybridized polynucleotide construct of claim 28, wherein said guide strand or said passenger strand comprises one or more of said internucleoside phosphotriesters, at least one of said internucleoside phosphotriesters being a bioreversible phosphotriester.

40. The hybridized polynucleotide construct of claim 39, wherein said bioreversible phosphotriester is a phosphate or a phosphorothioate substituted with -(Link A)-S—S—R^{E},
wherein
Link A is a divalent or trivalent linker comprising an sp³-hybridized carbon atom bonded to said phosphate or phosphorothioate and a carbon atom bonded to —S—S—, wherein, when Link A is a trivalent linker, the third valency of Link A combines with —S—S— and R^{E} to form optionally substituted C₃₋₉ heterocyclylene, and
R^{E} is optionally substituted C₂₋₈ alkyl; optionally substituted C₃₋₈ alkenyl; optionally substituted C₃₋₈ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O, and S; optionally substituted C₂₋₉ heterocyclyl having 1 to 4 heteroatoms selected from N, O, and S; optionally substituted (C₂₋₉ heterocyclyl)-C₁₋₄-alkyl having 1 to 4 heteroatoms selected from N, O, and S; or, when Link A is a trivalent linker, R^{E} combines with —S—S— and Link A to form optionally substituted C₃₋₉ heterocyclylene.

41. The hybridized polynucleotide construct of claim 40, wherein said bioreversible phosphotriester is a phosphate or a phosphorothioate substituted with a group that is 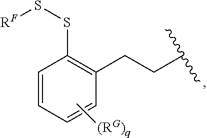
wherein
R^{F} is optionally substituted C₁₋₆ alkyl or optionally substituted C₆₋₁₄ aryl,
R^{G} is a halogen or optionally substituted C₁₋₆ alkyl, and
q is an integer from 0 to 4.

42. The hybridized polynucleotide construct of claim 39, wherein q is 0 and R^{F} is optionally substituted C₁₋₆ alkyl.

43. The hybridized polynucleotide construct of claim 28, wherein said guide strand or said passenger strand comprises one or more phosphonates.

44. The hybridized polynucleotide construct of claim 1, wherein said guide strand comprises 19 or more nucleosides.

45. The hybridized polynucleotide construct of claim 1, wherein said guide strand comprises fewer than 100 nucleosides.

46. (canceled)

47. (canceled)

48. The hybridized polynucleotide construct of claim 1, wherein said passenger strand comprises 19 or more nucleosides.

49. The hybridized polynucleotide construct of claim 1, wherein said passenger strand comprises fewer than 100 nucleosides.

50. (canceled)

51. (canceled)

52. A method of delivering a polynucleotide construct to a cell comprising contacting said cell with the hybridized polynucleotide construct of claim 1, wherein, after said contacting, said polynucleotide construct resides inside said cell.

53. A method of reducing the expression of a protein in a cell comprising contacting said cell with the hybridized polynucleotide construct of claim 1, wherein, after said contacting, expression of said protein in said cell is reduced.
